(19) United States
(12) Patent Application Publication
(10) Pub. No.: US 2022/0110910 A1
(43) Pub. Date: Apr. 14, 2022

(54) CANNABINOID-CONTAINING COMPLEX MIXTURES FOR THE TREATMENT OF CYTOKINE RELEASE SYNDROME WHILE PRESERVING KEY ANTI-VIRAL IMMUNE REACTIONS

(71) Applicant: GBS Global BioPharma, Inc., Ottawa (CA)

(72) Inventor: Andrea Lee Small-Howard, Norwalk, CA (US)

(21) Appl. No.: 17/406,035

(22) Filed: Aug. 18, 2021

Related U.S. Application Data
(60) Provisional application No. 63/067,271, filed on Aug. 18, 2020.

(51) Int. Cl. A61K 31/352; A61K 31/05; A61P 31/12; A61K 31/015

(52) U.S. Cl. CPC A61K 31/352; A61K 31/015; A61P 31/12; A61K 31/05

(57) ABSTRACT

Provided herein are cannabinoid-containing complex mixtures suitable for use as active pharmaceutical ingredients. The complex mixtures comprise one or more of a pDC modulating cannabinoid or terpene, a monocyte modulating cannabinoid or terpene, a T-cell modulating cannabinoid or terpene, and optionally, a lymphopenia-reducing lymphopenia cannabinoid. Also provided are methods of making the complex mixtures; pharmaceutical compositions comprising the complex mixture, and methods of using the pharmaceutical compositions for the treatment of a patient who has, or is at risk of developing, CRS, CSS, MAS, hyperinflammation, chronic inflammation, or a proinflammatory immune response. Also provided are methods of making the complex mixtures; pharmaceutical compositions comprising the complex mixture, and methods of using the pharmaceutical compositions for the treatment of ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

### 1. CROSS-REFERENCE TO RELATED APPLICATIONS

This application claims the benefit of U.S. Provisional Application No. 63/067,271, filed Aug. 18, 2020, which is hereby incorporated in its entirety by reference.

### 2. BACKGROUND

The human immune system is vital for identifying and dest46727roying viral pathogens; however, sometimes a viral infection evades destruction by normal immune processes but triggers a hyperinflammatory response. In this scenario, the inflammatory reactions that normally target and eradicate viral pathogens endanger the patient because of their severity and location in a major organ of the body like the heart, lungs, or brain target tissue.

In severe cases of COVID-19, the leading cause of death is respiratory failure from acute respiratory distress syndrome (ARDS) triggered by virally driven hyperinflammatory processes in the lungs. Some extreme cases of COVID-19 are also characterized as secondary haemophagocytic lymphohistiocytosis (sHLH), a hyperinflammatory condition caused by high levels of inflammatory cytokines that lead to multiorgan failure.

Cytokine panels taken from COVID-19 patients who had severe hyperinflammatory reactions confirmed that they had high levels of pro-inflammatory substances in their blood; including TNFα, IFNγ, IL-1β, IL-6, IL-12, IL-18, IL-33, TFNα, TGFβ, CCL2, CCL3, CCL5, CXCL8, CXCL9, CXCL10. This condition is also known as Cytokine Storm Syndrome (CSS) or Macrophage Activation Syndrome (MAS). However, treatment of virally-induced hyperinflammation in COVID-19 patients may not be as simple as using a broadly immunosuppressive strategy because the production of specific proinflammatory cytokines may be crucial for immune defense against SARS-CoV-2 infections or other viral infections.

Cytokine release syndrome (CRS) is a systemic inflammatory response that can be triggered by a variety of factors. For example, CRS represents one of the most frequent serious adverse effects of administration of immunotherapeutic agents, such as checkpoint inhibitors, bispecific T-cell engagers, and CAR-T cells. CRS can also be caused by an excessive activation of the immune system during an infection by a virus, such as an influenza virus or a coronavirus. CRS occurs when T lymphocytes and other cells of the adaptive immune system are activated and release inflammatory cytokines. IL-6, TNFα, and interferon (IFN)-γ are among the pro-inflammatory cytokines that are consistently found to be elevated in serum of patients with CRS. CRS can present with a variety of symptoms ranging from mild flu-like symptoms to severe life-threatening manifestations of the uncontrolled inflammatory response.

Although some cannabinoids have been tested individually, the individually tested cannabinoids have not proven comparable in efficacy to the use of the whole Cannabis plant or to more complex, plant-derived therapeutic mixtures. This may be due to the complexity of compounds present in each Cannabis plant, coupled with the huge variability existing in various Cannabis strains. It is likely that minor components of Cannabis plants (cannabinoids, terpenes, and other naturally occurring components) contribute significantly both positively or negatively to the overall therapeutic effects.

There is a need for novel and validated pharmacological agents for treating patients who have, or who are at risk for developing CRS without immune compromise of infected patients. There is a particular need for well-defined compositions of cannabinoids and/or terpene mixtures that are effective for immune modulation, for example, for the treatment of CRS and/or Macrophage Activation Syndrome (MAS) and other dangerous pro-inflammatory conditions.

### 3. SUMMARY

Provided herein are cannabinoid-containing complex mixtures suitable for use as active pharmaceutical ingredients. Plasmacytoid dendritic cells (pDC), monocytes, and T cells are critical cell types involved in the human immune response to viral infections; therefore, these cells were selected as therapeutic targets, in particular, from within human primary peripheral blood mononuclear cell (PBMC) mixtures. In addition, the pDC, monocytes, and T-cells all exhibit sensitivity to modulation by cannabinoids, so they were likely therapeutic targets based on their importance in fighting viral infections, combined with their sensitivity to cannabinoids and terpenes through multiple cannabinoid- and terpene-sensitive receptors. The cannabinoid-containing complex mixtures immunomodulate critical cytokines and immunological processes of these key immune cells.

The pDC produce massive amounts of IFNα in response to normal viral infections, but SARS-CoV-2, and related SARS-CoV-1, evades this anti-viral triggering event. The pDC also produce TNFα. Activated monocytes produce IL-1β, IL-6, and TNFα as a part of antiviral immunity. CD4+ T cells and CD8+ T cells produce TNFα, IL-2, IFNγ, and T cells are normally activated and proliferate during antiviral immunity.

Although the levels of the following cytokines are reportedly elevated in severe COVID-19 cases: TNFα, IFNγ, IL-1β, IL-6, IL-12, IL-18, IL-33, TFNα, TGFβ,β, CCL2, CCL3, CCL5, CXCL8, CXCL9, CXCL10, only a subset of these cytokines correlate with symptom severity. Specifically, IL-1 β, IL-6, IL-7, IL-1Ra, IL-10, IP-10, and TNF-α can be used to distinguish between mild, moderate, and severe COVID-19 cases. The rationally designed combination therapies in this application use cannabinoid containing complex mixtures designed to decrease levels of TNFα, IFNγ, IL-6, and IL-1β, while preserving (or augmenting) the anti-viral immune responses driven by IFNα produced by the pDC. In some instances, clinical lymphopenia is addressed with compounds that stimulate T cell proliferation.

As a result of viral-based tissue injury, the production of proinflammatory cytokines and the recruitment of proinflammatory macrophages leads to Cytokine Storm Syndrome (CSS) or Macrophage Activation Syndrome (MAS). These immune processes lead to clinical syndromes such as Cytokine Release Syndrome (CRS), Acute Respiratory Distress (ARD), or even the hyper-inflammatory condition known as secondary hemophagocytic lymphohistiocytosis (sHLH). The correlation of TNFα, IFNγ, IL-6, and IL-1β with disease severity provides strong evidence for their use as therapeutic targets in the relief of CRS, ARD, or sHLH in COVID-19 patients. For example, IL-1β is a pro-inflammatory cytokine and elevated IL-1β is central to ARDS and sHLH. IL-6 produced by activated monocytes compromises the ability of macrophages and dendritic cells to clear the viral pathogens and severely compromises the adaptive immune response. TNFα is elevated in the serum of COVID-19 patients, which may add to apoptosis and tissue damage in areas of virally-induced hyperinflammation, such as the lungs, heart, or kidneys. Clinically, elevated levels of both TNFα and IFNγ have correlated with significant tissue injury in the lungs and heart and are predictive of adverse outcomes in COVID-19 patients. IFNγ is known to cause tissue damage, and IFNγ plays a central role in activating macrophages through Jak/STAT signaling pathways, which contributes to the pathology of MAS. These activated macrophages are hypothesized to play a role in causing the cellular and tissue damage in the lungs, hearts, and kidneys of severe COVID-19 patients, which contributes to the pathology of MAS.

COVID-19 is also characterized by clinical lymphopenia, which involves a reduction in the numbers of T cells, B cells, and natural killer cells. The total number of neutrophils and leukocyte counts increase in the serum of COVID-19 patients; however, the total counts of CD4+ T cells, CD+8 T cells, regulatory T cells, memory T cells, natural killer cells, and B cells decrease significantly. Reduced numbers of cytotoxic CD8+ T-cells relative to helper CD4+ T-cells correlated with COVID-19 symptom severity.

In some cases, the therapeutic cannabinoid containing complex mixtures described herein are comprised of: 1) a tumor necrosis factor alpha (TNFα)-decreasing cannabinoid, 2) an interferon-gamma (IFNγ)-decreasing cannabinoid or terpene; and in some cases, 3) an interleukin-1 beta (IL-1β) and/or an interleukin-6 (IL-6)-decreasing cannabinoid; and 4) an optional lymphopenia-reducing cannabinoid.

In some cases, the therapeutic cannabinoid containing complex mixtures described herein are comprised of one or more of the following: a pDC-modulating cannabinoid or terpene, a monocyte-modulating cannabinoid or terpene, and a T-cell modulating cannabinoid or terpene. The present inventors found, through experimentation of 24 complex mixtures described in Tables 3A-3B and FIG. 24A-24E, that CCCMs can be categorized into 5 different categories related to immunomodulatory effects. The CCCMs of Example 3 that were tested were further categorized into 5 different groups, based on the resulting immunomodulatory effects for each CCCM shown in FIGS. 24A-24E, and provided below:

Category 1: Cytokine Release Syndrome Therapeutics Designed For Hyperinflammatory Responses. This category targets monocytes & T cells while preserving plasmacytoid dendritic cell responses to virus/bacteria (that may or may not lead to viral-CPG stimulated TNFα in monocytes at 6 h). The goal was to target the mid to later stages of the inflammatory and hyper-inflammatory processes to preserve key anti-viral immune reactions. This category contains therapeutic mixtures that suppress the mid to later phase immune responses (24 hr monocyte and 96 hr T cell functions) but preserve the anti-viral functions of the plasmacytoid dendritic cells (pDC) and the 6-hr TNFα response in monocytes. The therapeutic mixtures in this category were designed to address clinical syndromes such as Cytokine Release Syndrome (CRS), Cytokine Storm Syndrome (CSS), Macrophage Activation Syndrome (MAS), Acute Respiratory Distress (ARD), and the hyper-inflammatory condition known as secondary hemophagocytic lymphohistiocytosis (sHLH) because their pathologies are all related to unregulated overproduction of proinflammatory cytokines. Also, the mixtures in this category are potentially useful as therapeutic agents for treating the adverse side effects of checkpoint inhibitor therapies (cancer) and CAR-T-cell therapies (anti-cancer).

Category 2: Cytokine Release Syndrome Therapeutics Designed For T Cell-Related Hyperinflammation: Targets T cells while preserving plasmacytoid dendritic cell and monocyte responses to virus/bacteria. The mixtures in Category 2 are potentially useful as therapeutic agents for treating rheumatoid arthritis and multiple sclerosis, which are autoimmune disorders where hyperactivated T cells attack self-antigens in the joints (arthritis) or the myelin sheath of the neurons (MS). Also, these mixtures are potentially useful for treating the adverse side effects of CAR-T-cell therapies (anti-cancer).

Category 3: Broadly Immunomodulating. This category targets plasmacytoid dendritic cell, monocyte, and T cell functions. Within this category, two of the mixtures suppress immune responses in all three of the measured cell types. These CCCMs would be good candidates for the treatment of chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

The three mixtures with mostly anti-inflammatory effects (pDC and T cells), but an elevated inflammatory marker for the monocytes, may be useful to address conditions requiring suppression of specific cytokines, but where activated monocytes are needed to fight an active infection. These may include some forms of Cytokine Release Syndrome (CRS), Cytokine Storm Syndrome (CSS), Macrophage Activation Syndrome (MAS), Acute Respiratory Distress (ARD), and the hyper-inflammatory condition known as secondary hemophagocytic lymphohistiocytosis (sHLH) secondary to viral or bacterial infections such as severe forms of COVID-19.

Category 4: Immunosuppressive. This category targets plasmacytoid dendritic cell responses and/or T cells. Because these mixtures consistently downregulate early and later phases of pro-inflammatory responses, these therapeutic mixtures would be ideal for the treatment of chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

Category 5: Pro-Inflammatory. This category targets monocytes and produces TNFa early (6hours) but not later. The mixture in this category could be used to help fight localized infections. TNFα signals early in a response to an infection provide an important signal used to recruit white blood cells to the site of a localized infection.

Also provided are methods of making the cannabinoid containing complex mixtures; pharmaceutical compositions comprising the cannabinoid containing complex mixtures, and methods of using the pharmaceutical compositions for the treatment of a patient who has, or is at risk of developing, CRS,MAS, Cytokine Storm Syndrome (CSS), Respiratory Distress (ARD), the hyper-inflammatory condition known as secondary hemophagocytic lymphohistiocytosis (sHLH), adverse side effects of checkpoint inhibitor therapies (cancer) and CAR-T-cell therapies (anti-cancer), rheumatoid arthritis and multiple sclerosis, chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND), or localized infections.

Without wishing to be bound by a theory, it is believed that the cannabinoid containing complex mixtures provided herein are effective in treating CRS, CSS, MAS, and a variety of inflammatory conditions by specifically targeting and modulating pro-inflammatory cytokines and processes from multiple immune cells associated with the disease in a synergistic and collaborative manner. Specifically, these cannabinoid containing complex mixtures downregulate the production of TNFα, IFNγ, and IL-6, which have been shown to correlate with severe hyperinflammation/CRS/MAS, and these cannabinoid containing complex mixtures address lymphopenia by specifically increasing proliferation of cytotoxic CD8+ T-cells. In addition, these cannabinoid containing complex mixtures have been screened to remove potential agents that would reduce normal anti-viral immune functions that are necessary for fighting the disease. In particular, these cannabinoid containing complex mixtures were screened for their ability to preserve IFNα production by pDC. Preliminary clinical evidence suggests that treatment of COVID-19 with clinically-approved recombinant interferons may help alleviate symptoms and slightly reduce viral loads in SARS-CoV-1, SARS-CoV-2, and MERS infections. The cannabinoid containing complex mixtures within this set are also being developed as an adjunctive therapy to be delivered with clinically-approved IFN (alpha and beta) products for the most beneficial patient results—reduced CRS/MAS while preserving anti-viral immune activity.

Under normal conditions, the human immune system responds to viral infections with immunological responses from both the innate, cell-based systems (initially) and then (within 5-7 days) the adaptive, antibody-based immune systems. First, the plasmacytoid dendritic cells (pDC), which are the first line of defense against viruses in the innate immune system, recognize viral RNA or DNA by toll-like receptor-7 and toll-like-receptor-9 and secrete massive amounts of IFNα that activates an anti-viral immune responses in other leukocytes, such as CD14+ monocytes and cytotoxic CD8+ T-cells. The secretion of IFNα by pDCs is 1000-fold greater than from any other immune cell type, making the pDC's an integral part of human immune defenses against invading viruses. In response to IFNα, a small % of classical CD14⁺ monocytes become activated to CD14+ CD16+ monocytes. Activated CD14⁺ CD16⁺ monocytes fight viral infections through both phagocytosis and the secretion of the pro-inflammatory cytokines, TNFα, IL-1β and IL-6. Activated CD14⁺ CD16⁺ monocytes also bridge the innate and adaptive immune system through antigen presentation to T cells. Activated CD14⁺ CD16⁺ monocytes up-regulate co-stimulatory molecules including CD80 and CD86 upon pathogen exposure, which allows for appropriate activation of T cells. These activated CD14⁺ CD16⁺ monocytes may become infected and present viral antigen through CD80/CD86, which activates cytotoxic CD8⁺ T-cells. The activated CD14⁺ CD16⁺ monocytes may also mature to become macrophages, which may aid in the destruction of virally infected cells and viral particles through phagocytosis. Cytotoxic CD8⁺ T-cells identify infected cells by viral antigens presented on MHC type 1 and then destroy both the infected cell and the viral particles through phagocytosis. Each cytotoxic T cell has a TCR that can specifically recognize a viral antigen bound to an MHC molecule. The T cells releases cytotoxic factors to kill the infected cell and, therefore, prevent survival of the invading virus. Cytotoxic CD8+ T-cells also produce both IFNγ and TNFα to amplify a cytotoxic immune response. T-cell activation can signal the adaptive immune system to produce corresponding antibodies to neutralize the virus. Virus particles that are shed can be agglutinated by direct attachment of antibodies to the virus surface, or the antibodies can assist in antibody-mediated phagocytosis. The activation and proliferation of T-cells amplifies inflammatory signals. Upon activation, T cell responses polarize based on specific stimuli they encounter. In particular, stimulation with viral antigens leads to a cell-mediated (TH1) response while antigens from parasites favor a humoral, antibody-based (TH2) response. Two of the key cytokines that differentiate T-Helper 1 (TH1) vs T-Helper 2 (TH2) response polarization are IFNγ (TH1) and IL-4 (TH2). IFNγ produced by TH1 cells activates macrophages to clear viruses. IL-4 produced by TH2 cells stimulates B-cells to make IgE antibodies. In addition to cytokine secretion, T cells must proliferate to form effector and long-lived memory cells, a key process in the establishment of immunological memory. It is believed that alterations in T cell proliferation and/or cytokine secretion can significantly affect immune competence resulting in either immune suppression or immune enhancement contributing to immune hypersensitivity or autoimmunity.

Aspects of the present disclosure include an active pharmaceutical ingredient, comprising: (a) an interleukin-1β (IL-1β-decreasing and/or interleukin-6 (IL-6)-decreasing cannabinoid; (b) a tumor necrosis factor alpha (TNFα)-decreasing cannabinoid; (c) an interferon-gamma (IFNγ)-decreasing cannabinoid or terpene; and (d) optionally, a lymphopenia-reducing cannabinoid. The IL-6-decreasing cannabinoid may be a monocyte-modulating cannabinoid. The TNFα-decreasing cannabinoid may be one or more of: a pDC-modulating cannabinoid, a monocyte-modulating cannabinoid, and a T-cell modulating cannabinoid. The IFNγ-decreasing cannabinoid may be a T-cell modulating cannabinoid.

In some embodiments, the IL-1β-decreasing and/or the IL-6-decreasing cannabinoid is Cannabidiol (CBD), Cannabidivarin (CBDV), or a combination thereof.

In some embodiments, the TNFα-decreasing cannabinoid is Cannabinol (CBN).

In some embodiments, the active pharmaceutical ingredient further comprises a class 1 or class 2 interferon.

In some embodiments, the interferon (IFN) is selected from the group consisting of: IFNα 2a, IFNα 2b, IFNα n1, IFNα n3, IFNα con1, IFN-β 1a, IFNβ 1b, and IFNβ P-2a. In some embodiments, the interferon is selected from the group consisting of: Roferon, Intron A, Wellferon, Alferon, Infergen, Rebif, Betaferon, Avonex, Betaseron, Pegasys, Peglntron, and Sylatron.

In some embodiments, each of the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid is a synthetic or biosynthetic.

In some embodiments, the optional Lymphopenia-reducing cannabinoid is tetrahydrocannabinol (THC).

In some embodiments, the IFNγ-decreasing cannabinoid is Cannabigerol (CBG) or Cannabinol (CBN).

In some embodiments, the IFNγ-decreasing terpene is Phytol A.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at least 75% by weight of the active ingredient.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at least 80% by weight of the active ingredient.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at least 85% by weight of the active ingredient.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at least 90% by weight of the active ingredient.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at least 95% by weight of the active ingredient.

In some embodiments, the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid collectively constitute at 100% by weight of the active ingredient.

In some embodiments, all compounds in the active ingredient other than the IL-1β-decreasing and/or the IL-6-decreasing cannabinoid, the TNFα-decreasing cannabinoid, the optional IFNγ-decreasing cannabinoid or terpene, and the optional Lymphopenia-reducing cannabinoid are extractable from Cannabis sativa.

In some embodiments, the active pharmaceutical ingredient is substantially free of tetrahydrocannabinol (THC).

In some embodiments, the active pharmaceutical ingredient is substantially free of cannabichromene (CBC).

In some embodiments, the active pharmaceutical ingredient is substantially free of Cannabigerol (CBG).

In some embodiments, the active pharmaceutical ingredient is substantially free of Phytol A.

In some embodiments, the active pharmaceutical ingredient is substantially free of alpha-pinene.

In some embodiments, the active pharmaceutical ingredient is substantially free of trans-nerolidol.

In some embodiments, the IL-6-decreasing cannabinoid constitutes 0.01-99% by weight of the active ingredient; the TNFα-decreasing cannabinoid constitutes 0.01-99% by weight of the active ingredient; and the Lymphopenia-reducing cannabinoid constitutes 0.01-99% by weight of the active ingredient.

In some embodiments, the IL-1β-decreasing and/or the IL-6-decreasing cannabinoid constitutes 0.01-99% by weight of the active ingredient; the TNFα-decreasing cannabinoid constitutes 0.01-99% by weight of the active ingredient; the IFNγ-decreasing cannabinoid or terpene constitutes 0.01-99% by weight of the active ingredient, the Lymphopenia-reducing cannabinoid constitutes 0.01-99% by weight of the active ingredient.

In some embodiments, the active pharmaceutical ingredient comprises: CBD, and CBN; CBDV, and CBN; CBD, CBDV, and CBN; CBD, CBN, and CBG; CBDV, CBN, and CBG; CBD, CBDV, CBN, and CBG; CBD, CBN, and Phytol A; CBDV, CBN, and Phytol A; CBD, CBDV, CBN, and Phytol A; CBD, and CBN; CBDV, and CBN; CBD, CBDV, and CBN; CBD, CBN, CBG, and THC; CBDV, CBN, CBG, and THC; CBD, CBDV, CBN, CBG, and THC; CBD, CBN, Phytol A, and THC; CBDV, CBN, Phytol A, and THC; CBD, CBDV, CBN, Phytol A, and THC; CBD, CBN, and THC; CBDV, CBN, and THC; or CBD, CBDV, CBN, and THC.

In some embodiments, the active pharmaceutical ingredient comprises a molar ratio of: 1-part CBD:2-parts CBN; 1-part CBD:1-part CBN; 1-part CBDV: 10-parts CBN; 5-parts CBD: 1-part CBDV:10-part CBN; 10-parts CBD:1-part CBDV:10-parts CBN; 1-part CBD:1-part CBN; 2-parts CBD:1-part CBN; 1-part CBDV:5-parts CBN; 5-parts CBD:1-part CBDV:5-parts CBN; 10-parts CBD:1-part CBDV:5-parts CBN; 50-parts CBD:100-parts CBN:1-part CBG; 100-parts CBD:100-parts CBN:1-part CBG; 10-parts CBDV:100-parts CBN:1-part CBG; 50-parts CBD:10-parts CBDV:100-parts CBN:1-part CBG; 100-parts CBD:10-parts CBDV:100-parts CBN:1-part CBG; 50-parts CBD:50-parts CBN:1-part CBG; 100-parts CBD:50-parts CBN:1-part CBG; 10-parts CBDV:50-parts CBN:1-part CBG; 50-parts CBD:10-parts CBDV:50-parts CBN:1-part CBG; 100-parts CBD:10-parts CBDV:50-parts CBN:1-part CBG; 5,000-parts CBD:10,000-parts CBN:1-part Phytol A; 10,000-parts CBD:10,000-parts CBN:1-part Phytol A; 1,000-parts CBDV:10,000-parts CBN:1-part Phytol A; 5,000-parts CBD:1,000-parts CBDV:10,000-parts CBN:1-part Phytol A; 10,000-parts CBD:1,000-parts CBDV:10,000-parts CBN:1-part Phytol A; 5,000-parts CBD:5,000-parts CBN:1-part Phytol A; 10,000-parts CBD:5,000-parts CBN:1-part Phytol A; 1,000-parts CBDV:5,000-parts CBN:1-part Phytol A; 5,000-parts CBD:1,000-parts CBDV:5,000-parts CBN:1-part Phytol A; 10,000-parts CBD:1,000-parts CBDV:5,000-parts CBN:1-part Phytol A; 500-parts CBD:1-part CBN; 1,000-parts CBD:1-part CBN; 100-parts CBDV:1-part CBN; 500-parts CBD:100-parts CBDV:1-part CBN; 1,000-parts CBD:100-parts CBDV:1-part CBN; 500-parts CBD:1,000-parts CBN:10-parts CBG:1-part THC; 1,000-parts CBD:1,000-parts CBN:10-parts CBG:1-part THC; 100-parts CBDV:1,000-parts CBN:10-parts CBG:1-part THC; 500-parts CBD:100-parts CBDV:1,000-parts CBN:10-parts CBG:1-part THC; 1,000-parts CBD:100-parts CBDV:1,000-parts CBN:10-parts CBG:1-part THC; 500-parts CBD:500-parts CBN:10-parts CBG:1-part THC; 1,000-parts CBD:500-parts CBN:10-parts CBG:1-part THC; 100-parts CBDV:500-parts CBN:10-parts CBG:1-part THC;

500-parts CBD:100-parts CBDV:500-parts CBN:10-parts CBG:1-part THC; 1,000-parts CBD:100-parts CBDV:500-parts CBN:10-parts CBG:1-part THC; 5,000-parts CBD:10,000-parts CBN:1-part Phytol A:10-parts THC; 10,000-parts CBD:10,000-parts CBN:1-part Phytol A:10-parts THC; 1,000-parts CBDV:10,000-parts CBN:1-part Phytol A:10-parts THC; 5,000-parts CBD:1,000-parts CBDV:10,000-parts CBN:1-part Phytol A:10-parts THC; 10,000-parts CBD:1,000-parts CBDV:10,000-parts CBN:1-part Phytol A:10-parts THC; 5,000-parts CBD:5,000-parts CBN:1-part Phytol A:10-parts THC; 10,000-parts CBD:5,000-parts CBN:1-part Phytol A:10-parts THC; 1,000-parts CBDV:5,000-parts CBN:1-part Phytol A:10-parts THC; 5,000-parts CBD:1,000-parts CBDV:5,000-parts CBN:1-part Phytol A:10-parts THC; 10,000-parts CBD:1,000-parts CBDV:5,000-parts CBN:1-part Phytol A:10-parts THC; 500-parts CBD:1-part CBN:1-part THC; 1,000-parts CBD:1-part CBN:1-part THC; 100-parts CBDV:1-part CBN:1-part THC; 500-parts CBD:100-parts CBDV:1-part CBN:1-part THC; 1,000-parts CBD:100-parts CBDV:1-part CBN:1-part THC; 500-parts CBD:1,000-parts CBN:1-part THC; 1,000-parts CBD:1,000-parts CBN:1-part THC; 100-parts CBDV:1,000-parts CBN:1-part THC; 500:100:1,000:1 500-parts CBD:CBDV:CBN:1-part THC; 1,000-parts CBD:100-parts CBDV:1,000-parts CBN:1-part THC; 500-parts CBD:500-parts CBN:1-part THC; 1,000-parts CBD:500-parts CBN:1-part THC; 100-parts CBDV:500-parts CBN:1-part THC; 500-parts CBD:100-parts CBDV:500-parts CBN:1-part THC; or1,000-parts CBD:100-parts CBDV:500-parts CBN:1-part THC.

Aspects of the present disclosure provided herein further include a unit dosage form comprising the active pharmaceutical ingredient of the present disclosure.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 0.5 μM when administered.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 1 μM when administered.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 2 μM when administered.

In some embodiments, the IL-1β-decreasing and/or the IL-6-decreasing cannabinoid is Cannabidiol (CBD) and is present in an amount sufficient to achieve a Cₘₐₓ in plasma and/or target tissue of at least 5 μM when administered.

In some embodiments, the IL-1β-decreasing and/or the IL-6-decreasing cannabinoid is Cannabidiol (CBD) and is present in an amount sufficient to achieve a Cₘₐₓ in plasma and/or target tissue of at least 10 μm when administered.

In some embodiments, the IL-1β-decreasing and/or the IL-6-decreasing cannabinoid is a combination of Cannabidiol (CBD)and Cannabidivarin (CBDV), wherein the CBDV is present in an amount sufficient to achieve a mean peak concentration (Cₘₐₓ) in plasma and/or target tissue of at least 0.5 μM when administered and the CBD is present in an amount sufficient to achieve a Cₘₐₓ in plasma and/or target tissue of at least 5 μM when administered.

In some embodiments, the TNFα-decreasing cannabinoid is Cannabinol (CBN) and is present in an amount sufficient to achieve a Cₘₐₓ in plasma and/or target tissue of at least 5μM when administered.

In some embodiments, the TNFα-decreasing cannabinoid is Cannabinol (CBN) and is present in an amount sufficient to achieve a Cₘₐₓ in plasma and/or target tissue of at least 10 μM when administered.

In some embodiments, the IFNγ-decreasing cannabinoid is Cannabinol (CBN) and is present in an amount sufficient to achieve a Cₘₐₓ in plasma and/or target tissue of at least 0.001 μM when administered.

In some embodiments, the IFNγ-decreasing cannabinoid is Cannabinol (CBN) and is present in an amount sufficient to achieve a Cₘₐₓ in plasma and/or target tissue of at least 0.01 μM when administered.

In some embodiments, the IFNγ-decreasing cannabinoid is Cannabinol (CBN) and is present in an amount sufficient to achieve a Cₘₐₓ in plasma and/or target tissue of at least 0.1 μM when administered.

In some embodiments, the IFNγ-decreasing cannabinoid is Cannabigerol (CBG) and is present in an amount sufficient to achieve a Cₘₐₓ in plasma and/or target tissue of at least 0.01 μM when administered.

In some embodiments, the IFNγ-decreasing cannabinoid is Cannabigerol (CBG) and is present in an amount sufficient to achieve a Cₘₐₓ in plasma and/or target tissue of at least 0.1 μM when administered.

In some embodiments, the IFNγ-decreasing cannabinoid is Cannabigerol (CBG) and is present in an amount sufficient to achieve a Cₘₐₓ in plasma and/or target tissue of at least 0.2 μM when administered.

In some embodiments, the IFNγ-decreasing cannabinoid is Phytol A and is present in an amount sufficient to achieve a Cₘₐₓ in plasma and/or target tissue of at least 0.0005 μM when administered.

In some embodiments, the IFNγ-decreasing cannabinoid is Phytol A and is present in an amount sufficient to achieve a Cₘₐₓ in plasma and/or target tissue of at least 0.001 μM when administered.

In some embodiments, the Lymphopenia-reducing cannabinoid is tetrahydrocannabinol (THC) and is present in an amount sufficient to achieve a Cₘₐₓ in plasma and/or target tissue of at least 0.01 μM when administered.

In some embodiments, the Lymphopenia-reducing cannabinoid is tetrahydrocannabinol (THC) and is present in an amount sufficient to achieve Cₘₐₓ in plasma and/or target tissue of less than 0.02 μM when administered.

In some embodiments, the Lymphopenia-reducing cannabinoid is tetrahydrocannabinol (THC) and is present in an amount sufficient to achieve a Cₘₐₓ in plasma and/or target tissue of at least 0.01 μM but less than 0.02 μM when administered.

In some embodiments, the unit dosage form is formulated for oral administration.

In some embodiments, the unit dosage form is formulated for buccal administration, or for sublingual administration.

In some embodiments, the unit dosage form is formulated for administration by inhalation.

In some embodiments, the unit dosage form comprises: 0.7 mg to 28.3 mg of the IL-1β-decreasing and/or the IL-6-decreasing cannabinoid; 7.8 mg to 30 mg of the TNFα-decreasing cannabinoid.

In some embodiments, the unit dosage form comprises: 0.7 mg to 28.3 mg of the IL-1β-decreasing and/or the IL-6-decreasing cannabinoid; 7.8 mg to 30 mg of the TNFα-decreasing cannabinoid; and 0.0007 mg to 0.6 mg of the IFNγ-decreasing cannabinoid.

In some embodiments, the unit dosage form comprises: 0.7 mg to 28.3 mg of the IL-1β-decreasing and/or the IL-6-decreasing cannabinoid; 7.8 mg to 30 mg of the TNFα-decreasing cannabinoid; and 0.01 mg to 0.05 mg of the Lymphopenia-reducing cannabinoid.

In some embodiments, the unit dosage form comprises: 0.7 mg to 28.3 mg of the IL-1β-decreasing and/or the IL-6-decreasing cannabinoid; 7.8 mg to 30 mg of the TNFα-decreasing cannabinoid; 0.0007 mg to 0.6 mg of the IFNγ-decreasing cannabinoid; and 0.01 mg to 0.05 mg of the Lymphopenia-reducing cannabinoid.

In some embodiments, the unit dosage form comprises: 7.86 mg to 28.3 mg of dose of the IL-1β-decreasing and/or IL-6-decreasing cannabinoid, wherein the IL-1β-decreasing and/or the IL-6-decreasing cannabinoid is CBD.

In some embodiments, the unit dosage form comprises: 0.72 mg to 5.16 mg of the IL-1β-decreasing and/or IL-6-decreasing cannabinoid, wherein the IL-1β-decreasing and/or the IL-6-decreasing cannabinoid is CBDV.

In some embodiments, the unit dosage form comprises: 7.86 mg to 28.3 mg of CBD and 0.72 mg to 5.16 mg of CBDV, wherein a combination of CBD and CBDV is the IL-6-decreasing cannabinoid.

In some embodiments, the unit dosage form comprises: 7.76 mg to 27.9 mg of the TNFα-decreasing cannabinoid, wherein the TNFα-decreasing cannabinoid is CBN.

In some embodiments, the unit dosage form comprises: 0.001 mg to 0.3 mg of the IFNγ-decreasing cannabinoid, wherein the IFNγ-decreasing cannabinoid is Cannabinol (CBN).

In some embodiments, the unit dosage form comprises: 0.01 mg to 0.6 mg of the IFNγ-decreasing cannabinoid, wherein the IFNγ-decreasing cannabinoid is Cannabigerol (CBG).

In some embodiments, the unit dosage form comprises: 0.000741 mg to 0.00222 mg of the IFNγ-decreasing cannabinoid, wherein the IFNγ-decreasing cannabinoid is Phytol A.

In some embodiments, the unit dosage form comprises: 0.0157 mg to 0.0471 mg of the Lymphopenia-reducing cannabinoid, wherein the Lymphopenia-reducing cannabinoid is THC.

Aspects of the present disclosure provided herein further include a method of making an active pharmaceutical ingredient, comprising steps, in any order, of mixing: at least one the IL-1β-decreasing and/or interleukin-6 (IL-6)-decreasing cannabinoid; and at least one tumor necrosis factor alpha (TNFα)-decreasing cannabinoid; and optionally at least one lymphopenia—reducing lymphopenia cannabinoid. Aspects of the present disclosure provided herein further include a method of making an active pharmaceutical ingredient, comprising steps, in any order, of mixing at least one of: a pDC-modulating cannabinoid or terpene, a monocyte-modulating cannabinoid or terpene, and a T-cell-modulating cannabinoid or terpene.

In some embodiments, the IL-1β-decreasing and/or the IL-6-decreasing cannabinoid, the TNFα-decreasing cannabinoid, and the optional Lymphopenia-reducing cannabinoid is added to a Cannabis sativa extract.

In some embodiments, the method further comprising a preceding step of measuring the concentration in the Cannabis sativa extract of each the IL-1β-decreasing and/or IL-6-decreasing cannabinoid, TNFα-decreasing cannabinoid, and optional Lymphopenia-reducing cannabinoid.

In some embodiments, the IL-1β-decreasing and/or the IL-6-decreasing cannabinoid, the TNFα-decreasing cannabinoid, and optional Lymphopenia-reducing cannabinoid, are added to achieve a predetermined concentration in the active ingredient.

In some embodiments, the method further comprises a preceding step of preparing the Cannabis sativa extract.

In some embodiments, the Cannabis sativa extract is prepared from a Cannabis sativa strain selected to best approximate the determined composition of the active ingredient.

In some embodiments, each of the IL-1β-decreasing and/or the IL-6-decreasing cannabinoid, the TNFα-decreasing cannabinoid, and the Lymphopenia-reducing cannabinoid is synthetic or biosynthetic.

Aspects of the present disclosure provide herein further include the active ingredient of of the present disclosure or the unit dosage form of the present disclosure, produced by the method of the present disclosure.

Aspects of the present disclosure provide herein further include a pharmaceutical composition comprising the active ingredient of the present disclosure and a pharmaceutically acceptable carrier or diluent.

In some embodiments, the composition is an oil. In some embodiments, the composition is an emulsion. In some embodiments, the composition is a gel.

In some embodiments, the composition is an aerosol.

In some embodiments, the composition comprises a nanoparticle or nanoemulsion encapsulating the active ingredient.

In some embodiments, the composition is formulated for oral administration, for buccal administration, or for sublingual administration.

In some embodiments, the composition is formulated for administration by inhalation.

In some embodiments, the composition is formulated for administration by vaporizer.

In some embodiments, the composition is formulated for administration by nebulizer.

In some embodiments, the composition is formulated for administration by aerosolizer.

In some embodiments, the composition is formulated for intravenous, intramuscular, or subcutaneous administration.

In some embodiments, the composition is formulated for intrathecal or intracerebroventricular administration.

In some embodiments, the composition is formulated for topical administration.

In some embodiments, the active ingredient is present in the pharmaceutical composition at a concentration of 0.01 mg/ml.

In some embodiments, the active ingredient is present in the pharmaceutical composition at a concentration of at least 0.1 mg/ml.

In some embodiments, the active ingredient is present in the pharmaceutical composition at a concentration of at least 0.5 mg/ml.

In some embodiments, the active ingredient is present in the pharmaceutical composition at a concentration of at least 1 mg/ml.

In some embodiments, the active ingredient is present in the pharmaceutical composition at a concentration of at least 10 mg/ml.

In some embodiments, the active ingredient is present in the pharmaceutical composition at a concentration of at least 20 mg/ml.

Aspects of the present disclosure provided herein further include a method of treating a patient who has, or who is at risk for developing, cytokine release syndrome (CRS) and/or Macrophage Activation Syndrome (MAS), the method comprising: administering an effective amount of the active pharmaceutical ingredient of the present disclosure, the pharmaceutical composition of the present disclosure or the unit dosage form of the present disclosure to the patient who has, or who is at risk for developing, CRS or MAS.

In some embodiments, the patient has CRS. In some embodiments, the patient has MAS.

In some embodiments, the patient has acute lung inflammation (ALI).

In some embodiments, the patient has acute respiratory distress syndrome (ARDS).

In some embodiments, the patient has ALI with concomitant pneumonia or ARDS with concomitant pneumonia.

In some embodiments, the patient has lymphopenia.

In some embodiments, the patient has haemophagocytic lymphohistiocytosis (sHLH)

In some embodiments, the patient has acute renal injury.

In some embodiments, the patient has ischemia-reperfusion injury (IRI) or reoxygenation injury.

In some embodiments, the IRI is associated with coronary ischemia, brain ischemia, renal ischemia, or intestinal ischemia, in the patient.

In some embodiments, the patient has sepsis.

In some embodiments, the patient has a stroke.

In some embodiments, the patient has a confirmed or suspected viral infection.

In some embodiments, the infection is by a virus selected from the group consisting of coronavirus, influenza virus, rhinovirus, respiratory syncytial virus, metapneumovirus, adenovirus, and boca virus.

In some embodiments, the virus is a coronavirus selected from the group consisting of coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19).

In some embodiments, the coronavirus is SARS-CoV-2 (COVID-19).

In some embodiments, the virus is an influenza virus selected from the group consisting of parainfluenza virus 1, parainfluenza virus 2, influenza A virus, and influenza B virus.

In some embodiments, the patient has confirmed or suspected hypercoagulability.

In some embodiments, the patient is not hospitalized.

In some embodiments, the patient is hospitalized.

In some embodiments, the patient is not on a ventilator.

In some embodiments, the IL-1β-decreasing and/orthe IL-6-decreasing cannabinoid is Cannabidiol (CBD), Cannabidivarin (CBDV), or a combination thereof.

In some embodiments, the effective amount of the active pharmaceutical ingredient, pharmaceutical composition, or unit dose form comprises CBD in an amount sufficient to reduce the level of IL-6 secreted by activated CD14⁺ CD16⁺ monocytes.

In some embodiments, the effective amount of the active pharmaceutical ingredient, pharmaceutical composition, or unit dose form comprises CBD in an amount sufficient to reduce the level of IL-1β secreted by activated CD14⁺ CD16⁺ monocytes.

In some embodiments, the effective amount of the active pharmaceutical ingredient, pharmaceutical composition, or unit dose form comprises CBD in an amount sufficient to reduce phagocytosis by CD14⁺CD16⁺ monocytes.

In some embodiments, the effective amount of the active pharmaceutical ingredient, pharmaceutical composition, or unit dose form comprises CBDV in an amount sufficient to reduce the level of IL-1β secreted by activated CD14⁺ CD16⁺ monocytes.

In some embodiments, the effective amount of the active pharmaceutical ingredient, pharmaceutical composition, or unit dose form comprises CBDV in an amount sufficient to reduce the level of one or more co-stimulatory molecule expressed by CD14⁺CD16⁺ monocytes. In some embodiments, the one or more co-stimulatory molecule is selected from the group consisting of: HLA-DR, CD80, and CD86. In some embodiments, wherein the effective amount of the active pharmaceutical ingredient, pharmaceutical composition, or unit dose form comprises CBDV in an amount sufficient to suppress CD25 expression by cytotoxic CD8⁺ T cells. In some embodiments, the effective amount of the active pharmaceutical ingredient, pharmaceutical composition, or unit dose form comprises CBDV in an amount sufficient to suppress CD69 expression by cytotoxic CD8⁺ T cells.

In some embodiments, wherein the effective amount of the active pharmaceutical ingredient, pharmaceutical composition, or unit dose form comprises CBN in an amount sufficient to reduce the level of TNFα secreted by CD14⁺CD16⁺ monocytes. In some embodiments, the IFNγ-decreasing cannabinoid or terpene is cannabigerol (CBG). In some embodiments, the effective amount of the active pharmaceutical ingredient, pharmaceutical composition, or unit dose form comprises CBG in an amount sufficient to reduce the level of IFNγ secreted by T cells.

In some embodiments, wherein the effective amount of the active pharmaceutical ingredient, pharmaceutical composition, or unit dose form comprises CBN in an amount sufficient to reduce the level of IFNγ secreted by T cells. In some embodiments, the IFNγ-decreasing cannabinoid or terpene is Phytol A. In some embodiments, the effective amount of the active pharmaceutical ingredient, pharmaceutical composition, or unit dose form comprises Phytol A in an amount sufficient to reduce the level of IFNγ secreted by T cells.

In some embodiments, the optional Lymphopenia-reducing cannabinoid is tetrahydrocannabinol (THC). In some embodiments, the effective amount of the active pharmaceutical ingredient, pharmaceutical composition, or unit dose form comprises THC in an amount sufficient to reduce the level of interleukin-1 beta (IL-1) secreted by CD14⁺ -CD16⁺ monocytes. In some embodiments, the effective amount of the active pharmaceutical ingredient, pharmaceutical composition, or unit dose form comprises THC in an amount sufficient to reduce the level of TNFα secreted by CD14⁺ -CD16⁺ monocytes. In some embodiments, the effective amount of the active pharmaceutical ingredient, pharmaceutical composition, or unit dose form comprises THC in an amount sufficient to increase proliferation of helper CD4⁺ T cells. In some embodiments, the effective amount of the active pharmaceutical ingredient, pharmaceutical composition, or unit dose form comprises THC in an amount sufficient to increase proliferation of cytotoxic CD8⁺ T cells.

In some embodiments, the effective amount of the active pharmaceutical ingredient, pharmaceutical composition, or unit dose form is administered by inhalation. In some embodiments, the effective amount of the active pharmaceutical ingredient, pharmaceutical composition, or unit dose form is administered orally. In some embodiments, the effective amount of the active pharmaceutical ingredient, pharmaceutical composition, or unit dose form is administered by buccal administration. In some embodiments, the effective amount of the active pharmaceutical ingredient, pharmaceutical composition, or unit dose form is administered by sublingual administration. In some embodiments, the oral, buccal, or sublingual administration comprises nanoparticle administration. In some embodiments, the effective amount of the active pharmaceutical ingredient, pharmaceutical composition, or unit dose form is administered by injection. In some embodiments, the effective amount of the active pharmaceutical ingredient, pharmaceutical composition, or unit dose form is administered by topical application.

In some embodiments, the effective amount of the active pharmaceutical ingredient, pharmaceutical composition, or unit dose form is administered in an amount of less than 1 g per dose. In some embodiments, the effective amount of the active pharmaceutical ingredient, pharmaceutical composition, or unit dose form is administered in an amount of less than 500 mg per dose. In some embodiments, the effective amount of the active pharmaceutical ingredient, pharmaceutical composition, or unit dose form is administered in an amount of less than 100 mg per dose. In some embodiments, the effective amount of the active pharmaceutical ingredient, pharmaceutical composition, or unit dose form is administered in an amount of less than 10 mg per dose. In some embodiments, the effective amount of the active pharmaceutical ingredient, pharmaceutical composition, or unit dose form is administered in an amount ranging from 1 to 30 mg per dose.

In some embodiments, the effective amount of the active pharmaceutical ingredient, pharmaceutical composition, or unit dose form is administered p.r.n.

In some embodiments, the effective amount of the active pharmaceutical ingredient, pharmaceutical composition, or unit dose form is administered once a day. In some embodiments, the effective amount of the active pharmaceutical ingredient, pharmaceutical composition, or unit dose form is administered 2-4 times a day. In some embodiments, the effective amount of the active pharmaceutical ingredient, pharmaceutical composition, or unit dose form is administered 2-4 times a week. In some embodiments, the effective amount of the active pharmaceutical ingredient, pharmaceutical composition, or unit dose form is administered once a week. In some embodiments, the effective amount of the active pharmaceutical ingredient, pharmaceutical composition, or unit dose form is administered once every two weeks. In some embodiments the effective amount of the active pharmaceutical ingredient, pharmaceutical composition, or unit dose form is administered after a measured hyperinflammatory or pro-inflammatory response. In some embodiments the effective amount of the active pharmaceutical ingredient, pharmaceutical composition, or unit dose form is administered 6 hours after, 8 hours after, 10 hours after, 12 hours after, 14 hours after, 16 hours after, 20 hours after, 22 hours after, 24 hours after, 1 day after, 2 days after, 3 days after, 4 days after, 5 days after, 6 days after, 7 days after, 8 days after, 9 days after, or 10 days after a measured hyperinflammatory or pro-inflammatory response. In some embodiments, a hyperinflammatory or pro-inflammatory response can be measured using any known biomarkers for hyperinflammatory/pro-inflammatory responses, such as, but not limited to, TFNα and/or IFNα in pDCs, TFNα and/or IL-6 in monocytes, extracellular co-stimulatory molecules CD80 and CD86 from monocytes, TFNα, IFNγ, and/or IL-2 in CD4+ T cells, TFNα, IFNγ, and/or IL-2 in CD8+ T cells, and the like.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 0.5 μM when administered.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 1 μM when administered.

In some embodiments, the IL-1β-decreasing and/or the IL-6-decreasing cannabinoid is Cannabidivarin (CBDV) and is present in an amount sufficient to achieve a mean peak concentration (Cₘₐₓ) in plasma and/or target tissue of at least 2 μM when administered.

In some embodiments, the IL-1β-decreasing and/or the IL-6-decreasing cannabinoid is Cannabidiol (CBD) and is present in an amount sufficient to achieve a Cₘₐₓ in plasma and/or target tissue of at least 5 μM when administered.

In some embodiments, the IL-1β-decreasing and/or the IL-6-decreasing cannabinoid is Cannabidiol (CBD) and is present in an amount sufficient to achieve a Cₘₐₓ in plasma and/or target tissue of at least 10 μM when administered.

In some embodiments, the IL-1β-decreasing and/or the IL-6-decreasing cannabinoid is a combination of Cannabidiol (CBD)and Cannabidivarin (CBDV), wherein the CBDV is present in an amount sufficient to achieve a mean peak concentration (Cₘₐₓ) in plasma and/or target tissue of at least 0.5 μM when administered and the CBD is present in an amount sufficient to achieve a Cₘₐₓ in plasma and/or target tissue of at least 5 μM when administered.

In some embodiments, the TNFα-decreasing cannabinoid is Cannabinol (CBN) and is present in an amount sufficient to achieve a Cₘₐₓ in plasma and/or target tissue of at least 5 μM when administered.

In some embodiments, the TNFα-decreasing cannabinoid is Cannabinol (CBN) and is present in an amount sufficient to achieve a Cₘₐₓ in plasma and/or target tissue of at least 10 μM when administered.

In some embodiments, the IFNγ-decreasing cannabinoid is Cannabigerol (CBG) and is present in an amount sufficient to achieve a Cₘₐₓ in plasma and/or target tissue of at least 0.01 μM when administered.

In some embodiments, the IFNγ-decreasing cannabinoid is Cannabigerol (CBG) and is present in an amount sufficient to achieve a Cₘₐₓ in plasma and/or target tissue of at least 0.1 μM when administered.

In some embodiments, the IFNγ-decreasing cannabinoid is Cannabigerol (CBG) and is present in an amount sufficient to achieve a Cₘₐₓ in plasma and/or target tissue of at least 0.2 μM when administered.

In some embodiments, the IFNγ-decreasing cannabinoid is Cannabinol (CBN) and is present in an amount sufficient to achieve a Cₘₐₓ in plasma and/or target tissue of at least 0.001 μM when administered.

In some embodiments, the IFNγ-decreasing cannabinoid is Cannabinol (CBN) and is present in an amount sufficient to achieve a Cₘₐₓ in plasma and/or target tissue of at least 0.01 μM when administered.

In some embodiments, the IFNγ-decreasing cannabinoid is Cannabinol (CBN) and is present in an amount sufficient to achieve a Cₘₐₓ in plasma and/or target tissue of at least 0.1 μM when administered.

In some embodiments, the IFNγ-decreasing cannabinoid is Phytol A and is present in an amount sufficient to achieve a Cₘₐₓ in plasma and/or target tissue of at least 0.0005 μM when administered. 5

In some embodiments, the IFNγ-decreasing cannabinoid is Phytol A and is present in an amount sufficient to achieve a Cₘₐₓ in plasma and/or target tissue of at least 0.001 μM when administered.

In some embodiments, the Lymphopenia-reducing cannabinoid is tetrahydrocannabinol (THC) and is present in an amount sufficient to achieve a Cₘₐₓ in plasma and/or target tissue of at least 0.01 μM when administered.

In some embodiments, the Lymphopenia-reducing cannabinoid is tetrahydrocannabinol (THC) and is present in an amount sufficient to achieve Cₘₐₓ in plasma and/or target tissue of less than 0.02 μM when administered.

In some embodiments, the Lymphopenia-reducing cannabinoid is tetrahydrocannabinol (THC) and is present in an amount sufficient to achieve a Cₘₐₓ in plasma and/or target tissue of at least 0.01 μM but less than 0.02 μM when administered.

In some embodiments, the unit dosage form is formulated for oral administration.

In some embodiments, the unit dosage form is formulated for buccal administration, or for sublingual administration.

In some embodiments, the unit dosage form is formulated for administration by inhalation.

In some embodiments, the unit dosage form comprises: 7.9 mg to 28.3 mg of the IL-1β-decreasing and/or the IL-6-decreasing cannabinoid; 7.8 mg to 30 mg of the TNFα-decreasing cannabinoid.

In some embodiments, the unit dosage form comprises: 7.9 mg to 28.3 mg of the IL-1β-decreasing and/or the IL-6-decreasing cannabinoid; 7.8 mg to 30 mg of the TNFα-decreasing cannabinoid; and 0.0007 mg to 0.6 mg of the IFNγ-decreasing cannabinoid.

In some embodiments, the unit dosage form comprises:: 7.9 mg to 28.3 mg of the IL-1β-decreasing and/or the IL-6-decreasing cannabinoid; 7.8 mg to 30 mg of the TNFα-decreasing cannabinoid; and 0.01 mg to 0.05 mg of the Lymphopenia-reducing cannabinoid.

In some embodiments, the unit dosage form comprises: 7.9 mg to 28.3 mg of the IL-1β-decreasing and/or the IL-6-decreasing cannabinoid; 7.8 mg to 30 mg of the TNFα-decreasing cannabinoid; 0.0007 mg to 0.6 mg of the IFNγ-decreasing cannabinoid; and 0.01 mg to 0.05 mg of the Lymphopenia-reducing cannabinoid.

In some embodiments, the unit dosage form comprises: 7.86 mg to 28.3 mg of dose of the IL-1β-decreasing and/or IL-6-decreasing cannabinoid, wherein the IL-1β-decreasing and/or the IL-6-decreasing cannabinoid is CBD.

In some embodiments, the unit dosage form comprises: 0.72 mg to 5.16 mg of the IL-1β-decreasing and/or IL-6-decreasing cannabinoid, wherein the IL-1β-decreasing and/or the IL-6-decreasing cannabinoid is CBDV.

In some embodiments, the unit dosage form comprises: 7.86 mg to 28.3 mg of CBD and 0.72 mg to 5.16 mg of CBDV, wherein a combination of CBD and CBDV is the IL-1β-decreasing and/or the IL-6-decreasing cannabinoid.

In some embodiments, the unit dosage form comprises: 7.76 mg to 27.9 mg of the TNFα-decreasing cannabinoid, wherein the TNFα-decreasing cannabinoid is CBN.

In some embodiments, the unit dosage form comprises: 0.01 mg to 0.6 mg of the IFNγ-decreasing cannabinoid, wherein the IFNγ-decreasing cannabinoid is Cannabigerol (CBG).

In some embodiments, the unit dosage form comprises: 0.001 mg to 0.3 mg of the IFNγ-decreasing cannabinoid, wherein the IFNγ-decreasing cannabinoid is Cannabinol (CBN).

In some embodiments, the unit dosage form comprises: 0.000741 mg to 0.00222 mg of the IFNγ-decreasing cannabinoid, wherein the IFNγ-decreasing cannabinoid is Phytol A.

In some embodiments, the unit dosage form comprises: 0.0157 mg to 0.0471 mg of the Lymphopenia-reducing cannabinoid, wherein the Lymphopenia-reducing cannabinoid is THC.

In some embodiments, the patient has a body temperature greater than 37.5° C. prior to administration of the active pharmaceutical ingredient, pharmaceutical composition, or unit dose form.

In some embodiments, the body temperature of the patient is measured at one or more sites selected from the group consisting of an oral cavity, a rectal cavity, axilla area, and tympanic membrane.

In some embodiments, the patient has a pre-treatment C reactive protein (CRP) level greater than 2 mg/L.

In some embodiments, the patient has a pre-treatment CRP level greater than 5 mg/L, 10 mg/L, 15 mg/L, 20 mg/L, 25 mg/L, 30 mg/L, 35 mg/L, or 40 mg/L.

In some embodiments, the patient has a pre-treatment serum IL-6 level of at least 2 pg/ml. In some embodiments, the patient has a pre-treatment serum IL-6 level of at least 2.5 pg/ml, 3 μg/ml, 4 μg/ml, 5 μg/ml, 10 μg/ml, 20 μg/ml, 30 μg/ml, 40 μg/ml, 50 μg/ml, 60 μg/ml, 70 μg/ml, 80 μg/ml, 90 μg/ml, 100 μg/ml, 150 μg/ml or 200 μg/ml.

In some embodiments, the patient has a pre-treatment neutrophil-to-lymphocyte ratio (NLR) greater than 2.0.

In some embodiments, the patient has a pre-treatment NLR greater than 2.8, 2.9, 3.0, 3.1, 3.2, 3.3, 3.4, 3.5, 3.6, 3.7, 3.8, 3.9, or 4.0.

In some embodiments, the patient has a pre-treatment respiration rate on ambient air of fewer than 12 breaths or more than 25 breaths per minute.

In some embodiments, the patient has a pre-treatment oxygen saturation level on ambient air of no more than 95%, 94%, 93%, 92%, 91%, 90%, 89%, 88%, 87%, 86%, 85%, 84%, 83%, 82%, 81%, 80%, or 75%.

In some embodiments, the patient has a pre-treatment D-Dimer level that is elevated above baseline. In some embodiments, the patient has a pre-treatment sepsis-induced coagulopathy (SIC) total score of 4 or more with total score of prothrombin time and coagulation exceeding 2.

In some embodiments, the method reduces the body temperature of the patient below pre-treatment levels. In some embodiments, the method reduces the patient's serum CRP levels below pre-treatment levels. In some embodiments, the post-treatment CRP level is no more than 45 mg/L, 40 mg/L, 35 mg/L, 30 mg/L, 25 mg/L, 20 mg/L, 15 mg/L, 10 mg/L, 5 mg/L, or 1 mg/L. In some embodiments, the method reduces the CRP level by at least 10%, 20%, 30%, 40%, 50%, 60%, 70%, 80%, or 90% as compared to pre-treatment levels.

In some embodiments, the method reduces, in the patient, one or more pro-inflammatory cytokine serum levels below pre-treatment levels, wherein the one or more pro-inflammatory cytokines is selected from the group consisting of: IL-6, IL-1β, TNFα; IFNγ, IFNα, IL-17A; IL-17F, and IL-2.

In some embodiments, the method reduces, in the patient, one or more co-stimulatory molecules, such as CD80 and CD86, below pre-treatment levels. In some embodiments, the extracellular CD80 and/or CD86 level is decreased by at least 10%, at least 20%, at least 30%, at least 40%, at least 50%, at least 60%, at least 70%, at least%, or at least 90% as compared to pre-treatment levels.

In some embodiments, the method reduces the patient's serum IL-6 levels below pre-treatment levels. In some embodiments, the serum IL-6 level is decreased by at least 10% as compared to pre-treatment levels. In some embodiments, the serum IL-6 level is decreased by at least 20%, 30%, 40%, 50%, 60%, 70%, 80%, or 90% as compared to pre-treatment levels.

In some embodiments, the serum TNFα level is decreased by at least 10% as compared to pre-treatment levels. In some embodiments, the serum TNFα level is decreased by at least 20%, 30%, 40%, 50%, 60%, 70%, 80%, or 90% as compared to pre-treatment levels.

In some embodiments, the serum IFNα level is decreased by at least 10% as compared to pre-treatment levels. In some embodiments, the serum IFNα level is decreased by at least 20%, 30%, 40%, 50%, 60%, 70%, 80%, or 90% as compared to pre-treatment levels.

In some embodiments, the serum IFNγ level is decreased by at least 10% as compared to pre-treatment levels. In some embodiments, the serum IFNγ a level is decreased by at least 20%, 30%, 40%, 50%, 60%, 70%, 80%, or 90% as compared to pre-treatment levels.

In some embodiments, the serum IL-2 level is decreased by at least 10% as compared to pre-treatment levels. In some embodiments, the serum IL-2 level is decreased by at least 20%, 30%, 40%, 50%, 60%, 70%, 80%, or 90% as compared to pre-treatment levels.

In some embodiments, the patient has a reduced serum level of immunoglobulin G (IgG) as compared to pre-treatment levels. In some embodiments, the patient has a reduced serum level of immunoglobulin G (IgG) by at least 10% as compared to pre-treatment levels. In some embodiments, the patient has a post-treatment NLR less than 3.18. In some embodiments, the administration of the active pharmaceutical ingredient, pharmaceutical composition, or unit dose form increases the NLR by at least 10%, 20%, 30%, 40%, 50%, 60%, 70%, 80%, or 90% as compared to pre-treatment levels.

In some embodiments, the method increases the respiration rate of the patient. In some embodiments, the patient has a post-treatment respiration rate between 12 to 20 breaths per minute. In some embodiments, the method increases the oxygen saturation level of the patient on ambient air by 1%, 2%, 3%, 4%, 5%, 6%, 7%, 8%, 9%,10%, 11%, 12%, 13%, 14%, 15%, 16%, 17%, 18%, 19%, 20%, 25%, or 30% as compared to pre-treatment levels.

In some embodiments, the method reduces the patient's need for supplemental oxygen.

In some embodiments, the patient is older than 50, 51, 52, 53, 54, 55, 56, 57, 58, or 59 years of age. In some embodiments, the patient is older than 60 years of age. In some embodiments, the patient is younger than 60, 59, 58, 57, 56, 55, 54, 53, 52, 51, or 50 years of age.

Aspects of the present disclosure provide an active pharmaceutical ingredient, comprising: (a) a T-cell-modulating cannabinoid or terpene; and (b) a monocyte-modulating cannabinoid or terpene.

In some embodiments, the T-cell modulating cannabinoid or terpene is a CD4+ T-cell modulating cannabinoid or terpene, a CD8+ T-cell modulating cannabinoid or terpene, or a CD4+ and CD8+ T-cell modulating cannabinoid or terpene. In some embodiments, the T-cell modulating cannabinoid or terpene comprises a single cannabinoid or terpene. In some embodiments, the T-cell modulating cannabinoid or terpene comprises two or more cannabinoids or terpenes. In some embodiments, the monocyte-modulating cannabinoid or terpene comprises a single cannabinoid or terpene. In some embodiments, the monocyte-modulating cannabinoid or terpene comprises two or more cannabinoids or terpenes.

In some embodiments, the T-cell modulating cannabinoid or terpene comprises two or more cannabinoids or terpenes. In some embodiments, the T-cell modulating cannabinoid or terpene is selected from one or more of: cannabidiol (CBD), Cannabidivarin (CBDV), Cannabinol (CBN), phytol A, and tetrahydrocannabinol (THC). In some embodiments, the monocyte modulating cannabinoid or terpene is selected from one or more of cannabidiol (CBD), Cannabidivarin (CBDV), Cannabinol (CBN), Phytol A, and tetrahydrocannabinol (THC). In some embodiments, the active pharmaceutical ingredient comprises two or more of cannabidiol (CBD), Cannabidivarin (CBDV), Cannabinol (CBN), phytol A, and tetrahydrocannabinol (THC).

In some embodiments, the active pharmaceutical ingredient consists essentially of: CBD and CBN; CBDV, CBD, and CBN; CBD, and CBN; CBDV, CBN, and CBG; CBDV, CBN, Phytol A, and THC; CBDV and CBN; or CBD, CBN, and THC. In some embodiments, the active pharmaceutical ingredient consists essentially of: 1 part CBD:1-part CBN;1-part CBDV:5-parts CBD:5-parts CBN; 500-parts CBD:1-part CBN; 10-parts CBDV:50-parts CBN:1-part CBG; 1,000-parts CBDV:5,000-parts CBN:1-part Phytol A:10-parts THC; 1-part CBDV:5-parts CBN; or 500-parts CBD:1-part CBN:1 part THC.

In some embodiments, each of the T-cell modulating cannabinoid or terpene and the monocyte-modulating cannabinoid or terpene is synthetic or biosynthetic.

In some embodiments, the active pharmaceutical ingredient further comprises an optional Lymphopenia-reducing cannabinoid. In some embodiments, the optional Lymphopenia-reducing cannabinoid is tetrahydrocannabinol (THC).

In some embodiments, the T-cell modulating cannabinoid or terpene, and the monocyte-modulating cannabinoid or terpene collectively constitute at least 75% by weight of the active ingredient. In some embodiments, the active pharmaceutical ingredient is substantially free of tetrahydrocannabinol (THC). In some embodiments, the T-cell modulating cannabinoid or terpene constitutes 0.01-99% by weight of the active ingredient; the monocyte modulating cannabinoid or terpene constitutes 0.01-99% by weight of the active ingredient; and the optional Lymphopenia-reducing cannabinoid constitutes 0.01-99% by weight of the active ingredient.

In some embodiments, the T-cell modulating cannabinoid or terpene is a CD4+ T-cell targeting cannabinoid or terpene, a CD8+ T-cell-targeting cannabinoid or terpene. In some embodiments, the T-cell modulating cannabinoid or terpene is a CD4+ and CD8+ T-cell-targeting cannabinoid or terpene. In some embodiments, the T-cell modulating cannabinoid or terpene comprises two or more cannabinoids or terpenes, wherein at least one cannabinoid or terpene is a CD4+ T-cell-targeting cannabinoid or terpene. In some embodiments, the T-cell modulating cannabinoid or terpene comprises two or more cannabinoids or terpenes, wherein at least one cannabinoid or terpene is a CD8+ T-cell-targeting cannabinoid or terpene.

Aspects of the present disclosure provide a unit dosage form comprising the active pharmaceutical ingredient. In some embodiments, the T-cell modulating cannabinoid or terpene is present in an amount sufficient to achieve a mean peak concentration (Cmax) in plasma and/or target tissue of at least 0.001 μM, at least 0.01 μM, at least 0.1 μM, at least 1 μM, or at least 5 μM, when administered. In some embodiments, the monocyte-modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the unit dosage form comprises:(a) 7 mg to 29 mg of CBD, and 0.001 mg to 28 mg of CBN; (b) 0.7 mg to 5.2 mg dose of CBDV, 7 mg to 29 mg of CBD, and 0.001 mg to 28 mg of CBN; (c) 7 mg to 29 mg of CBD, and 0.001 mg to 28 mg of CBN; (d) 0.7 mg to 5.2 mg of CBDV, 0.001 mg to 28 mg of CBN, and 0.01 mg to 0.6 mg of CBG; (e) 0.7 mg to 5.2 mg of CBDV, 0.001 mg to 28 mg of CBN, 0.0007 mg to 0.002 mg of Phytol A, and 0.01 mg to 0.05 mg of THC; (f) 0.7 mg to 5.2 mg of CBDV and 0.001 mg to 28 mg of CBN; or (g) 7 mg to 29 mg of CBD, 0.001 mg to 28 mg of CBN, and 0.01 mg to 0.05 mg of THC.

Aspects of the present disclosure include a method of making an active pharmaceutical ingredient, comprising steps, in any order, of mixing: at least one T-cell modulating cannabinoid or terpene; and at least one monocyte-cell modulating cannabinoid or terpene.

In some embodiments, each of the T-cell modulating cannabinoid or terpene and monocyte-modulating cannabinoid or terpene is synthetic or biosynthetic.

Aspects of the present disclosure provide a pharmaceutical composition comprising the active ingredient and a pharmaceutically acceptable carrier or diluent. In some embodiments, the composition is an oil, an emulsion, a gel, or an aerosol. In some embodiments, the composition comprises a nanoparticle or nanoemulsion encapsulating the active ingredient. In some embodiments, the active ingredient is present in the pharmaceutical composition at a concentration of at least 0.01 mg/ml, at least 0.1 mg/ml, at least 0.5 mg/ml, at least 1 mg/ml, at least 10 mg/ml, or at least 20 mg/ml.

Aspects of the present disclosure provide a method of treating a patient who has, or who is at risk for developing, cytokine release syndrome (CRS) and/or Macrophage Activation Syndrome (MAS), the method comprising: administering an effective amount of the active pharmaceutical ingredient, the pharmaceutical composition or the unit dosage form to the patient who has, or who is at risk for developing, CRS or MAS.

In some embodiments, the patient has hyperinflammation. In some embodiments, the hyperinflammation is measured by a pre-treatment level of one or more of: IFNα, TNFα, IL-6, CD80, CD86, IL-2, and IFNy. In some embodiments, the patient has CRS. In some embodiments, the patient has MAS.

In some embodiments, the patient has at least one of: acute lung inflammation (ALI), acute respiratory distress syndrome (ARDS), lymphopenia, haemophagocytic lymphohistiocytosis (sHLH), acute renal injury, ischemia-reperfusion injury (IRI) or reoxygenation injury. In some embodiments, the patient has ALI with concomitant pneumonia or ARDS with concomitant pneumonia. In some embodiments, the IRI is associated with coronary ischemia, brain ischemia, renal ischemia, or intestinal ischemia, in the patient. In some embodiments, the patient has sepsis. In some embodiments, the patient has a stroke. In some embodiments, the patient has a confirmed or suspected viral infection. In some embodiments, infection is by a virus selected from the group consisting of coronavirus, influenza virus, rhinovirus, respiratory syncytial virus, metapneumovirus, adenovirus, and boca virus.

In some embodiments, the virus is a coronavirus selected from the group consisting of coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19). In some embodiments, the coronavirus is SARS-CoV-2 (COVID-19). In some embodiments, the virus is an influenza virus selected from the group consisting of parainfluenza virus 1, parainfluenza virus 2, influenza A virus, and influenza B virus.

In some embodiments, the effective amount of the active pharmaceutical ingredient, pharmaceutical composition, or unit dose form reduces the level of IL-6 secreted by activated CD14+ CD16+ monocytes. In some embodiments, the effective amount of the active pharmaceutical ingredient, pharmaceutical composition, or unit dose form reduces phagocytosis by CD14+CD16+ monocytes. In some embodiments, the effective amount of the active pharmaceutical ingredient, pharmaceutical composition, or unit dose form reduces the level of TNFα by CD14+CD16+ monocytes. In some embodiments, the effective amount of the active pharmaceutical ingredient, pharmaceutical composition, or unit dose form reduces the level of one or more co-stimulatory molecule expressed by CD14+CD16+ monocytes.

In some embodiments, the one or more co-stimulatory molecule is selected from the group consisting of: HLA-DR, CD80, and CD86. In some embodiments, the effective amount of the active pharmaceutical ingredient, pharmaceutical composition, or unit dose form suppresses TNFα expression by CD4+ T cells, CD8+ T cells, or CD4+ and CD8+ T cells. In some embodiments, the effective amount of the active pharmaceutical ingredient, pharmaceutical composition, or unit dose form reduces the level of IFNγ expression by CD4+ T cells, CD8+ T cells, or CD4+ and CD8+ T cells. In some embodiments, the effective amount of the active pharmaceutical ingredient, pharmaceutical composition, or unit dose form reduces the level of IL-2 secreted by T cells. In some embodiments, the T cell is selected from a CD4+ T cell, a CD8+ T cell, or a CD4+ and CD8+ T cell.

In some embodiments, the effective amount of the active pharmaceutical ingredient, pharmaceutical composition, or unit dose form preserves a plasmacytoid dendritic cell (pDC) cell response to a virus or bacteria. In some embodiments, the effective amount of the active pharmaceutical ingredient, pharmaceutical composition, or unit dose form has no effect on a plasmacytoid dendritic cell (pDC) cell response to a virus or bacteria. In some embodiments, the the effective amount of the active pharmaceutical ingredient, pharmaceutical composition, or unit dose form is administered by inhalation. In some embodiments, the effective amount of the active pharmaceutical ingredient, pharmaceutical composition, or unit dose form is administered orally, by buccal administration, or by sublingual administration . In some embodiments, the the oral, buccal, or sublingual administration comprises nanoparticle administration. In some embodiments, the effective amount of the active pharmaceutical ingredient, pharmaceutical composition, or unit dose form is administered by injection.

In some embodiments, the effective amount of the active pharmaceutical ingredient, pharmaceutical composition, or unit dose form is administered by topical application. In some embodiments, the effective amount of the active pharmaceutical ingredient, pharmaceutical composition, or unit dose form is administered in an amount of less than 1 g per dose. In some embodiments, the effective amount of the active pharmaceutical ingredient, pharmaceutical composition, or unit dose form is administered in an amount of less than 500 mg per dose, less than 100 mg per dose, or less than 10 mg per dose. In some embodiments, the effective amount of the active pharmaceutical ingredient, pharmaceutical composition, or unit dose form is administered in an amount ranging from 1 to 30 mg per dose. In some embodiments, the effective amount of the active pharmaceutical ingredient, pharmaceutical composition, or unit dose form is administered p.r.n. In some embodiments, the effective amount of the active pharmaceutical ingredient, pharmaceutical composition, or unit dose form is administered after a measured increase in an inflammatory response as compared to a patient that does not have, or is not at risk for developing, CRS or MAS. In some embodiments, the effective amount of the active pharmaceutical ingredient, pharmaceutical composition, or unit dose form is administered 6 hours after, 24 hours after, or 4 days after a measured increase in an inflammatory response as compared to a patient that does not have, or is not at risk for developing, CRS or MAS. In some embodiments, the effective amount of the active pharmaceutical ingredient, pharmaceutical composition, or unit dose form is administered once a day, 2-4 times a day, 2-4 times a week, once a week, or once every two weeks. In some embodiments, the T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM when administered. In some embodiments, the monocyte-modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM when administered.

In some embodiments, the unit dose form comprises: (a) 7 mg to 29 mg of CBD, and 0.001 mg to 28 mg of CBN; (b) 0.7 mg to 5.2 mg of CBDV, 7 mg to 29 mg of CBD, and 0.001 mg to 28 mg of CBN; (c) 7 mg to 29 mg of CBD, and 0.001 mg to 28 mg of CBN; (d) 0.7 mg to 5.2 mg of CBDV, 0.001 mg to 28 mg of CBN, and 0.01 mg to 0.6 mg of CBG; (e) 0.7 mg to 5.2 mg of CBDV, 0.001 mg to 28 mg of CBN, 0.0007 mg to 0.002 mg of Phytol A, and 0.01 mg to 0.05 mg of THC; (f) 0.7 mg to 5.2 mg of CBDV and 0.001 mg to 28 mg of CBN; or (g) 7 mg to 29 mg of CBD, 0.001 mg to 28 mg of CBN, and 0.01 mg to 0.05 mg of THC.

Aspects of the present disclosure provide an active pharmaceutical ingredient, comprising: one or more T-cell-modulating cannabinoids.

Aspects of the present disclosure provide an active pharmaceutical ingredient, comprising: two or more T-cell-modulating cannabinoids.

In some embodiments, the the two or more T-cell- decreasing cannabinoids is a CD4+ T-cell decreasing cannabinoid or terpene, a CD8+ T-cell reducing cannabinoid or terpene, or a CD4+ and CD8+ T-cell modulating cannabinoid or terpene. In some embodiments, the two or more cannabinoids comprise Cannabidivarin (CBDV) and Cannabinol (CBN). In some embodiments, the two or more cannabinoids consist essentially of 100-parts Cannabidivarin (CBDV) and 1-part Cannabinol (CBN). In some embodiments, the two or more cannabinoids consist essentially of Cannabidivarin (CBDV) and 1-part Cannabinol (CBN). In some embodiments, the T-cell modulating cannabinoid or terpene is synthetic or biosynthetic. In some embodiments, the active pharmaceutical ingredient further comprises an optional Lymphopenia-reducing cannabinoid. In some embodiments, the optional Lymphopenia-decreasing cannabinoid is tetrahydrocannabinol (THC).

In some embodiments, the two or more T-cell modulating cannabinoids collectively constitute at least 75% by weight of the active ingredient. In some embodiments, all compounds in the active ingredient other than the two or more T-cell modulating cannabinoids are extractable from Cannabis sativa.

In some embodiments, the active pharmaceutical ingredient is substantially free of tetrahydrocannabinol (THC). In some embodiments, the two or more T-cell modulating cannabinoids constitute 0.01-99% by weight of the active ingredient.

Aspects of the present disclosure provide a unit dosage form comprising the active pharmaceutical ingredient. In some embodiments, at least one of the two or more T-cell modulating cannabinoids are present in an amount sufficient to achieve a mean peak concentration (Cmax) in plasma and/or target tissue of at least 0.01 μM when administered. In some embodiments, the at least one of the two or more T-cell modulating cannabinoids is CBN.

In some embodiments, at least one of the two or more T-cell modulating cannabinoids is present in an amount sufficient to achieve a mean peak concentration (Cₘₐₓ) in plasma and/or target tissue of at least 1 μM when administered. In some embodiments, at least one of the two or more T-cell modulating cannabinoids is CBDV. In some embodiments, the unit dosage form is formulated for oral administration, buccal administration, or for sublingual administration. In some embodiments, the unit dosage form is formulated for administration by inhalation. In some embodiments, the unit dose form comprises: (a) 0.001 mg to 28 mg of CBN and 0.7 mg to 5.2 mg of CBDV.

Aspects of the present disclosure provide of making an active pharmaceutical ingredient, comprising steps, in any order, of mixing: two or more T-cell modulating cannabinoids.

In some embodiments, the two or more T-cell decreasing cannabinoids are added to a Cannabis sativa extract. In some embodiments, each of the two or more T-cell modulating cannabinoids is synthetic or biosynthetic. In some embodiments, the active pharmaceutical ingredient or the unit dosage form is produced by the method of making the active pharmaceutical ingredient.

Aspects of the present disclosure provide a pharmaceutical composition comprising the active ingredient and a pharmaceutically acceptable carrier or diluent.

In some embodiments, the composition is an oil, an emulsion, a gel, or an aerosol. In some embodiments, the composition comprises a nanoparticle or nanoemulsion encapsulating the active ingredient. In some embodiments, the active ingredient is present in the pharmaceutical composition at a concentration of 0.01 mg/ml.

In some embodiments, the active ingredient is present in the pharmaceutical composition at a concentration of at least 0.1 mg/ml. In some embodiments, the active ingredient is present in the pharmaceutical composition at a concentration of at least 0.5 mg/ml. In some embodiments, the active ingredient is present in the pharmaceutical composition at a concentration of at least 1 mg/ml.

In some embodiments, the active ingredient is present in the pharmaceutical composition at a concentration of at least 10 mg/ml. In some embodiments, the active ingredient is present in the pharmaceutical composition at a concentration of at least 20 mg/ml.

Aspects of the present disclosure provide a method of treating a patient who has, or who is at risk for developing, cytokine release syndrome (CRS) and/or Macrophage Activation Syndrome (MAS), the method comprising: administering an effective amount of the active ingredient, the pharmaceutical composition or the unit dosage form to the patient who has, or who is at risk for developing, CRS or MAS.

In some embodiments, the patient has T-cell related hyperinflammation. In some embodiments, the T-cell related hyperinflammation is measured by a pre-treatment level of TNFα, IL-2, and IFNγ secreted by CD4+ T cells, CD8+ T cells, or both CD4+ and CD8+ T cells. In some embodiments, the patient has CRS.

In some embodiments, the patient has MAS. In some embodiments, the patient has one or more of: acute lung inflammation (ALI), acute respiratory distress syndrome (ARDS), ALI with concomitant pneumonia or ARDS with concomitant pneumonia, lymphopenia, haemophagocytic lymphohistiocytosis (sHLH), acute renal injury, ischemia-reperfusion injury (IRI) or reoxygenation injury.

In some embodiments, the IRI is associated with coronary ischemia, brain ischemia, renal ischemia, or intestinal ischemia, in the patient. In some embodiments, the patient has sepsis. In some embodiments, the patient has a stroke. In some embodiments, the patient has a confirmed or suspected viral infection.

In some embodiments, the infection is by a virus selected from the group consisting of coronavirus, influenza virus, rhinovirus, respiratory syncytial virus, metapneumovirus, adenovirus, and boca virus. In some embodiments, the virus is a coronavirus selected from the group consisting of coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19). In some embodiments, the coronavirus is SARS-CoV-2 (COVID-19).

In some embodiments, the virus is an influenza virus selected from the group consisting of parainfluenza virus 1, parainfluenza virus 2, influenza A virus, and influenza B virus. In some embodiments, the effective amount of the active pharmaceutical ingredient, pharmaceutical composition, or unit dose form reduces the level TNFα secreted by T cells.

In some embodiments, the effective amount of the active pharmaceutical ingredient, pharmaceutical composition, or unit dose form reduces the level IFNγ secreted by T cells. In some embodiments, the effective amount of the active pharmaceutical ingredient, pharmaceutical composition, or unit dose form preserves a plasmacytoid dendritic cell (pDC) cell response to a virus or bacteria. In some embodiments, the effective amount of the active pharmaceutical ingredient, pharmaceutical composition, or unit dose form has no effect on a plasmacytoid dendritic cell (pDC) cell response to a virus or bacteria.

In some embodiments, the effective amount of the active pharmaceutical ingredient, pharmaceutical composition, or unit dose form is administered by inhalation. In some embodiments, the effective amount of the active pharmaceutical ingredient, pharmaceutical composition, or unit dose form is administered orally, by buccal administration, by or sublingual administration.

In some embodiments, the oral, buccal, or sublingual administration comprises nanoparticle administration. In some embodiments, the effective amount of the active pharmaceutical ingredient, pharmaceutical composition, or unit dose form is administered by injection. In some embodiments, the effective amount of the active pharmaceutical ingredient, pharmaceutical composition, or unit dose form is administered by topical application.

In some embodiments, the effective amount of the active pharmaceutical ingredient, pharmaceutical composition, or unit dose form is administered in an amount of less than 1 g per dose. In some embodiments, the effective amount of the active pharmaceutical ingredient, pharmaceutical composition, or unit dose form is administered in an amount of less than 500 mg per dose, less than 100 mg per dose, or less than 10 mg per dose. In some embodiments, the effective amount of the active pharmaceutical ingredient, pharmaceutical composition, or unit dose form is administered in an amount ranging from 1 to 30 mg per dose.

In some embodiments, the effective amount of the active pharmaceutical ingredient, pharmaceutical composition, or unit dose form is administered p.r.n. In some embodiments, the effective amount of the active pharmaceutical ingredient, pharmaceutical composition, or unit dose form is administered after a measured increase in an inflammatory response as compared to a patient that does not have, or is not at risk for developing, CRS or MAS. In some embodiments, the effective amount of the active pharmaceutical ingredient, pharmaceutical composition, or unit dose form is administered 6 hours after, 24 hours after, or 4 days after a measured increase in an inflammatory response as compared to a patient that does not have, or is not at risk for developing, CRS or MAS.

An active pharmaceutical ingredient comprising: (a) a plasmacytoid dendritic cell-(pDC) modulating cannabinoid or terpene; (b) a monocyte-modulating cannabinoid or terpene; and (c) a T-cell-modulating cannabinoid or terpene.

In some embodiments, the pDC modulating cannabinoid or terpene comprises a single cannabinoid or terpene. In some embodiments, the pDC modulating cannabinoid or terpene comprises two or more cannabinoids or terpenes. In some embodiments, the monocyte-modulating cannabinoid or terpene comprises a single cannabinoid or terpene. In some embodiments, the monocyte-modulating cannabinoid or terpene comprises two or more cannabinoids or terpenes.

In some embodiments, the T-cell modulating cannabinoid or terpene is a CD4+ T-cell modulating cannabinoid or terpene, a CD8+ T-cell modulating cannabinoid or terpene, or a CD4+ and CD8+ T-cell modulating cannabinoid or terpene.

In some embodiments, the T-cell modulating cannabinoid or terpene comprises a single cannabinoid or terpene. In some embodiments, the T-cell modulating cannabinoid or terpene comprises two or more cannabinoids or terpenes. In some embodiments, the pDC modulating cannabinoid or terpene is selected from one or more of cannabidiol (CBD), Cannabidivarin (CBDV), Cannabinol (CBN), Cannabigerol (CBG), phytol A, and tetrahydrocannabinol (THC).

In some embodiments, the T-cell modulating cannabinoid or terpene is selected from one or more of: cannabidiol (CBD), Cannabidivarin (CBDV), Cannabinol (CBN), Cannabigerol (CBG), Phytol A, and tetrahydrocannabinol (THC). In some embodiments, the monocyte modulating cannabinoid or terpene is selected from one or more of cannabidiol (CBD), Cannabidivarin (CBDV), Cannabinol (CBN), Cannabigerol (CBG), phytol A, and tetrahydrocannabinol (THC).

In some embodiments, the active pharmaceutical ingredient comprises two or more of cannabidiol (CBD), Cannabidivarin (CBDV), Cannabinol (CBN), Cannabigerol (CBG), Phytol A, and tetrahydrocannabinol (THC).

In some embodiments, the active pharmaceutical ingredient consists essentially of: CBD, CBN, and THC; CBDV, CBN: and Phyol A;CBDV, CBD, CBN, and CBG; CBD, CBN, and CBG; or CBDV, CBD, CBN, and THC.

In some embodiments, the active pharmaceutical ingredient consists essentially of:

500-parts CBD:500-parts CBN: 1-part THC; 1,000-parts CBDV:5,000-parts CBN:1-part:Phytol A; 10-parts CBDV:50-parts: CBD 50: CBN: 1-part:CBG; 50-parts CBD:50-parts CBN:1-part CBG; or 100-parts CBDV:500-parts CBD:1-part CBN:1-part THC.

In some embodiments, each of the pDC-modulating cannabinoid or terpene, T-cell modulating cannabinoid or terpene and the monocyte-modulating cannabinoid or terpene is synthetic or biosynthetic.

In some embodiments, the active pharmaceutical ingredient further comprises an optional Lymphopenia-reducing cannabinoid. In some embodiments, the optional Lymphopenia-reducing cannabinoid is tetrahydrocannabinol (THC).

In some embodiments, the pDC-modulating cannabinoid or terpene, T-cell modulating cannabinoid or terpene and the monocyte-modulating cannabinoid or terpene collectively constitute at least 75% by weight of the active ingredient.

In some embodiments, all compounds in the active ingredient other than the pDC-modulating cannabinoid or terpene, T-cell modulating cannabinoid or terpene and the monocyte-modulating cannabinoid or terpene are extractable from Cannabis sativa.

In some embodiments, the active pharmaceutical ingredient is substantially free of tetrahydrocannabinol (THC). In some embodiments, the pDC modulating cannabinoid or terpene constitutes 0.01-99% by weight of the active ingredient;the T-cell modulating cannabinoid or terpene constitutes 0.01-99% by weight of the active ingredient; the monocyte modulating cannabinoid or terpene constitutes 0.01-99% by weight of the active ingredient; and the optional Lymphopenia-reducing cannabinoid constitutes 0.01-99% by weight of the active ingredient.

In some embodiments, the pDC modulating cannabinoid or terpene is a pDC-targeting cannabinoid or terpene. In some embodiments, the monocyte modulating cannabinoid or terpene is a monocyte-targeting cannabinoid or terpene.

In some embodiments, the T-cell modulating cannabinoid or terpene is a CD4+ T-cell targeting cannabinoid or terpene. In some embodiments, the T-cell modulating cannabinoid or terpene is a CD8+ T-cell-targeting cannabinoid or terpene. In some embodiments, the T-cell modulating cannabinoid or terpene is a CD4+ and CD8+ T-cell-targeting cannabinoid or terpene.

In some embodiments, the T-cell modulating cannabinoid or terpene comprises two or more cannabinoids or terpenes, wherein at least one cannabinoid or terpene is a CD4+ T-cell-targeting cannabinoid or terpene. In some embodiments, the T-cell modulating cannabinoid or terpene comprises two or more cannabinoids or terpenes, wherein at least one cannabinoid or terpene is a CD8+ T-cell-targeting cannabinoid or terpene.

Aspects of the present disclosure provide a unit dosage form comprising the active pharmaceutical ingredient.

In some embodiments, the pDC modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the pDC modulating cannabinoid or terpene is present in an amount sufficient to achieve a mean peak concentration (Cₘₐₓ) in plasma and/or target tissue of at least0.001 μM, at least 0.01 μM, at least 0.1 μM, at least 1 μM, or at least 5 μM, when administered In some embodiments, the monocyte-modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered. In some embodiments, the T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiment, the unit dose form comprises (a) 7 mg to 29 mg of CBD, 0.001 mg to 28 mg of CBN, and 0.01 mg to 0.05 mg of THC (b) 0.7 mg to 5.2 mg of CBDV, 0.001 mg to 28 mg of CBN, and 0.0007 mg to 0.002 mg of Phytol A; (c) 0.7 mg to 5.2 mg of CBDV, 7 mg to 29 mg of CBD, 0.001 mg to 28 mg of CBN, and 0.01 mg to 0.6 mg of CBG; (d) 7 mg to 29 mg of CBD, 0.001 mg to 28 mg of CBN, and 0.01 mg to 0.6 mg of CBG; or (e) 0.7 mg to 5.2 mg of CBDV, 7 mg to 29 mg of CBD, 0.001 mg to 28 mg of CBN, and 0.01 mg to 0.05 mg of THC.

Aspects of the present disclosure provide a method of making an active pharmaceutical ingredient, comprising steps, in any order, of mixing: at least one pDC modulating cannabinoid or terpene; at least one monocyte-cell modulating cannabinoid or terpene; and at least one T-cell modulating cannabinoid or terpene.

In some embodiments, the at least one pDC modulating cannabinoid or terpene, at least one T-cell modulating cannabinoid or terpene, and at least one monocyte-modulating cannabinoid or terpene is added to a Cannabis sativa extract. In some embodiments, the Cannabis sativa extract is prepared from a Cannabis sativa strain selected to best approximate the determined composition of the active ingredient.

In some embodiments, each of the pDC modulating cannabinoid or terpene, T-cell modulating cannabinoid or terpene, and monocyte-modulating cannabinoid or terpene is synthetic or biosynthetic. In some embodiments, the active ingredient or the unit dosage form is produced by the method of making the active ingredient or unit dosage.

Aspects of the present disclosure provide a pharmaceutical composition comprising the active ingredient and a pharmaceutically acceptable carrier or diluent.

In some embodiments, the composition is an oil, an emulsion, a gel, or an aerosol. In some embodiments, the composition comprises a nanoparticle or nanoemulsion encapsulating the active ingredient.

In some embodiments, the active ingredient is present in the pharmaceutical composition at a concentration of 0.01 mg/ml. In some embodiments, the active ingredient is present in the pharmaceutical composition at a concentration of at least 0.1 mg/ml. In some embodiments, the active ingredient is present in the pharmaceutical composition at a concentration of at least 0.5 mg/ml. In some embodiments, the active ingredient is present in the pharmaceutical composition at a concentration of at least 1 mg/ml.

In some embodiments, the active ingredient is present in the pharmaceutical composition at a concentration of at least 10 mg/ml. In some embodiments, the active ingredient is present in the pharmaceutical composition at a concentration of at least 20 mg/ml.

Aspects of the present disclosure provide a method of modulating an immune response, the method comprising: administering an effective amount of the active pharmaceutical ingredient, the pharmaceutical composition or the unit dosage form to the patient who requires modulation of an immune response.

In some embodiments, the patient has a condition selected from: an ulcer, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND). In some embodiments, the patient has a condition selected from: Cytokine Release Syndrome (CRS), Cytokine Storm Syndrome (CSS), Macrophage Activation Syndrome (MAS), Acute Respiratory Distress (ARD), secondary hemophagocytic lymphohistiocytosis (sHLH), and sHLH secondary to viral or bacterial infections such as severe forms of COVID-19.

In some embodiments, the effective amount of the active pharmaceutical ingredient, pharmaceutical composition, or unit dose form reduces the level of TNFα secreted by pDCs. In some embodiments, the effective amount of the active pharmaceutical ingredient, pharmaceutical composition, or unit dose form reduces the level of IFNα secreted by pDCs. In some embodiments, the effective amount of the active pharmaceutical ingredient, pharmaceutical composition, or unit dose form reduces the level of IL-6 secreted by activated CD14+ CD16+ monocytes. In some embodiments, the effective amount of the active pharmaceutical ingredient, pharmaceutical composition, or unit dose form reduces phagocytosis by CD14+CD16+ monocytes. In some embodiments, the effective amount of the active pharmaceutical ingredient, pharmaceutical composition, or unit dose form reduces the level of TNFα secreted by CD14+CD16+ monocytes.

In some embodiments, the effective amount of the active pharmaceutical ingredient, pharmaceutical composition, or unit dose form reduces the level of one or more co-stimulatory molecule expressed by CD14+CD16+ monocytes. In some embodiments, the one or more co-stimulatory molecule is selected from the group consisting of: HLA-DR, CD80, and CD86.

In some embodiments, the effective amount of the active pharmaceutical ingredient, pharmaceutical composition, or unit dose form suppresses TNFα expression by CD4+ T cells, CD8+ T cells, or CD4+ and CD8+ T cells. In some embodiments, the effective amount of the active pharmaceutical ingredient, pharmaceutical composition, or unit dose form reduces the level of IFNγ expression by CD4+ T cells, CD8+ T cells, or CD4+ and CD8+ T cells.

In some embodiments, the the effective amount of the active pharmaceutical ingredient, pharmaceutical composition, or unit dose form reduces the level of IL-2 secreted by T cells.

In some embodiments, the the T cell is selected from a CD4+ T cell, a CD8+ T cell, or a CD4+ and CD8+ T cell.

In some embodiments, the effective amount of the active pharmaceutical ingredient, pharmaceutical composition, or unit dose form is administered by inhalation.

In some embodiments, the the effective amount of the active pharmaceutical ingredient, pharmaceutical composition, or unit dose form is administered orally.

In some embodiments, the the effective amount of the active pharmaceutical ingredient, pharmaceutical composition, or unit dose form is administered by buccal administration.

In some embodiments, the the effective amount of the active pharmaceutical ingredient, pharmaceutical composition, or unit dose form is administered by sublingual administration.

In some embodiments, the wherein the oral, buccal, or sublingual administration comprises nanoparticle administration.

In some embodiments, the the effective amount of the active pharmaceutical ingredient, pharmaceutical composition, or unit dose form is administered by injection.

In some embodiments, the effective amount of the active pharmaceutical ingredient, pharmaceutical composition, or unit dose form is administered by topical application.

In some embodiments, the effective amount of the active pharmaceutical ingredient, pharmaceutical composition, or unit dose form is administered in an amount of less than 1 g per dose.

In some embodiments, the effective amount of the active pharmaceutical ingredient, pharmaceutical composition, or unit dose form is administered in an amount of less than 500 mg per dose, less than 100 mg per dose, less than 10 mg per dose.

In some embodiments, the effective amount of the active pharmaceutical ingredient, pharmaceutical composition, or unit dose form is administered in an amount ranging from 1 to 30 mg per dose.

In some embodiments, the effective amount of the active pharmaceutical ingredient, pharmaceutical composition, or unit dose form is administered p.r.n.

In some embodiments, the effective amount of the active pharmaceutical ingredient, pharmaceutical composition, or unit dose form is administered after a measured increase in an inflammatory response as compared to a patient that does not have an immune response.

In some embodiments, the effective amount of the active pharmaceutical ingredient, pharmaceutical composition, or unit dose form is administered 6 hours after, 24 hours after, or 4 days after a measured increase in an inflammatory response as compared to a patient that does not have an immune response.

In some embodiments, the effective amount of the active pharmaceutical ingredient, pharmaceutical composition, or unit dose form is administered once a day, 2-4 times a day, once a week, 2-4 times a week, or once every two weeks.

In some embodiments, the pDC modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the monocyte-modulating cannabinoid or terpene is present in an amount sufficient to achieve a mean peak concentration (Cmax) in plasma and/or target tissue of at least 0.001 μM, at least 0.01 μM, at least 0.1 μM, at least 1 μM, or at least 5 μM, when administered.

In some embodiments, the T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the method comprises 7 mg to 29 mg of CBD, 0.001 mg to 28 mg of CBN, and 0.01 mg to 0.05 mg of THC; 0.7 mg to 5.2 mg of CBDV, 0.001 mg to 28 mg of CBN: and 0.0007 mg to 0.002 mg of Phytol A; 0.7 mg to 5.2 mg of CBDV, 7 mg to 29 mg of CBD, 0.001 mg to 28 mg of CBN, and 0.01 mg to 0.6 mg of CBG; CBD, 0.001 mg to 28 mg of CBN, and 0.01 mg to 0.6 mg of CBG CBG; or 0.7 mg to 5.2 mg of CBDV, 7 mg to 29 mg of CBD, 0.001 mg to 28 mg of CBN, and 0.01 mg to 0.05 mg of THC.

Aspects of the present disclosure provide an active pharmaceutical ingredient comprising: (a) a plasmacytoid dendritic cell-(pDC) modulating cannabinoid or terpene; and (b) optionally a T-cell-modulating cannabinoid or terpene.

In some embodiments, the pDC modulating cannabinoid or terpene comprises a single cannabinoid or terpene.

In some embodiments, the pDC modulating cannabinoid or terpene comprises two or more cannabinoids or terpenes.

In some embodiments,the T-cell modulating cannabinoid or terpene is a CD4+ T-cell modulating cannabinoid or terpene, a CD8+ T-cell modulating cannabinoid or terpene, or a CD4+ and CD8+ T-cell modulating cannabinoid or terpene.

In some embodiments, the T-cell modulating cannabinoid or terpene comprises a single cannabinoid or terpene.

In some embodiments, the T-cell modulating cannabinoid or terpene comprises two or more cannabinoids or terpenes.

In some embodiments, the T-cell modulating cannabinoid or terpene comprises two or more cannabinoids or terpenes.

In some embodiments, T-cell modulating cannabinoid or terpene is selected from one or more of: cannabidiol (CBD), Cannabidivarin (CBDV), Cannabinol (CBN), Cannabigerol (CBG), phytol A, and tetrahydrocannabinol (THC).

In some embodiments, the pDC modulating cannabinoid or terpene is selected from one or more of cannabidiol (CBD), Cannabidivarin (CBDV), Cannabinol (CBN), Cannabigerol (CBG), phytol A, and tetrahydrocannabinol (THC).

In some embodiments, the active pharmaceutical ingredient comprises two or more of cannabidiol (CBD), Cannabidivarin (CBDV), Cannabinol (CBN), Cannabigerol (CBG), phytol A, and tetrahydrocannabinol (THC).

In some embodiments, the active pharmaceutical ingredient consists essentially of: CBDV, CBD, and CBN; CBD, CBDV, CBN, Phytol A, and THC; CBD, CBN, Phytol A, and THC; CBDV, CBD, CBN, and Phytol A; CBDV, CBD, CBN, CBG, and THC; CBDV, CBN, and THC; CBDV, CBD, CBN, and THC; CBD, CBN, and Phytol A; CBD, CBN, CBG, and THC; or CBDV, CBN, and THC.

In some embodiments, the active pharmaceutical ingredient consists essentially of: 100-parts CBDV:500 CBD:1-part CBN; 5,000-parts CBD:1,000-parts CBDV:5,000-parts CBN:1-part Phytol A:10-parts THC; 5,000-parts CBD:5,000-parts CBN:1-part Phytol A:10-parts THC; 1,000-parts CBDV:5,000-parts CBD:5,000-parts CBN:1-part Phytol A; 100-parts CBDV:500-parts CBD:500-parts CBN:10-parts CBG:1-part THC; 100-parts CBDV:1-part CBN:1-part THC; 100-parts CBDV:500-parts CBD:500-parts CBN:1-part THC; 5,000-parts CBD:5,000-parts CBN:1-part Phytol A; 500-parts CBD:500-parts CBN:10-parts CBG:1-part THC; or 100-parts CBDV:500-parts CBN:1-part THC.

In some embodiments, each of the pDC-modulating cannabinoid or terpene, and optionally the T-cell modulating cannabinoid or terpene is synthetic or biosynthetic.

In some embodiments, the active pharmaceutical ingredient further comprises an optional Lymphopenia-reducing cannabinoid.

In some embodiments, the optional Lymphopenia-reducing cannabinoid is tetrahydrocannabinol (THC).

In some embodiments, the pDC-modulating cannabinoid or terpene, and optionally the T-cell modulating cannabinoid or terpene collectively constitute at least 75% by weight of the active ingredient.

In some embodiments, all compounds in the active ingredient other than the pDC-modulating cannabinoid or terpene, and optionally the T-cell modulating cannabinoid or terpene are extractable from Cannabis sativa.

In some embodiments, the active pharmaceutical ingredient is substantially free of tetrahydrocannabinol (THC).

In some embodiments, the pDC modulating cannabinoid or terpene constitutes 0.01-99% by weight of the active ingredient; and the optional T-cell modulating cannabinoid or terpene constitutes 0.01-99% by weight of the active ingredient.

In some embodiments, the pDC modulating cannabinoid or terpene is a pDC-targeting cannabinoid or terpene.

In some embodiments, the optional T-cell modulating cannabinoid or terpene is a CD4+ T-cell targeting cannabinoid or terpene.

In some embodiments, the optional T-cell modulating cannabinoid or terpene is a CD8+ T-cell-targeting cannabinoid or terpene.

In some embodiments, the optional T-cell modulating cannabinoid or terpene is a CD4+ and CD8+ T-cell-targeting cannabinoid or terpene.

In some embodiments, the optional T-cell modulating cannabinoid or terpene comprises two or more cannabinoids or terpenes, wherein at least one cannabinoid or terpene is a CD4+ T-cell-targeting cannabinoid or terpene.

In some embodiments, the optional T-cell modulating cannabinoid or terpene comprises two or more cannabinoids or terpenes, wherein at least one cannabinoid or terpene is a CD8+ T-cell-targeting cannabinoid or terpene.

Aspects of the present disclosure provide a unit dosage form comprising the active pharmaceutical ingredient.

In some embodiments, the pDC modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the optional T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the optional T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the unit dose comprises 0.7 mg to 5.2 mg of CBDV, 7 mg to 29 mg of CBD, and 0.001 mg to 28 mg of CBN; 7 mg to 29 mg of CBD, 0.7 mg to 5.2 mg of CBDV, 0.001 mg to 28 mg of CBN, 0.0007 mg to 0.002 mg of Phytol A, and 0.01 mg to 0.05 mg of THC; 7 mg to 29 mg of CBD, 0.001 mg to 28 mg of CBN, 0.0007 mg to 0.002 mg of Phytol A, and 0.01 mg to 0.05 mg of THC; 0.7 mg to 5.2 mg of CBDV, 7 mg to 29 mg of CBD, 0.001 mg to 28 mg of CBN, and 0.0007 mg to 0.002 mg of Phytol A; 0.7 mg to 5.2 mg of CBDV, 7 mg to 29 mg of CBD, 0.001 mg to 28 mg of CBN, 0.01 mg to 0.6 mg of CBG, and 0.01 mg to 0.05 mg of THC; 0.7 mg to 5.2 mg of CBDV, CBN, and 0.01 mg to 0.05 mg of THC; 0.7 mg to 5.2 mg of CBDV, 7 mg to 29 mg of CBD, 0.001 mg to 28 mg of CBN, and 0.01 mg to 0.05 mg of THC; 7 mg to 29 mg of CBD, 0.001 mg to 28 mg of CBN, and 0.0007 mg to 0.002 mg of Phytol A; 7 mg to 29 mg of CBD, 0.001 mg to 28 mg of CBN, 0.01 mg to 0.6 mg of CBG, and 0.01 mg to 0.05 mg of THC; or 0.7 mg to 5.2 mg of CBDV, 0.001 mg to 28 mg of CBN, and 0.01 mg to 0.05 mg of THC.

Aspects of the present disclosure provide a method of making an active pharmaceutical ingredient, comprising steps, in any order, of mixing: at least one pDC modulating cannabinoid or terpene; and optionally at least one T-cell modulating cannabinoid or terpene.

In some embodiments, the at least one pDC modulating cannabinoid or terpene and at least one optional T-cell modulating cannabinoid or terpene is added to a Cannabis sativa extract.

In some embodiments, each of the pDC modulating cannabinoid or terpene, and optional T-cell modulating cannabinoid or terpene is synthetic or biosynthetic.

Aspects of the present disclosure provide a pharmaceutical composition comprising the active ingredient and a pharmaceutically acceptable carrier or diluent.

In some embodiments, the composition is an oil, an emulsion, a gel, or an aerosol.

In some embodiments, the composition comprises a nanoparticle or nanoemulsion encapsulating the active ingredient.

In some embodiments, the active ingredient is present in the pharmaceutical composition at a concentration of 0.01 mg/ml.

In some embodiments, the active ingredient is present in the pharmaceutical composition at a concentration of at least 0.1 mg/ml.

In some embodiments, the active ingredient is present in the pharmaceutical composition at a concentration of at least 0.5 mg/ml.

In some embodiments, the active ingredient is present in the pharmaceutical composition at a concentration of at least 1 mg/ml.

In some embodiments, the active ingredient is present in the pharmaceutical composition at a concentration of at least 10 mg/ml.

In some embodiments, the active ingredient is present in the pharmaceutical composition at a concentration of at least 20 mg/ml.

Aspects of the present disclosure provide a method of suppressing an immune response in a patient, the method comprising: administering an effective amount of the active pharmaceutical ingredient, the pharmaceutical composition or the unit dosage form to the patient who has inflammation.

In some embodiments, the patient has a chronic inflammatory disease.

In some embodiments, the chronic inflammatory disease is selected from: an ulcer, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

In some embodiments, the effective amount of the active pharmaceutical ingredient, pharmaceutical composition, or unit dose form reduces the level of TNFα secreted by pDCs.

In some embodiments, the effective amount of the active pharmaceutical ingredient, pharmaceutical composition, or unit dose form reduces the level of IFNα secreted by pDCs.

In some embodiments, the effective amount of the active pharmaceutical ingredient, pharmaceutical composition, or unit dose form suppresses TNFα expression by CD4+ T cells, CD8+ T cells, or CD4+ an8+ T cells.

In some embodiments, the effective amount of the active pharmaceutical ingredient, pharmaceutical composition, or unit dose form reduces the level of IFNγ expression by CD4+ T cells, CD8+ T cells, or CD4+ and CD8+ T cells.

In some embodiments, the effective amount of the active pharmaceutical ingredient, pharmaceutical composition, or unit dose form reduces the level of IL-2 secreted by T cells.

In some embodiments, the T cell is selected from a CD4+ T cell, a CD8+ T cell, or a CD4+ and CD8+ T cell.

In some embodiments, the effective amount of the active pharmaceutical ingredient, pharmaceutical composition, or unit dose form is administered by inhalation.

In some embodiments, the effective amount of the active pharmaceutical ingredient, pharmaceutical composition, or unit dose form is administered orally.

In some embodiments, the effective amount of the active pharmaceutical ingredient, pharmaceutical composition, or unit dose form is administered by buccal administration.

In some embodiments, the effective amount of the active pharmaceutical ingredient, pharmaceutical composition, or unit dose form is administered by sublingual administration.

In some embodiments, the oral, buccal, or sublingual administration comprises nanoparticle administration.

In some embodiments, the effective amount of the active pharmaceutical ingredient, pharmaceutical composition, or unit dose form is administered by injection.

In some embodiments, the effective amount of the active pharmaceutical ingredient, pharmaceutical composition, or unit dose form is administered by topical application.

In some embodiments, the effective amount of the active pharmaceutical ingredient, pharmaceutical composition, or unit dose form is administered in an amount of less than 1 g per dose.

In some embodiments, the effective amount of the active pharmaceutical ingredient, pharmaceutical composition, or unit dose form is administered in an amount of less than 500 mg per dose, less than 100 mg per dose, or less than 10 mg per dose.

In some embodiments, the effective amount of the active pharmaceutical ingredient, pharmaceutical composition, or unit dose form is administered in an amount ranging from 1 to 30 mg per dose.

In some embodiments, the effective amount of the active pharmaceutical ingredient, pharmaceutical composition, or unit dose form is administered p.r.n.

In some embodiments, the effective amount of the active pharmaceutical ingredient, pharmaceutical composition, or unit dose form is administered after a measured increase in a pro-inflammatory response as compared to a patient that does not have a pro-inflammatory response.

In some embodiments, the effective amount of the active pharmaceutical ingredient, pharmaceutical composition, or unit dose form is administered 6 hours after, 24 hours after, or 4 days after a measured increase in a pro-inflammatory response as compared to a patient that does not have a pro-inflammatory response.

In some embodiments, the effective amount of the active pharmaceutical ingredient, pharmaceutical composition, or unit dose form is administered once a day, 2-4 times a day, once a week, 2-4 times a week, or once every two weeks.

In some embodiments, the pDC modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the optional T-cell modulating cannabinoid or terpene is present in an amount sufficient to achieve a mean peak concentration (Cₘₐₓ) in plasma and/or target tissue of at least 0.001 μM, at least 0.01 μM, at least 0.1 μM, at least 1 μM, or at least 5 μM, when administered.

In some embodiments, the active pharmaceutical ingredient comprising: 7 mg to 29 mg of CBD, 0.001 mg to 28 mg of CBN, and 0.01 mg to 0.05 mg of THC; 0.7 mg to 5.2 mg of CBDV, 0.001 mg to 28 mg of CBN: and 0.0007 mg to 0.002 mg of Phytol A; 0.7 mg to 5.2 mg of CBDV, 7 mg to 29 mg of CBD, 0.001 mg to 28 mg of CBN, and 0.01 mg to 0.6 mg of CBG; CBD, 0.001 mg to 28 mg of CBN, and 0.01 mg to 0.6 mg of CBG CBG; or 0.7 mg to 5.2 mg of CBDV, 7 mg to 29 mg of CBD, 0.001 mg to 28 mg of CBN, and 0.01 mg to 0.05 mg of THC.

Aspects of the present disclosure provide an active pharmaceutical ingredient comprising two or more monocyte-modulating cannabinoids.

In some embodiments, the monocyte modulating cannabinoids are synthetic or biosynthetic.

In some embodiments, the active pharmaceutical ingredient comprises two or more of cannabidiol (CBD), Cannabidivarin (CBDV), Cannabinol (CBN), Cannabigerol (CBG), phytol A, and tetrahydrocannabinol (THC).

In some embodiments, the active pharmaceutical ingredient comprises: CBDV, CBN, CBG, and THC.

In some embodiments, the active pharmaceutical ingredient consists essentially of: 100-pars CBDV :500-parts CBN:10-parts CBG: and 1-part THC.

In some embodiments, the two or more monocyte modulating cannabinoids collectively constitute at least 75% by weight of the active ingredient.

Aspects of the present disclosure provide a unit dosage form comprising the active pharmaceutical ingredient.

In some embodiments, at least one of the two or more monocyte modulating cannabinoids are present in an amount sufficient to achieve a mean peak concentration (Cₘₐₓ) in plasma and/or target tissue of at least 0.001 μM, at least 0.001 μM, at least 0.1 μM, at least 1 uM, or at least 5 μM when administered.

Aspects of the present disclosure provide a method of making an active pharmaceutical ingredient, comprising steps, in any order, of mixing: two or more monocyte-modulating cannabinoids.

Aspects of the present disclosure provide a pharmaceutical composition comprising the active ingredient of and a pharmaceutically acceptable carrier or diluent.

In some embodiments, the composition is an oil, an emulsion, a gel, or an aerosol.

In some embodiments, the composition comprises a nanoparticle or nanoemulsion encapsulating the active ingredient.

In some embodiments, the active ingredient is present in the pharmaceutical composition at a concentration of 0.01 mg/ml.

In some embodiments, the active ingredient is present in the pharmaceutical composition at a concentration of at least 0.1 mg/ml.

T In some embodiments, the active ingredient is present in the pharmaceutical composition at a concentration of at least 0.5 mg/ml.

In some embodiments, the active ingredient is present in the pharmaceutical composition at a concentration of at least 1 mg/ml.

In some embodiments, the active ingredient is present in the pharmaceutical composition at a concentration of at least 10 mg/ml.

In some embodiments, the active ingredient is present in the pharmaceutical composition at a concentration of at least 20 mg/ml.

Aspects of the present disclosure provide a method of inducing a proinflammatory response in a patient the method comprising: administering an effective amount of the active pharmaceutical ingredient, the pharmaceutical composition or the unit dosage form to the patient who has a localized infection.

In some embodiments, the effective amount of the active pharmaceutical ingredient, pharmaceutical composition, or unit dose form reduces the level of TNFα secreted by CD14+CD16+monocytes.

In some embodiments, the methods of the present disclosure further comprises administering an effective amount of at least one second therapeutic agent selected from the group consisting of an antiviral agent, an antibacterial agent, an IL-6 antagonist, an angiotensin receptor blocker (ARB), granulocyte/macrophage colony stimulating factor (GM-CSF) antagonist, hydroxychloroquine, chloroquine, and COVID-19 immune serum or plasma.

In some embodiments, the at least one second therapeutic agent is an antiviral agent. In some embodiments, the antiviral agent is favipiravir. In some embodiments, the antiviral agent is remdesivir. In some embodiments, the at least one second therapeutic agent is an antibacterial agent selected from the group consisting of azithromycin, tobramycin, aztreonam, ciprofloxacin, meropenem, cefepime, cetadizine, imipenem, piperacillin-tazobactam, amikacin, gentamicin, and levofloxacin. In some embodiments, the at least one second therapeutic agent is an IL-6 antagonist selected from the group consisting of an anti-IL-6 antibody or an antigen binding fragment thereof, an anti-IL-6 receptor antibody or an antigen binding fragment thereof, and a JAK/STAT inhibitor. In some embodiments, the at least one second therapeutic agent is a GM-CSF antagonist. In some embodiments, the GM-CSF antagonist is gemsilumab.

These and other aspects of the invention are described in further detail below.

### 4. BRIEF DESCRIPTION OF THE DRAWINGS

FIG. 1 provides a summary table of the newly reported immunomodulating activity shown in the present application by each cannabis-based compound on each immune cell type evaluated. Plasmacytoid dendritic cells (pDC), monocytes, and T cells were targeted because of their importance in the human immune response to viral infections and their responsiveness to cannabinoids and terpenes through receptor mediated immune modulating reactions. The table summarizes the number of immune parameters that were significantly affected by exposure to a specific compound (left-most column) in a statistically significant manner for a given cell type (top row). They were also letter-coded to indicate if the change involved a: decreased cytokines or immune processes (“A”), mixed effect on cytokines or immune processes (“B”), or increased cytokines or immune processes (“C”). As indicated in the second row, the immune parameters measured per cell type are as follows: 1) pDC cells: IFNα and TNFα cytokine levels; 2) activated monocytes: IL-1β, IL-6, and TNFα cytokine levels, phagocytic activity, CD80, CD86, and HLA-DR cell co-stimulation marker levels; and 3) CD4+ and CD8+ T cells: IFNγ and IL-4 cytokine levels, T cell proliferation and activation markers CD25 and CD69. Novel findings of the present application regarding the immune modulating activities of this broad sampling of cannabis-based compounds revealed new strategies for addressing CRS/MAS while preserving anti-viral immunity, as described further below.

FIG. 2 illustrates critical novel findings of the present application behind the rational development of cannabinoid containing complex mixtures for CRS/MAS. The key cytokines and immune processes targeted for the treatment of CRS/MAS while preserving anti-viral immunity are listed in the top row. The cannabinoids and terpenes (left-most column) were tested over the following range: 0.001, 0.01, 0.1, 1.0, and 10 μM. Those cannabinoids and terpenes identified by these experiments as having a statistically significant impact on these target cytokines (IL-1β, IL-6, TNFα, IFNα, and IFNγ) and anti-viral immune processes (IFNα production by pDC and T cell proliferation) were indicated in the table based on the cytokine or process that achieved statistical significance. They were also letter-coded to indicate if the change involved: decreased cytokines or immune processes (“M”), mixed effect on cytokines or immune processes (“N”), or increased cytokines or immune processes (“O”). The therapeutic range of concentrations was provided for those cannabinoids and terpenes that were included in the cannabinoid containing complex mixtures for the treatment of CRS, MAS, ARD, sHLH. However, “contra-indicated” is noted in the therapeutic range column for those compounds that the data set excluded from use in the complex mixtures and that data is included in this application. Additional information pertaining to the rational design of the cannabinoid containing complex mixtures is provided in the notes section. Significant experimental results related to the rational-design of these cannabinoid-containing complex mixtures are illustrated as FIGS. 5 through 23.

FIG. 3A and FIG. 3B provide examples of molar ratios of selected cannabinoids and/or terpenes used in cannabinoid containing complex mixtures for the treatment of CRS/MAS by reducing particular proinflammatory cytokines, while preserving anti-viral immune pathways and reducing lymphopenia.

FIG. 4 provides examples of dose ranges of each cannabinoid or terpene composition for achieving desired concentrations for therapeutic effects in cannabinoid containing complex mixtures.

FIGS. 5A-5D demonstrate the IL-1β- and IL-6-decreasing potential of cannabidiol (CBD) in monocytes. The phagocytic activity of monocytes was also reduced by CBD. PBMC were isolated from human peripheral blood and pre-treated with the indicated concentrations of cannabidiol for 30 minutes, followed by stimulation with 15 μg/mL CpG-ODN for 6 hours (FIG. 5C) or 1000 ng/mL LPS for 24 hours (FIG. 5B). Supernatants were collected and IL-1β quantified by ELISA (FIG. 5A). Cells were non-enzymatically released from culture plates and stained for CD14 (FIG. 5B) and assessed for the percentage of TNFα+ monocytes (FIG. 5B), or IL-6+ monocytes (FIG. 5B) by intracellular staining. For FIG. 5D, PBMC were isolated from human peripheral blood and pre-treated with the indicated concentrations of cannabidiol for 30 minutes, followed by treatment with 3.0 mg/mL of pHrodo Green BioParticles (FIG. 5D). In FIG. 5D, cells were treated with 3.0 mg/mL of pHrodo Green BioParticles and assessed for phagocytosis monocytes by flow cytometry within 2 hours. Data are normalized to each donor's vehicle control when appropriate and represented as mean±SEM. N=4-6 donors per treatment group. ****p<0.0001 when compared to VH (FIGS. 5A-5C), or ***p<0.001 when compared to VH (FIG. 5D), by repeated measure one-way ANOVA with Dunnett's posttest.

FIGS. 6A-6C illustrate the IL-1β- and IL-6-decreasing potential of cannabidivarin (CBDV) in monocytes; as well as, their effect on phagocytosis. CBDV significantly decreased IL-1β (10 μM ), IL-6 (1-10 μM), and phagocytosis (10 μM) in monocytes. CBDV will not be formulated at concentrations (equivalent dosages) near 0.01 μM CBDV where IL-1β is increased in monocytes. PBMC were isolated from human peripheral blood and pre-treated with the indicated concentrations of cannabidivarin for 30 minutes, followed by stimulation with 1000 ng/mL LPS for 24 hours (FIGS. 6A, 6B). Cells were non-enzymatically released from culture plates and stained for CD14 (FIG. 6B) and assessed for the percentage of IL-6+ monocytes (FIG. 6B) by intracellular staining. Supernatants were collected and IL-1β quantified by ELISA (FIG. 6A). For FIG. 6C, PBMC were isolated from human peripheral blood and pre-treated with the indicated concentrations of cannabidivarin for 30 minutes, followed by treatment with 3.0 mg/mL of pHrodo Green BioParticles (FIG. 6C). In FIG. 6C, cells were treated with 3.0 mg/mL of pHrodo Green BioParticles and assessed for phagocytosis monocytes by flow cytometry within 2 hours. Data are normalized to each donor's vehicle control when appropriate and represented as mean±SEM. N=4-6 donors per treatment group. *p<0.05, **p<0.01, ***p<0.001, ****P<0.0001 when compared to VH (FIGS. 6A, 6C), or *p<0.05, ***p<0.001 when compared to VH (FIG. 6B) by repeated measure one-way ANOVA with Dunnett's posttest.

FIGS. 7A-7C show additional data relevant to the formulation of therapeutic mixtures. Cannabidivarin (CBDV) significantly decreased CD80+(10 μM) & HLA-DR (10 μM) co-stimulatory markers in monocytes, but increased the CD86+ (10 μM) co-stimulatory marker, so the therapeutic range for CBDV was centered around 1 μM to optimize its IL-6 decreasing potential. PBMC were isolated from human peripheral blood and pre-treated with the indicated concentrations of cannabidivarin for 30 minutes, followed by stimulation with 1000 ng/mL LPS (FIGS. 7A-7C). Cells suspensions were stimulated for 2 days with 1000 ng/mL LPS, following non-enzymatic release from culture plates and surface stained for HLA-DR, CD80, and CD86 to assess percentage or levels (mean fluorescence intensity—MFI) of CD80+ (FIG. 7A), CD86+ (FIG. 7B), or HLA-DR+ (FIG. 7C) monocytes. Data are normalized to each donor's vehicle control when appropriate and presented as mean±SEM. N=4-6 donors per treatment group. *p<0.05, ***p<0.001 when compared to VH by repeated measure one-way ANOVA with Dunnett's posttest.

FIGS. 8A-8C provide additional data on the effects of CBDV on CD4+ and CD8+ T cells. At 0.01 μM, CBDV significantly decreased IFNγ, but also increased IL-1β (FIG. 6A), which is contra-indicated. At 10 μM CBDV also decreased CD25+ and CD69+ T cell activation markers in CD8+ T cells. The therapeutic range for CBDV was centered around 1 μM to optimize its IL-6 (FIG. 6B) decreasing potential. PBMC were isolated from human peripheral blood and pre-treated with the indicated concentrations of cannabidivarin for 30 minutes on culture plates coated with 5 μg/mL anti-CD3, followed by stimulation with 5 μg/mL anti-CD28 and supplemented with 5 ng/mL IL-2. Cells were cultured for 24 hours (FIGS. 8B, 8C), or 5 days (FIG. 8A). Cells were non-enzymatically released from the culture plates and stained for CD25, and CD69 and assessed for percentage of CD25+ (FIG. 8B) or CD69+(FIG. 8C) CD8+ T cells. Cells were further stimulated with 50 ng/mL PMA and 1000 ng/mL ionomycin, Golgi blocked for 6 hours and assessed for intracellular IFNγ (FIG. 8A) CD8+ T cells. Data are normalized to each donor's vehicle control when appropriate and presented as mean±SEM. N=4-6 donors per treatment group. *p<0.05, **p<0.01, ***p<0.001, ****p<0.0001 when compared to VH by repeated measure one-way ANOVA with Dunnett's posttest.

FIGS. 9A-9B reveal the TNFα decreasing potential of cannabinol (CBN). At 10 μM, CBN significantly decreased TNFα, as well as decreasing phagocytosis in monocytes. PBMC were isolated from human peripheral blood and pre-treated with the indicated concentrations of cannabinol for 30 minutes, followed by stimulation with 15 μg/mL CpG-ODN for 6 hours (FIG. 9A). Cells were non-enzymatically released from culture plates and assessed for the percentage of TNFα+ monocytes (FIG. 9A) by intracellular staining. For FIG. 9B, PBMC were isolated from human peripheral blood and pre-treated with the indicated concentrations of cannabinol for 30 minutes, followed by treatment with 3.0 mg/mL of pHrodo Green BioParticles (FIG. 9B). In FIG. 9B, cells were treated with 3.0 mg/mL of pHrodo Green BioParticles and assessed for phagocytosis monocytes by flow cytometry within 2 hours. Data are normalized to each donor's vehicle control when appropriate and presented as mean±SEM. N=4-6 donors per treatment group. Data are normalized to each donor's vehicle control when appropriate and represented as mean±SEM. N=4-6 donors per treatment group. *p<0.05 when compared to VH (FIG. 9A), or **p<0.01 when compared to VH (FIG. 9B), by repeated measure one-way ANOVA with Dunnett's posttest.

FIGS. 10A-10B demonstrate the IFNγ decreasing potential of cannabinol (CBN) in both CD4+ T cells and CD8+ T cells. At between 0.001-0.1 μM, CBN significantly decreased IFNγ in both CD4+ and CD8+ T cells. PBMC were isolated from human peripheral blood and pre-treated with the indicated concentrations of cannabinol for 30 minutes in culture plates coated with 5 μg/mL anti-CD3, followed by stimulation with 5 μg/mL anti-CD28 and supplemented with 5 ng/mL IL-2. Cells were cultured for 5 days (FIG. 10A). Cells were further stimulated with 50 ng/mL PMA and 1000 ng/mL ionomycin, Golgi blocked for 6 hours and assessed for intracellular IFNγ (FIG. 10A) CD4+ T cells or IFNγ (FIG. 10B) CD8+ T cells. Data are normalized to each donor's vehicle control when appropriate and presented as mean±SEM. N=4-6 donors per treatment group. *p<0.05, **p<0.01 when compared to VH (FIG. 10A), or *p<0.05, ***p<0.001 when compared to VH (FIG. 10B), by repeated measure one-way ANOVA with Dunnett's posttest.

FIGS. 11A-11C demonstrate the IFNγ decreasing potential of cannabigerol (CBG) in T cells. At between 0.01-1 μM, CBG significantly decreased IFNγ in both CD4+ and CD8+ T cells. At between 0.1-1 μM, CBG significantly decreased IL-4 levels in CD4+ T cells. PBMC were isolated from human peripheral blood and pre-treated with the indicated concentrations of cannabigerol for 30 minutes in culture plates coated with 5 μg/mL anti-CD3, followed by stimulation with 5 μg/mL anti-CD28 and supplemented with 5 ng/mL IL-2. Cells were cultured for 5 days (FIGS. 11A-11B). Cells were further stimulated with 50 ng/mL PMA and 1000 ng/mL ionomycin, Golgi blocked for 6 hours and assessed for intracellular IFNγ (FIG. 11A) and IL-4 (FIG. 11B) CD4+ T cells. For FIG. 11C, cells were cultured for 5 days. Cells were further stimulated with 50 ng/mL PMA and 1000 ng/mL ionomycin, Golgi blocked for 6 hours and assessed for intracellular IFNγ (FIG. 11C) CD8+ T cells. Data are normalized to each donor's vehicle control when appropriate and presented as mean±SEM. N=4-6 donors per treatment group. *p<0.05, **p<0.01 when compared to VH (FIGS. 11A-11B), or *p<0.05 when compared to VH (FIG. 11C) by repeated measure one-way ANOVA with Dunnett's posttest.

FIGS. 12A-12C show additional data relevant to the selection of the therapeutic range for CBG. At 10 μM, CBG significantly decreased IL-1β, but increased HLA-DR (10 μM) CD86+(1-10 μM) co-stimulatory markers on monocytes. The therapeutic range for CBG was selected to optimize the IFNγ decreasing potential (FIGS. 11A-11C) in the cannabinoid containing complex mixtures (CCCM). PBMC were isolated from human peripheral blood and pre-treated with the indicated concentrations of cannabigerol for 30 minutes, followed by stimulation with 1000 ng/mL LPS for 24 hours (FIG. 12A). Supernatants were collected and IL-1β quantified by ELISA (FIG. 12A). For FIGS. 12B-12C, PBMC were isolated from human peripheral blood and pre-treated with the indicated concentrations of cannabigerol for 30 minutes, followed by stimulation with 1000 ng/mL LPS (FIGS. 12C). Cells suspensions were stimulated for 2 days with 1000 ng/mL LPS, following non-enzymatic release from culture plates and surface stained for HLA-DR, and CD86 to assess percentage or levels (mean fluorescence intensity—MFI) of HLA-DR+ (FIG. 12B), or CD86+ (FIG. 12C) monocytes. Data are normalized to each donor's vehicle control when appropriate and represented as mean±SEM. N=4-6 donors per treatment group. *p<0.05 when compared to VH (FIG. 12A), or *p<0.05, **p<0.01, ****p<0.0001 when compared to VH (FIG. 12B-12C) by repeated measure one-way ANOVA with Dunnett's posttest.

FIGS. 13A-13B show the IFNγ decreasing potential of Phytol A. At 0.001 μM, phytol A significantly decreased IFNγ in CD4+ T cells. PBMC were isolated from human peripheral blood and pre-treated with the indicated concentrations of phytol for 30 minutes in culture plates coated with 5 μg/mL anti-CD3, followed by stimulation with 5 μg/mL anti-CD28 and supplemented with 5 ng/mL IL-2. Cells were cultured for 5 days (FIG. 13A-FIG. 13B). Cells were further stimulated with 50 ng/mL PMA and 1000 ng/mL ionomycin, golgi blocked for 6 hours and assessed for intracellular IFNγ from within CD4+ T cells (FIG. 13A) or IFNγ from within CD8+ T cells (FIG. 13B). Data are normalized to each donor's vehicle control when appropriate and presented as mean±SEM. N=4-6 donors per treatment group. **p<0.01 when compared to VH by repeated measure one-way ANOVA with Dunnett's posttest.

FIGS. 14A-14D demonstrate the Lymphopenia decreasing and T cell proliferating effect of tetrahydrocannabinol (THC) on T cells across the range of concentrations tested: 0.001, 0.01, 0.1, 1.0, and 10.0 μM THC. PBMC were isolated from human peripheral blood and pre-treated with the indicated concentrations of THC for 30 minutes in culture plates coated with 5 μg/mL anti-CD3, followed by stimulation with 5 μg/mL anti-CD28 and supplemented with 5 ng/mL IL-2. Cells were cultured for 5 days. Cells were stained for CD3, CD4, CD8, and CFSE and proliferation analyzed by FlowJo Proliferation tool to assess the population of CD4⁺ T cells that divided (FIG. 14A) and the average number of cell divisions (FIG. 14C). Cells were also stained for CD3, CD4, CD8, and CFSE and proliferation analyzed by FlowJo Proliferation tool to assess the population of CD8⁺ T cells that divided (FIG. 14B) and the average number of cell divisions (FIG. 14D). Data are normalized to each donor's vehicle control when appropriate and presented as mean±SEM. N=4-6 donors per treatment group. *p<0.05, when compared to VH by repeated measure one-way ANOVA with Dunnett's posttest.

FIGS. 15A-15B provide additional data relevant to establishing the therapeutic range of THC within the CCCM. At 10 μM, THC significantly decreased both TNFα and IFNα in plasmacytoid dendritic cells (pDC). Decreasing TNFα levels is therapeutically desirable, however, decreasing IFNα is contra-indicated. Therefore, we will be using THC at a lower concentration to preserve IFNα production in pDC that is an essential trigger for anti-viral immunity. THC will be used at 0.01 μM for its lymphopenia decreasing (FIGS. 14A-B) effects. PBMC were isolated from human peripheral blood and pre-treated with the indicated concentrations of THC for 30 minutes, followed by stimulation with 15 μg/mL CpG-ODN for 6 hours. Cells were non-enzymatically released from culture plates and stained for CD303, a highly specific marker for plasmacytoid dendritic cells, and assessed for the percentage of TNFa (FIG. 15A) in pDC exposed to cannabis-derived compounds relative to the amount of TNFα in vehicle exposed control pDC cell by intracellular staining, and also assessed for the percentage of IFNα (FIG. 15B) in pDC exposed to cannabis-derived compounds relative to the amount of IFNα in vehicle exposed control pDC cells by intracellular staining. Data are normalized to each donor's vehicle control and represented as mean±SEM. N=4-6 donors per treatment group. *p<0.05, **p<0.01 when compared to vehicle (VH) by repeated measure one-way ANOVA with Dunnett's posttest.

FIG. 16 provides additional data on immunomodulation by tetrahydrocannabinol (THC). At 10 μM, THC significantly decreased IL-1β levels in monocytes. PBMC were isolated from human peripheral blood and pre-treated with the indicated concentrations of THC for 30 minutes, followed by stimulation with 1000 ng/mL LPS for 24 hours. Supernatants were collected and IL-1β quantified by ELISA. Data are normalized to each donor's vehicle control when appropriate and represented as mean±SEM. N=4-6 human donors per treatment group. ****p<0.0001 when compared to VH by repeated measure one-way ANOVA with Dunnett's posttest.

FIGS. 17A-17B provide additional data on immunomodulation by tetrahydrocannabinol (THC). At 10 μM, THC significantly decreased TNFα and the co-stimulatory marker, CD80+, in monocytes. PBMC were isolated from human peripheral blood and pre-treated with the indicated concentrations of THC for 30 minutes, followed by stimulation with 15 μg/mL CpG-ODN for 6 hours (FIG17A) or 1000 ng/mL LPS for 2 days (FIG. 17B). Cells were non-enzymatically released from culture plates and assessed for the percentage of TNFα+ monocytes (FIG. 17A) by intracellular staining, or surface stained for CD14, HLA-DR, CD80, and CD86 to assess percentage or levels (mean fluorescence intensity—MFI) of CD80⁺ (FIG. 17B). THC did not cause a statistically significant change in the levels of the other two co-stimulatory markers (CD86+ or HLA-DR+). Data are normalized to each donor's vehicle control when appropriate and represented as mean±SEM. N=4-6 donors per treatment group. **p<0.01, ***p<0.001 when compared to VH by repeated measure one-way ANOVA with Dunnett's posttest.

FIGS. 18A-18B illustrate why alpha-Pinene (aP) was contra-indicated. Alpha-Pinene significantly decreased IFNα levels in plasmacytoid dendritic cells (pDC), which potentially decreases the pDC signal necessary to trigger anti-viral immunity. Lowering TNFα is therapeutically desired, but not at the expense of the IFNα. Because both happen at the same concentration range, aP may not be used in the cannabinoid containing complex mixtures. PBMC were isolated from human peripheral blood and pre-treated with the indicated concentrations of alpha-pinene for 30 minutes, followed by stimulation with 15 μg/mL CpG-ODN for 6 hours. Cells were non-enzymatically released from culture plates and stained for CD303 and assessed for the percentage of IFNα⁺ pDC (FIG. 18A) or TNFα⁺ pDC (FIG. 18B) by intracellular staining. Data are normalized to each donor's vehicle control when appropriate and represented as mean±SEM. N=4-6 donors per treatment group. *p<0.05 when compared to VH by repeated measure one-way ANOVA with Dunnett's posttest.

FIG. 19 illustrates why cannabichromene (CBC) was contra-indicated. At 0.1 μM, CBC significantly increased TNFα in monocytes, which is not therapeutically desired due to the pro-inflammatory role of this cytokine. PBMC were isolated from human peripheral blood and pre-treated with the indicated concentrations of cannabichromene for 30 minutes, followed by stimulation with 15 μg/mL CpG-ODN for 6 hours. Cells were non-enzymatically released from culture plates and stained for CD303 and assessed for the percentage of TNFα+ monocytes (FIG. 19) by intracellular staining. Data are normalized to each donor's vehicle control when appropriate and represented as mean±SEM. N=4-6 donors per treatment group. *p<0.05, when compared to VH by repeated measure one-way ANOVA with Dunnett's posttest.

FIGS. 20A-20B further illustrate why cannabichromene was contra-indicated. At 10 μM, CBC significantly decreased T cell proliferation, which would acerbate Lymphopenia. PBMC were isolated from human peripheral blood and pre-treated with the indicated concentrations of cannabichromene for 30 minutes in culture plates coated with 5 μg/mL anti-CD3, followed by stimulation with 5 μg/mL anti-CD28 and supplemented with 5 ng/mL IL-2. Cells were cultured for 4 days. Cells were non-enzymatically released from culture plates and stained for CD4 and CD8 and proliferation analyzed by FlowJo Proliferation tool to assess the population of CD4+ T cells that divided (FIG. 20A) or the population of CD8+ T cells that divided (FIG. 20B). Data are normalized to each donor's vehicle control when appropriate and presented as mean±SEM. N=4-6 donors per treatment group. *p<0.05, **p<0.01 when compared to VH (FIG. 21A) by repeated measure one-way ANOVA with Dunnett's posttest.

FIGS. 21A-21C illustrates another reason that cannabichromene (CBC) is contra-indicated. CBC significantly decreased IL-6 levels (10 μM) in monocytes, but it also increased the co-stimulatory markers HLA-DR (10 μM) & CD86+ (1-10 μM) on monocytes. PBMC were isolated from human peripheral blood and pre-treated with the indicated concentrations of cannabichromene for 30 minutes, followed by stimulation with 1000 ng/mL LPS for 24 hours (FIG. 21A). Cells were non-enzymatically released from culture plates, stained for CD14+ and assessed for the percentage of IL-6+ monocytes (FIG. 21A) by intracellular staining. For FIGS. 21B and 21C, cells suspensions were stimulated for 2 days with 1000 ng/mL LPS, following non-enzymatic release from culture plates and surface stained for HLA-DR, and CD86 to assess percentage or levels (mean fluorescence intensity—MFI) of HLA-DR+ (FIG. 21B), or CD86+ (FIG. 21C) monocytes. Data are normalized to each donor's vehicle control when appropriate and represented as mean±SEM. N=4-6 donors per treatment group. *p<0.05, ***p<0.001 when compared to VH (FIG. 21A), or *p<0.05, **p<0.01, ***p<0.001 when compared to VH (FIGS. 21B, 21C) by repeated measure one-way ANOVA with Dunnett's posttest.

FIGS. 22A-22C illustrates why linalool was contra-indicated. Linalool significantly increased IL-1β in monocytes. PBMC were isolated from human peripheral blood and pre-treated with the indicated concentrations of linalool for 30 minutes, followed by stimulation with 1000 ng/mL LPS for 24 hours (FIGS. 22A, 22C) or 15 μg/mL CpG-ODN for 6 hours (FIG. 22B). Cells were non-enzymatically released from culture plates and stained for CD14 and assessed for the percentage of TNFα⁺ monocytes (FIG. 22B) or IL-6⁺ monocytes (FIG. 22C) by intracellular staining. Supernatants were collected and IL-1β quantified by ELISA (FIG. 22A). Data are normalized to each donor's vehicle control when appropriate and represented as mean±SEM. N=4-6 donors per treatment group. *p<0.05 when compared to VH by repeated measure one-way ANOVA with Dunnett's posttest.

FIGS. 23A-23C demonstrates why trans-nerolidol (tN) was contra-indicated. Because tN significantly increased IL-1β levels in monocytes, it is contra-indicated for our cannabinoid containing complex mixtures. In addition, tN increased the activation marker, CD25+. PBMC were isolated from human peripheral blood and pre-treated with the indicated concentrations of trans-nerolidol for 30 minutes, followed by stimulation with 1000 ng/mL LPS for 24 hours (FIG. 23A). Supernatants were collected and IL-1β quantified by ELISA (FIG. 23A). For FIGS. 23B and 23C, PBMC were isolated from human peripheral blood and pre-treated with the indicated concentrations of trans-nerolidol for 30 minutes in culture plates coated with 5 μg/mL anti-CD3, followed by stimulation with 5 μg/mL anti-CD28 and supplemented with 5 ng/mL IL-2. Cells were cultured for 24 hours (FIGS. 23B, 23C). Cells were non-enzymatically released from culture plates and stained for CD25 and assessed for percentage of CD25⁺ (FIG. 23B) CD4⁺ T cells or CD25⁺ (FIG. 23C) CD8⁺ T cells. Data are normalized to each donor's vehicle control when appropriate and presented as mean±SEM. N=4-6 donors per treatment group. *p<0.05, **p<0.01 when compared to VH by repeated measure one-way ANOVA with Dunnett's posttest.

FIGS. 24A-24E provide summary tables organized by the proposed clinical indications for the cannabinoid-based therapeutic mixtures based on their immunomodulating activity, as demonstrated in the present application by the activity of each cannabis-based therapeutic mixture on the critical immune cell types we evaluated. Although these experiments were conducted in a co-culture system including all of the primary human peripheral blood mononuclear cells (PBMCs), row two of the tables indicate the critical immune cell type measured by flow cytometry at the time of the final measurement. Plasmacytoid dendritic cells (pDC), monocytes (Mø), and T cells (CD4+ and CD8+ T cells) were targeted because of their importance in the human immune response to viral infections and their responsiveness to cannabinoids and terpenes through receptor mediated immune modulating reactions. The timing of the measurements of the cytokines/immune biomarkers is indicated in row three. The early cytokine biomarkers were measured 6 hours after immune stimulation and co-incubation with these therapeutic mixtures. The MID cytokine and biomarkers were measured 24 hours after immune stimulation and co-incubation with these therapeutic mixtures. The later cytokine and biomarkers were measured 96 hours/4 days after immune stimulation and co-incubation with these therapeutic mixtures. The cytokines and biomarkers measured are listed in row four. In the second column of these tables, the anti-inflammatory potential and/or immunomodulatory potential is summarized based on the total number of immune parameters that were significantly affected by exposure to each mixture (left-most column) in a statistically significant manner for a given cell type (second row). If a mixture (column one) had a statistically-significant anti-inflammatory response in an assay for a particular cytokine or biomarker (row four), there is an “X” in the corresponding location in the grid. If a mixture (column one) had a statistically-significant pro-inflammatory response in an assay for a particular cytokine or biomarker (row four), there is an “*” in the corresponding location in the grid. FIG. 24A. CYTOKINE RELEASE SYNDROME THERAPEUTICS DESIGNED FOR HYPERINFLAMMATORY RESPONSES: These seven cannabinoid-based therapeutic mixtures targeted immune responses in monocytes & T cells while preserving plasmacytoid dendritic cell responses to virus/bacteria, which may or may not lead to viral-CPG stimulated TNFα in monocytes at 6 h. This category contains therapeutic mixtures designed to suppress the mid to later phase immune responses (24 hr monocyte and 96 hr T cell functions) but preserve the anti-viral functions of the plasmacytoid dendritic cells (pDC) and the 6-hr TNFα response in monocytes. Preserving the early-stage functions may be important in not leaving patients unprotected against opportunistic viral infections while suppressing the pro-inflammatory activities such as TNFα, IFNγ, IL-6 that correlate with tissue damage and adverse outcomes. FIG. 24B. CYTOKINE RELEASE SYNDROME THERAPEUTICS DESIGNED FOR T CELL-RELATED HYPERINFLAMMATION: This representative cannabinoid-based therapeutic mixture targets T cells while preserving plasmacytoid dendritic cell and monocyte responses to virus/bacteria. FIG. 24C. BROADLY IMMUNOMODULATING: These five cannabinoid-based therapeutic mixtures target plasmacytoid dendritic cell, monocyte, and T cell functions, which offers full coverage across the time and cell spectrum measured. FIG. 24D. IMMUNOSUPRESSIVE: These ten cannabinoid-based therapeutic mixtures targets plasmacytoid dendritic cell responses and/or T cells. They provide consistent suppression of pro-inflammatory cytokines and biomarkers at early and later stage inflammation, which could be used for the treatment of chronic inflammatory conditions. FIG. 24E. PRO-INFLAMMATORY: This representative cannabinoid-based therapeutic mixture targets monocytes and creates a pro-inflammatory response.

FIG. 25. The effects of Cannabinoid-based Therapeutic Mixtures on Interferon alpha (IFNα) in the Early Phase Immune Response to a Viral Stimulus 6 Hours post CpG Addition in plasmacytoid Dendritic Cells (pDC). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 26. The effects of Cannabinoid-based Therapeutic Mixtures on Tumor Necrosis Factor alpha (TNFα) in the Early Phase Immune Response to a Viral Stimulus 6 Hours post CpG. Addition in plasmacytoid Dendritic Cells (pDC). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 27. The effects of Cannabinoid-based Therapeutic Mixtures on Tumor Necrosis Factor alpha (TNFα) in the Early Phase Immune Response to a Viral Stimulus 6 Hours post CpG Addition in Monocytes (Mø). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 28. The effects of Cannabinoid-based Therapeutic Mixtures on Tumor Necrosis Factor alpha (TNFα) in the Mid-term Anti-Pathogen Response, 24 Hours after LPS Immune Stimulation of Monocytes (Mø). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 29. The effects of Cannabinoid-based Therapeutic Mixtures on Interleukin 6 (IL-6) in the Mid-term Anti-Pathogen Response, 24 Hours after LPS Immune Stimulation of Monocytes (Mø). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 30. The effects of Cannabinoid-based Therapeutic Mixtures on Interleukin 1 beta (IL-1β) in the Mid-term Anti-Pathogen Response, 24 Hours after LPS Immune Stimulation of Monocytes (Mø). Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 31. The effects of Cannabinoid-based Therapeutic Mixtures on CD80+ Cell Surface Expression of Monocytes (Mø) in the Mid-term Anti-Pathogen Response, 24 Hours after LPS. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 32. The effects of Cannabinoid-based Therapeutic Mixtures on CD86+ Expression of Monocytes (Mø) in the Mid-term Anti-Pathogen Response, 24 Hours after LPS Immune Stimulation. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 33. The effects of Cannabinoid-based Therapeutic Mixtures on Tumor Necrosis Factor alpha (TNFα) levels from CD4+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 34. The effects of Cannabinoid-based Therapeutic Mixtures on Interleukin 2 (IL-2) levels from CD4+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. PBMC suspensions were pre-treated with our cannabinoid-based therapeutic mixtures for 30 minutes prior to stimulation. The designated stimulation of the T cell populations in the PBMC involved adding the PBMC suspension to 96 well tissue culture plates pre-coated with 5.0 μg/mL anti-CD3 (clone UCHT1, Biolegend, San Diego, Calif.), stimulated with 5.0 μg/mL anti-CD28 (clone 28.2, Biolegend), supplemented with 5.0 ng/mL IL-2 (Roche Applied Science, Indianapolis, Ind.), and incubated at 37° C. and 5.0% CO2. After 4 days of incubation with the stimulus (and cannabinoid-based therapeutic mixtures in the treatment groups), the PBMC were further stimulated with 50 ng/mL phorbol 12-myristate 13-acetate (PMA) and 1000 ng/mL ionomycin (Io), and then Golgi transport inhibitors were also added for 4 hours before the cells were harvested. Cells were fixed in BD CytoFix. Then these cells were permeabilized in BD Permwash and incubated with appropriate antibodies for intercellular staining of IL-2. Using flow cytometry, these T cells were identified as CD3+ the T cell subset was identified by CD4+ expression, and then assessed for intracellular IL-2. Untreated (Unt.) samples were physically manipulated the same as the other experimental cell groups, but they were untreated by any of the compounds. Stimulated cells (Stim.) were not pre-incubated with any of the cannabinoid-based therapeutic mixtures, but they were stimulated and processed for IL-2 measurements the same as for the cannabinoid-based therapeutic mixture groups. The Positive Control (Pos. Cont.) group was pre-incubated with the vehicle control from the cannabinoid-based therapeutic mixtures, then it was stimulated and processed as for the cannabinoid-based therapeutic mixtures. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 35. The effects of Cannabinoid-based Therapeutic Mixtures on Interferon gamma (IFNγ) levels from CD4+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 36. The effects of Cannabinoid-based Therapeutic Mixtures on Tumor Necrosis Factor alpha (TNFα) levels from CD8+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 37. The effects of Cannabinoid-based Therapeutic Mixtures on Interleukin 2 (IL-2) levels from CD8+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 38. The effects of Cannabinoid-based Therapeutic Mixtures on Interferon gamma (IFNγ) levels from CD8+ T Cells in Later Immune Response, Measured 4 days post CD3/CD28 activation, Restimulation with PMA/IO. Each data point represents the mean response of 6 individual donors. Each of the treatment groups were compared with the positive control derived from the same individual donor. All of the sample values presented in the bar graph were normalized to the Positive Control group, which represents 100% of a hyperinflammatory reaction in the presence of the vehicle control for the cannabinoid-based therapeutic mixtures. A paired t-test was used to determine statistical significance of the experimental group relative to the positive control group [* denotes p=<0.05; ** denotes p=<0.01; *** denotes p=<0.001; and **** denotes p=<0.0001]. The table below the bar graph provides the amount (in μM) of each cannabinoid or terpene in the cannabinoid-based therapeutic mixtures above. The lines above the bar graph visually indicate which cannabinoids and terpenes are in the mixtures in the bar graph below.

FIG. 39 provides an illustration of the sensitivity to changes in components and the relative amounts of components in the Cannabinoid Containing Complex Mixtures relative to their effectiveness as immunomodulating agents. See the boxes around the mixtures for specific examples of the sensitivity to immunomodulatory changes.

FIG. 40 provides a further illustration of the sensitivity to changes in components and the relative amounts of components in the Cannabinoid Containing Complex Mixtures relative to their effectiveness as immunomodulating agents. See the boxes around the mixtures for specific examples of the sensitivity to immunomodulatory changes.

FIG. 41 provides a further illustration of the sensitivity of the mixtures to changes in their components and the relative amounts of components in the CCCM relative to their effectiveness as immunomodulating agents. See the boxes around the mixtures for specific examples of the sensitivity to immunomodulatory changes.

FIG. 42 provides a further illustration of the sensitivity of the mixtures to changes in their components and the relative amounts of components in the CCCM, relative to their effectiveness as immunomodulating agents. See the boxes around the mixtures for specific examples of the sensitivity to immunomodulatory changes.

FIG. 43 shows Early versus Late: Innate versus Adaptive Immune Responses to Viral Infections. The plasmacytoid dendritic cells, monocytes, and T cells were selected as the target cells for the Cannabinoid Containing Complex Mixtures (CCCM). These three cells were chosen as representatives within the PBMC (all cells tested in a co-culture) because of their critical roles in inflammatory responses, especially in inflammation secondary to mounting a viral response. The plasmacytoid dendritic cells (pDC) play an early role in anti-viral inflammation as a part of the innate immune response. The pDC are extremely sensitive to viral genomic pathogen associated molecular patterns (PAMPs) due to their PAMP-sensitive receptors. The pDCs are known for their robust secretion of interferon alpha (IFNα), which really amplifies the anti-viral inflammatory response. The secretion of IFNα by pDCs is 1000-fold greater than from any other immune cell type, providing an important first line of defense against invading viruses. Monocytes are early to middle responders that importantly bridge the innate and the adaptive (antibody based) immune system through antigen presentation to T cells. Monocytes up-regulate co-stimulatory molecules including CD80 and CD86 upon pathogen exposure, which allows for appropriate activation of T cells. T cells play a key role in later response as a part of the adaptive immune response, which is critically important in the appropriate response to pathogens. T cells are composed of two major populations, helper (CD4+) and cytotoxic (CD8+) T cells, which can be further subdivided based on function and the profile of regulatory factors (cytokines) they produce.

FIG. 44 shows early versus Late: Innate versus Adaptive Immune Responses to Viral Infections. In order to assess the effects of Cannabinoid Containing Complex Mixtures (CCCM), the following key cytokines and inflammatory markers were measured in the indicated cell types across the early to late response timetable. Measured at 6 hours post-stimulation of an inflammatory response in our experiments, the early inflammatory indicators are interferon alpha (IFNα) and tumor necrosis factor alpha (TNFα) from the plasmacytoid dendritic cells, as well as tumor necrosis factor alpha TNFα from the monocytes. Measured at 24 hours post-stimulation of an inflammatory response, the early to midterm inflammatory markers were TNFα, interleukin six (IL-6), interleukin 1 beta (IL-1β), CD80+ cell marker, and CD86+ cell marker in monocytes. Measured at 96 hours (4 days) post-stimulation of an inflammatory response, the later inflammatory markers were TNFα, interleukin two (IL-2), and interferon gamma (IFNγ) levels in CD4+ and CD8+ T cells.

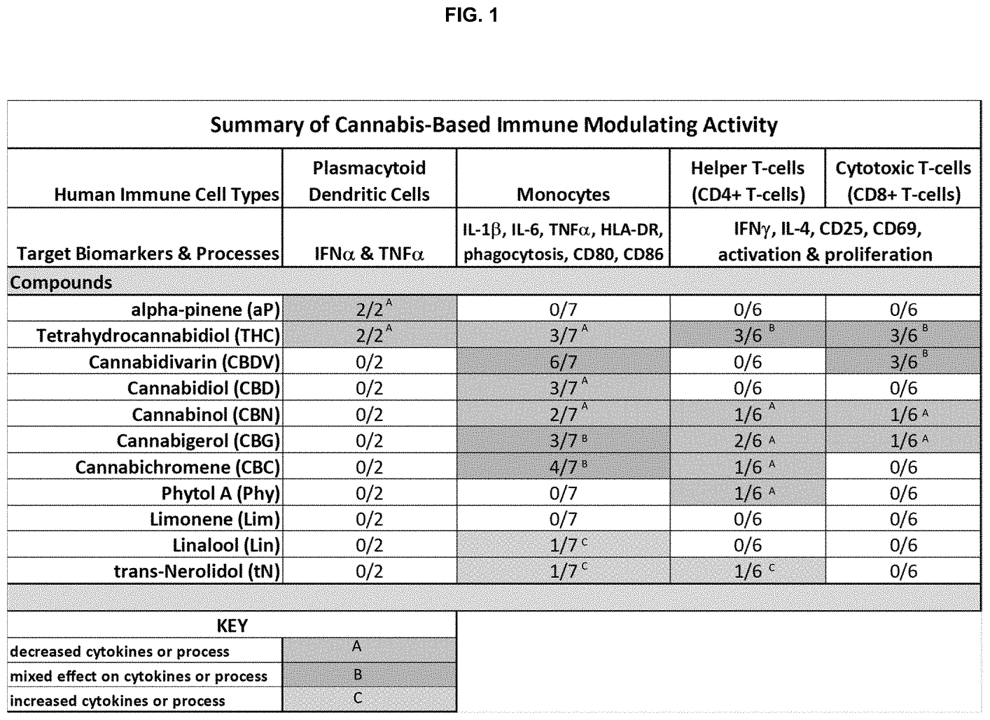

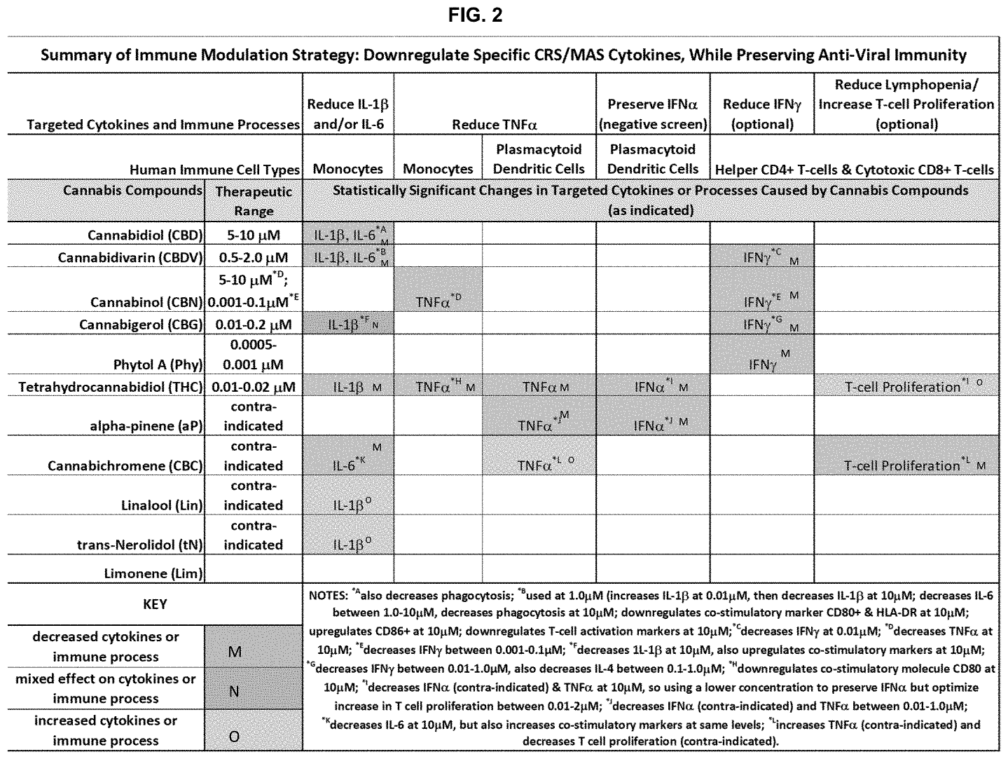

The figures depict various embodiments of the present invention for purposes of illustration only. One skilled in the art will readily recognize from the following discussion that alternative embodiments of the structures and methods illustrated herein may be employed without departing from the principles of the invention described herein.

### 5. DETAILED DESCRIPTION

### 5.1. DEFINITIONS

Unless defined otherwise, all technical and scientific terms used herein have the meaning commonly understood by a person skilled in the art to which this invention belongs. As used herein, the following terms have the meanings ascribed to them below.

A “terpene” means limonene, linalool, nerolidol (e.g., trans-nerolidol), pinene (e.g., alpha-pinene), or phytol (e.g., phytol A). The terpene can be obtained by chemical synthesis, chemical modification, commercially, or obtained from plant materials derived from one or more Cannabis plants.

An “interleukin-6 (IL-6)-decreasing cannabinoid” is a cannabinoid or terpene that can decrease IL-6 levels or suppress secretion of IL-6 by activated CD14⁺ CD16⁺ monocytes when applied to PBMC in vitro. An IL-6 decreasing cannabinoid includes a monocyte-modulating cannabinoid (e.g., the cannabinoid is capable of decreasing intracellular IL-6 in monocytes). In specific embodiments, the IL-6-decreasing cannabinoid is Cannabidiol (CBD), Cannabidivarin (CBDV), or a combination thereof.

An “interleukin-1β (IL-1β)-decreasing cannabinoid” is a cannabinoid or terpene that can decrease IL-1β levels or suppress secretion of IL-1β by activated CD14⁺ CD16⁺ monocytes when applied to PBMC in vitro. An IL-1β decreasing cannabinoid includes a monocyte-modulating cannabinoid (e.g., the cannabinoid is capable of decreasing intracellular IL-1β in monocytes). In specific embodiments, the IL-1β -decreasing cannabinoid is Cannabidiol (CBD), Cannabidivarin (CBDV), or a combination thereof.

An “interleukin-6 (IL-6) and interleukin-1β (IL-β)-decreasing cannabinoid” is a cannabinoid or terpene that can decrease IL-6 and IL-1β levels or suppress secretion of IL-6 and/or IL-1β by activated CD14⁺ CD16⁺ monocytes when applied to PBMC in vitro. An IL-6 and IL-1β decreasing cannabinoid includes a monocyte-modulating cannabinoid (e.g., the cannabinoid is capable of decreasing intracellular IL-6 and IL-1β in monocytes). In specific embodiments, the IL-6 and IL-1β -decreasing cannabinoid is Cannabidiol (CBD), Cannabidivarin (CBDV), or a combination thereof.

A “tumor necrosis factor alpha (TNFα)-decreasing cannabinoid” is a cannabinoid that can decrease TNFα level or secretion TNFα by pDCs or activated CD14⁺CD16⁺ monocytes when applied to PBMC in vitro. A TNFα decreasing cannabinoid includes a pDC-modulating cannabinoid, a monocyte-modulating cannabinoid, and/or a T-cell modulating cannabinoid (e.g., the cannabinoid is capable of decreasing intracellular TNFα in pDCs, monocytes, and/or T cells). In specific embodiments, the TNFα-decreasing cannabinoid is Cannabinol (CBN).

An “interferon-gamma (IFNγ)-decreasing cannabinoid or terpene” is a cannabinoid or terpene that can decrease IFNγ level or secretion of IFNγ by pDCs, activated CD14⁺CD16⁺ monocytes, or T-cells when applied to PBMC in vitro. A IFNγ decreasing cannabinoid can include T-cell (e.g., CD4+ and/or CD8+ T cell) modulating cannabinoid (e.g., the cannabinoid is capable of decreasing intracellular IFNγ in T-cells). In specific embodiments, the IFNγ-decreasing cannabinoid is Cannabigerol (CBG). In other embodiments, the IFNγ-decreasing cannabinoid is Phytol A.

A “lymphopenia-reducing cannabinoid” is a cannabinoid that has an activating and/or proliferating effect on helper CD4⁺ T cells and cytotoxic CD8⁺ T cells when applied to PBMC in vitro. The lymphopenia-reducing cannabinoid can reduce and/or prevent lymphopenia, which is a condition of having an abnormally low level of lymphocytes in the blood. In specific embodiments, the lymphopenia-reducing is tetrahydrocannabinol (THC) or trans-nerolidol.

A “pDC-modulating cannabinoid or terpene” is a cannabinoid or terpene that has modulating properties acting on pDC cells. In some embodiments, a pDC modulating cannabinoid or terpene can include a pDC modulating cannabinoid or terpene that targets pDC cells and provides for reduced levels of TNFα and IFNα.

A “monocyte-modulating cannabinoid or terpene” is a cannabinoid or terpene that has modulating properties acting on monocytes. In some embodiments, a monocyte modulating cannabinoid or terpene can include a monocyte modulating cannabinoid or terpene that targets monocytes and provides for reduced levels of intracellular TNFα expression, intracellular IL-6 expression, intracellular IL-1β expression, extracellular CD80 secretion, and extracellular CD86 secretion.

A “T-cell-modulating cannabinoid or terpene” is a cannabinoid or terpene that has modulating properties that acting on CD4+ or CD8+ T cells. In some embodiments, a T-cell modulating cannabinoid or terpene can include a CD4+ or CD8+modulating cannabinoid or terpene that targets CD4+ or CD8+ T cells (or both) and provides for reduced levels of intracellular TNFα expression, intracellular IL-2 expression, and intracellular IFNγ expression.

A pharmaceutically active ingredient is “substantially free” of a compound if the ingredient contains less than 0.3% (w/v) of the compound. For example, a pharmaceutically active ingredient is “substantially free of THC” if the ingredient contains less than 0.3% (w/v) of delta-9 tetrahydrocannabinol. A pharmaceutical composition comprising a pharmaceutically active ingredient is “substantially free of THC” if the pharmaceutical composition contains less than 0.3% (w/v) of delta-9 tetrahydrocannabinol.

A “Cannabis sativa extract” is a composition obtained from Cannabis sativa plant materials by fluid and/or gas extraction, for example by supercritical fluid extraction (SFE) with CO₂. The Cannabis sativa extract typically contains major cannabinoids, minor cannabinoids, terpenes, phytocannabinoids, and secondary metabolites. For example, the Cannabis sativa extract can include one or more of bisabolol, humulene, terpinene, caryophyllene, camphene, geraniol, guaiol, isopulegoll, ocimene, cymene, eucalyptol, terpinolene, and myrcene.

A “synthetic” cannabinoid or terpene is a cannabinoid or terpene made by chemical synthesis. The synthetic cannabinoid or terpene may be identical to a naturally occurring cannabinoid or terpene.

A “biosynthetic” cannabinoid or terpene is a cannabinoid or terpene made by a living organism or a laboratory process modeled after reactions in living organisms. The biosynthetic cannabinoid or terpene may be identical to a naturally occurring cannabinoid or terpene.

### 5.2. OTHER INTERPRETATIONAL CONVENTIONS

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 5.3. OVERVIEW OF EXPERIMENTAL RESULTS

The present disclosure provides complex mixtures containing cannabinoids and/or terpenes for treatment of CRS,MAS, Cytokine Storm Syndrome (CSS), Respiratory Distress (ARD), the hyper-inflammatory condition known as secondary hemophagocytic lymphohistiocytosis (sHLH), adverse side effects of checkpoint inhibitor therapies (cancer) and CAR-T-cell therapies (anti-cancer), rheumatoid arthritis and multiple sclerosis, chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND), or localized infections.

In the experiments described in this disclosure in Examples 1-2, certain cannabinoids and terpenes have been demonstrated to be effective in targeting and modulating activities of major immune cells that are implicated in the pathology of CRS or MAS, such as activated CD14⁺ CD16⁺ monocytes, plasmacytoid dendritic cells, and activated T-cells, with the goal that these cells both produce cytokines that contribute to the pathology of CRS and MAS, but also help preserve broader immune functions; especially anti-viral immunity while fighting a viral pathogen such as SARS-CoV-2. The cannabinoid containing complex mixtures were generated by combining cannabinoids and terpenes assigned to four different groups based on their modulatory effects and target immune cells—(i) an interleukin-lbeta (IL-1β)-decreasing and/or an interleukin-6 (IL-6)-decreasing cannabinoid; (ii) a tumor necrosis factor-alpha (TNFα)-decreasing cannabinoid; and (iii) an interferon-gamma (IFNγ)-decreasing cannabinoid or terpene, and (iv) a lymphopenia-reducing (T cell proliferation-stimulating) cannabinoid. Some of the cannabinoids and terpenes showed different modulatory effects, e.g., suppression or activation, at different ranges of concentrations.

By providing an active pharmaceutical ingredient containing compounds with distinct immune-modulatory activities selected from (i) an interleukin-1 beta (IL-1β)-decreasing and/or an interleukin-6 (IL-6)-decreasing cannabinoid; (ii) a tumor necrosis factor-alpha (TNFα)-decreasing cannabinoid; (iii) an interferon-gamma (IFNγ)-decreasing cannabinoid or terpene (optional), and (iv) a lymphopenia-reducing (T cell stimulating) cannabinoid (optional), the present disclosure provides an improved way of treating CRS/MAS by targeting those immune cell types that are most likely involved in the pathology. These immune cells also contain cannabinoid-sensitive and terpene-sensitive receptors. The present disclosure further provides dose ranges of different compounds in the composition that are expected to provide the desired therapeutic outcome.

The cannabinoid containing complex mixtures of cannabinoids and/or terpenes described herein downregulate the production of at least one of IL-1β, IFNγ, IL-2, IFNα, IL-6, and TNFα, which have been shown to correlate with severe hyperinflammation/cytokine storm syndrome, macrophage activation syndrome, and they address lymphopenia by specifically increasing proliferation of cytotoxic CD8⁺ T-cells. The complex cannabinoid and/or terpene mixtures of described herein (i) reduce the levels of pro-inflammatory cytokines that contribute to clinical pathology in patients who have, or who are at risk for developing CRS/MAS, including IL-6 and TNFα; (ii) reduce the IFNγ cytokine, which is implicated in tissue damage in CRS/MAS; (iii) increase cytotoxic CD8⁺ T-cell proliferation and stimulation of appropriate T-cell activity; and (iv) preserve the anti-viral response function of the plasmacytoid dendritic cells by protection of exogenous IFNα production and/or supplementation with recombinant interferons such as IFNα.

These cannabinoid containing complex mixtures can be used in the treatment of a patient who has, or who is at risk for developing, CRS or MAS and other dangerous pro-inflammatory conditions. These cannabinoid containing complex mixtures can be used for modulating an immune response in a patient, for example, in a patient who has hyperinflammation/pro inflammatory immune response. These cannabinoid containing complex mixtures can be used for modulating an immune response in a patient, for example, in a patient who requires modulation of an immune response.

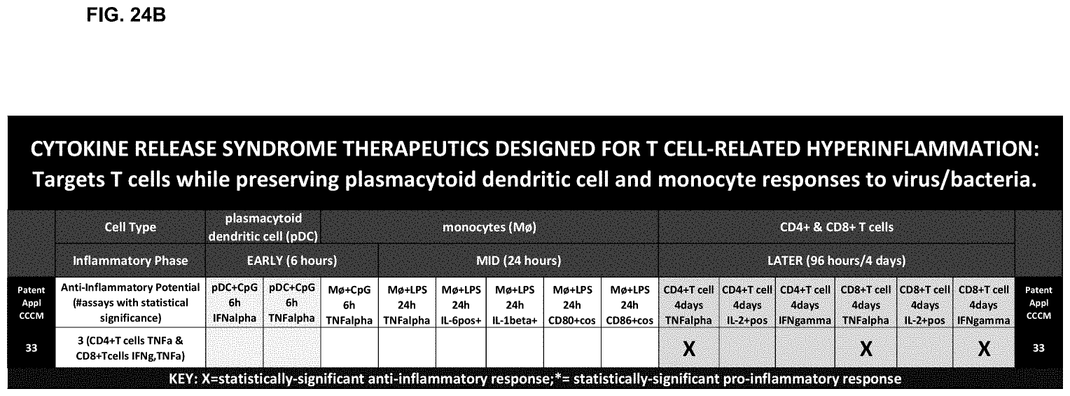

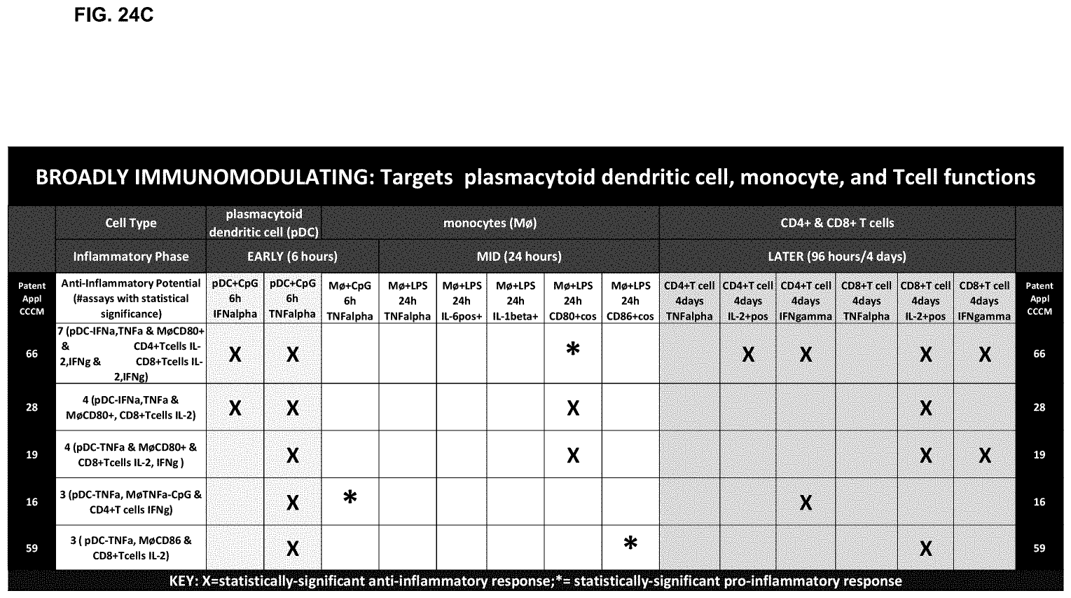

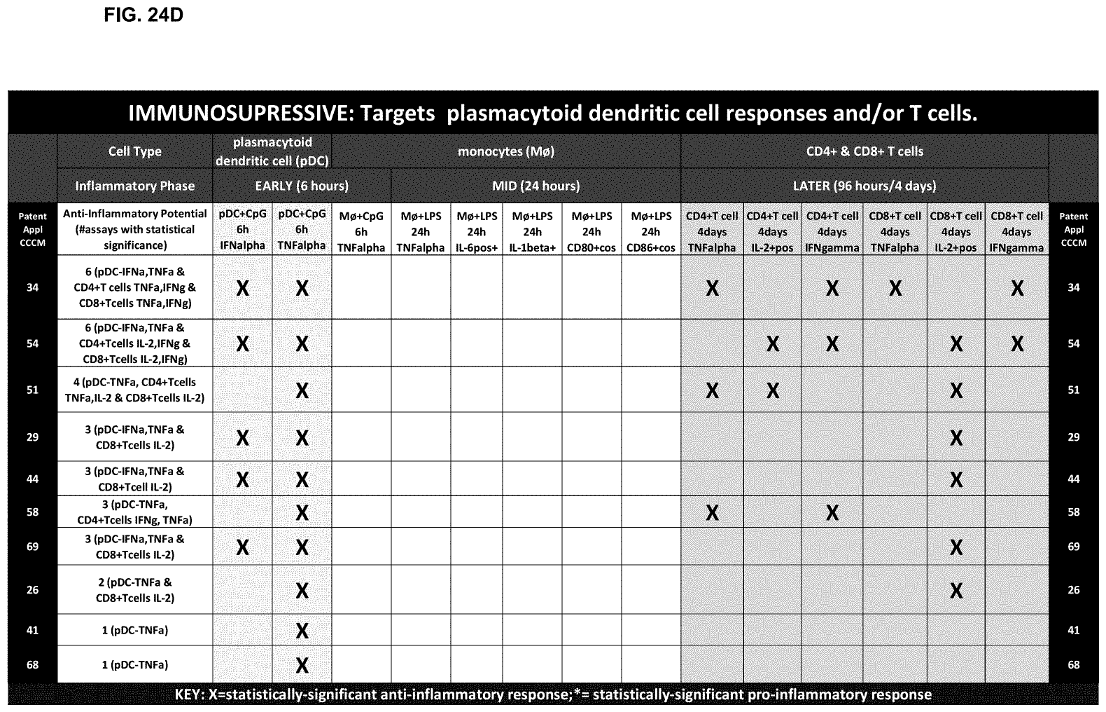

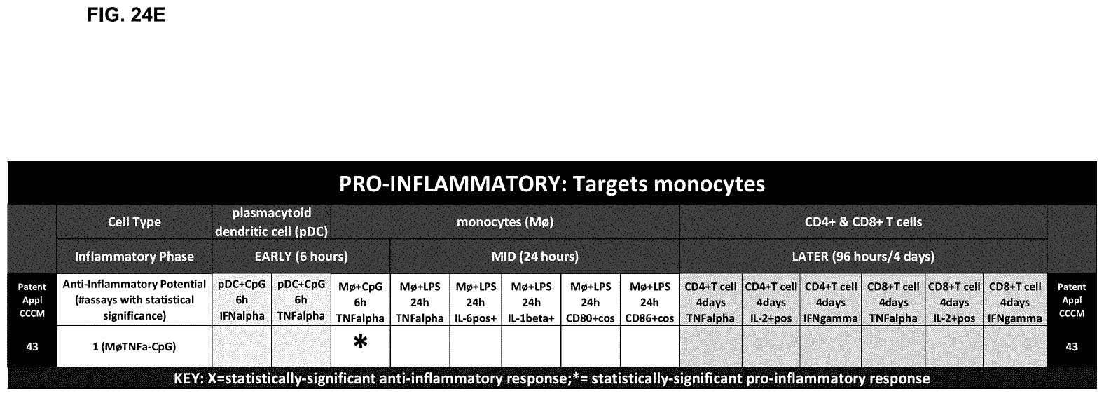

The present inventors found, as shown in Example 3, where cannabinoid containing complex mixtures (CCCM) were tested, the CCCMs were effective in targeting and modulating activities of major immune cells that are implicated in the pathology of CRS or MAS, such as activated CD14⁺ CD16⁺ monocytes, plasmacytoid dendritic cells, and activated T-cells. The cannabinoid containing complex mixtures were initially generated by combining cannabinoids and terpenes assigned to three different groups based on their modulatory effects and target immune cells—(i) an interleukin-1beta (IL-1β)-decreasing and/or an interleukin-6 (IL-6)-decreasing cannabinoid; (ii) a tumor necrosis factor-alpha (TNFα)-decreasing cannabinoid; and (iii) an interferon-gamma (IFNγ)-decreasing cannabinoid or terpene. Some of the cannabinoids and/or terpene mixtures showed different modulatory effects, e.g., suppression or activation, at different ranges of concentrations. Based on these results, the CCCMs tested were categorized into 5 different groups, based on the resulting immunomodulatory effects for each CCCM shown in FIGS. 24A-24E, and provided below:

Category 1: Cytokine Release Syndrome Therapeutics Designed For Hyperinflammatory Responses. This category targets monocytes & T cells while preserving plasmacytoid dendritic cell responses to virus/bacteria (that may or may not lead to viral-CPG stimulated TNFα in monocytes at 6 h). The goal was to target the mid to later stages of the inflammatory and hyper-inflammatory processes to preserve key anti-viral immune reactions. This category contains therapeutic mixtures that suppress the mid to later phase immune responses (24 hr monocyte and 96 hr T cell functions) but preserve the anti-viral functions of the plasmacytoid dendritic cells (pDC) and the 6-hr TNFα response in monocytes. The therapeutic mixtures in this category were designed to address clinical syndromes such as Cytokine Release Syndrome (CRS), Cytokine Storm Syndrome (CSS), Macrophage Activation Syndrome (MAS), Acute Respiratory Distress (ARD), and the hyper-inflammatory condition known as secondary hemophagocytic lymphohistiocytosis (sHLH) because their pathologies are all related to unregulated overproduction of proinflammatory cytokines. Also potentially useful for adverse side effects of checkpoint inhibitor therapies (cancer) and CAR-T-cell therapies (anti-cancer).

Category 2: Cytokine Release Syndrome Therapeutics Designed For T Cell-Related Hyperinflammation: Targets T cells while preserving plasmacytoid dendritic cell and monocyte responses to virus/bacteria. Potential clinical use for rheumatoid arthritis and multiple sclerosis, which are autoimmune disorders where hyperactivated T cells attack self-antigens in the joints (arthritis) or the myelin sheath of the neurons (MS). Also potentially useful for adverse side effects of CAR-T-cell therapies (anti-cancer).

Category 3: Broadly Immunomodulating. This category targets plasmacytoid dendritic cell, monocyte, and T cell functions. Within this category, two of the mixtures suppress immune responses in all three of the measured cell types. These CCCMs would be candidates for the treatment of chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

The three mixtures with mostly anti-inflammatory effects (pDC and T cells), but an elevated inflammatory marker for the monocytes, may be useful to address conditions requiring suppression of specific cytokines, but where activated monocytes are needed to fight an active infection. These may include some forms of Cytokine Release Syndrome (CRS), Cytokine Storm Syndrome (CSS), Macrophage Activation Syndrome (MAS), Acute Respiratory Distress (ARD), and the hyper-inflammatory condition known as secondary hemophagocytic lymphohistiocytosis (sHLH) secondary to viral or bacterial infections such as severe forms of COVID-19.

Category 4: Immunosuppressive. This category targets plasmacytoid dendritic cell responses and/or T cells. Because these mixtures consistently downregulate early and later phases of pro-inflammatory responses, these therapeutic mixtures would be ideal for the treatment of chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND).

Category 5: Pro-Inflammatory. This category targets monocytes and produces TNFα early (6hours) but not later. The CCCMs in this category could be used to help fight localized infections. TNFα signals early provide an important signal used to recruit white blood cells to the site of a localized infection.

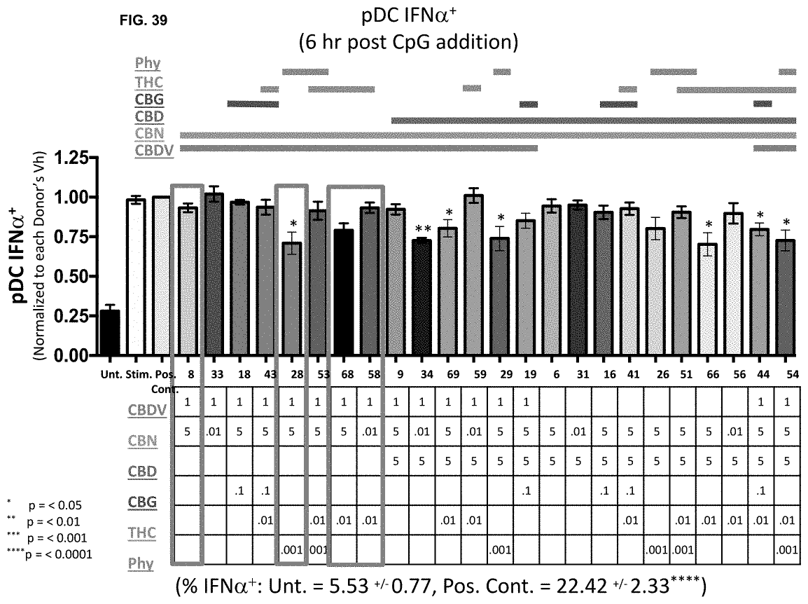

Additionally, the present inventors surprisingly found, as shown in FIGS. 39-42, the sensitivity in immunomodulatory effects of the CCCMs based on a change in at least one of the components of the CCCM or a change in the relative amounts of the components. For example, as shown in FIG. 39, CCCM mixture 8 containing 1 μM of CBDV and 5 μM of CBN is trending toward a reduction in IFNα, but is not significantly changed relative to the positive control. As seen in the results for CCCM mixture 28, adding just 0.001 μM of the terpene Phytol to the 1 μM of CBDV and 5 μM of CBN (like in CCCM mixture 8), causes a statistically significant reduction in IFNα. In another example of FIG. 39, as seen in CCCM mixture 58, changing the level of CBN from 5 μM to 0.01 μM CBN dramatically reduces the anti-inflammatory potential of the mixture. Taken together, these examples illustrate that the compositions and relative amounts of cannabinoids and terpenes in the mixtures had to be carefully deduced from rigorous experimentation on both individual ingredients and the mixtures.

### 5.4. PHARMACEUTICALLY ACTIVE INGREDIENT

5.4.1. Cannabinoids and Terpenes

Accordingly, in a first aspect, active pharmaceutical ingredients (also referred to herein synonymously as “active ingredients” and “pharmaceutically active ingredients”) are provided that comprise at least a first interleukin-lbeta (IL-1β)-decreasing and/or an interleukin-6 (IL-6)-decreasing cannabinoid, at least a first TNFα-decreasing cannabinoid, optionally at least a first IFNγ-decreasing cannabinoid or terpene, and optionally at least a first Lymphopenia-reducing cannabinoid.

Active pharmaceutical ingredients are provided that comprise one or more of a: pDC-modulating cannabinoid or terpene, a monocyte-modulating cannabinoid or terpene, and a T-cell modulating cannabinoid or terpene. A pDC-modulating cannabinoid is a TNFα-decreasing cannabinoid or terpene and/or a IFNα-decreasing cannabinoid or terpene. A monocyte-modulating cannabinoid is a TNFα-decreasing cannabinoid or terpene, an IL-6 decreasing cannabinoid or terpene, an IL-1β-decreasing cannabinoid or terpene, a CD80-decreasing cannabinoid or terpene, and/or a CD86-decreasing cannabinoid or terpene. A T-cell modulating cannabinoid or terpene is a IFNγ-decreasing cannabinoid or terpene, a TNFα-decreasing cannabinoid or terpene, and/or an IL-2-decreasing cannabinoid or terpene.

In some embodiments, the active pharmaceutical ingredient comprises a T-cell-modulating cannabinoid or terpene; and a monocyte-modulating cannabinoid or terpene. In some embodiments, the active pharmaceutical ingredient comprises two or more T-cell-modulating cannabinoids or terpenes (e.g., CD4 T-cell modulating cannabinoid or terpene and a CD8+ T cell cannabinoid or terpene). In some embodiments, the active pharmaceutical ingredient comprises a plasmacytoid dendritic cell-(pDC) modulating cannabinoid or terpene; a monocyte-modulating cannabinoid or terpene; and a T-cell-modulating cannabinoid or terpene. In some embodiments, the active pharmaceutical ingredient comprises a plasmacytoid dendritic cell-(pDC) modulating cannabinoid or terpene and optionally a T-cell-modulating cannabinoid or terpene. In some embodiments, the active pharmaceutical ingredient comprises two or more monocyte-modulating cannabinoids.

In some embodiments, the pDC modulating cannabinoid or terpene comprises two or more (e.g., three or more, four or more, five or more, six or more, etc.) T-cell modulating cannabinoids or terpenes. In some embodiments, the monocyte modulating cannabinoid or terpene comprises two or more (e.g., three or more, four or more, five or more, six or more, etc.)T-cell modulating cannabinoids or terpenes. In some embodiments, the T-cell modulating cannabinoid or terpene comprises two or more (e.g., three or more, four or more, five or more, six or more, etc.) T-cell modulating cannabinoids or terpenes. In some embodiments, the T-cell modulating cannabinoid or terpene comprises two or more T-cell modulating cannabinoids or terpenes. In some embodiments, the T-cell modulating cannabinoid or terpene comprises a CD4+ T-cell modulating cannabinoid or terpene, a CD8+ T-cell modulating cannabinoid or terpene, or a CD4+ and CD8+ T-cell modulating cannabinoid or terpene.

In some embodiments, the pDC modulating cannabinoid or terpene comprises a TNFα-decreasing cannabinoid. In some embodiments, the pDC modulating cannabinoid or terpene comprises a IFNα-decreasing cannabinoid. In some embodiments, the pDC modulating cannabinoid or terpene comprises a TNFα-increasing cannabinoid. In some embodiments, the pDC modulating cannabinoid or terpene comprises a IFNα-increasing cannabinoid.

In some embodiments, the monocyte modulating cannabinoid or terpene comprises a TNFα-decreasing cannabinoid. In some embodiments, the monocyte modulating cannabinoid or terpene comprises a TNFα-increasing cannabinoid.

In some embodiments, the monocyte modulating cannabinoid or terpene comprises an IL-6-decreasing cannabinoid. In some embodiments, the monocyte modulating cannabinoid or terpene comprises an IL-6-increasing cannabinoid.

In some embodiments, the monocyte modulating cannabinoid or terpene comprises an CD80-decreasing cannabinoid or terpene. In some embodiments, the monocyte modulating cannabinoid or terpene comprises an CD80-increasing cannabinoid or terpene.

In some embodiments, the monocyte modulating cannabinoid or terpene comprises an CD86-decreasing cannabinoid or terpene. In some embodiments, the monocyte modulating cannabinoid or terpene comprises an CD86-increasing cannabinoid or terpene.

In some embodiments, the T-cell modulating cannabinoid or terpene comprises a TNFα-decreasing cannabinoid or terpene. In some embodiments, the T-cell modulating cannabinoid or terpene comprises a TNFα -increasing cannabinoid or terpene.

In some embodiments, the T-cell modulating cannabinoid or terpene comprises a IFNγ-decreasing cannabinoid. In some embodiments, the T-cell modulating cannabinoid or terpene comprises a IFNγ-increasing cannabinoid or terpene.

In some embodiments, the T-cell modulating cannabinoid or terpene comprises an IL-2-decreasing cannabinoid or terpene. In some embodiments, the T-cell modulating cannabinoid or terpene comprises an IL-2-increasing cannabinoid or terpene or terpene. In certain embodiments, the IL-2 decreasing cannabinoid or terpene is selected from CBD, CBN, CBG, CBGA, CBDV, CBDVA, THC, and phytol A.

In some embodiments, the active pharmaceutical ingredient comprises an IL-1β-decreasing and/or IL-6-decreasing cannabinoid and a TNFα-decreasing cannabinoid. In some embodiments, the pharmaceutically active ingredient comprises an IL-1β-decreasing and/or IL-6-decreasing cannabinoid, a TNFα-decreasing cannabinoid, and an IFNγ-decreasing cannabinoid or terpene. In some embodiments, the pharmaceutically active ingredient comprises an IL-1β-decreasing and/or IL-6-decreasing cannabinoid, a TNFα-decreasing cannabinoid, and a Lymphopenia-reducing cannabinoid. In some embodiments, the pharmaceutically active ingredient comprises an IL-1β-decreasing and/or IL-6-decreasing cannabinoid, a TNFα-decreasing cannabinoid, an IFNγ-decreasing cannabinoid or terpene, and a Lymphopenia-reducing cannabinoid. In some embodiments, the pharmaceutically active ingredient comprises one or more of: an IFNα decreasing cannabinoid, a TNFα-decreasing cannabinoid, an IFNγ-decreasing cannabinoid or terpene, and a Lymphopenia-reducing cannabinoid.

In some embodiments, the IL-1β-decreasing cannabinoid is Cannabidiol (CBD), Cannabidivarin (CBDV), or a combination thereof. In certain embodiments, the IL-1β-decreasing cannabinoid is CBD. In certain embodiments, the IL-1β-decreasing cannabinoid is CBDV. In various embodiments, the IL-1β-decreasing cannabinoid is a combination of CBD and CBDV.

In some embodiments, the IL-6-decreasing cannabinoid is Cannabidiol (CBD), Cannabidivarin (CBDV), or a combination thereof. In certain embodiments, the IL-6-decreasing cannabinoid is CBD. In certain embodiments, the IL-6-decreasing cannabinoid is CBDV. In various embodiments, the IL-6-decreasing cannabinoid is a combination of CBD and CBDV.

In some embodiments, the IL-1β-decreasing and/or IL-6-decreasing cannabinoid is Cannabidiol (CBD), Cannabidivarin (CBDV), or a combination thereof. In certain embodiments, the IL-1β-decreasing and/or IL-6-decreasing cannabinoid is CBD. In certain embodiments, the IL-1β-decreasing and/or IL-6-decreasing cannabinoid is CBDV. In various embodiments, the IL-1β-decreasing and/or IL-6-decreasing cannabinoid is a combination of CBD and CBDV.

In some embodiments, the TNFα-decreasing cannabinoid is Cannabinol (CBN).

In some embodiments, the Lymphopenia-reducing cannabinoid is tetrahydrocannabinol (THC).

In some embodiments, the IFNγ-decreasing cannabinoid is Cannabigerol (CBG).

In some embodiments, the IFNγ-decreasing cannabinoid is Cannabinol (CBN).

In some embodiments, the IFNγ-decreasing cannabinoid is Phytol A.

In some embodiments, the IFNγ-decreasing cannabinoid is CBN or CBDV.

In some embodiments, the active pharmaceutical ingredient comprises or consists essentially of: CBD, and CBN; CBD, and CBN; CBDV, and CBN; CBD, CBDV, and CBN; CBD, CBDV, and CBN; CBD, and CBN; CBD, CBN, CBG, and THC; CBDV, and CBN; CBD, CBDV, and CBN; CBD, CBDV, and CBN; CBD, CBN, and CBG; CBD, CBN, and CBG; CBDV, CBN, and CBG; CBD, CBDV, CBN, andCBG; CBD, CBDV, CBN, and CBG; CBD, CBN, and CBG; CBD, CBN, and CBG; CBDV, CBN, and CBG; CBD, CBDV, CBN, and CBG; CBD, CBDV, CBN, and CBG; CBD, CBN, and Phytol A; CBD, CBN, and Phytol A; CBDV, CBN, and Phytol A; CBD, CBDV, CBN, and Phytol A; CBD, CBDV, CBN, and Phytol A; CBD, CBN, and Phytol A; CBD, CBN, and Phytol A; CBDV, CBN, and Phytol A; CBD, CBDV, CBN, and Phytol A; CBD, CBDV, CBN, and Phytol A; CBD, and CBN; CBD, and CBN; CBDV, and CBN; CBD, CBDV, and CBN; or CBD, CBDV, and CBN.

In some embodiments, the active pharmaceutical ingredient comprises or consists essentially of CBD, and CBN. In some embodiments, the active pharmaceutical ingredient comprises or consists essentially of CBD, and CBN. In some embodiments, the active pharmaceutical ingredient comprises or consists essentially of CBDV, and CBN. In some embodiments, the active pharmaceutical ingredient comprises or consists essentially of CBD, CBDV, and CBN. In some embodiments, the active pharmaceutical ingredient comprises or consists essentially of CBD, CBDV, and CBN. In some embodiments, the active pharmaceutical ingredient comprises or consists essentially of CBD, and CBN. In some embodiments, the active pharmaceutical ingredient comprises or consists essentially of CBD, CBN, CBG, and THC. In some embodiments, the active pharmaceutical ingredient comprises or consists essentially of CBDV, and CBN. In some embodiments, the active pharmaceutical ingredient comprises or consists essentially of CBD, CBDV, and CBN. In some embodiments, the active pharmaceutical ingredient comprises or consists essentially of CBD, CBDV, and CBN. In some embodiments, the active pharmaceutical ingredient comprises or consists essentially of CBD, CBN, and CBG. In some embodiments, the active pharmaceutical ingredient comprises or consists essentially of CBD, CBN, and CBG. In some embodiments, the active pharmaceutical ingredient comprises or consists essentially of CBDV, CBN, and CBG. In some embodiments, the active pharmaceutical ingredient comprises or consists essentially of CBD, CBDV, CBN, and CBG. In some embodiments, the active pharmaceutical ingredient comprises or consists essentially of CBD, CBDV, CBN, and CBG. In some embodiments, the active pharmaceutical ingredient comprises or consists essentially of CBD, CBN, and CBG. In some embodiments, the active pharmaceutical ingredient comprises or consists essentially of CBD, CBN, and CBG. In some embodiments, the active pharmaceutical ingredient comprises or consists essentially of CBDV, CBN, and CBG. In some embodiments, the active pharmaceutical ingredient comprises or consists essentially of CBD, CBDV, CBN, and CBG. In some embodiments, the active pharmaceutical ingredient comprises or consists essentially of CBD, CBDV, CBN, and CBG. In some embodiments, the active pharmaceutical ingredient comprises or consists essentially of CBD, CBN, and Phytol A. In some embodiments, the active pharmaceutical ingredient comprises or consists essentially of CBD, CBN, and Phytol A. In some embodiments, the active pharmaceutical ingredient comprises or consists essentially of CBDV, CBN, and Phytol A. In some embodiments, the active pharmaceutical ingredient comprises CBD, CBDV, CBN, and Phytol A. In some embodiments, the active pharmaceutical ingredient comprises or consists essentially of CBD, CBDV, CBN, and Phytol A. In some embodiments, the active pharmaceutical ingredient comprises or consists essentially of CBD, CBN, and Phytol A. In some embodiments, the active pharmaceutical ingredient comprises or consists essentially of CBD, CBN, and Phytol A. In some embodiments, the active pharmaceutical ingredient comprises or consists essentially of CBDV, CBN, and Phytol A. In some embodiments, the active pharmaceutical ingredient comprises or consists essentially of CBD, CBDV, CBN, and Phytol A. In some embodiments, the active pharmaceutical ingredient comprises CBD, CBDV, CBN, and Phytol A. In some embodiments, the active pharmaceutical ingredient comprises or consists essentially of CBD, and CBN. In some embodiments, the active pharmaceutical ingredient comprises or consists essentially of CBD, and CBN. In some embodiments, the active pharmaceutical ingredient comprises or consists essentially of CBDV, and CBN. In some embodiments, the active pharmaceutical ingredient comprises CBD, CBDV, and CBN. In some embodiments, the active pharmaceutical ingredient comprises or consists essentially of CBD, CBDV, and CBN.

In some embodiments, the active pharmaceutical ingredient comprises a molar ratio of: 1-part CBD:2-parts CBN; 1-part CBD:1-part CBN; 1-part CBDV: 10-parts CBN; 5-parts CBD: 1-part CBDV:10-part CBN; 10-parts CBD:1-part CBDV:10-parts CBN; 1-part CBD:1-part CBN; 2-parts CBD:1-part CBN; 1-part CBDV:5-parts CBN; 5-parts CBD:1-part CBDV:5-parts CBN; 10-parts CBD:1-part CBDV:5-parts CBN; 50-parts CBD:100-parts CBN:1-part CBG; 100-parts CBD:100-parts CBN:1-part CBG; 10-parts CBDV:100-parts CBN:1-part CBG; 50-parts CBD:10-parts CBDV:100-parts CBN:1-part CBG; 100-parts CBD:10-parts CBDV:100-parts CBN:1-part CBG; 50-parts CBD:50-parts CBN:1-part CBG; 100-parts CBD:50-parts CBN:1-part CBG; 10-parts CBDV:50-parts CBN:1-part CBG; 50-parts CBD:10-parts CBDV:50-parts CBN:1-part CBG; 100-parts CBD:10-parts CBDV:50-parts CBN:1-part CBG; 5,000-parts CBD:10,000-parts CBN:1-part Phytol A; 10,000-parts CBD:10,000-parts CBN:1-part Phytol A; 1,000-parts CBDV:10,000-parts CBN:1-part Phytol A; 5,000-parts CBD:1,000-parts CBDV:10,000-parts CBN:1-part Phytol A; 10,000-parts CBD:1,000-parts CBDV:10,000-parts CBN:1-part Phytol A; 5,000-parts CBD:5,000-parts CBN:1-part Phytol A; 10,000-parts CBD:5,000-parts CBN:1-part Phytol A; 1,000-parts CBDV:5,000-parts CBN:1-part Phytol A; 5,000-parts CBD:1,000-parts CBDV:5,000-parts CBN:1-part Phytol A; 10,000-parts CBD:1,000-parts CBDV:5,000-parts CBN:1-part Phytol A; 500-parts CBD:1-part CBN; 1,000-parts CBD:1-part CBN; 100-parts CBDV:1-part CBN; 500-parts CBD:100-parts CBDV:1-part CBN; 1,000-parts CBD:100-parts CBDV:1-part CBN; 500-parts CBD:1,000-parts CBN:10-parts CBG:1-part THC; 1,000-parts CBD:1,000-parts CBN:10-parts CBG:1-part THC; 100-parts CBDV:1,000-parts CBN:10-parts CBG:1-part THC; 500-parts CBD:100-parts CBDV:1,000-parts CBN:10-parts CBG:1-part THC; 1,000-parts CBD:100-parts CBDV:1,000-parts CBN:10-parts CBG:1-part THC; 500-parts CBD:500-parts CBN:10-parts CBG:1-part THC; 1,000-parts CBD:500-parts CBN:10-parts CBG:1-part THC; 100-parts CBDV:500-parts CBN:10-parts CBG:1-part THC; 500-parts CBD:100-parts CBDV:500-parts CBN:10-parts CBG:1-part THC; 1,000-parts CBD:100-parts CBDV:500-parts CBN:10-parts CBG:1-part THC; 5,000-parts CBD:10,000-parts CBN:1-part Phytol A:10-parts THC; 10,000-parts CBD:10,000-parts CBN:1-part Phytol A:10-parts THC; 1,000-parts CBDV:10,000-parts CBN:1-part Phytol A:10-parts THC; 5,000-parts CBD:1,000-parts CBDV:10,000-parts CBN:1-part Phytol A:10-parts THC; 10,000-parts CBD:1,000-parts CBDV:10,000-parts CBN:1-part Phytol A:10-parts THC; 5,000-parts CBD:5,000-parts CBN:1-part Phytol A:10-parts THC; 10,000-parts CBD:5,000-parts CBN:1-part Phytol A:10-parts THC; 1,000-parts CBDV:5,000-parts CBN:1-part Phytol A:10-parts THC; 5,000-parts CBD:1,000-parts CBDV:5,000-parts CBN:1-part Phytol A:10-parts THC; 10,000-parts CBD:1,000-parts CBDV:5,000-parts CBN:1-part Phytol A:10-parts THC; 500-parts CBD:1-part CBN:1-part THC; 1,000-parts CBD:1-part CBN:1-part THC; 100-parts CBDV:1-part CBN:1-part THC; 500-parts CBD:100-parts CBDV:1-part CBN:1-part THC; 1,000-parts CBD:100-parts CBDV:1-part CBN:1-part THC; 500-parts CBD:1,000-parts CBN:1-part THC; 1,000-parts CBD:1,000-parts CBN:1-part THC; 100-parts CBDV:1,000-parts CBN:1-part THC; 500:100:1,000:1 500-parts CBD:CBDV:CBN:1-part THC; 1,000-parts CBD:100-parts CBDV:1,000-parts CBN:1-part THC; 500-parts CBD:500-parts CBN:1-part THC; 1,000-parts CBD:500-parts CBN:1-part THC; 100-parts CBDV:500-parts CBN:1-part THC; 500-parts CBD:100-parts CBDV:500-parts CBN:1-part THC; or1,000-parts CBD:100-parts CBDV:500-parts CBN:1-part THC.

In some embodiments, the active pharmaceutical ingredient comprises a molar ratio of: 1-part CBD:2-parts CBN; 1-part CBD:1-part CBN; 1-part CBDV:10-parts CBN; 5-parts CBD:1-part CBDV:10-parts CBN; 10-parts CBD:1-part CBDV:10-parts CBN; 1-part CBD:1-part CBN; 2-parts CBD:1-part CBN; 1-part CBDV:5-parts CBN; 5-parts CBD:1-part CBDV:5-parts CBN; 10-parts CBD:1-part CBDV:5-parts CBN; 50-parts CBD:100-parts CBN:1-part CBG; 100-parts CBD:100-parts CBN:1-parts CBG;10-parts CBDV:100-parts CBN:1-part CBG; 50-parts CBD:10-parts CBDV:100-parts CBN:1-part CBG; 100-parts CBD:10-parts CBDV:100-parts CBN:1-part CBG; 50-parts CBD:50-parts CBN:1-part CBG; 100-parts CBD:50-parts CBN:1-part CBG; 10-parts CBDV:50-parts CBN:1-part CBG; 50-parts CBD:10-parts CBDV:50-parts CBN:1-part CBG; 100-parts CBD:10-parts CBDV:50-parts CBN:1-part CBG; 5,000-parts CBD: 10,000-parts CBN:1-part Phytol A; 10,000-parts CBD:10,000-part CBN:1-part Phytol A; 1,000-parts CBDV:10,000-parts CBN:1-part Phytol A; 5,000-parts CBD:1,000-parts CBDV:10,000-parts CBN:1-part Phytol A; 10,000-parts CBD:1,000-parts CBDV:10,000-parts CBN:1-part Phytol A; 5,000-parts CBD:5,000-parts CBN:1-part Phytol A; 10,000-parts CBD:5,000-parts CBN:1-part -part Phytol A; 1,000-parts CBDV:5,000-parts CBN:1-part Phytol A; 5,000-parts CBD:1,000-parts CBDV:5,000-parts CBN:1-part Phytol A; 10,000-parts CBD:1,000-parts CBDV:5,000-parts CBN:1-part Phytol A; 500-parts CBD:1-part CBN; 1,000-parts CBD:1-part

CBN; 100-parts CBDV:1-part CBN; 500-parts CBD:100-parts CBDV:1-part CBN; 1,000-parts CBD:100-parts CBDV:1-part CBN; 500-parts CBD:1,000-parts CBN:1-part THC; 1,000-parts CBD:1,000-parts CBN:1-part THC; 100-parts CBDV:1,000-parts CBN:1-part THC; 500:100:1,000:1 500-parts CBD:CBDV:CBN:1-part THC; 1,000-parts CBD:100-parts CBDV:1,000-parts CBN:1-part THC; 500-parts CBD:500-parts CBN:1-part THC; 1,000-parts CBD:500-parts CBN:1-part THC; 100-parts CBDV:500-parts CBN:1-part THC; 500-parts CBD:100-parts CBDV:500-parts CBN:1-part THC; or 1,000-parts CBD:100-parts CBDV:500-parts CBN:1-part THC.

In some embodiments, the active pharmaceutical ingredient comprises a molar ratio of 1-part CBD:2-parts CBN. In some embodiments, the active pharmaceutical ingredient comprises a molar ratio of 1-part CBD:1-part CBN. In some embodiments, the active pharmaceutical ingredient comprises a molar ratio of 1-part CBDV:10-parts CBN. In some embodiments, the active pharmaceutical ingredient comprises a molar ratio of 5-parts CBD:1-part CBDV:10-parts CBN. In some embodiments, the active pharmaceutical ingredient comprises a molar ratio of 10-parts CBD:1-part CBDV:10-parts CBN. In some embodiments, the active pharmaceutical ingredient comprises a molar ratio of 1-part CBD:1-part CBN. In some embodiments, the active pharmaceutical ingredient comprises a molar ratio of 2-parts CBD:1-part CBN. In some embodiments, the active pharmaceutical ingredient comprises a molar ratio of 1-part CBDV:5-parts CBN. In some embodiments, the active pharmaceutical ingredient comprises a molar ratio of 5-parts CBD:1-part CBDV:5-parts CBN. In some embodiments, the active pharmaceutical ingredient comprises a molar ratio of 10-parts CBD:1-part CBDV:5-parts CBN. In some embodiments, the active pharmaceutical ingredient comprises a molar ratio of 50-parts CBD:100-parts CBN:1-part CBG. In some embodiments, the active pharmaceutical ingredient comprises a molar ratio of 100-parts CBD:100-parts CBN:1-parts CBG. In some embodiments, the active pharmaceutical ingredient comprises a molar ratio of 10-parts CBDV:100-parts CBN:1-part CBG. In some embodiments, the active pharmaceutical ingredient comprises a molar ratio of 50-parts CBD:10-parts CBDV:100-parts CBN:1-part CBG. In some embodiments, the active pharmaceutical ingredient comprises a molar ratio of 100-parts CBD:10-parts CBDV:100-parts CBN:1-part CBG. In some embodiments, the active pharmaceutical ingredient comprises a molar ratio of 50-parts CBD:50-parts CBN:1-part CBG. In some embodiments, the active pharmaceutical ingredient comprises a molar ratio of 100-parts CBD:50-parts CBN:1-part CBG. In some embodiments, the active pharmaceutical ingredient comprises a molar ratio of 10-parts CBDV:50-parts CBN:1-part CBG. In some embodiments, the active pharmaceutical ingredient comprises a molar ratio of 50-parts CBD:10-parts CBDV:50-parts CBN:1-part CBG. In some embodiments, the active pharmaceutical ingredient comprises a molar ratio of 100-parts CBD:10-parts CBDV:50-parts CBN:1-part CBG. In some embodiments, the active pharmaceutical ingredient comprises a molar ratio of 5,000-parts CBD: 10,000-parts CBN:1-part Phytol A. In some embodiments, the active pharmaceutical ingredient comprises a molar ratio of 10,000-parts CBD:10,000-part CBN:1-part Phytol A. In some embodiments, the active pharmaceutical ingredient comprises a molar ratio of 1,000-parts CBDV:10,000-parts CBN:1-part Phytol A. In some embodiments, the active pharmaceutical ingredient comprises a molar ratio of 5,000-parts CBD:1,000-parts CBDV:10,000-parts CBN:1-part Phytol A. In some embodiments, the active pharmaceutical ingredient comprises a molar ratio of 10,000-parts CBD:1,000-parts CBDV:10,000-parts CBN:1-part Phytol A. In some embodiments, the active pharmaceutical ingredient comprises a molar ratio of 5,000-parts CBD:5,000-parts CBN:1-part Phytol A. In some embodiments, the active pharmaceutical ingredient comprises a molar ratio of 10,000-parts CBD:5,000-parts CBN:1-part -part Phytol A. In some embodiments, the active pharmaceutical ingredient comprises a molar ratio of 1,000-parts CBDV:5,000-parts CBN:1-part Phytol A. In some embodiments, the active pharmaceutical ingredient comprises a molar ratio of 5,000-parts CBD:1,000-parts CBDV:5,000-parts CBN:1-part Phytol A. In some embodiments, the active pharmaceutical ingredient comprises a molar ratio of 10,000-parts CBD:1,000-parts CBDV:5,000-parts CBN:1-part Phytol A. In some embodiments, the active pharmaceutical ingredient comprises a molar ratio of 500-parts CBD:1-part CBN. In some embodiments, the active pharmaceutical ingredient comprises a molar ratio of 1,000-parts CBD:1-part CBN. In some embodiments, the active pharmaceutical ingredient comprises a molar ratio of 100-parts CBDV:1-part CBN. In some embodiments, the active pharmaceutical ingredient comprises a molar ratio of 500-parts CBD:100-parts CBDV:1-part CBN. In some embodiments, the active pharmaceutical ingredient comprises a molar ratio of 1,000-parts CBD:100-parts CBDV:1-part CBN.

In some embodiments, the active pharmaceutical ingredient comprises CBD, CBN, CBG, and THC; CBD, CBN, CBG, and THC; CBDV, CBN, CBG, and THC; CBD, CBDV, CBN, CBG, and THC; CBD, CBDV, CBN, CBG, and THC; CBD, CBN, CBG, and THC; CBD, CBN, CBG, and THC; CBDV, CBN, CBG, and THC; CBD, CBDV, CBN, CBG, and THC; CBD, CBDV, CBN, CBG, and THC; CBD, CBN, Phytol A, and THC; CBD, CBN, Phytol A, and THC; CBDV, CBN, Phytol A, and THC; CBD, CBDV, CBN, Phytol A, and THC; CBD, CBDV, CBN, Phytol A, and THC; CBD, CBN, Phytol A, THC; CBD, CBN, Phytol A, and THC; CBDV, CBN, Phytol A, and THC; CBD, CBDV, CBN, Phytol A, and THC; CBD, CBDV, CBN, Phytol A, and THC; CBD, CBN, and THC; CBD, CBN, and THC; CBDV, CBN, and THC; CBD, CBDV, CBN, and THC; or CBD, CBDV, CBN, and THC.

In some embodiments, the active pharmaceutical ingredient comprises CBD, CBN, CBG, and THC. In some embodiments, the active pharmaceutical ingredient comprises CBD, CBN, CBG, and THC. In some embodiments, the active pharmaceutical ingredient comprises CBDV, CBN, CBG, and THC. In some embodiments, the active pharmaceutical ingredient comprises CBD, CBDV, CBN, CBG, and THC. In some embodiments, the active pharmaceutical ingredient comprises CBD, CBDV, CBN, CBG, and THC. In some embodiments, the active pharmaceutical ingredient comprises CBD, CBN, CBG, and THC. In some embodiments, the active pharmaceutical ingredient comprises CBD, CBN, CBG, and THC. In some embodiments, the active pharmaceutical ingredient comprises CBDV, CBN, CBG, and THC. In some embodiments, the active pharmaceutical ingredient comprises CBD, CBDV, CBN, CBG, and THC. In some embodiments, the active pharmaceutical ingredient comprises CBD, CBDV, CBN, CBG, and THC. In some embodiments, the active pharmaceutical ingredient comprises CBD, CBN, Phytol A, and THC. In some embodiments, the active pharmaceutical ingredient comprises CBD, CBN, Phytol A, and THC. In some embodiments, the active pharmaceutical ingredient comprises CBDV, CBN, Phytol A, and THC. In some embodiments, the active pharmaceutical ingredient comprises CBD, CBDV, CBN, Phytol A, and THC. In some embodiments, the active pharmaceutical ingredient comprises CBD, CBDV, CBN, Phytol A, and THC. In some embodiments, the active pharmaceutical ingredient comprises CBD, CBN, Phytol A, THC. In some embodiments, the active pharmaceutical ingredient comprises CBD, CBN, Phytol A, and THC. In some embodiments, the active pharmaceutical ingredient comprises CBDV, CBN, Phytol A, and THC. In some embodiments, the active pharmaceutical ingredient comprises CBD, CBDV, CBN, Phytol A, and THC. In some embodiments, the active pharmaceutical ingredient comprises CBD, CBDV, CBN, Phytol A, and THC. In some embodiments, the active pharmaceutical ingredient comprises CBD, CBN, and THC. In some embodiments, the active pharmaceutical ingredient comprises CBD, CBN, and THC. In some embodiments, the active pharmaceutical ingredient comprises CBDV, CBN, and THC. In some embodiments, the active pharmaceutical ingredient comprises CBD, CBDV, CBN, and THC. In some embodiments, the active pharmaceutical ingredient comprises CBD, CBDV, CBN, and THC.

In some embodiments, the active pharmaceutical ingredient comprises a molar ratio of 500-parts CBD:1,000-parts CBN:10-parts CBG:1-part THC; 1,000-parts CBD:1,000-parts CBN:10-parts CBG:1-part THC; 100-parts CBDV:1,000-parts CBN:10-parts CBG:1-part THC; 500-parts CBD:100-parts CBDV:1,000-parts CBN:10-parts CBG:1-part THC; 1,000-parts CBD:100-parts CBDV:1,000-parts CBN:10-parts CBG:1-part THC; 500-parts CBD:500-parts CBN:10-parts CBG:1-part THC;1,000-parts CBD:500-parts CBN:10-parts CBG:1-part THC; 100-parts CBDV:500-parts CBN:10-parts CBG:1-part THC; 500-parts CBD:100-parts CBDV:500-parts CBN:10-parts CBG:1-part THC; 1,000-parts CBD:100-parts CBN:500-parts CBDV:10-parts CBG:1-part THC; 5,000-parts CBD:10,000-parts CBN:1-part Phytol A:10-parts THC; 10,000-parts CBD:10,000-parts CBN:1-part Phytol A:10-parts THC; 1,000-parts CBDV:10,000-parts CBN:1-part Phytol A:10-parts THC; 5,000-parts CBD:1,000-parts CBDV:10,000-parts CBN:1-part Phytol A:10-parts THC; 10,000-parts CBD:1,000-parts CBDV:10,000-parts CBN:1-part Phytol A:10 THC; 5,000-parts CBD: 5,000-parts CBN:1-part Phytol A:10-parts THC; 10,000-parts CBD:5,000-parts CBN:1-part Phytol A:10-parts THC; 1,000-parts CBDV:5,000-parts CBN:1-part Phytol A:10-parts THC;5,000-parts CBD:1,000-parts CBDV:5,000-parts CBN:1-part Phytol A:10-parts THC; 10,000-parts CBD:1,000-parts CBDV:5,000-parts CBN:1-part Phytol A:10-parts THC; 500-parts CBD:1-part CBN:1-part THC; 1,000-parts CBD:1-part CBN:1-part THC; 100-parts CBDV:1-part CBN:1-part THC; 500-parts CBD:100-parts CBDV:1-part CBN:1-part THC; 1,000-parts CBD:100-parts CBDV:1-part CBN:1-part THC; 500-parts CBD:1,000-parts CBN:1-part THC; 1,000-parts CBD:1,000-parts CBN:1-part THC; 100-parts CBDV:1,000-parts CBN:1-part THC; 500:100:1,000:1 500-parts CBD:CBDV:CBN:1-part THC; 1,000-parts CBD:100-parts CBDV:1,000-parts CBN:1-part THC; 500-parts CBD:500-parts CBN:1-part THC; 1,000-parts CBD:500-parts CBN:1-part THC; 100-parts CBDV:500-parts CBN:1-part THC; 500-parts CBD:100-parts CBDV:500-parts CBN:1-part THC; or 1,000-parts CBD:100-parts CBDV:500-parts CBN:1-part THC.

In some embodiments, the active pharmaceutical ingredient comprises a molar ratio of 500-parts CBD:1,000-parts CBN:10-parts CBG:1-part THC. In some embodiments, the active pharmaceutical ingredient comprises a molar ratio of 1,000-parts CBD:1,000-parts CBN:10-parts CBG:1-part THC. In some embodiments, the active pharmaceutical ingredient comprises a molar ratio of 100-parts CBDV:1,000-parts CBN:10-parts CBG:1-part THC. In some embodiments, the active pharmaceutical ingredient comprises a molar ratio of 500-parts CBD:100-parts CBDV:1,000-parts CBN:10-parts CBG:1-part THC. In some embodiments, the active pharmaceutical ingredient comprises a molar ratio of 1,000-parts CBD:100-parts CBDV:1,000-parts CBN:10-parts CBG:1-part THC. In some embodiments, the active pharmaceutical ingredient comprises a molar ratio of 500-parts CBD:500-parts CBN:10-parts CBG:1-part THC. In some embodiments, the active pharmaceutical ingredient comprises a molar ratio of 1,000-parts CBD:500-parts CBN:10-parts CBG:1-part THC. In some embodiments, the active pharmaceutical ingredient comprises a molar ratio of 100-parts CBDV:500-parts CBN:10-parts CBG:1-part THC. In some embodiments, the active pharmaceutical ingredient comprises a molar ratio of 500-parts CBD:100-parts CBDV:500-parts CBN:10-parts CBG:1-part THC. In some embodiments, the active pharmaceutical ingredient comprises a molar ratio of 1,000-parts CBD:100-parts CBN:500-parts CBDV:10-parts CBG:1-part THC. In some embodiments, the active pharmaceutical ingredient comprises a molar ratio of 5,000-parts CBD:10,000-parts CBN:1-part Phytol A:10-parts THC. In some embodiments, the active pharmaceutical ingredient comprises a molar ratio of 10,000-parts CBD:10,000-parts CBN:1-part Phytol A:10-parts THC. In some embodiments, the active pharmaceutical ingredient comprises a molar ratio of 1,000-parts CBDV:10,000-parts CBN:1-part Phytol A:10-parts THC. In some embodiments, the active pharmaceutical ingredient comprises a molar ratio of 5,000-parts CBD:1,000-parts CBDV:10,000-parts CBN:1-part Phytol A:10-parts THC. In some embodiments, the active pharmaceutical ingredient comprises a molar ratio of 10,000-parts CBD:1,000-parts CBDV:10,000-parts CBN:1-part Phytol A:10 THC. In some embodiments, the active pharmaceutical ingredient comprises a molar ratio of 5,000-parts CBD: 5,000-parts CBN:1-part Phytol A:10-parts THC. In some embodiments, the active pharmaceutical ingredient comprises a molar ratio of 10,000-parts CBD:5,000-parts CBN:1-part Phytol A:10-parts THC. In some embodiments, the active pharmaceutical ingredient comprises a molar ratio of 1,000-parts CBDV:5,000-parts CBN:1-part Phytol A:10-parts THC. In some embodiments, the active pharmaceutical ingredient comprises a molar ratio of 5,000-parts CBD:1,000-parts CBDV:5,000-parts CBN:1-part Phytol A:10-parts THC. In some embodiments, the active pharmaceutical ingredient comprises a molar ratio of 10,000-parts CBD:1,000-parts CBDV:5,000-parts CBN:1-part Phytol A:10-parts THC. In some embodiments, the active pharmaceutical ingredient comprises a molar ratio of 500-parts CBD:1-part CBN:1-part THC. In some embodiments, the active pharmaceutical ingredient comprises a molar ratio of 1,000-parts CBD:1-part CBN:1-part THC. In some embodiments, the active pharmaceutical ingredient comprises a molar ratio of 100-parts CBDV:1-part CBN:1-part THC. In some embodiments, the active pharmaceutical ingredient comprises a molar ratio of 500-parts CBD:100-parts CBDV:1-part CBN:1-part THC. In some embodiments, the active pharmaceutical ingredient comprises a molar ratio of 1,000-parts CBD:100-parts CBDV:1-part CBN:1-part THC. In some embodiments, the active pharmaceutical ingredient comprises a molar ratio of 500-parts CBD:1,000-parts CBN:1-part THC. In some embodiments, the active pharmaceutical ingredient comprises a molar ratio of 1,000-parts CBD:1,000-parts CBN:1-part THC. In some embodiments, the active pharmaceutical ingredient comprises a molar ratio of 100-parts CBDV:1,000-parts CBN:1-part THC. In some embodiments, the active pharmaceutical ingredient comprises a molar ratio of 500:100:1,000:1 500-parts CBD:CBDV:CBN:1-part THC. In some embodiments, the active pharmaceutical ingredient comprises a molar ratio of 1,000-parts CBD:100-parts CBDV:1,000-parts CBN:1-part THC. In some embodiments, the active pharmaceutical ingredient comprises a molar ratio of 500-parts CBD:500-parts CBN:1-part THC. In some embodiments, the active pharmaceutical ingredient comprises a molar ratio of 1,000-parts CBD:500-parts CBN:1-part THC. In some embodiments, the active pharmaceutical ingredient comprises a molar ratio of 100-parts CBDV:500-parts CBN:1-part THC. In some embodiments, the active pharmaceutical ingredient comprises a molar ratio of 500-parts CBD:100-parts CBDV:500-parts CBN:1-part THC. In some embodiments, the active pharmaceutical ingredient comprises a molar ratio of or 1,000-parts CBD:100-parts CBDV:500-parts CBN:1-part THC.

In some embodiments, the IL-1β-decreasing and/or IL-6-decreasing cannabinoid, the TNFα-decreasing cannabinoid, the IFNγ-decreasing cannabinoid or terpene, and the Lymphopenia-reducing cannabinoid are synthetic or biosynthetic compound. In certain embodiments, the IL-1β-decreasing and/or IL-6-decreasing cannabinoid is a synthetic or biosynthetic compound. In certain embodiments, the TNFα-decreasing cannabinoid is a synthetic or biosynthetic compound. In certain embodiments, the IFNγ-decreasing cannabinoid or terpene is a synthetic or biosynthetic compound. In certain embodiments, the Lymphopenia-reducing is a synthetic or biosynthetic compound.

5.4.2. Relative Content

In typical embodiments, the IL-1β-decreasing and/or IL-6-decreasing cannabinoid, the TNFα-decreasing cannabinoid, the IFNγ-decreasing cannabinoid or terpene, and the Lymphopenia-reducing cannabinoid, collectively constitute 5-100% by weight (wt %) of the active ingredient.

In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 5-10 wt % of the active ingredient, 10-15 wt % of the active ingredient, 15-20 wt % of the active ingredient, 20-25 wt % of the active ingredient, 25-30 wt % of the active ingredient, 30-35 wt % of the active ingredient, or 35-40 wt % of the active ingredient. In certain embodiments, IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 5 wt %, at least 10 wt %, at least 15 wt %, at least 20 wt %, at least 25 wt %, at least 30 wt %, at least 35 wt %, but each case no more than 40 wt %, of the active ingredient.

In typical embodiments, IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 5-100% by weight of the active ingredient.

In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 5-10 wt % of the active ingredient, 10-15 wt % of the active ingredient, 15-20 wt % of the active ingredient, 20-25 wt % of the active ingredient, 25-30 wt % of the active ingredient, 30-35 wt % of the active ingredient, or 35-40 wt % of the active ingredient. In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 40-45 wt %, 40-45 wt %, 45-50 wt %, 50-55 wt %, 55-60 wt %, 60-65 wt %, 65-70 wt %, 70-75 wt %, 75-80 wt %, 80-95 wt %, or 95-100 wt %, of the active ingredient. In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 99 wt %, of the active ingredient.

In typical embodiments, the terpenes constitute 0-99% by weight of the active ingredient. In embodiments in which at least a terpene is present, the terpenes collectively constitute 0.005-0.05 wt % of the active ingredient.

In certain embodiments, the IL-1β-decreasing and/or the IL-6-decreasing cannabinoids constitute at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, or at least 65 wt %, at least 70 wt %, at least 75 wt %, at least 80 wt %, at least 85 wt %, at least 90 wt %, at least 95 wt %, or at least 99 wt %, of the active ingredient. In certain embodiments, the IL-1β-decreasing and/or the IL-6-decreasing cannabinoids constitute 8.66-68.7% by weight of the active ingredient.

In certain embodiments, the TNFα-decreasing cannabinoids constitute at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, or at least 65 wt %, at least 70 wt %, at least 75 wt %, at least 80 wt %, at least 85 wt %, at least 90 wt %, at least 95 wt %, or at least 99 wt %, of the active ingredient. In certain embodiments, the TNFα-decreasing cannabinoid constitutes 31.1-90.9% by weight of the active ingredient.

In certain embodiments, the IFNγ-decreasing cannabinoids or terpenes constitute at least 0.001 wt %, at least 0.01 wt %, at least 0.1 wt %, at least 1 wt %, at least 5 wt %, at least 10 wt %, at least 15 wt %, or at least 20 wt % of the active ingredient. In certain embodiments, the IFNγ-decreasing cannabinoids or terpenes constitute less than 0.001 wt %, less than 0.01 wt %, less than 0.1 wt %, less than 1 wt %, less than 5 wt %, less than 10 wt %, less than 15 wt %, or less than 20 wt % of the active ingredient. In certain embodiments, the IFNγ-decreasing cannabinoids or terpenes constitutes 0.005-9.8% by weight of the active ingredient.

In certain embodiments, the Lymphopenia-reducing cannabinoids constitute at least 0.001 wt %, at least 0.01 wt %, at least 0.1 wt %, at least 1 wt %, at least 5 wt %, at least 10 wt %, at least 15 wt %, or at least 20 wt % of the active ingredient. In certain embodiments, the Lymphopenia-reducing cannabinoids constitute less than 0.001 wt %, less than 0.01 wt %, less than 0.1 wt %, less than 1 wt %, less than 5 wt %, less than 10 wt %, less than 15 wt %, or less than 20 wt % of the active ingredient. In certain embodiments, the lymphopenia-reducing cannabinoid constitutes 0.047-1.3% by weight of the active ingredient.

In certain embodiments, the IL-1β-decreasing and/or the IL-6-decreasing cannabinoid constitutes 8.66-68.7% by weight of the active ingredient; and the TNFα-decreasing cannabinoid constitutes 31.25-90.9% by weight of the active ingredient.

In certain embodiments, the IL-1β-decreasing and/or IL-6-decreasing cannabinoid constitutes 9.08-68.3% by weight of the active ingredient; the TNFα-decreasing cannabinoid constitutes 31.1-90.9% by weight of the active ingredient; and the IFNγ-decreasing cannabinoid or terpene constitutes 0.005-9.8% by weight of the active ingredient.

In certain embodiments, the IL-1β-decreasing and/or IL-6-decreasing cannabinoid constitutes 9.08-68.3% by weight of the active ingredient; the TNFα-decreasing cannabinoid constitutes 31.23-90.8% by weight of the active ingredient; and the Lymphopenia-reducing cannabinoid constitutes 0.047-0.26%by weight of the active ingredient.

In certain embodiments, the IL-1β-decreasing and/or IL-6-decreasing cannabinoid constitutes 9.08-68.3% by weight of the active ingredient; the TNFα-decreasing cannabinoid constitutes 31.1-47.4% by weight of the active ingredient; the IFNγ-decreasing cannabinoid or terpene constitutes 0.005-1.6% by weight of the active ingredient; and the Lymphopenia-reducing cannabinoid constitutes 0.047-0.26% by weight of the active ingredient.

In certain embodiments, the IL-1β-decreasing and/or the IL-6-decreasing cannabinoid, and the TNFα-decreasing cannabinoid, collectively constitute 5-10% (w/v) of the active ingredient, 10-15% (w/v) of the active ingredient, 15-20% (w/v) of the active ingredient, 20-25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid and the TNFα-decreasing cannabinoid, collectively constitute at least 5% (w/v), at least 10% (w/v), at least 15% (w/v), at least 20% (w/v), at least 25% (w/v), at least 30% (w/v), at least 35% (w/v), at least 40% (w/v), at least 45% (w/v), at least 50% (w/v), at least 55% (w/v), at least 60% (w/v), at least 70% (w/v), at least 75% (w/v), at least 80% (w/v), at least 85% (w/v), at least 95% (w/v), or at least 99% (w/v), of the active ingredient.

In certain embodiments, the IL-1β-decreasing and/or the IL-6-decreasing cannabinoid and the TNFα-decreasing cannabinoid, collectively constitute 5-10% (w/v) of the active ingredient, 10-15% (w/v) of the active ingredient, 15-20% (w/v) of the active ingredient, 20 25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, and the TNFα-decreasing cannabinoid, collectively constitute 40-45% (w/v), 45-50% (w/v), 50-55% (w/v), 55-60% (w/v), 60-65% (w/v), 65-70% (w/v), 70-75% (w/v), 75-80% (w/v), 80-85% (w/v), 85-90% (w/v), 90-95% (w/v), 95-99.9% (w/v), or 95-100% (w/v), of the active ingredient. In certain embodiments, the IL-1β-decreasing and/or the IL-6-decreasing cannabinoid, and the TNFα-decreasing cannabinoid, collectively constitute at least 5% (w/v), 10% (w/v), 15% (w/v), 20% (w/v), 25% (w/v), 30% (w/v), 35% (w/v), 40% (w/v), 45% (w/v) or 50% (w/v), but in each case no more than 70% (w/v), of the active ingredient.

In certain embodiments, the IL-1β-decreasing and/or the IL-6-decreasing cannabinoid, the TNFα-decreasing cannabinoid, and the IFNγ-decreasing cannabinoid or terpene, collectively constitute 5-10% (w/v) of the active ingredient, 10-15% (w/v) of the active ingredient, 15-20% (w/v) of the active ingredient, 20-25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, and the IFNγ-decreasing cannabinoid or terpene, collectively constitute at least 5% (w/v), at least 10% (w/v), at least 15% (w/v), at least 20% (w/v), at least 25% (w/v), at least 30% (w/v), at least 35% (w/v), at least 40% (w/v), at least 45% (w/v), at least 50% (w/v), at least 55% (w/v), at least 60% (w/v), at least 70% (w/v), at least 75% (w/v), at least 80% (w/v), at least 85% (w/v), at least 95% (w/v), or at least 99% (w/v), of the active ingredient.

In certain embodiments, the IL-1β-decreasing and/or the IL-6-decreasing cannabinoid, the TNFα-decreasing cannabinoid, and the IFNγ-decreasing cannabinoid or terpene, collectively constitute 5-10% (w/v) of the active ingredient, 10-15% (w/v) of the active ingredient, 15-20% (w/v) of the active ingredient, 20 25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, and the IFNγ-decreasing cannabinoid or terpene, collectively constitute 40-45% (w/v), 45-50% (w/v), 50-55% (w/v), 55-60% (w/v), 60-65% (w/v), 65-70% (w/v), 70-75% (w/v), 75-80% (w/v), 80-85% (w/v), 85-90% (w/v), 90-95% (w/v), 95-99.9% (w/v), or 95-100% (w/v), of the active ingredient. In certain embodiments, the IL-1β-decreasing and/or the IL-6-decreasing cannabinoid, the TNFα-decreasing cannabinoid, and the IFNγ-decreasing cannabinoid or terpene, collectively constitute at least 5% (w/v), 10% (w/v), 15% (w/v), 20% (w/v), 25% (w/v), 30% (w/v), 35% (w/v), 40% (w/v), 45% (w/v) or 50% (w/v), but in each case no more than 70% (w/v), of the active ingredient.

In certain embodiments, the IL-1β-decreasing and/or the IL-6-decreasing cannabinoid, the TNFα-decreasing cannabinoid, and the Lymphopenia-reducing cannabinoid, collectively constitute 5-10% (w/v) of the active ingredient, 10-15% (w/v) of the active ingredient, 15-20% (w/v) of the active ingredient, 20-25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, and the Lymphopenia-reducing cannabinoid, collectively constitute at least 5% (w/v), at least 10% (w/v), at least 15% (w/v), at least 20% (w/v), at least 25% (w/v), at least 30% (w/v), at least 35% (w/v), at least 40% (w/v), at least 45% (w/v), at least 50% (w/v), at least 55% (w/v), at least 60% (w/v), at least 70% (w/v), at least 75% (w/v), at least 80% (w/v), at least 85% (w/v), at least 95% (w/v), or at least 99% (w/v), of the active ingredient.

In certain embodiments, the IL-1β-decreasing and/or the IL-6-decreasing cannabinoid, the TNFα-decreasing cannabinoid, and the Lymphopenia-reducing cannabinoid, collectively constitute 5-10% (w/v) of the active ingredient, 10-15% (w/v) of the active ingredient, 15-20% (w/v) of the active ingredient, 20 25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, and the Lymphopenia-reducing cannabinoid, collectively constitute 40-45% (w/v), 45-50% (w/v), 50-55% (w/v), 55-60% (w/v), 60-65% (w/v), 65-70% (w/v), 70-75% (w/v), 75-80% (w/v), 80-85% (w/v), 85-90% (w/v), 90-95% (w/v), 95-99.9% (w/v), or 95-100% (w/v), of the active ingredient. In certain embodiments, the IL-1β-decreasing and/or the IL-6-decreasing cannabinoid, the TNFα-decreasing cannabinoid, and the Lymphopenia-reducing cannabinoid, collectively constitute at least 5% (w/v), 10% (w/v), 15% (w/v), 20% (w/v), 25% (w/v), 30% (w/v), 35% (w/v), 40% (w/v), 45% (w/v) or 50% (w/v), but in each case no more than 70% (w/v), of the active ingredient.

In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 5-10% (w/v) of the active ingredient, 10-15% (w/v) of the active ingredient, 15-20% (w/v) of the active ingredient, 20-25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 5% (w/v), at least 10% (w/v), at least 15% (w/v), at least 20% (w/v), at least 25% (w/v), at least 30% (w/v), at least 35% (w/v), at least 40% (w/v), at least 45% (w/v), at least 50% (w/v), at least 55% (w/v), at least 60% (w/v), at least 70% (w/v), at least 75% (w/v), at least 80% (w/v), at least 85% (w/v), at least 95% (w/v), or at least 99% (w/v), of the active ingredient.

In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 5-10% (w/v) of the active ingredient, 10-15% (w/v) of the active ingredient, 15-20% (w/v) of the active ingredient, 20 25% (w/v) of the active ingredient, 25-30% (w/v) of the active ingredient, 30-35% (w/v) of the active ingredient, or 35-40% (w/v) of the active ingredient. In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 40-45% (w/v), 45-50% (w/v), 50-55% (w/v), 55-60% (w/v), 60-65% (w/v), 65-70% (w/v), 70-75% (w/v), 75-80% (w/v), 80-85% (w/v), 85-90% (w/v), 90-95% (w/v), 95-99.9% (w/v), or 95-100% (w/v), of the active ingredient. In certain embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 5% (w/v), 10% (w/v), 15% (w/v), 20% (w/v), 25% (w/v), 30% (w/v), 35% (w/v), 40% (w/v), 45% (w/v) or 50% (w/v), but in each case no more than 70% (w/v), of the active ingredient.

5.4.3. Absolute Content

In some embodiments, the pharmaceutically active ingredient consists of an IL-1β-decreasing and/or IL-6-decreasing cannabinoid, a TNFα-decreasing cannabinoid, an IFNγ-decreasing cannabinoid or terpene, and a Lymphopenia-reducing cannabinoid. In these embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 100 wt % of the pharmaceutically active ingredient.

In some embodiments, the pharmaceutically active ingredient consists of one or more of: a pDC-modulating cannabinoid or terpene, a monocyte-modulating cannabinoid or terpene, and a T-cell modulating cannabinoid or terpene. In some embodiment, the IL-6 decreasing cannabinoid is a monocyte modulating cannabinoid. In some embodiment, the TNFα decreasing cannabinoid is at least one of: a monocyte modulating cannabinoid, a T cell modulating cannabinoid, and a pDC modulating cannabinoid.

In some embodiments, the pharmaceutically active ingredient consists of: an IL-1β-decreasing and/or an IL-6-decreasing cannabinoid, and a TNFα-decreasing cannabinoid. In these embodiments, the IL-1β-decreasing and/or the IL-6-decreasing cannabinoid, and the TNFα-decreasing cannabinoid, collectively constitute 100 wt % of the pharmaceutically active ingredient.

In some embodiments, the pharmaceutically active ingredient consists of: an IL-1β-decreasing and/or an IL-6-decreasing cannabinoid, a TNFα-decreasing cannabinoid, and an IFNγ-decreasing cannabinoid or terpene. In these embodiments, the IL-1β-decreasing and/or the IL-6-decreasing cannabinoid, the TNFα-decreasing cannabinoid, and the IFNγ-decreasing cannabinoid or terpene, collectively constitute 100 wt % of the pharmaceutically active ingredient.

In some embodiments, the pharmaceutically active ingredient consists of: an IL-1β-decreasing and/or an IL-6-decreasing cannabinoid, a TNFα-decreasing cannabinoid, and a Lymphopenia-reducing cannabinoid. In these embodiments, the IL-1β-decreasing and/or the IL-6-decreasing cannabinoid, the TNFα-decreasing cannabinoid, and the Lymphopenia-reducing cannabinoid, collectively constitute 100 wt % of the pharmaceutically active ingredient.

In some embodiments, the active ingredient consists essentially of an IL-1β-decreasing and/or an IL-6-decreasing cannabinoid, a TNFα-decreasing cannabinoid, an IFNγ-decreasing cannabinoid or terpene, and a Lymphopenia-reducing cannabinoid. In some embodiments, the active ingredient consists essentially of one or more the IL-1β-decreasing and/or IL-6-decreasing cannabinoids, one or more TNFα-decreasing cannabinoids, one or more IFNγ-decreasing cannabinoids or terpenes, and one or more Lymphopenia-reducing cannabinoids.

In some embodiments, the active ingredient consists essentially of an IL-1β-decreasing and/or an IL-6-decreasing cannabinoid, and a TNFα-decreasing cannabinoid. In some embodiments, the active ingredient consists essentially of one or more IL-1β-decreasing and/or IL-6-decreasing cannabinoids, and one or more TNFα-decreasing cannabinoids.

In some embodiments, the active ingredient consists essentially of: an IL-1β-decreasing and/or an IL-6-decreasing cannabinoid, a TNFα-decreasing cannabinoid decreasing cannabinoid, and an IFNγ-decreasing cannabinoid or terpene. In some embodiments, the active ingredient consists essentially of one or more IL-1β-decreasing and/or IL-6-decreasing cannabinoids, one or more TNFα-decreasing cannabinoids, and one or more IFNγ-decreasing cannabinoids or terpenes.

In some embodiments, the active ingredient consists essentially of: an IL-1β-decreasing and/or an IL-6-decreasing cannabinoid, a TNFα-decreasing cannabinoid decreasing cannabinoid, and a Lymphopenia-reducing cannabinoid. In some embodiments, the active ingredient consists essentially of one or more IL-1β-decreasing and/or IL-6-decreasing cannabinoids, one or more TNFα-decreasing cannabinoids, and one or more Lymphopenia-reducing cannabinoids.

In other embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute less than 100% by weight (wt %) of the pharmaceutically active ingredient.

In various embodiments, the IL-1β-decreasing and/or the IL-6-decreasing cannabinoid, and the TNFα-decreasing cannabinoid, collectively constitute less than 100% by weight (wt %) of the pharmaceutically active ingredient.

In various embodiments, the IL-1β-decreasing cannabinoid and/or the IL-6-decreasing cannabinoid, the TNFα-decreasing cannabinoid, and the IFNγ-decreasing cannabinoid or terpene, collectively constitute less than 100% by weight (wt %) of the pharmaceutically active ingredient.

In other embodiments, the IL-1β-decreasing and/or the IL-6-decreasing cannabinoid, the TNFα-decreasing cannabinoid, and the Lymphopenia-reducing cannabinoid, collectively constitute less than 100% by weight (wt %) of the pharmaceutically active ingredient.

In some embodiments, the pharmaceutically active ingredient consists of an IL-1β-decreasing and/or an IL-6-decreasing cannabinoid, a TNFα-decreasing cannabinoid, an IFNγ-decreasing cannabinoid or terpene, and a Lymphopenia-reducing cannabinoid. In these embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute 100% (w/v) of the pharmaceutically active ingredient.

In other embodiments, the IL-1β-decreasing and/or the IL-6-decreasing cannabinoid, the TNFα-suppressing cannabinoid, the IFNγ-decreasing cannabinoid or terpene, and the Lymphopenia-reducing cannabinoid, collectively constitute less than 100% (w/v) of the pharmaceutically active ingredient.

5.4.4. Other Components

In some embodiments, the IL-1β-decreasing and/or the IL-6-decreasing cannabinoid, the

TNFα-decreasing cannabinoid, the IFNγ-decreasing cannabinoid or terpene, and the Lymphopenia-reducing cannabinoid, collectively constitute less than 100% by weight (wt %) of the pharmaceutically active ingredient.

In various such embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 75% by weight, but less than 100 wt %, of the pharmaceutically active ingredient. In specific embodiments, the IL-1I3-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 80%, at least at least 85%, at least 90%, at least 91%, at least 92%, at least 93%, at least 94%, or at least 95% by weight, but less than 100 wt %, of the active ingredient. In particular embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 96%, at least 97%, at least 98%, or at least 99% by weight, but less than 100 wt %, of the active ingredient.

In embodiments in which the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, constitute less than 100% by weight (wt %) of the pharmaceutically active ingredient, the active ingredient further comprises compounds other than the IL-1β-decreasing and/or the IL-6-decreasing cannabinoid, the TNFα-decreasing cannabinoid, the IFNγ-decreasing cannabinoid or terpene, and the Lymphopenia-reducing cannabinoid. In typical such embodiments, all other compounds in the active ingredient are extractable from Cannabis sativa, or can be biosynthetic or synthetic cannabinoid or terpene compounds. In specific embodiments, all other compounds in the active ingredient are present in an extract made from Cannabis sativa.

In some embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute less than 100% (w/v) of the pharmaceutically active ingredient.

In various such embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 75% (w/v), but less than 100% (w/v), of the pharmaceutically active ingredient. In specific embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 80%, at least at least 85%, at least 90%, at least 91%, at least 92%, at least 93%, at least 94%, or at least 95% (w/v), but less than 100% (w/v), of the active ingredient. In particular embodiments, the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute at least 96%, at least 97%, at least 98%, or at least 99% (w/v), but less than 100% (w/v), of the active ingredient.

In embodiments in which the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, collectively constitute less than 100% (w/v) of the pharmaceutically active ingredient, the active ingredient further comprises compounds other than the IL-6-decreasing cannabinoid, the TNFα-decreasing cannabinoid, the IFNγ-decreasing cannabinoid or terpene, and the Lymphopenia-reducing cannabinoid. In typical such embodiments, all other compounds in the active ingredient are extractable from Cannabis sativa or can be biosynthetic or synthetic homologues of cannabinoids or terpenes. In specific embodiments, all other compounds in the active ingredient are present in an extract made from Cannabis sativa.

In other embodiments, the compounds other than the IL-1β-decreasing and/or the IL-6-decreasing cannabinoid, the TNFα-decreasing cannabinoid, the IFNγ-decreasing cannabinoid or terpene, and the Lymphopenia-reducing cannabinoid, can include a Class 1 or Class 2 interferon (IFN). In certain embodiments, the Class 1 or Class 2 IFN is selected from IFNα 2a, IFNα 2b, IFNα n1, IFNα n3, IFNα con1, IFN-β 1a, IFNβ 1b, and IFNβ P-2a.

In various embodiments, the Class 1 or Class 2 IFN is IFNα 2a. In certain embodiments, the Class 1 or Class 2 IFN is IFNα 2b. In certain embodiments, the Class 1 or Class 2 IFN is IFNα n1. In certain embodiments, the Class 1 or Class 2 IFN is IFNα n3. In certain embodiments, the Class 1 or Class 2 IFN is IFNα coni. In certain embodiments, the Class 1 or Class 2 IFN is IFN-β 1a. In certain embodiments, the Class 1 or Class 2 IFN is IFNβ 1b. In certain embodiments, the Class 1 or Class 2 IFN is IFNβ P-2a.

In certain embodiments, the Class 1 or Class 2 IFN is selected from Roferon, Intron A, Wellferon, Alferon, Infergen, Rebif, Betaferon, Avonex, Betaseron, Pegasys, Peglntron, and Sylatron. In various embodiments, the Class 1 or Class 2 IFN is Roferon. In various embodiments, the Class 1 or Class 2 IFN is Intron A. In various embodiments, the Class 1 or Class 2 IFN is Wellferon. In various embodiments, the Class 1 or Class 2 IFN is Alferon. In various embodiments, the Class 1 or Class 2 IFN is Infergen. In various embodiments, the Class 1 or Class 2 IFN is Rebif. In various embodiments, the Class 1 or Class 2 IFN is Betaferon. In various embodiments, the Class 1 or Class 2 IFN is Avonex. In various embodiments, the Class 1 or Class 2 IFN is Betaseron. In various embodiments, the Class 1 or Class 2 IFN is Pegasys. In various embodiments, the Class 1 or Class 2 IFN is Peglntron. In various embodiments, the Class 1 or Class 2 IFN is Sylatron.

5.4.5. Cannabichromene (CBC) Content

In various embodiments, the active ingredient is substantially free of CBC. In some embodiments, the active ingredient is free of CBC.

5.4.6. Alpha-Pinene Content (aP)

In various embodiments, the active ingredient is substantially free of Alpha Pinene. In some embodiments, the active ingredient is free of Alpha-Pinene.

5.4.7. Trans-Nerolidol Content

In various embodiments, the active ingredient is substantially free of Trans-Nerolidol.

In various embodiments, the active ingredient is free of Trans-Nerolidol.

5.4.8. Linalool Content

In various embodiments, the active ingredient is substantially free of Linalool. In some embodiments, the active ingredient is free of Linalool.

5.4.9. Process for preparing active ingredient

In some embodiments, the pharmaceutically active ingredient is prepared by mixing chemically pure IL-1β-decreasing and/or IL-6-decreasing cannabinoids, TNFα-decreasing cannabinoids, IFNγ-decreasing cannabinoids or terpenes, and Lymphopenia-reducing cannabinoids, to desired final concentrations. Each of the IL-1β-decreasing and/or IL-6-decreasing cannabinoids, TNFα-decreasing cannabinoids, IFNγ-decreasing cannabinoids or terpenes, and Lymphopenia-reducing cannabinoids, can independently be chemically or biologically synthesized, either by total synthesis or by synthetic modification of an intermediate, purified from a compositional mixture such as a Cannabis sativa extract, or, purchased commercially.

In other embodiments, the pharmaceutically active ingredient is prepared from a starting compositional mixture by adjusting to predetermined desired final concentrations any one or more of the IL-1β-decreasing and/or the IL-6-decreasing cannabinoids, TNFα-decreasing cannabinoids, IFNγ-decreasing cannabinoids or terpenes, and Lymphopenia-reducing cannabinoids.

In certain embodiments, the starting compositional mixture is a Cannabis sativa extract.

In certain embodiments, the starting composition mixture include synthetic or biosynthetic cannabinoids or terpenes.

In certain embodiments, the starting compositional mixture is a Cannabis sativa extract and one or more of the IL-1β-decreasing and/or IL-6-decreasing cannabinoids, TNFα-decreasing cannabinoids, IFNγ-decreasing cannabinoids or terpenes, and Lymphopenia-reducing cannabinoids, is added to the mixture to achieve predetermined desired final concentrations.

Typically in such embodiments, the process further comprises the earlier step of determining the concentration of each desired IL-1β-decreasing and/or IL-6-decreasing cannabinoids, TNFα-decreasing cannabinoids, IFNγ-decreasing cannabinoids or terpenes, and Lymphopenia-reducing cannabinoids, in the starting compositional mixture.

In certain of these embodiments, the process further comprises the still earlier step of preparing a Cannabis sativa extract. Methods of preparing Cannabis sativa extracts are described in U.S. Pat. Nos. 6,403,126, 8,895,078, and 9,066,910; Doorenbos et al., Cultivation, extraction, and analysis of cannabis sativa L., Annals of The New York Academy of Sciences, 191, 3-14 (1971); Fairbairn and Liebmann, The extraction and estimation of the cannabinoids in Cannabis sativa L. and its products, Journal of Pharmacy and Pharmacology, 25, 150-155 (1973); Oroszlan and Verzar-petri, Separation, quantitation and isolation of cannabinoids from cannabis sativa L. by overpressured layer chromatography, Journal of Chromatography A, 388, 217-224 (1987), the disclosures of which are incorporated herein by reference in their entireties. In particular embodiments, the extraction method is chosen to provide an extract that has a content of IL-6-decreasing cannabinoids, TNFα-decreasing cannabinoids, IFNγ-decreasing cannabinoids or terpenes, and Lymphopenia-reducing cannabinoids, that best approximates the predetermined composition of the active ingredient.

In some embodiments, the process further comprises a first step of selecting a Cannabis sativa strain.

In certain embodiments, the strain selected has a typical content in the plant as a whole, or in an extractable portion thereof, of IL-1β-decreasing and/or IL-6-decreasing cannabinoids, TNFα-decreasing cannabinoids, IFNγ-decreasing cannabinoids or terpenes, and Lymphopenia-reducing cannabinoids that best approximates the predetermined composition of the active ingredient. In certain embodiments, the strain selected is one that is capable of providing an extract that best approximates the predetermined composition of the active ingredient. In specific embodiments, the strain selected has a typical content in the plant, extractable portion thereof, or extract thereof, that best approximates the predetermined weight ratios of desired IL-1β-decreasing and/or IL-6-decreasing cannabinoids, TNFα-decreasing cannabinoids, IFNγ-decreasing cannabinoids or terpenes, and Lymphopenia-reducing cannabinoids. In specific embodiments, the strain selected has a typical content in the plant, extractable portion thereof, or extract thereof, that requires adjustment in concentration of the fewest number of the desired IL-1β-decreasing and/or IL-6-decreasing cannabinoids, TNFα-decreasing cannabinoids, IFNγ-decreasing cannabinoids or terpenes, and Lymphopenia-reducing cannabinoids. In specific embodiments, the strain selected has a typical content in the plant, extractable portion thereof, or extract thereof, that requires the least expensive adjustment in concentration of the desired IL-1β-decreasing cannabinoid and/or IL-6-decreasing cannabinoids, TNFα-decreasing cannabinoids, IFNγ-decreasing cannabinoids or terpenes, and Lymphopenia-reducing cannabinoids.

5.4.10. Product By Process

In typical embodiments, the pharmaceutically active ingredient is prepared by one of the processes described in Section 4.4.11 above.

In embodiments in which the pharmaceutically active ingredient is prepared from a starting compositional mixture by adjusting to predetermined desired final concentrations any one or more of the IL-1β-decreasing and/or IL-6-decreasing cannabinoids, TNFα-decreasing cannabinoids, IFNγ-decreasing cannabinoids or terpenes, and Lymphopenia-reducing cannabinoids, all compounds in the active ingredient other than the IL-1β-decreasing and/or IL-6-decreasing cannabinoids, TNFα-decreasing cannabinoids, IFNγ-decreasing cannabinoids or terpenes, and Lymphopenia-reducing cannabinoids, are present within the starting compositional mixture.

In embodiments in which the starting compositional mixture is a Cannabis sativa extract, all compounds in the active ingredient other than the IL-1β-decreasing and/or IL-6-decreasing cannabinoids, TNFα-decreasing cannabinoids, IFNγ-decreasing cannabinoids or terpenes, and Lymphopenia-reducing cannabinoids, are present within the Cannabis sativa extract.

### 5.5. PHARMACEUTICAL COMPOSITIONS

In another aspect, pharmaceutical compositions are provided. The pharmaceutical composition comprises the pharmaceutically active ingredient disclosed herein and a pharmaceutically acceptable carrier or diluent.

5.5.1. Content of Pharmaceutically Active Ingredient

In typical embodiments, the active ingredient is present in the pharmaceutical composition at a concentration of at least 0.0001 mg/ml, at least 0.001 mg/ml, at least 0.01 mg/ml, at least 0.1 mg/ml, at least 0.5 mg/ml, or at least 1 mg/ml. In certain embodiments, the active ingredient is present in the pharmaceutical composition at a concentration of at least 1 mg/ml, at least 2 mg/ml, at least 3 mg/ml, at least 4 mg/ml, at least 5 mg/ml, at least 10 mg/ml, at least 15 mg/ml, at least 20 mg/ml, or at least 25 mg/ml. In certain embodiments, the active ingredient is present in the pharmaceutical composition at a concentration of at least 30 mg/ml, at least 35 mg/ml, at least 40 mg/ml, at least 45 mg/ml or at least 50 mg/ml.

5.5.2. Formulation Generally

The pharmaceutical composition can be in any form appropriate for human or veterinary medicine, including a liquid, an oil, an emulsion, a gel, a colloid, an aerosol or a solid.

The pharmaceutical composition can be formulated for administration by any route of administration appropriate for human or veterinary medicine, including enteral and parenteral routes of administration.

In various embodiments, the pharmaceutical composition is formulated for sublingual administration. In various embodiments, the pharmaceutical composition is formulated for buccal administration.

In some embodiments, the pharmaceutical composition is administered p.r.n.

In various embodiments, the pharmaceutical composition is formulated for administration by inhalation. In certain of these embodiments, the pharmaceutical composition is formulated for administration by a vaporizer. In certain of these embodiments, the pharmaceutical composition is formulated for administration by a nebulizer. In certain of these embodiments, the pharmaceutical composition is formulated for administration by an aerosolizer.

In various embodiments, the pharmaceutical composition is formulated for administration by a nanoparticle or nanoemulsion encapsulating the active ingredient.

In certain embodiments, the pharmaceutical composition can be encapsulated via nanoparticles, which may improve their stability. In certain embodiments, nanoparticles may be in a polymer matrix. In certain embodiments, the nanoparticles are lipid nanoparticles, that have a lipid monolayer enclosing a solid lipid core, dendrimers (nano sized three-dimensional branched molecules of polymer), nanotubes (sequence of nanoscale C60 atoms arranged in a long thin cylindrical structure), or nanoshells (concentric sphere consisting of a dielectric core and a metal shell).

In some embodiments, nanoemulsions include sub-micron sized emulsions. Nanoemulsion is an emulsion system having the droplet size in nanometer scale in which oil or water droplets are finely dispersed in the opposite phase with the help of a suitable surfactant to stabilize the system. The average droplet size usually ranges from 0.1 to 500 nm. The size of the droplets varies depending on the drug particles, mechanical energy, composition and relative amount of the surfactants. Nanoemulsions are also known as miniemulsions, fine-dispersed emulsions, submicron emulsions etc., which can be either O/W (oil in water) or W/O (water in oil) emulsion.

In various embodiments, the pharmaceutical composition is formulated for oral administration.

In some embodiments, the pharmaceutical composition is formulated for intravenous, intramuscular, or subcutaneous administration.

In some embodiments, the pharmaceutical composition is formulated for intrathecal or intracerebroventricular administration.

In some embodiments, the pharmaceutical composition is formulated for topical administration.

5.5.3. Pharmacological Compositions Adapted for Oral/Buccal/Sublingual Administration

Formulations for oral, buccal or sublingual administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral, buccal or sublingual administration (capsules, tablets, pills, dragees, powders, granules, and the like), one or more therapeutic agents may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption acancelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

5.5.4. Pharmacological Compositions Adapted for Administration By Inhalation

In some embodiments, unit dosage forms of the active pharmaceutical ingredient described herein are provided that are adapted for administration of the pharmaceutical composition by vaporizer, nebulizer, or aerosolizer. In some embodiments, the dosage form is a vial, an ampule, ly scored to allow user opening. In particular embodiments, the nebulizer is a jet nebulizer or an ultrasonic nebulizer.

Inhalable compositions are generally administered in an aqueous solution e.g., as a nasal or pulmonary spray. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering the solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; M. Naef et al. Development and pharmacokinetic characterization of pulmonal and intravenous delta-9-tetrahydrocannabinol (THC) in humans, J. Pharm. Sci. 93, 1176-84 (2004); and in U.S. Pat. Nos. 4,778,810; 6,080,762; 7,052,678; and 8,277,781 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Mucosal formulations are administered as dry powder formulations e.g., comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 micron mass median equivalent aerodynamic diameter (MMEAD), commonly about 1 micron MMEAD, and more typically about 2 micron MMEAD. Maximum particle size appropriate for deposition within the nasal passages is often about 10 micron MMEAD, commonly about 8 micron MMEAD, and more typically about 4 micron MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

5.5.5. Pharmacological Compositions Adapted for Injection

For intravenous, intramuscular, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In various embodiments, the unit dosage form is a vial, ampule, bottle, or pre-filled syringe. In some embodiments, the unit dosage form contains 0.01 mg, 0.1 mg, 0.5 mg, 1 mg, 2.5 mg, 5 mg, 10 mg, 12.5 mg, 25 mg, 50 mg, 75 mg, or 100 mg of the cannabinoid composition. In some embodiments, the unit dosage form contains 125 mg, 150 mg, 175 mg, or 200 mg of the cannabinoid composition. In some embodiments, the unit dosage form contains 250 mg of the cannabinoid composition.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition.

In particular embodiments, the unit dosage form is a vial containing 1 ml of the cannabinoid composition at a concentration of 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or 26 mg/ml, 27 mg/ml, 28 mg/ml, 29 mg/ml, 30 mg/ml or 30 mg/ml. In some embodiments, the unit dosage form is a vial containing 2 ml of the cannabinoid composition at a concentration of 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or 26 mg/ml, 27 mg/ml, 28 mg/ml, 29 mg/ml, 30 mg/ml or 40 mg/ml.

In some embodiments, the pharmaceutical composition in the unit dosage form is in solid form, such as a lyophilate, suitable for solubilization.

Unit dosage form embodiments suitable for subcutaneous, intradermal, or intramuscular administration include preloaded syringes, auto-injectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

5.5.6. Pharmacological Compositions Adapted for Topical Administration

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the cannabinoid-containing complex mixtures featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). The cannabinoid-containing complex mixtures featured in the invention may be encapsulated within liposomes or may form complexes thereto, in particular to cationic liposomes. Alternatively, the cannabinoid-containing complex mixtures may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof.

### 5.6. DOSE RANGES, GENERALLY

In vivo and/or in vitro assays may optionally be employed to help identify optimal dosage ranges for use. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

### 5.7. UNIT DOSAGE FORMS

The active pharmaceutical ingredient may conveniently be presented in unit dosage form.

In some embodiments, the IL-1β-decreasing and/or IL-6-decreasing cannabinoid of the unit dosage form is Cannabidivarin (CBDV) and is present in an amount sufficient to achieve a mean peak concentration (Cₘₐₓ) in plasma and/or target tissue of at least 0.5 μM when administered.

A target tissue can be any tissue or organ in the body, such as, but not limited to, heart, lungs, liver, kidney, brain. In some embodiments, the target tissue is the tissue or organ that is affected by CRS or MAS. In some embodiments, the target tissue is a tissue or organ that has an increased amount of IL-1β, IL-6, TNFα, IL-2, and/or IFNα inflammatory cytokines.

In certain embodiments, the IL-1β-decreasing and/or IL-6-decreasing cannabinoid of the unit dosage form is Cannabidivarin (CBDV) and is present in an amount sufficient to achieve a mean peak concentration (Cₘₐₓ) in plasma and/or target tissuetarget tissue of at least 1 when administered. In certain embodiments, the IL-1β-decreasing and/or IL-6-decreasing cannabinoid of the unit dosage form is Cannabidivarin (CBDV) and is present in an amount sufficient to achieve a mean peak concentration (Cₘₐₓ) in plasma and/or target tissue of at least 2 μM when administered. In some embodiments, the IL-1β-decreasing and/or IL-6-decreasing cannabinoid of the unit dosage form is Cannabidivarin (CBDV) and is present in an amount sufficient to achieve a mean peak concentration (Cₘₐₓ) in plasma and/or target tissue of at least 0.1 μM, 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, at least 2.1 μM, at least 2.2 μM, at least 2.3 μM, at least 2.4 μM, at least 2.5 μM, at least 2.7 μM, at least 2.8 μM, at least 2.9 μM, or at least 3 μM when administered.

In certain embodiments, the IL-1β-decreasing and/or IL-6-decreasing cannabinoid of the unit dosage form is CBDV and is present in an amount sufficient to achieve a Cₘₐₓ in plasma and/or target tissue of 0.1 μM to 3 μM when administered. In certain embodiments, the IL-1β-decreasing and/or IL-6-decreasing cannabinoid of the unit dosage form is CBDV and is present in an amount sufficient to achieve a Cₘₐₓ in plasma and/or target tissue of 0.5 μM to 2 μM when administered.

In certain embodiments, the IL-1β-decreasing and/or IL-6-decreasing cannabinoid of the unit dosage form is Cannabidiol (CBD) and is present in an amount sufficient to achieve a Cmax in plasma and/or target tissue of at least 5 μM when administered. In certain embodiments, the IL-1β-decreasing and/or the IL-6-decreasing cannabinoid of the unit dosage form is Cannabidiol (CBD) and is present in an amount sufficient to achieve a Cₘₐₓ in plasma and/or target tissue of at least 10 μM when administered. In certain embodiments, the IL-1β-decreasing and/or the IL-6-decreasing cannabinoid of the unit dosage form is Cannabidiol (CBD) and is present in an amount sufficient to achieve a Cₘₐₓ in plasma and/or target tissue of at least at least 1 μM, at least 2 μM , at least 3 μM , at least 4 μM , at least 5 μM, at least 6 μM, at least 7 μM, at least 8 μM, at least 9 μM, at least 10 μM, at least 11 μM, at least 12 μM, at least 13 μM, at least 14 μM, or at least 15 μM, when administered.

In certain embodiments, the IL-1β-decreasing and/or the IL-6-decreasing cannabinoid of the unit dosage form is CBD and is present in an amount sufficient to achieve a Cₘₐₓ in plasma and/or target tissue of 1 μM to 15 μM when administered. In certain embodiments, the IL-6-decreasing cannabinoid of the unit dosage form is CBD and is present in an amount sufficient to achieve a Cmax in plasma and/or target tissue of 5 μM to 10 μM when administered.

In certain embodiments, the IL-1β-decreasing and/or the IL-6-decreasing cannabinoid of the unit dosage form is a combination of Cannabidiol (CBD)and Cannabidivarin (CBDV), wherein the CBDV is present in an amount sufficient to achieve a mean peak concentration (Cmax) in plasma and/or target tissue of at least 0.5 μM when administered and the CBD is present in an amount sufficient to achieve a Cₘₐₓ in plasma and/or target tissue of at least 5 μM when administered.

In some embodiments, the IL-1β-decreasing cannabinoid of the unit dosage form is Cannabidivarin (CBDV) and is present in an amount sufficient to achieve a mean peak concentration (Cmax) in plasma and/or target tissue of at least 0.5 μM when administered. In certain embodiments, the IL-1β-decreasing cannabinoid of the unit dosage form is Cannabidivarin (CBDV) and is present in an amount sufficient to achieve a mean peak concentration (Cmax) in plasma and/or target tissue of at least 1 μM when administered. In certain embodiments, the IL-1β-decreasing cannabinoid of the unit dosage form is Cannabidivarin (CBDV) and is present in an amount sufficient to achieve a mean peak concentration (Cmax) in plasma and/or target tissue of at least 2 μM when administered. In some embodiments, the IL-1β-decreasing cannabinoid of the unit dosage form is Cannabidivarin (CBDV) and is present in an amount sufficient to achieve a mean peak concentration (Cmax) in plasma and/or target tissue of at least 0.1 μM, 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, at least 2.1 μM, at least 2.2 μM, at least 2.3 μM, at least 2.4 μM, at least 2.5 μM, at least 2.7 μM, at least 2.8 μM, at least 2.9 μM, or at least 3 μM when administered.

In certain embodiments, the IL-1β-decreasing cannabinoid of the unit dosage form is CBDV and is present in an amount sufficient to achieve a Cmax in plasma and/or target tissue of 0.1 μM to 3 μM when administered. In certain embodiments, the IL-1β-decreasing cannabinoid of the unit dosage form is CBDV and is present in an amount sufficient to achieve a Cmax in plasma and/or target tissue of 0.5 μM to 2 μM when administered.

In certain embodiments, the IL-1β-decreasing cannabinoid of the unit dosage form is Cannabidiol (CBD) and is present in an amount sufficient to achieve a Cmax in plasma and/or target tissue of at least 5 μM when administered. In certain embodiments, the IL-1β-decreasing cannabinoid of the unit dosage form is Cannabidiol (CBD) and is present in an amount sufficient to achieve a Cmax in plasma and/or target tissue of at least 10 μM when administered. In certain embodiments, the IL-1β-decreasing cannabinoid of the unit dosage form is Cannabidiol (CBD) and is present in an amount sufficient to achieve a Cmax in plasma and/or target tissue of at least at least 1 μM, at least 2 μM , at least 3 μM , at least 4 μM , at least 5 μM, at least 6 μM, at least 7 μM, at least 8 μM, at least 9 μM, at least 10 μM, at least 11 μM, at least 12 μM, at least 13 μM, at least 14 μM, or at least 15 μM, when administered.

In certain embodiments, the IL-1β-decreasing cannabinoid of the unit dosage form is CBD and is present in an amount sufficient to achieve a Cmax in plasma and/or target tissue of 1 μM to 15 μM when administered. In certain embodiments, the IL-6-decreasing cannabinoid of the unit dosage form is CBD and is present in an amount sufficient to achieve a Cmax in plasma and/or target tissue of 5 μM to 10 μM when administered.

In certain embodiments, the IL-1β-decreasing cannabinoid of the unit dosage form is a combination of Cannabidiol (CBD)and Cannabidivarin (CBDV), wherein the CBDV is present in an amount sufficient to achieve a mean peak concentration (Cmax) in plasma and/or target tissue of at least 0.5 μM when administered and the CBD is present in an amount sufficient to achieve a Cmax in plasma and/or target tissue of at least 5 μM when administered.

In some embodiments, the IL-6-decreasing cannabinoid of the unit dosage form is Cannabidivarin (CBDV) and is present in an amount sufficient to achieve a mean peak concentration (Cmax) in plasma and/or target tissue of at least 0.5 μM when administered. In certain embodiments, the IL-6-decreasing cannabinoid of the unit dosage form is Cannabidivarin (CBDV) and is present in an amount sufficient to achieve a mean peak concentration (Cmax) in plasma and/or target tissue of at least 1 μM when administered. In certain embodiments, the IL-6-decreasing cannabinoid of the unit dosage form is Cannabidivarin (CBDV) and is present in an amount sufficient to achieve a mean peak concentration (Cmax) in plasma and/or target tissue of at least 2 μM when administered. In some embodiments, the IL-6-decreasing cannabinoid of the unit dosage form is Cannabidivarin (CBDV) and is present in an amount sufficient to achieve a mean peak concentration (Cₘₐₓ) in plasma and/or target tissue of at least 0.1 μM, 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, at least 2.1 μM, at least 2.2 μM, at least 2.3 μM, at least 2.4 μM, at least 2.5 μM, at least 2.7 μM, at least 2.8 μM, at least 2.9 μM, or at least 3 μM when administered.

In certain embodiments, the IL-6-decreasing cannabinoid of the unit dosage form is CBDV and is present in an amount sufficient to achieve a Cmax in plasma and/or target tissue of 0.1 μM to 3 μM when administered. In certain embodiments, the IL-6-decreasing cannabinoid of the unit dosage form is CBDV and is present in an amount sufficient to achieve a Cmax in plasma and/or target tissue of 0.5 μM to 2 μM when administered.

In certain embodiments, the IL-6-decreasing cannabinoid of the unit dosage form is Cannabidiol (CBD) and is present in an amount sufficient to achieve a Cmax in plasma and/or target tissue of at least 5 μM when administered. In certain embodiments, the IL-6-decreasing cannabinoid of the unit dosage form is Cannabidiol (CBD) and is present in an amount sufficient to achieve a Cmax in plasma and/or target tissue of at least 10 μM when administered. In certain embodiments, the IL-6-decreasing cannabinoid of the unit dosage form is Cannabidiol (CBD) and is present in an amount sufficient to achieve a Cₘₐₓ in plasma and/or target tissue of at least at least 1 μM, at least 2 μM , at least 3 μM , at least 4 μM , at least 5 μM, at least 6 μM, at least 7 uM, at least 8 μM, at least 9 μM, at least 10 μM, at least 11 μM, at least 12 μM, at least 13 μM, at least 14 μM, or at least 15 μM, when administered.

In certain embodiments, the IL-6-decreasing cannabinoid of the unit dosage form is CBD and is present in an amount sufficient to achieve a Cₘₐₓ in plasma and/or target tissue of 1 uM to 15 μM when administered. In certain embodiments, the IL-6-decreasing cannabinoid of the unit dosage form is CBD and is present in an amount sufficient to achieve a Cmax in plasma and/or target tissue of 5 μM to 10 μM when administered.

In certain embodiments, the IL-6-decreasing cannabinoid of the unit dosage form is a combination of Cannabidiol (CBD)and Cannabidivarin (CBDV), wherein the CBDV is present in an amount sufficient to achieve a mean peak concentration (Cmax) in plasma and/or target tissue of at least 0.5 μM when administered and the CBD is present in an amount sufficient to achieve a Cmax in plasma and/or target tissue of at least 5 μM when administered.

In some embodiments, the TNFα-decreasing cannabinoid of the unit dosage form is Cannabinol (CBN) and is present in an amount sufficient to achieve a Cmax in plasma and/or target tissue of at least 5 μM when administered. In certain embodiments, the TNFα-decreasing cannabinoid of the unit dosage form is Cannabinol (CBN) and is present in an amount sufficient to achieve a Cₘₐₓ in plasma and/or target tissue of at least 10 μM when administered. In certain embodiments, the TNFα-decreasing cannabinoid of the unit dosage form is Cannabinol (CBN) and is present in an amount sufficient to achieve a Cₘₐₓ in plasma and/or target tissue of at least 1 μM, at least 2 μM , at least 3 μM , at least 4 μM , at least 5 μM, at least 6 μM, at least 7 μM, at least 8 μM, at least 9 μM, at least 10 μM, at least 11 μM, at least 12 μM, at least 13 μM, at least 14 μM, or at least 15 μM, when administered.

In certain embodiments, the TNFα-decreasing cannabinoid of the unit dosage form is Cannabinol (CBN) and is present in an amount sufficient to achieve a Cmax in plasma and/or target tissue of 1 to 15 μM when administered. In some embodiments, the TNFα-decreasing cannabinoid of the unit dosage form is Cannabinol (CBN) and is present in an amount sufficient to achieve a Cmax in plasma and/or target tissue of 5 to 10 μM when administered.

In some embodiments, the IFNγ-decreasing cannabinoid of the unit dosage form is Cannabigerol (CBG) and is present in an amount sufficient to achieve a Cₘₐₓ in plasma and/or target tissue of at least 0.01 μM when administered. In certain embodiments, the IFNγ-decreasing cannabinoid of the unit dosage form is Cannabigerol (CBG) and is present in an amount sufficient to achieve a Cₘₐₓ in plasma and/or target tissue of at least 0.1 μM when administered. In certain embodiments, the IFNγ-decreasing cannabinoid of the unit dosage form is Cannabigerol (CBG) and is present in an amount sufficient to achieve a Cₘₐₓ in plasma and/or target tissue of at least 0.2 μM when administered. In some embodiments, the IFNγ-decreasing cannabinoid of the unit dosage form is Cannabigerol (CBG) and is present in an amount sufficient to achieve a Cₘₐₓ in plasma and/or target tissue of at least 0.005 μM, 0.006 μM, 0.008 μM, 0.009 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.1 μM, at least 1.2 μM, at least 1.3 at least 1.4 at least 1.5 at least 1.6 at least 1.7 at least 1.8 μM, at least 1.9 or at least 2 when administered.

In some embodiments, the IFNγ-decreasing cannabinoid of the unit dosage form is Cannabigerol (CBG) and is present in an amount sufficient to achieve a Cₘₐₓ in plasma and/or target tissue of 0.01 μM to 0.2 μM when administered. In some embodiments, the IFNγ-decreasing cannabinoid of the unit dosage form is Cannabigerol (CBG) and is present in an amount sufficient to achieve a Cmax in plasma and/or target tissue of 0.005 μM to 5 μM when administered.

In some embodiments, the IFNγ-decreasing cannabinoid of the unit dosage form is Cannabinol (CBN) and is present in an amount sufficient to achieve a Cmax in plasma and/or target tissue of at least 0.001 μM when administered. In certain embodiments, the IFNγ-decreasing cannabinoid of the unit dosage form is Cannabinol (CBN) and is present in an amount sufficient to achieve a Cmax in plasma and/or target tissue of at least 0.01 μM when administered. In certain embodiments, the IFNγ-decreasing cannabinoid of the unit dosage form is Cannabinol (CBN) and is present in an amount sufficient to achieve a Cmax in plasma and/or target tissue of at least 0.1 μM when administered. In some embodiments, the IFNγ-decreasing cannabinoid of the unit dosage form is Cannabinol (CBN) and is present in an amount sufficient to achieve a Cmax in plasma and/or target tissue of at least 0.0005 μM, 0.0006 μM, 0.0007 μM, 0.0008 μM, 0.0009 μM, 0.001 μM, 0.002 μM, 0.003 μM, 0.004 μM, 0.005 μM, 0.006 μM, 0.008 μM, 0.009 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, at least 0.3 μM, at least 0.4 μM, at least 0.5 μM, at least 0.6 μM, at least 0.7 μM, at least 0.8 μM, at least 0.9 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2 μM, when administered.

In some embodiments, the IFNγ-decreasing cannabinoid of the unit dosage form is Cannabinol (CBN) and is present in an amount sufficient to achieve a Cmax in plasma and/or target tissue of 0.01 μM to 0.2 μM when administered. In some embodiments, the IFNγ-decreasing cannabinoid of the unit dosage form is Cannabinol (CBN) and is present in an amount sufficient to achieve a Cmax in plasma and/or target tissue of 0.005 μM to 5 μM when administered.

In some embodiments, the IFNγ-decreasing cannabinoid of the unit dosage form is Phytol A and is present in an amount sufficient to achieve a Cmax in plasma and/or target tissue of at least 0.0005 μM when administered. In some embodiments, the IFNγ-decreasing cannabinoid of the unit dosage form is Phytol A and is present in an amount sufficient to achieve a Cmax in plasma and/or target tissue of at least 0.001 μM when administered. In certain embodiments, the IFNγ-decreasing cannabinoid of the unit dosage form is Phytol A and is present in an amount sufficient to achieve a Cₘₐₓ in plasma and/or target tissue of at least 0.00005 μM, at least 0.00006 μM, at least 0.00007 μM, at least 0.00008 μM, at least 0.00009 μM, at least 0.0001 μM, at least 0.0002 μM, at least 0.0003 μM, at least 0.0004 μM, at least 0.0005 μM, at least 0.0006 μM, at least 0.0007 μM, at least 0.0008 μM, at least 0.0009 μM, at least 0.001 μM, at least 0.002 μM, at least 0.003 μM, at least 0.004 μM, at least 0.005 μM, at least 0.006 μM, at least 0.007 μM, at least 0.008 μM, at least 0.009 μM, at least 0.01 μM, at least 0.02 μM, at least 0.03 μM, at least 0.04 μM, at least 0.05 μM, at least 0.06 μM, at least 0.07 μM, at least 0.08 μM, at least 0.09 μM, or at least 0.1 μM.

In some embodiments, the IFNγ-decreasing cannabinoid of the unit dosage form is Phytol A and is present in an amount sufficient to achieve a Cmax in plasma and/or target tissue of 0.00005 μM to 0.01 μM when administered. In some embodiments, the IFNγ-decreasing cannabinoid of the unit dosage form is Phytol A and is present in an amount sufficient to achieve a Cmax in plasma and/or target tissue of 0.0005 μM to 0.001 μM when administered.

In some embodiments, the Lymphopenia-reducing cannabinoid of the unit dosage form is tetrahydrocannabinol (THC) and is present in an amount sufficient to achieve a Cmax in plasma and/or target tissue of at least 0.01 μM when administered. In certain embodiments, the Lymphopenia-reducing cannabinoid of the unit dosage form is tetrahydrocannabinol (THC) and is present in an amount sufficient to achieve Cₘₐₓ in plasma and/or target tissue of less than 0.02 μM when administered.

In some embodiments, the Lymphopenia-reducing cannabinoid of the unit dosage form is tetrahydrocannabinol (THC) and is present in an amount sufficient to achieve a Cₘₐₓ in plasma and/or target tissue of at least 0.01, at least 0.011 μM , at least 0.012 μM , at least 0.013 μM, 0.014 μM, 0.015 μM, 0.016 μM, 0.017 μM, 0.018 μM, 0.019 μM, or at least 0.02 μM when administered.

In certain embodiments, the Lymphopenia-reducing cannabinoid of the unit dosage form is tetrahydrocannabinol (THC) and is present in an amount sufficient to achieve a Cₘₐₓ in plasma and/or target tissue of at least 0.01 μM but less than 0.02 μM when administered.

In some embodiments, the unit dosage form comprises: 0.2 mg to 35 mg of the IL-1β-decreasing and/or IL-6-decreasing cannabinoid or terpene; 2 mg to 35 mg of the TNFα-decreasing cannabinoid; 0.0002 mg to 10 mg of dose of the IFNγ-decreasing cannabinoid, and 0.001 mg to 0.5 mg of the Lymphopenia-reducing cannabinoid. In certain embodiments, the unit dosage form comprises: 0.7 mg to 28.3 mg of the IL-1β-decreasing and/or IL-6-decreasing cannabinoid or terpene; 7.8 mg to 30 mg of the TNFα-decreasing cannabinoid; 0.0007 mg to 5.70 mg of dose of the IFNγ-decreasing cannabinoid, and 0.01 mg to 0.05 mg of the Lymphopenia-reducing cannabinoid.

In some embodiments, the unit dosage form comprises: 0.2 mg to 35 mg of the IL-1β-decreasing and/or IL-6-decreasing cannabinoid or terpene; and 2 mg to 35 mg dose of the TNFα-decreasing cannabinoid. In certain embodiments, the unit dosage form comprises: 0.7 mg to 28.3 mg of the IL-1β-decreasing and/or IL-6-decreasing cannabinoid or terpene; and 7.8 mg to 30 mg of the TNFα-decreasing cannabinoid.

In some embodiments, the unit dosage form comprises: 0.2 mg to 35 mg of the IL-1β-decreasing and/or IL-6-decreasing cannabinoid or terpene; 2 mg to 35 mg of the TNFα-decreasing cannabinoid; and 0.0002 mg to 10 mg of dose of the IFNγ-decreasing cannabinoid. In certain embodiments, the unit dosage form comprises: 0.7 mg to 28.3 mg of the IL-1β-decreasing and/or IL-6-decreasing cannabinoid or terpene; 7.8 mg to 30 mg of the TNFα-decreasing cannabinoid; and 0.0007 mg to 5.70 mg of dose of the IFNγ-decreasing cannabinoid.

In some embodiments, the unit dosage form comprises: 0.2 mg to 35 mg of the IL-1β-decreasing and/or IL-6-decreasing cannabinoid or terpene; 2 mg to 35 mg of the TNFα-decreasing cannabinoid; and 0.001 mg to 0.5 mg of the Lymphopenia-reducing cannabinoid. In certain embodiments, the unit dosage form comprises: 0.7 mg to 28.3 mg of the IL-1β-decreasing and/or IL-6-decreasing cannabinoid or terpene; 7.8 mg to 30 mg of the TNFα-decreasing cannabinoid; and 0.01 mg to 0.05 mg of the Lymphopenia-reducing cannabinoid.

In some embodiments, the unit dosage form comprises 3 mg to 35 mg dosage of the IL-1β-decreasing and/or IL-6-decreasing cannabinoid, where the IL-1β-decreasing and/or IL-6-decreasing cannabinoid is CBD. In certain embodiments, the unit dosage form comprises 7.86 mg to 28.3 mg dosage of the IL-1β-decreasing and/or IL-6-decreasing cannabinoid, where the IL-1β-decreasing and/or IL-6-decreasing cannabinoid is CBD. In certain embodiments, the unit dosage form comprises 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, or 35 mg dosage of the IL-1β-decreasing and/or IL-6-decreasing cannabinoid, where the IL-1β-decreasing and/or IL-6-decreasing cannabinoid is CBD.

In some embodiments, the unit dosage form comprises 0.2 mg to 10 mg dosage of the IL-1β-decreasing and/or IL-6-decreasing cannabinoid, where the IL-6-decreasing cannabinoid is CBDV. In certain embodiments, the unit dosage form comprises 0.72 mg to 5.16 mg dosage of the IL-1β-decreasing and/or IL-6-decreasing cannabinoid, where the IL-1β-decreasing and/or IL-6-decreasing cannabinoid is CBDV. In certain embodiments, the unit dosage form comprises 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, or 10 mg dosage of the IL-1β-decreasing and/or IL-6-decreasing cannabinoid, where the IL-1β-decreasing and/or IL-6-decreasing cannabinoid is CBDV.

In some embodiments, the unit dosage form comprises 2 mg to 35 mg dosage of the TNFα-decreasing cannabinoid, where the TNFα-decreasing cannabinoid is CBN. In certain embodiments, the unit dosage form comprises 7.76 mg to 27.9 mg dosage of the TNFα-decreasing cannabinoid, where the TNFα-decreasing cannabinoid is CBN. In certain embodiments, the unit dosage form comprises 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, or 35 mg dosage of the TNFα-decreasing cannabinoid, where the TNFα-decreasing cannabinoid is CBN.

In some embodiments, the unit dosage form comprises 0.2 mg to 10 mg dosage of the IFNγ-suppressing cannabinoid or terpene, where the IFNγ-decreasing cannabinoid or terpene is CBG. In certain embodiments, the unit dosage form comprises 0.791 mg to 5.70 mg dosage of the IFNγ-suppressing cannabinoid or terpene, where the IFNγ-suppressing cannabinoid or terpene is CBG. In certain embodiments, the unit dosage form comprises 0.006 mg to 1 mg dosage of the IFNγ-suppressing cannabinoid or terpene, where the IFNγ-suppressing cannabinoid or terpene is CBG. In certain embodiments, the unit dosage form comprises 0.009 mg to 0.8 mg dosage of the IFNγ-suppressing cannabinoid or terpene, where the IFNγ-suppressing cannabinoid or terpene is CBG. In certain embodiments, the unit dosage form comprises 0.01 mg to 0.6 mg dosage of the IFNγ-suppressing cannabinoid or terpene, where the IFNγ-suppressing cannabinoid or terpene is CBG. In certain embodiments, the unit dosage form comprises 0.005 mg, 0.006 mg, 0.007 mg, 0.008 mg, 0.009 mg, 0.01 mg, 0.012 mg, 0.013 mg, 0.014 mg, 0.015 mg, 0.016 mg, 0.017 mg, 0.018 mg, 0.019 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, or 10 mg dosage of the IFNγ-decreasing cannabinoid or terpene, where the IFNγ-decreasing cannabinoid or terpene is CBG.

In some embodiments, the unit dosage form comprises 0.0005 mg to 0.5 mg dosage of the IFNγ-suppressing cannabinoid or terpene, where the IFNγ-suppressing cannabinoid or terpene is CBN. In certain embodiments, the unit dosage form comprises 0.001 mg to 0.3 mg dosage of the IFNγ-suppressing cannabinoid or terpene, where the IFNγ-suppressing cannabinoid or terpene is CBN. In certain embodiments, the unit dosage form comprises 0.0005 mg, 0.0006 mg, 0.0007 mg, 0.0008 mg, 0.0009 mg, 0.001 mg, 0.0012 mg, 0.0013 mg, 0.0014 mg, 0.0015 mg, 0.0016 mg, 0.0017 mg, 0.0018 mg, 0.0019 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.22 mg, 0.24 mg, 0.26 mg, 0.28 mg, 0.3 mg, 0.4 mg, 0.5 mg, 0.6 mg, 0.7 mg, 0.8 mg, 0.9 mg, or 1 mg, dosage of the IFNγ-decreasing cannabinoid or terpene, where the IFNγ-decreasing cannabinoid or terpene is CBN.

In some embodiments, the unit dosage form comprises 0.0002 mg to 0.05 mg dosage of the IFNγ-suppressing cannabinoid or terpene, where the IFNγ-suppressing cannabinoid or terpene is Phytol A. In certain embodiments, the unit dosage form comprises 0.0002 mg. 0.0003 mg, to 0.0022 mg dosage of the IFNγ-suppressing cannabinoid or terpene, where the IFNγ-suppressing cannabinoid or terpene is Phytol A. In certain embodiments, the unit dosage form comprises 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, or 0.05 mg dosage of the IFNγ-decreasing cannabinoid or terpene, where the IFNγ-decreasing cannabinoid or terpene is Phytol A.

In some embodiments, the unit dosage form comprises 0.001 mg to 0.1 mg dosage of the Lymphopenia-reducing cannabinoid, where the Lymphopenia-reducing cannabinoid is THC. In certain embodiments, the unit dosage form comprises 0.01 mg to 0.05 mg dosage of the Lymphopenia-reducing cannabinoid, where the Lymphopenia-reducing cannabinoid is THC. In certain embodiments, the unit dosage form comprises 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, or 0.1 mg dosage of the Lymphopenia-reducing cannabinoid, where the Lymphopenia-reducing cannabinoid is THC.

In some embodiments, the unit dosage form comprises 14 mg dosage of the IL-1β-decreasing and/or IL-6-decreasing cannabinoid, wherein the IL-1β-decreasing and/or IL-6-decreasing cannabinoid is CBD. In some embodiments, the unit dosage form comprises 28 mg dosage of the IL-1β-decreasing and/or IL-6-decreasing cannabinoid, wherein the IL-1β-decreasing and/or IL-6-decreasing cannabinoid is CBD.

In some embodiments, the unit dosage form comprises 1.3 mg dosage of the I IL-1β-decreasing and/or L-6-decreasing cannabinoid, wherein the IL-1β-decreasing and/or IL-6-decreasing cannabinoid is CBDV. In some embodiments, the unit dosage form comprises 2.6 mg dosage of the IL-1β-decreasing and/or IL-6-decreasing cannabinoid, wherein the IL-1β-decreasing and/or IL-6-decreasing cannabinoid is CBDV. In some embodiments, the unit dosage form comprises 5.1 mg dosage of the IL-1β-decreasing and/or IL-6-decreasing cannabinoid, wherein the IL-1β-decreasing and/or IL-6-decreasing cannabinoid is CBDV.

In some embodiments, the unit dosage form comprises 14 mg dosage of the TNFα-decreasing cannabinoid, wherein the TNFα-decreasing cannabinoid is CBN. In some embodiments, the unit dosage form comprises 28 mg dosage of the TNFα-decreasing cannabinoid, wherein the TNFα-decreasing cannabinoid is CBN.

In some embodiments, the unit dosage form comprises 0.028 mg dosage of the IFNγ-decreasing cannabinoid or terpene, wherein the IFNγ-decreasing cannabinoid or terpene is CBG. In some embodiments, the unit dosage form comprises 0.28 mg dosage of the IFNγ-decreasing cannabinoid or terpene, wherein the IFNγ-decreasing cannabinoid or terpene is CBG. In some embodiments, the unit dosage form comprises 0.57 mg dosage of the IFNγ-decreasing cannabinoid or terpene, wherein the IFNγ-decreasing cannabinoid or terpene is CBG.

In some embodiments, the unit dosage form comprises 0.0028 mg dosage of the IFNγ-decreasing cannabinoid or terpene, wherein the IFNγ-decreasing cannabinoid or terpene is CBN. In some embodiments, the unit dosage form comprises 0.028 mg dosage of the IFNγ-decreasing cannabinoid or terpene, wherein the IFNγ-decreasing cannabinoid or terpene is CBN. In some embodiments, the unit dosage form comprises 0.27 mg dosage of the IFNγ-decreasing cannabinoid or terpene, wherein the IFNγ-decreasing cannabinoid or terpene is CBN.

In some embodiments, the unit dosage form comprises 0.001 mg dosage of the IFNγ-decreasing cannabinoid or terpene, wherein the IFNγ-decreasing cannabinoid or terpene is Phytol A. In some embodiments, the unit dosage form comprises 0.002 mg dosage of the IFNγ-decreasing cannabinoid or terpene, wherein the IFNγ-decreasing cannabinoid or terpene is Phytol A.

In some embodiments, the unit dosage form comprises 0.02 mg dosage of the Lymphopenia-reducing, wherein the Lymphopenia-reducing is THC. In some embodiments, the unit dosage form comprises 0.05 mg dosage of the Lymphopenia-reducing, wherein the Lymphopenia-reducing is THC.

In various embodiments, the unit dosage form is adapted for administration by inhalation. In certain of these embodiments, the unit dosage form is adapted for administration by a vaporizer. In certain of these embodiments, the unit dosage form is adapted for administration by a nebulizer. In certain of these embodiments, the unit dosage form is adapted for administration by an aerosolizer.

In some embodiments, the unit dosage form comprises 0.5 mg to 16 mg of inhalation dose of the IL-1β-decreasing and/or IL-6-decreasing cannabinoid. In some embodiments, the unit dosage form comprises 1 mg to 40 mg of inhalation dose of the IL-1β-decreasing and/or IL-6-decreasing cannabinoid. In certain embodiments, the unit dosage form comprises 14 mg of inhalation dose of the IL-1β-decreasing and/or IL-6-decreasing cannabinoid, wherein the IL-1β-decreasing and/or IL-6-decreasing cannabinoid is CBD. In certain embodiments, the unit dosage form comprises 28 mg of inhalation dose of the IL-1β-decreasing and/or IL-6-decreasing cannabinoid, wherein the IL-1β-decreasing and/or IL-6-decreasing cannabinoid is CBD. In certain embodiments, the unit dosage form comprises 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, or 40 mg, of inhalation dose of the IL-1β-decreasing and/or IL-6-decreasing cannabinoid, wherein the IL-1β-decreasing and/or IL-6-decreasing cannabinoid is CBD. In certain embodiments, the unit dosage form comprises 1.3 mg of inhalation dose of the IL-1β-decreasing and/or the IL-6-decreasing cannabinoid, wherein the IL-1β-decreasing and/or the IL-6-decreasing cannabinoid is CBDV. In certain embodiments, the unit dosage form comprises 2.6 mg of inhalation dose of the IL-1β-decreasing and/or IL-6-decreasing cannabinoid, wherein the IL-1β-decreasing and/or the IL-6-decreasing cannabinoid is CBDV. In certain embodiments, the unit dosage form comprises 5.2 mg of inhalation dose of the IL-1β-decreasing and/or the IL-6-decreasing cannabinoid, wherein the IL-1β-decreasing and/or the IL-6-decreasing cannabinoid is CBDV. In certain embodiments, the unit dosage form comprises 1 mg, 1.2 mg, 1.4 mg ,1.6 mg, 2 mg, 2.2 mg, 2.4 mg, 2.6 mg, 2.8 mg, 3 mg, 3.2 mg, 3.4 mg, 3.6 mg, 3.8 mg, 4 mg, 4.2 mg, 4.4 mg, 4.6 mg, 4.8 mg, 5 mg, 5.2 mg, 5.4 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, or 8 mg, of inhalation dose of the IL-1β-decreasing and/or the IL-6-decreasing cannabinoid, wherein the IL-1β-decreasing and/or the IL-6-decreasing cannabinoid is CBDV.

In some embodiments, the unit dosage form comprises 10 mg to 40 mg of inhalation dose of the TNFα-decreasing cannabinoid, wherein the TNFα-decreasing cannabinoid is CBN. In certain embodiments, the unit dosage form comprises 14 mg to 28 mg of inhalation dose of the TNFα-decreasing cannabinoid, wherein the TNFα-decreasing cannabinoid is CBN. In some embodiments, the unit dosage form comprises 28 mg of inhalation dose of the TNFα-decreasing cannabinoid, wherein the TNFα-decreasing cannabinoid is CBN. In some embodiments, the unit dosage form comprises 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg, of inhalation dose of the TNFα-decreasing cannabinoid, wherein the TNFα-decreasing cannabinoid is CBN.

In certain embodiments, the unit dosage form comprises 14 mg of inhalation dose of the TNFα-decreasing cannabinoid, wherein the TNFα-decreasing cannabinoid is CBN. In certain embodiments, the unit dosage form comprises 28 mg of inhalation dose of the TNFα-decreasing cannabinoid, wherein the TNFα-decreasing cannabinoid is CBN. In certain embodiments, the unit dosage form comprises 28 mg of inhalation dose of the TNFα-decreasing cannabinoid, wherein the TNFα-decreasing cannabinoid is CBN.

In some embodiments, the unit dosage form comprises 0.0001 mg to 3 mg of inhalation dose of the IFNγ-decreasing cannabinoid. In certain embodiments, the unit dosage form comprises 0.0013 mg to 6 mg of inhalation dose of the IFNγ-decreasing cannabinoid.

In certain embodiments, the unit dosage form comprises 1.4 mg of inhalation dose of the IFNγ-decreasing cannabinoid, wherein the IFNγ-decreasing cannabinoid is CBG. In certain embodiments, the unit dosage form comprises 0.02 mg of inhalation dose of the IFNγ-decreasing cannabinoid, wherein the IFNγ-decreasing cannabinoid is CBG. In certain embodiments, the unit dosage form comprises 0.028 mg of inhalation dose of the IFNγ-decreasing cannabinoid, wherein the IFNγ-decreasing cannabinoid is CBG. In certain embodiments, the unit dosage form comprises 0.03 mg of inhalation dose of the IFNγ-decreasing cannabinoid, wherein the IFNγ-decreasing cannabinoid is CBG. In certain embodiments, the unit dosage form comprises 0.5 mg of inhalation dose of the IFNγ-decreasing cannabinoid, wherein the IFNγ-decreasing cannabinoid is CBG. In certain embodiments, the unit dosage form comprises 0.6 mg of inhalation dose of the IFNγ-decreasing cannabinoid, wherein the IFNγ-decreasing cannabinoid is CBG. In certain embodiments, the unit dosage form comprises 0.0.005 mg, 0.01 mg, 0.012 mg, 0.014 mg, 0.016 mg, 0.018 mg, 0.02 mg, 0.022 mg, 0.024 mg, 0.026 mg, 0.028 mg, 0.03 mg, 0.032 mg, 0.034 mg, 0.04 mg, 0.042 mg, 0.044 mg, 0.048 mg, 0.05 mg, 0.052 mg, 0.054 mg, 0.056 mg, 0.057 mg, 0.058 mg, 0.06 mg, 0.062 mg, 0.064 mg, 0.066 mg, 0.068 mg, 0.07 mg, 0.072 mg, 0.074 mg, 0.074 mg, 0.076 mg, 0.078 mg, 0.08 mg, 0.082 mg, 0.084 mg, 0.086 mg, 0.088 mg, 0.09 mg, 0.1 mg, 0.2 mg, 0.3 mg, 0.4 mg, 0.5 mg, 0.51 mg, 0.52 mg, 0.53 mg, 0.54 mg, 0.55 mg, 0.56 mg, 0.57 mg, 0.58 mg, 0.58 mg, 0.6 mg, 0.62 mg, 0.64 mg, 0.66 mg, 0.68 mg, 0.7 mg, 0.72 mg, 0.74 mg, 0.76 mg, 0.78 mg, 0.8 mg, 0.82 mg, 0.84 mg, 0.86 mg, 0.88 mg, 0.9 mg, 0.92 mg, 0.94 mg, 0.96 mg, 0.98 mg, or 1 mg, of inhalation dose of the IFNγ-decreasing cannabinoid, wherein the IFNγ-decreasing cannabinoid is CBG.

In some embodiments, the unit dosage form comprises 0.0005 mg to 0.5 mg of inhalation dose of the IFNγ-suppressing cannabinoid or terpene, where the IFNγ-suppressing cannabinoid or terpene is CBN. In certain embodiments, the unit dosage form comprises 0.001 mg to 0.3 mg of inhalation dose of the IFNγ-suppressing cannabinoid or terpene, where the IFNγ-suppressing cannabinoid or terpene is CBN. In certain embodiments, the unit dosage form comprises 0.0005 mg, 0.0006 mg, 0.0007 mg, 0.0008 mg, 0.0009 mg, 0.001 mg, 0.0012 mg, 0.0013 mg, 0.0014 mg, 0.0015 mg, 0.0016 mg, 0.0017 mg, 0.0018 mg, 0.0019 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.22 mg, 0.24 mg, 0.26 mg, 0.28 mg, 0.3 mg, 0.4 mg, 0.5 mg, 0.6 mg, 0.7 mg, 0.8 mg, 0.9 mg, or 1 mg, of inhalation dose of the IFNγ-decreasing cannabinoid or terpene, where the IFNγ-decreasing cannabinoid or terpene is CBN.

In certain embodiments, the unit dosage form comprises 0.001 mg of inhalation dose of the IFNγ-decreasing terpene, wherein the IFNγ-decreasing terpene is Phytol A. In certain embodiments, the unit dosage form comprises 0.002 mg of inhalation dose of the IFNγ-decreasing terpene, wherein the IFNγ-decreasing terpene is Phytol A. In certain embodiments, the unit dosage form comprises 0.0002 mg. 0.0003 mg, to 0.0022 mg of inhalation dosage of the IFNγ-suppressing cannabinoid or terpene, where the IFNγ-suppressing cannabinoid or terpene is Phytol A. In certain embodiments, the unit dosage form comprises 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, or 0.05 mg of inhalation dosage of the IFNγ-decreasing cannabinoid or terpene, where the IFNγ-decreasing cannabinoid or terpene is Phytol Ain certain embodiments, the unit dosage form comprises 0.02 to 0.05 mg of inhalation dose of the Lymphopenia-reducing cannabinoid. In certain embodiments, the unit dosage form comprises 0.02 mg of inhalation dose of the Lymphopenia-reducing cannabinoid, wherein the Lymphopenia-reducing cannabinoid is THC. In certain embodiments, the unit dosage form comprises 0.05 mg of inhalation dose of the Lymphopenia-reducing cannabinoid, wherein the Lymphopenia-reducing cannabinoid is THC. In certain embodiments, the unit dosage form comprises 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, or 1 mg, of inhalation dose of the Lymphopenia-reducing cannabinoid, wherein the Lymphopenia-reducing cannabinoid is THC.

In various embodiments, the unit dosage form is adapted for administration by buccal or for sublingual administration.

In some embodiments, the unit dosage form comprises 1 mg to 24 mg of buccal or sublingual dose of the IL-1β-decreasing and/or the IL-6-decreasing cannabinoid. In some embodiments, the unit dosage form comprises 2.5 mg to 21 mg of buccal or sublingual dose of the IL-1β-decreasing and/or the IL-6-decreasing cannabinoid. In certain embodiments, the unit dosage form comprises 14 mg, 21 mg, or 28 mg of buccal or sublingual dose of the IL-1β-decreasing and/or the IL-6-decreasing cannabinoid, wherein the IL-1β-decreasing and/or the IL-6-decreasing cannabinoid is CBD. In certain embodiments, the unit dosage form comprises 8 mg, 9 mg, 10 mg, 11 m g, 12 mg, 13 mg, 14 mg, 16 mg, 17 mg, 18 mg, 20 mg, 21 mg, 22 mg, 23 mg, 24 mg, 25 mg, 26 mg, 28 mg, or 30 mg of buccal or sublingual dose of the IL-1β-decreasing and/or IL-6-decreasing cannabinoid, wherein the IL-1β-decreasing and/or the IL-6-decreasing cannabinoid is CBD. In certain embodiments, the unit dosage form comprises 14 mg, 21 mg, or28 mg of buccal or sublingual dose of the IL-1β-decreasing and/or the IL-6-decreasing cannabinoid, wherein the IL-1β-decreasing and/or IL-6-decreasing cannabinoid is CBD. In certain embodiments, the unit dosage form comprises 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 4 mg, 5 mg, or 5.5 mg of buccal or sublingual dose of the IL-1β-decreasing and/or IL-6-decreasing cannabinoid, wherein the IL-1β-decreasing and/or the IL-6-decreasing cannabinoid is CBDV.

In some embodiments, the unit dosage form comprises 10 mg to 30 mg of buccal or sublingual dose of the TNFα-decreasing cannabinoid. In some embodiments, the unit dosage form comprises 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, or 30 mg of buccal or sublingual dose of the TNFα-decreasing cannabinoid, wherein the TNFα-decreasing cannabinoid is CBN.

In some embodiments, the unit dosage form comprises 0.0001 mg to 10 mg of buccal or sublingual dose of the IFNγ-decreasing cannabinoid. In certain embodiments, the unit dosage form comprises 0.001 mg to 0.002 mg of buccal or sublingual dose of the IFNγ-decreasing cannabinoid, wherein the IFNγ-decreasing cannabinoid is Phytol A. In certain embodiments, the unit dosage form comprises 0.001 mg or 0.002 mg of buccal or sublingual dose of the IFNγ-decreasing cannabinoid, wherein the IFNγ-decreasing cannabinoid is Phytol A. In certain embodiments, the unit dosage form comprises 0.0002 mg. 0.0003 mg, to 0.0022 mg of buccal or sublingual dosage of the IFNγ-suppressing cannabinoid or terpene, where the IFNγ-suppressing cannabinoid or terpene is Phytol A. In certain embodiments, the unit dosage form comprises 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, or 0.05 mg of buccal or sublingual dosage of the IFNγ-decreasing cannabinoid or terpene, where the IFNγ-decreasing cannabinoid or terpene is Phytol A.

In certain embodiments, the unit dosage form comprises 1.4 mg of buccal or sublingual dose of the IFNγ-decreasing cannabinoid, wherein the IFNγ-decreasing cannabinoid is CBG. In certain embodiments, the unit dosage form comprises 0.02 mg of buccal or sublingual dose of the IFNγ-decreasing cannabinoid, wherein the IFNγ-decreasing cannabinoid is CBG. In certain embodiments, the unit dosage form comprises 0.028 mg of buccal or sublingual dose of the IFNγ-decreasing cannabinoid, wherein the IFNγ-decreasing cannabinoid is CBG. In certain embodiments, the unit dosage form comprises 0.03 mg of buccal or sublingual dose of the IFNγ-decreasing cannabinoid, wherein the IFNγ-decreasing cannabinoid is CBG. In certain embodiments, the unit dosage form comprises 0.5 mg of buccal or sublingual dose of the IFNγ-decreasing cannabinoid, wherein the IFNγ-decreasing cannabinoid is CBG. In certain embodiments, the unit dosage form comprises 0.6 mg of buccal or sublingual dose of the IFNγ-decreasing cannabinoid, wherein the IFNγ-decreasing cannabinoid is CBG. In certain embodiments, the unit dosage form comprises 0.0.005 mg, 0.01 mg, 0.012 mg, 0.014 mg, 0.016 mg, 0.018 mg, 0.02 mg, 0.022 mg, 0.024 mg, 0.026 mg, 0.028 mg, 0.03 mg, 0.032 mg, 0.034 mg, 0.04 mg, 0.042 mg, 0.044 mg, 0.048 mg, 0.05 mg, 0.052 mg, 0.054 mg, 0.056 mg, 0.057 mg, 0.058 mg, 0.06 mg, 0.062 mg, 0.064 mg, 0.066 mg, 0.068 mg, 0.07 mg, 0.072 mg, 0.074 mg, 0.074 mg, 0.076 mg, 0.078 mg, 0.08 mg, 0.082 mg, 0.084 mg, 0.086 mg, 0.088 mg, 0.09 mg, 0.1 mg, 0.2 mg, 0.3 mg, 0.4 mg, 0.5 mg, 0.51 mg, 0.52 mg, 0.53 mg, 0.54 mg, 0.55 mg, 0.56 mg, 0.57 mg, 0.58 mg, 0.58 mg, 0.6 mg, 0.62 mg, 0.64 mg, 0.66 mg, 0.68 mg, 0.7 mg, 0.72 mg, 0.74 mg, 0.76 mg, 0.78 mg, 0.8 mg, 0.82 mg, 0.84 mg, 0.86 mg, 0.88 mg, 0.9 mg, 0.92 mg, 0.94 mg, 0.96 mg, 0.98 mg, or 1 mg, of buccal or sublingual dose of the IFNγ-decreasing cannabinoid, wherein the IFNγ-decreasing cannabinoid is CBG.

In some embodiments, the unit dosage form comprises 0.0005 mg to 0.5 mg of buccal or sublingual dose of the IFNγ-suppressing cannabinoid or terpene, where the IFNγ-suppressing cannabinoid or terpene is CBN. In certain embodiments, the unit dosage form comprises 0.001 mg to 0.3 mg of buccal or sublingual dose of the IFNγ-suppressing cannabinoid or terpene, where the IFNγ-suppressing cannabinoid or terpene is CBN. In certain embodiments, the unit dosage form comprises 0.0005 mg, 0.0006 mg, 0.0007 mg, 0.0008 mg, 0.0009 mg, 0.001 mg, 0.0012 mg, 0.0013 mg, 0.0014 mg, 0.0015 mg, 0.0016 mg, 0.0017 mg, 0.0018 mg, 0.0019 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.22 mg, 0.24 mg, 0.26 mg, 0.28 mg, 0.3 mg, 0.4 mg, 0.5 mg, 0.6 mg, 0.7 mg, 0.8 mg, 0.9 mg, or 1 mg, of buccal or sublingual dose of the IFNγ-decreasing cannabinoid or terpene, where the IFNγ-decreasing cannabinoid or terpene is CBN.

In some embodiments, the unit dosage form comprises 0.03 mg to 0.05 mg of buccal or sublingual dose of the Lymphopenia-reducing cannabinoid, wherein the Lymphopenia-reducing cannabinoid is THC. In certain embodiments, the unit dosage form comprises 0.02 mg of buccal or sublingual dose of the Lymphopenia-reducing cannabinoid, wherein the Lymphopenia-reducing 5 cannabinoid is THC. In certain embodiments, the unit dosage form comprises 0.035 mg of buccal or sublingual dose of the Lymphopenia-reducing cannabinoid, wherein the Lymphopenia-reducing cannabinoid is THC. In certain embodiments, the unit dosage form comprises 0.047 mg of buccal or sublingual dose of the Lymphopenia-reducing cannabinoid, wherein the Lymphopenia-reducing cannabinoid is THC. In certain embodiments, the unit dosage form comprises 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, or 1 mg, of buccal or sublingual dose of the Lymphopenia-reducing cannabinoid, wherein the Lymphopenia-reducing cannabinoid is THC.

In various embodiments, the unit dosage form is adapted for administration by oral administration.

In some embodiments, the unit dosage form comprises 0.5 mg to 40 mg of oral dose of the IL-1β-decreasing and/or the IL-6-decreasing cannabinoid. In certain embodiments, the unit dosage form comprises 5 mg to 28 mg of oral dose of the IL-1β-decreasing and/or IL-6-decreasing cannabinoid. In certain embodiments, the unit dosage form comprises 28 mg of oral dose of the IL-1β-decreasing and/or IL-6-decreasing cannabinoid, wherein the IL-1β-decreasing and/or the IL-6-decreasing cannabinoid is CBD. In certain embodiments, the unit dosage form comprises 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, or 40 mg, of oral dose of the IL-1β-decreasing and/or the IL-6-decreasing cannabinoid, wherein the IL-6-decreasing cannabinoid is CBD. In certain embodiments, the unit dosage form comprises 5 mg of oral dose of the IL-1β-decreasing and/or IL-6-decreasing cannabinoid, wherein the IL-1β-decreasing and/or the IL-6-decreasing cannabinoid is CBDV. In certain embodiments, the unit dosage form comprises 1 mg, 1.2 mg, 1.4 mg ,1.6 mg, 2 mg, 2.2 mg, 2.4 mg, 2.6 mg, 2.8 mg, 3 mg, 3.2 mg, 3.4 mg, 3.6 mg, 3.8 mg, 4 mg, 4.2 mg, 4.4 mg, 4.6 mg, 4.8 mg, 5 mg, 5.2 mg, 5.4 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, or 8 mg, of oral dose of the IL-11:3-decreasing and/or the IL-6-decreasing cannabinoid, wherein the IL-1β-decreasing and/or the IL-6-decreasing cannabinoid is CBDV. In certain embodiments, the unit dosage form comprises 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 4 mg, 5 mg, or 5.5 mg of oral dose of the IL-1β-decreasing and/or the IL-6-decreasing cannabinoid, wherein the IL-1β-decreasing and/or the IL-6-decreasing cannabinoid is CBDV.

In some embodiments, the unit dosage form comprises 10 mg to 40 mg of inhalation dose of the TNFα-decreasing cannabinoid, wherein the TNFα-decreasing cannabinoid is CBN. In certain embodiments, the unit dosage form comprises 14 mg to 28 mg of inhalation dose of the TNFα-decreasing cannabinoid, wherein the TNFα-decreasing cannabinoid is CBN.

In some embodiments, the unit dosage form comprises 28 mg of oral dose of the TNFα-decreasing cannabinoid, wherein the TNFα-decreasing cannabinoid is CBN. In some embodiments, the unit dosage form comprises 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg, of oral dose of the TNFα-decreasing cannabinoid, wherein the TNFα-decreasing cannabinoid is CBN.

In some embodiments, the unit dosage form comprises 27 mg to 30 mg of oral dose of the TNFα-decreasing cannabinoid, wherein the TNFα-decreasing cannabinoid is CBN. In certain embodiments, the unit dosage form comprises 27 mg of oral dose of the TNFα-decreasing cannabinoid, wherein the TNFα-decreasing cannabinoid is CBN. In certain embodiments, the unit dosage form comprises 28 mg of oral dose of the TNFα-decreasing cannabinoid, wherein the TNFα-decreasing cannabinoid is CBN.

In some embodiments, the unit dosage form comprises 0.0002 mg to 8 mg of oral dose of the IFNγ-decreasing cannabinoid. In certain embodiments, the unit dosage form comprises 0.002 mg to 6 mg of oral dose of the IFNγ-decreasing cannabinoid.

In certain embodiments, the unit dosage form comprises 1.4 mg of oral dose of the IFNγ-decreasing cannabinoid, wherein the IFNγ-decreasing cannabinoid is CBG. In certain embodiments, the unit dosage form comprises 0.02 mg of oral dose of the IFNγ-decreasing cannabinoid, wherein the IFNγ-decreasing cannabinoid is CBG. In certain embodiments, the unit dosage form comprises 0.028 mg of oral dose of the IFNγ-decreasing cannabinoid, wherein the IFNγ-decreasing cannabinoid is CBG. In certain embodiments, the unit dosage form comprises 0.03 mg of oral dose of the IFNγ-decreasing cannabinoid, wherein the IFNγ-decreasing cannabinoid is CBG. In certain embodiments, the unit dosage form comprises 0.5 mg of oral dose of the IFNγ-decreasing cannabinoid, wherein the IFNγ-decreasing cannabinoid is CBG. In certain embodiments, the unit dosage form comprises 0.6 mg of oral dose of the IFNγ-decreasing cannabinoid, wherein the IFNγ-decreasing cannabinoid is CBG. In certain embodiments, the unit dosage form comprises 0.0.005 mg, 0.01 mg, 0.012 mg, 0.014 mg, 0.016 mg, 0.018 mg, 0.02 mg, 0.022 mg, 0.024 mg, 0.026 mg, 0.028 mg, 0.03 mg, 0.032 mg, 0.034 mg, 0.04 mg, 0.042 mg, 0.044 mg, 0.048 mg, 0.05 mg, 0.052 mg, 0.054 mg, 0.056 mg, 0.057 mg, 0.058 mg, 0.06 mg, 0.062 mg, 0.064 mg, 0.066 mg, 0.068 mg, 0.07 mg, 0.072 mg, 0.074 mg, 0.074 mg, 0.076 mg, 0.078 mg, 0.08 mg, 0.082 mg, 0.084 mg, 0.086 mg, 0.088 mg, 0.09 mg, 0.1 mg, 0.2 mg, 0.3 mg, 0.4 mg, 0.5 mg, 0.51 mg, 0.52 mg, 0.53 mg, 0.54 mg, 0.55 mg, 0.56 mg, 0.57 mg, 0.58 mg, 0.58 mg, 0.6 mg, 0.62 mg, 0.64 mg, 0.66 mg, 0.68 mg, 0.7 mg, 0.72 mg, 0.74 mg, 0.76 mg, 0.78 mg, 0.8 mg, 0.82 mg, 0.84 mg, 0.86 mg, 0.88 mg, 0.9 mg, 0.92 mg, 0.94 mg, 0.96 mg, 0.98 mg, or 1 mg, of oral dose of the IFNγ-decreasing cannabinoid, wherein the IFNγ-decreasing cannabinoid is CBG.

In some embodiments, the unit dosage form comprises 0.0005 mg to 0.5 mg of oral dose of the IFNγ-suppressing cannabinoid or terpene, where the IFNγ-suppressing cannabinoid or terpene is CBN. In certain embodiments, the unit dosage form comprises 0.001 mg to 0.3 mg of oral dose of the IFNγ-suppressing cannabinoid or terpene, where the IFNγ-suppressing cannabinoid or terpene is CBN. In certain embodiments, the unit dosage form comprises 0.0005 mg, 0.0006 mg, 0.0007 mg, 0.0008 mg, 0.0009 mg, 0.001 mg, 0.0012 mg, 0.0013 mg, 0.0014 mg, 0.0015 mg, 0.0016 mg, 0.0017 mg, 0.0018 mg, 0.0019 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.22 mg, 0.24 mg, 0.26 mg, 0.28 mg, 0.3 mg, 0.4 mg, 0.5 mg, 0.6 mg, 0.7 mg, 0.8 mg, 0.9 mg, or 1 mg, of oral dose of the IFNγ-decreasing cannabinoid or terpene, where the IFNγ-decreasing cannabinoid or terpene is CBN.

In certain embodiments, the unit dosage form comprises 0.0022 mg of oral dose of the IFNγ-decreasing terpene, wherein the IFNγ-decreasing terpene is Phytol A. In certain embodiments, the unit dosage form comprises 0.0002 mg. 0.0003 mg, to 0.0022 mg of oral dosage of the IFNγ-suppressing cannabinoid or terpene, where the IFNγ-suppressing cannabinoid or terpene is Phytol A. In certain embodiments, the unit dosage form comprises 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, or 0.05 mg of oral dosage of the IFNγ-decreasing cannabinoid or terpene, where the IFNγ-decreasing cannabinoid or terpene is Phytol A.

In some embodiments, the unit dosage form comprises 0.03 mg to 0.05 mg of oral dose of the Lymphopenia-reducing cannabinoid, wherein the Lymphopenia-reducing cannabinoid is THC. In certain embodiments, the unit dosage form comprises 0.047 mg of oral dose of the Lymphopenia-reducing cannabinoid, wherein the Lymphopenia-reducing cannabinoid is THC. In certain embodiments, the unit dosage form comprises 0.04 mg of oral dose of the Lymphopenia-reducing cannabinoid, wherein the Lymphopenia-reducing cannabinoid is THC. In certain embodiments, the unit dosage form comprises 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, or 1 mg, of oral dose of the Lymphopenia-reducing cannabinoid, wherein the Lymphopenia-reducing cannabinoid is THC.

In some embodiments, the unit dosage form is adapted for intravenous, intramuscular, or subcutaneous administration.

In some embodiments, the unit dosage form is adapted for intrathecal or intracerebroventricular administration.

In some embodiments, the unit dosage form is formulated for topical administration.

The amount of active ingredient which can be combined with a carrier material to produce a single dosage form will generally be that amount of the compound which produces a therapeutic effect.

### 5.8. METHODS OF TREATMENT

In another aspect, methods are presented for treating a patient having a disease responsive to the cannabinoid-containing complex mixtures described herein. The method comprises administering to the subject a therapeutically effective amount of cannabinoid-containing complex mixtures described herein. In a particular aspect, the present disclosure provides methods of treating a patient who has, or who is at risk for developing, cytokine release syndrome (CRS), CSS, or macrophage activation syndrome (MAS). In some embodiment, the patient has CRS, CSS, or MAS with hyperinflammation. In some embodiment, the patient has CRS, CSS, or MAS with T-cell related hyperinflammation. In some embodiment, the patient has In some aspects, the present disclosure provides methods of treating a patient who has, or is at risk of getting, an immune response. In some aspects, the present disclosure provides methods of treating a patient who has, or is at risk of having inflammationresponse. Thus, the present disclosure provides methods of suppressing an immune response to a patient who has inflammationresponse or hyperinflammation.

In some embodiments, the active pharmaceutical ingredient, a pharmaceutical composition, or a unit dosage form described in the present disclosure includes CCCMs useful for treating one or more of: CRS, MAS, CSS, Acute Respiratory Distress (ARD), the hyper-inflammatory condition known as secondary hemophagocytic lymphohistiocytosis (sHLH), adverse side effects of checkpoint inhibitor therapies (cancer) and CAR-T-cell therapies (anti-cancer), rheumatoid arthritis, multiple sclerosis, chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND), secondary hemophagocytic lymphohistiocytosis (sHLH) secondary to viral or bacterial infections such as severe forms of COVID-19, and a localized infection.

The methods comprise administering a therapeutically effective amount of an active pharmaceutical ingredient, a pharmaceutical composition, or a unit dosage form described herein.

In typical embodiments, the cannabinoid and/or terpene-containing complex mixtures are administered in the form of a pharmaceutical composition as described above. These methods are particularly aimed at therapeutic and prophylactic treatments of animals, and more particularly, humans.

The terms “treatment”, “treating”, and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

By the term “therapeutically effective dose” or “effective amount” is meant a dose or amount that produces the desired effect for which it is administered. The exact dose or amount will depend on the purpose of the treatment, and will be ascertainable by one skilled in the art using known techniques (see, e.g., Lloyd (1999) The Art, Science and Technology of Pharmaceutical Compounding).

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “ameliorating” refers to any therapeutically beneficial result in the treatment of a disease state.

The actual amount administered, and rate and time-course of administration, will depend on the nature and severity of disease being treated. Prescription of treatment, e.g. decisions on dosage etc., is within the responsibility of general practitioners and other medical professionals, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, the effective amount of the active pharmaceutical ingredient, pharmaceutical composition, or unit dose is administered by inhalation, orally, by buccal administration, by sublingual administration, by injection or by topical application.

In some embodiments, the effective amount of the active pharmaceutical ingredient, pharmaceutical composition, or unit dose in an amount sufficient to treat a person who has, or is at risk for developing, CRS.

In some embodiments, the IL-1β-decreasing and/or IL-6-decreasing cannabinoid, the TNFα-decreasing cannabinoid, the IFNγ-decreasing cannabinoid or terpene, and the Lymphopenia-reducing cannabinoid, is administered in an amount less than lg, less than 500 mg, less than 100 mg, less than 10 mg per dose.

In some embodiments, the active pharmaceutical ingredient, pharmaceutical composition, or unit dose is administered once a day, 2-4 times a day, 2-4 times a week, once a week, or once every two weeks.

An active pharmaceutical ingredient, pharmaceutical composition, or unit dose can be administered alone or in combination with other treatments, either simultaneously or sequentially dependent upon the condition to be treated.

Current inventions provide novel compositions comprising mixtures of the IL-1β-decreasing and/or IL-6-decreasing cannabinoid, the TNFα-decreasing cannabinoid, the IFNγ-decreasing cannabinoid or terpene, and the Lymphopenia-reducing cannabinoid. The present inventors have demonstrated that the compositions have significant immunological effects, and they can have therapeutic effects on inflammation. Furthermore, we have identified specific combinations of cannabinoids and/or terpenes that exert significant synergistic effects. This invention further provides methods of treating hyperinflammation, associated with, for example, CRS, MAS, CSS, Acute Respiratory Distress (ARD), the hyper-inflammatory condition known as secondary hemophagocytic lymphohistiocytosis (sHLH), adverse side effects of checkpoint inhibitor therapies (cancer) and CAR-T-cell therapies (anti-cancer), rheumatoid arthritis, multiple sclerosis, chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND), secondary hemophagocytic lymphohistiocytosis (sHLH) secondary to viral or bacterial infections such as severe forms of COVID-19, and a localized infection, using the pharmacological compositions identified herein. This invention further provides methods of treating hyperinflammation, methods of treating an immune response, methods of eliciting an immunosuppressive response, and methods of eliciting a pro-inflammatory response. The methods of the present disclosure can be good candidates to address clinical syndromes such as Cytokine Release Syndrome (CRS), Cytokine Storm Syndrome (CSS), Macrophage Activation Syndrome (MAS), Acute Respiratory Distress (ARD), and the hyper-inflammatory condition known as secondary hemophagocytic lymphohistiocytosis (sHLH) because their pathologies are all related to unregulated overproduction of proinflammatory cytokines, adverse side effects of checkpoint inhibitor therapies (cancer) and CAR-T-cell therapies (anti-cancer), rheumatoid arthritis and multiple sclerosis, which are autoimmune disorders where hyperactivated T cells attack self-antigens in the joints (arthritis) or the myelin sheath of the neurons (MS), chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND). The methods of the present disclosure can also be used to help fight localized infections

In some embodiments, the IL-1β-decreasing and/or IL-6-decreasing cannabinoid is Cannabidiol (CBD), Cannabidivarin (CBDV), or a combination thereof.

In some embodiments, the effective amount of the active pharmaceutical ingredient, pharmaceutical composition, or unit dose form comprises CBD in an amount sufficient to reduce the level of IL-6 secreted by activated CD14+ CD16+ monocytes.

In some embodiments, the effective amount of the active pharmaceutical ingredient, pharmaceutical composition, or unit dose form comprises CBD in an amount sufficient to reduce the level of IL-1β secreted by activated CD14+ CD16+ monocytes.

In some embodiments, the effective amount of the active pharmaceutical ingredient, pharmaceutical composition, or unit dose form comprises CBD in an amount sufficient to reduce phagocytosis by CD14+CD16+ monocytes.

In some embodiments, the effective amount of the active pharmaceutical ingredient, pharmaceutical composition, or unit dose form comprises CBDV in an amount sufficient to reduce the level of IL-1β secreted by activated CD14+ CD16+ monocytes.

In some embodiments, the effective amount of the active pharmaceutical ingredient, pharmaceutical composition, or unit dose form comprises CBDV in an amount sufficient to reduce the level of one or more co-stimulatory molecule expressed by CD14+CD16+ monocytes. In certain embodiments, the one or more co-stimulatory molecule is selected from the group consisting of: HLA-DR, CD80, and CD86.

In some embodiments, the effective amount of the active pharmaceutical ingredient, pharmaceutical composition, or unit dose form comprises CBDV in an amount sufficient to suppress CD25 expression by cytotoxic CD8+ T cells.

In some embodiments, the effective amount of the active pharmaceutical ingredient, pharmaceutical composition, or unit dose form comprises CBDV in an amount sufficient to suppress CD69 expression by cytotoxic CD8+ T cells.

In some embodiments, the TNFα-decreasing cannabinoid is CBN.

In some embodiments, the effective amount of the active pharmaceutical ingredient, pharmaceutical composition, or unit dose form comprises CBN in an amount sufficient to reduce the level of TNFα secreted by CD14+CD16+ monocytes.

In some embodiments, the IFNγ-decreasing cannabinoid or terpene is CBG. In some embodiments, the effective amount of the active pharmaceutical ingredient, pharmaceutical composition, or unit dose form comprises CBG in an amount sufficient to reduce the level of IFNγ secreted by T cells. In certain embodiments, the effective amount of the active pharmaceutical ingredient, pharmaceutical composition, or unit dose form comprises CBG in an amount sufficient to reduce the level of IFNγ secreted by CD4+ and/or CD8+ T cells.

In some embodiments, the IFNγ-decreasing terpene is Phytol A. In certain embodiments, the effective amount of the active pharmaceutical ingredient, pharmaceutical composition, or unit dose form comprises Phytol A in an amount sufficient to reduce the level of IFNγ secreted by T cells. In certain embodiments, the effective amount of the active pharmaceutical ingredient, pharmaceutical composition, or unit dose form comprises Phytol A in an amount sufficient to reduce the level of IFNγ secreted by CD4+ and/or CD8+ T cells.

In some embodiments, the Lymphopenia-reducing cannabinoid is tetrahydrocannabinol (THC).

In some embodiments, the effective amount of the active pharmaceutical ingredient, pharmaceutical composition, or unit dose form comprises THC in an amount sufficient to reduce the level of interleukin-1 beta (IL-1β) and/or interleukin-6 (IL-6) secreted by CD14+-CD16+ monocytes.

In some embodiments, the effective amount of the active pharmaceutical ingredient, pharmaceutical composition, or unit dose form comprises THC in an amount sufficient to reduce the level of TNFα secreted by CD14+-CD16+ monocytes.

In some embodiments, the effective amount of the active pharmaceutical ingredient, pharmaceutical composition, or unit dose form comprises THC in an amount sufficient to increase proliferation of helper CD4+ T cells.

In some embodiments, the effective amount of the active pharmaceutical ingredient, pharmaceutical composition, or unit dose form comprises THC in an amount sufficient to increase proliferation of cytotoxic CD8+ T cells.

### 5.8.1. Pre-Treatment Symptoms and Signs

In the methods described herein, the patient has or is at risk of developing CRS or Macrophage Activation Syndrome (MAS).

5.8.1.1. Pre-Treatment Serum CRP and IL-6 Levels

In some embodiments, the patient has elevated pre-treatment levels of serum C-reactive protein (CRP).

In some embodiments, the patient has a pre-treatment CRP level of at least 2 mg/L. In some embodiments, the patient has a pre-treatment CRP level of at least 5 mg/L. In some embodiments, the patient's pre-treatment CRP level is at least 2 mg/L, 2.5 mg/L, 3 mg/L, 3.5 mg/L, 4 mg/L, 4.5 mg/L, or 5 mg/L. In some embodiments, the patient has pre-treatment CRP levels of at least 7.5 mg/L, 10 mg/L, 12.5 mg/L, or 15 mg/L. In certain embodiments, the patient's pre-treatment CRP level is at least 7.5 mg/L. In certain embodiments, the patient has a pre-treatment CRP level of at least 10 mg/L. In certain embodiments, the patient has a pre-treatment CRP level of at least 12.5 mg/L. In certain embodiments, the patient has a pre-treatment CRP level of at least 15 mg/L. In certain preferred embodiments, the patient has a pre-treatment CRP level of at least 10 mg/L. In some embodiments, the patient has pre-treatment CRP levels of at least 20 mg/L, 25 mg/L, 30 mg/L, 35 mg/L, 40 mg/L, 45 mg/L, or 50 mg/L. In certain embodiments, the patient's pre-treatment CRP level is at least 20 mg/L. In certain embodiments, the patient has a pre-treatment CRP level of at least 25 mg/L. In certain embodiments, the patient has a pre-treatment CRP level of at least 30 mg/L. In certain embodiments, the patient has a pre-treatment CRP level of at least 35 mg/L. In certain embodiments, the patient's pre-treatment CRP level is at least 40 mg/L. In certain embodiments, the patient has a pre-treatment CRP level of at least 45 mg/L. In certain embodiments, the patient has a pre-treatment CRP level of at least 50 mg/L. In certain preferred embodiments, the patient has a pre-treatment CRP level of at least 40 mg/L.

In some embodiments of the methods described herein, the patient has elevated pre-treatment serum levels of IL-6.

In some embodiments, the patient has a pre-treatment serum IL-6 level of at least 2 μg/ml. In various embodiments, the patient has a pre-treatment serum IL-6 level of at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 11 μg/ml, at least 12 μg/ml, at least 13 μg/ml, at least 14 μg/ml, or at least 15 μg/ml. In certain embodiments, the patient has a pre-treatment serum IL-6 level of at least 2.5 μg/ml. In certain embodiments, the patient has a pre-treatment serum IL-6 level of at least 4 μg/ml. In certain embodiments, the patient has a pre-treatment serum IL-6 level of at least 5 μg/ml. In certain embodiments, the patient has a pre-treatment serum IL-6 level of at least 7.5 μg/ml. In certain embodiments, the patient has a pre-treatment serum IL-6 level of at least 10 μg/ml. In certain embodiments, the patient has a pre-treatment serum IL-6 level of at least 12.5 μg/ml. In certain embodiments, the patient has a pre-treatment serum IL-6 level of at least 15 μg/ml. In some embodiments, the patient has a pre-treatment serum IL-6 level of at least 20 μg/ml. In various embodiments, the patient has a pre-treatment serum IL-6 level of at least 20 μg/ml, at least 30 μg/ml, at least 40 μg/ml, at least 50 pg/ml, at least 60 μg/ml, at least 70 μg/ml, at least 80 μg/ml, at least 90 μg/ml, at least 100 μg/ml, at least 150 μg/ml, or at least 200 μg/ml. In certain embodiments, the patient has a pre-treatment serum IL-6 level of at least 30 μg/ml. In certain embodiments, the patient has a pre-treatment serum IL-6 level of at least 40 μg/ml. In certain embodiments, the patient has a pre-treatment serum IL-6 level of at least 50 μg/ml. In certain embodiments, the patient has a pre-treatment serum IL-6 level of at least 75 μg/ml. In certain embodiments, the patient has a pre-treatment serum IL-6 level of at least 100 μg/ml. In certain embodiments, the patient has a pre-treatment serum IL-6 level of at least 150 μg/ml. In certain embodiments, the patient has a pre-treatment serum IL-6 level of at least 200 μg/ml.

In some embodiments, the patient has elevated pre-treatment serum levels of CRP and elevated pre-treatment IL-6 levels. In certain embodiments, the patient has a pre-treatment serum IL-6 level of at least 2 μg/ml and a pre-treatment CRP level of at least 2 mg/L. In certain embodiments, the patient has a pre-treatment serum IL-6 level of at least 2 μg/ml and a pre-treatment CRP level of at least 2.5 mg/L. In certain embodiments, the patient has a pre-treatment serum IL-6 level of at least 2 μg/ml and a pre-treatment CRP level of at least 5 mg/L. In certain embodiments, the patient has a pre-treatment serum IL-6 level of at least 2 μg/ml and a pre-treatment CRP level of at least 10 mg/L. In certain embodiments, the patient has a pre-treatment serum IL-6 level of at least 4 μg/ml and a pre-treatment CRP level of at least 2 mg/L. In certain embodiments, the patient has a pre-treatment serum IL-6 level of at least 4 μg/ml and a pre-treatment CRP level of at least 2.5 mg/L. In certain embodiments, the patient has a pre-treatment serum IL-6 level of at least 4 μg/ml and a pre-treatment CRP level of at least 5 mg/L. In certain embodiments, the patient has a pre-treatment serum IL-6 level of at least 4 μg/ml and a pre-treatment CRP level of at least 10 mg/L. In certain embodiments, the patient has a pre-treatment serum IL-6 level of at least 5 μg/ml and a pre-treatment CRP level of at least 2 mg/L. In certain embodiments, the patient has a pre-treatment serum IL-6 level of at least 5 μg/ml and a pre-treatment CRP level of at least 2.5 mg/L. In certain embodiments, the patient has a pre-treatment serum IL-6 level of at least 5 μg/ml and a pre-treatment CRP level of at least 5 mg/L. In certain embodiments, the patient has a pre-treatment serum IL-6 level of at least 5 μg/ml and a pre-treatment CRP level of at least 10 mg/L. In certain embodiments, the patient has a pre-treatment serum IL-6 level of at least 10 μg/ml and a pre-treatment CRP level of at least 2 mg/L. In certain embodiments, the patient has a pre-treatment serum IL-6 level of at least 10 μg/ml and a pre-treatment CRP level of at least 2.5 mg/L. In certain embodiments, the patient has a pre-treatment serum IL-6 level of at least 10 μg/ml and a pre-treatment CRP level of at least 5 mg/L. In certain embodiments, the patient has a pre-treatment serum IL-6 level of at least 10 μg/ml and a pre-treatment CRP level of at least 10 mg/L.

In some embodiments, the patient has a pre-treatment serum IL-6 level of at least 10 pg/ml and a pre-treatment CRP level of at least 10 mg/L. In certain embodiments, the patient has a pre-treatment serum IL-6 level of at least 10 μg/ml and a pre-treatment CRP level of at least 20 mg/L. In certain embodiments, the patient has a pre-treatment serum IL-6 level of at least 10 pg/ml and a pre-treatment CRP level of at least 30 mg/L. In certain embodiments, the patient has a pre-treatment serum IL-6 level of at least 10 μg/ml and a pre-treatment CRP level of at least 40 mg/L. In certain embodiments, the patient has a pre-treatment serum IL-6 level of at least 20 pg/ml and a pre-treatment CRP level of at least 10 mg/L. In certain embodiments, the patient has a pre-treatment serum IL-6 level of at least 20 μg/ml and a pre-treatment CRP level of at least 20 mg/L. In certain embodiments, the patient has a pre-treatment serum IL-6 level of at least 20 pg/ml and a pre-treatment CRP level of at least 30 mg/L. In certain embodiments, the patient has a pre-treatment serum IL-6 level of at least 20 μg/ml and a pre-treatment CRP level of at least 40 mg/L. In certain embodiments, the patient has a pre-treatment serum IL-6 level of at least 30 pg/ml and a pre-treatment CRP level of at least 10 mg/L. In certain embodiments, the patient has a pre-treatment serum IL-6 level of at least 30 μg/ml and a pre-treatment CRP level of at least 20 mg/L. In certain embodiments, the patient has a pre-treatment serum IL-6 level of at least 30 pg/ml and a pre-treatment CRP level of at least 30 mg/L. In certain embodiments, the patient has a pre-treatment serum IL-6 level of at least 30 μg/ml and a pre-treatment CRP level of at least 40 mg/L. In certain embodiments, the patient has a pre-treatment serum IL-6 level of at least 40 pg/ml and a pre-treatment CRP level of at least 10 mg/L. In certain embodiments, the patient has a pre-treatment serum IL-6 level of at least 40 μg/ml and a pre-treatment CRP level of at least 20 mg/L. In certain embodiments, the patient has a pre-treatment serum IL-6 level of at least 40 pg/ml and a pre-treatment CRP level of at least 30 mg/L. In certain embodiments, the patient has a pre-treatment serum IL-6 level of at least 40 μg/ml and a pre-treatment CRP level of at least 40 mg/L.

5.8.1.2. CRS

In some embodiments, the patient has cytokine release syndrome (CRS) or macrophage activation syndrome (MAS). In some embodiments, the patient is at risk for CRS or MAS. In some embodiments, the patient has infection by a virus, such as an influenza virus or a coronavirus.

5.8.1.3. Hypercoagulability

In some embodiments, the patient has confirmed or suspected hypercoagulability. Hypercoagulability has been shown to be linked to certain inflammatory conditions related to the pro-inflammatory cytokines IL-6, IL-1β, and/or IL-8 (see e.g., Bester and Pretorius, Effects of IL-1β, IL-6, and IL-8 on erythrocytes, platelets, and clot viscoelasticity. Scientific Reports (2016) 6:32188, μgs 1-10). Thus, patients who have increased amounts of pro-inflammatory cytokine IL-6 can induce hypercoagulability in the patient.

5.8.1.4. Confirmed or suspected viral lung infection

In various embodiments of the methods described herein, the patient has a confirmed or suspected viral infection. In some embodiments, the infection is by a virus selected from the group consisting of: coronavirus, influenza virus, rhinovirus, respiratory syncytial virus, metapneumovirus, adenovirus, and boca virus.

In certain embodiments, the virus is an influenza virus. In particular embodiments, the influenza virus is selected from the group consisting of: parainfluenza virus 1, parainfluenza virus 2, influenza A virus, and influenza B virus.

In certain embodiments, the virus is a coronavirus. In particular embodiments, the coronavirus is selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2. In some embodiments, the coronavirus is SARS-CoV-2.

In particular embodiments, the CRS is caused by infection with severe acute respiratory syndrome beta coronavirus (SARS-CoV) and the patient has severe acute respiratory syndrome (SARS). In particular embodiments, the CRS is caused by infection with middle eastern respiratory syndrome virus (MERS-CoV) and the patient has middle east respiratory syndrome (MERS). In particular embodiments, the CRS is caused by infection with SARS-CoV-2 virus and the patient has coronavirus disease 2019 (COVID-19).

In various embodiments, viral infection has been or is concomitantly confirmed by detection of viral genetic material in a fluid sample from the patient. In some embodiments, viral infection has not been or is not concomitantly confirmed by detection of viral genetic material in a fluid sample from the patient but is presumed based on clinical presentation and history. In particular embodiments, treatment is initiated before confirmation by detection of viral genetic material. In specific embodiments, treatment is initiated before confirmation by detection of viral genetic material, and viral infection is later confirmed by detection of viral genetic material or virus-specific gM and/or IgG in the patient's serum.

5.8.1.1. Lymphopenia

In some embodiments, the patient has lymphopenia.

5.8.1.2. Haemophagocytic lymphohistiocytosis (sHLH)

In some embodiments, the patient has haemophagocytic lymphohistiocytosis (sHLH). In some embodiments, the patient is at risk for developing sHLH.

5.8.1.3. Acute Lung Inflammation (ALI)

In some embodiments, the patient has ALI.

5.8.1.4. Respiratory Distress Syndrome (ARDS)

In some embodiments, the patient has ARDS.

5.8.1.5. Ischemia

In some embodiments, the patient has acute renal injury.

In some embodiments, the patient has ischemia-reperfusion injury (IRI) or reoxygenation injury.

In some embodiments, the IRI is associated with coronary ischemia, brain ischemia, renal ischemia, or intestinal ischemia, in the patient.

5.8.1.6. Sepsis

In some embodiments, the patient has sepsis.

5.8.1.7. Stroke

In some embodiments, the patient has a stroke.

5.8.1.8. Fever

5.8.1.9. Other

In some embodiments, the patient has adverse side effects of checkpoint inhibitor therapies (cancer) and CAR-T-cell therapies (anti-cancer).

In some embodiments, the patient has rheumatoid arthritis.

In some embodiments, the patient has multiple sclerosis.

In some embodiments, the patient has a chronic inflammatory diseases, such as ulcers, systemic lupus erythematosus (SLE), rheumatoid arthritis (RA), psoriasis, multiple sclerosis (MS), Alzheimer's disease (AD), Parkinson's disease (PD), and the human immunodeficiency virus-associated neurocognitive disorder (HAND), secondary hemophagocytic lymphohistiocytosis (sHLH) secondary to viral or bacterial infections such as severe forms of COVID-19.

In some embodiments, the patient has a localized infection.

In some embodiments, the patient has fever. In some embodiments, the patient has a body temperature greater than 37.5° C. In some embodiments, the body temperature is 37.6° C. or greater, 37.7° C. or greater, 37.8° C. or greater, 37.9° C. or greater, 38° C. or greater , 38.1° C. or greater, 38.2° C. or greater, 38.3° C. or greater, 38.4° C. or greater, 38.5° C. or greater, 38.6° C. or greater , 38.7° C. or greater , 38.8° C. or greater , 38.9° C. or greater , 39° C. or greater , 39.1° C. or greater , 39.2° C. or greater , 39.3° C. or greater , 39.4° C. or greater , 39.5° C. or greater , 39.6° C. or greater , 39.7° C. or greater , 39.8° C. or greater , 39.9° C. or greater , 40° C. or greater, 40.1° C. or greater, 40.2° C. or greater, 40.3° C. or greater, 40.4° C. or greater, 40.5° C. or greater, 40.6° C. or greater, 40.7° C. or greater, 40.8° C. or greater, 40.9° C. or greater, 41° C. or greater, or 42° C. or greater. In some embodiments, the patient has a body temperature greater than 37.5° C. for 24 hours or more, 48 hours or more, 72 hours or more, 96 hours or more, 5 days or more, 6 days or more, 1 week or more, 1.5 weeks or more, or 2 weeks or more. In typical embodiments, the body temperature is measured from clinically accessible measurement sites on the patient. In various embodiments, the measurement site is the patient's forehead, temple, and/or other external body surfaces. In some embodiments, the measurement site is the oral cavity, rectal cavity, axilla area, or tympanic membrane.

5.8.1.10. Reduced blood oxygen saturation

In some embodiments, the patient has a blood oxygen saturation level (SpO2) of less than 95%. In some embodiments, the patient has a blood oxygen saturation level (SpO2) of less than 94%. In some embodiments, the patient has a blood oxygen saturation level (SpO2) of 93% or less. In some embodiments, the patient has an Sp02 level of 92% or less, 91% or less, 90% or less, 85% or less, 80% or less, 75% or less, 70% or less, 65% or less, 60% or less, 55% or less, 50% or less, 45% or less, 40% or less, 35% or less, 30% or less, or 25% or less. In some embodiments, the patient requires mechanical ventilation and/or supplemental oxygen.

5.8.1.11. Pneumonia

In some embodiments, the patient has pneumonia.

In some embodiments, the patient has ALI with concomitant pneumonia or ARDS with concomitant pneumonia.

5.8.1.12. Hospitalization

In some embodiments, the patient is hospitalized.

5.8.1.13. Mechanical or assisted ventilation

In some embodiments, the patient is on a ventilator. In some embodiments, the patient is not on a ventilator.

5.8.1.14. Pre-treatment d-Dimer and Sepsis-Induced Coagulopathy (SIC) Score

In certain embodiments, the patient has elevated pre-treatment levels of d-dimer above baseline (e.g. >1 μg/ml or elevated at or above the upper limit of normal). In certain embodiments, the patient has elevated pre-treatment levels of sepsis-induced coagulopathy (SIC) total score of 4 or more with total score of prothrombin time and coagulation exceeding 2, or a score at or above the upper limit of normal.

5.8.1.15. Pre-Treatment Lymphocyte Count

In some embodiments, the patient has a pre-treatment median lymphocyte count per mm3 of 1000 or less. In certain embodiments, the patient has a pre-treatment median lymphocyte count per mm3 of 950 or less. In certain embodiments, the patient has a pre-treatment median lymphocyte count per mm3 of 900 or less, 850 or less, 800 or less, 750 or less, 700 or less, 650 or less, or 600 or less. In particular embodiments, the patient has a pre-treatment median lymphocyte count per mm3 of 800. In particular embodiments, the patient has a pre-treatment median lymphocyte count per mm3 or 700.

5.8.1.16. Pre-Treatment Platelet Count

In some embodiments, the patient has a pre-treatment median platelet count per mm3 of 175,000 or less. In certain embodiments, the patient has a pre-treatment median platelet count per mm3 of 172,000 or less. In certain embodiments, the patient has a pre-treatment median platelet count per mm3 of 170,000 or less, 165,000 or less, 160,000 or less, 155,000 or less, 150,000 or less, 145,000 or less, 140,000 or less, 135,000 or less, 130,000 or less, 125,000 or less, 120,000 or less, 115,000 or less, 110,000 or less, 105,000 or less, or 100,000 or less. In particular embodiments, the patient has a pre-treatment median platelet count per mm3 of 160,000 or less. In particular embodiments, has a pre-treatment median platelet count per mm3 or 140,000 or less.

5.8.1.17. Pre-Treatment Neutrophil-to-Lymphocyte Ratio

In some embodiments, the patient has a pre-treatment neutrophil-to-lymphocyte ratio (NLR) greater than 2.0. In some embodiments, the patient has a pre-treatment NLR greater than 3.0. In some embodiments, the patient has a pre-treatment NLR greater than 2.8, 2.9, 3.0, 3.1, 3.2, 3.3, 3.4, 3.5, 3.6, 3.7, 3.8, or 3.9. In some embodiment, the patient has a pre-treatment NLR greater than 4.0.

5.8.1.18. Pre-Treatment Lactate dehydrogenase

In some embodiments, the patient has elevated pre-treatment levels of lactate dehydrogenase at or above baseline (e.g. 250 units/liter, or elevated at or above the upper limit of normal). In some embodiments, the patient has elevated pre-treatment levels of lactate dehydrogenase at or above 250 units/liter, 300 units/liter, 350 units/liter, 350 units/liter, 400 units/liter, 450 units/liter, or 500 units/liter.

### 5.8.2. Patient Age

In some embodiments, the patient is older than 60 years old. In some embodiments, the patient is older than 50 years old. In some embodiments, the patient is older than 50, 51, 52, 53, 54, 55, 56, 57, 58, 59, or 60 years old. In some embodiments, the patient is younger than 60, 59, 58, 57, 56, 55, 54, 53, 52, 51, or 50 years old. In some embodiments the patient is a young adult between the age of 20-35. In some embodiments, the patient is middle aged, between the age of 35-50. In some embodiments, the patient is a teenager between the age of 13-19. In some embodiments, the patient is a child between the age of 5-12. In alternative embodiments, the patient is a toddler between the age of 1-4. In further embodiments, the patient is an infant between the age of newborn to one year old.

### 5.8.3. Additional Agents

In some embodiments, the method further includes administering an effective amount of at least one second therapeutic agent.

In certain embodiments, the second therapeutic agent is selected from an antiviral agent, an antibacterial agent, an IL-6 antagonist, an angiotensin receptor blocker (ARB), granulocyte/macrophage colony stimulating factor (GM-CSF) antagonist, hydroxychloroquine, chloroquine, and COVID-19 immune serum or plasma.

5.8.3.1. IL-6 Antagonists

In some embodiments, the second therapeutic agent is an IL-6 antagonist. In certain embodiments, the IL-6 antagonist disclosed herein is an anti-IL-6 antibody or antigen-binding fragment thereof. In some embodiments, the IL-6 antagonist disclosed herein is a JAK or STAT inhibitor. In some embodiments, the IL-6 antagonist disclosed herein is an IL-6 antagonist peptide.

5.8.3.1.1. Anti-IL-6 Antibodies

In various embodiments, the IL-6 antagonist is an anti-IL-6 antibody or antigen-binding fragment thereof.

In typical embodiments, the anti-IL-6 antibody or antigen-binding fragment thereof neutralizes the biological activity of human IL-6. In some embodiments, the neutralizing antibody prevents binding of IL-6 to the IL-6 receptor. In certain embodiments, the neutralizing antibody prevents binding of IL-6 to the soluble IL-6 receptor. In certain embodiments, the neutralizing antibody prevents binding of IL-6 to the membrane-bound IL-6 receptor. In certain embodiments, the neutralizing antibody prevents binding of IL-6 to both the soluble IL-6 receptor and the membrane-bound IL-6 receptor.

In some embodiments, the IL-6 antagonist is an anti-IL-6 monoclonal antibody. In some embodiments, the IL-6 antagonist is a polyclonal composition comprising a plurality of species of anti-IL-6 antibodies, each of the plurality having unique CDRs.

In some embodiments, the anti-IL-6 antibody is selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza). In some embodiments, the antigen-binding fragment is a fragment of an antibody selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

In some embodiments, the antigen binding fragment of an anti-IL-6 antibody is a Fab, Fab′, F(ab′)2 , Fv, scFv, (scFv)2, single chain antibody molecule, dual variable domain antibody, single variable domain antibody, linear antibody, or V domain antibody. In some embodiments, the antigen-binding fragment of an IL-6 antibody is a Fab′. In some embodiments, the antigen-binding fragment of an IL-6 antibody is a Fab. In some embodiments, the antigen-binding fragment of an IL-6 antibody is a F(ab′)2. In some embodiments, the antigen-binding fragment of an IL-6 antibody is a Fab. In some embodiments, the antigen-binding fragment of an IL-6 antibody is an Fv. In some embodiments, the antigen-binding fragment of an IL-6 antibody is a Fab. In some embodiments, the antigen-binding fragment of an IL-6 antibody is a scFv. In some embodiments, the antigen-binding fragment of an IL-6 antibody is a Fab. In some embodiments, the antigen-binding fragment of an IL-6 antibody is an (scFv)2.

In some embodiments, the anti-IL-6 antibody comprises a heavy chain constant region of a class selected from IgG, IgA, IgD, IgE, and IgM. In certain embodiments, the anti-IL-6 antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6 antigen-binding fragment.

In some embodiments, the antibody is bispecific or multispecific, with at least one of the antigen-binding sites having specificity for IL-6.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6 antibody has a KD for binding human IL-6 of less than 100 nM. In some embodiments, the anti-IL-6 antibody has a KD for binding human IL-6 of less than 75 nM, 50 nM, 25 nM, 20 nM, 15 nM, or 10 nM. In particular embodiments, the anti-IL-6 antibody has a KD for binding human IL-6 of less than 5 nM, 4 nM, 3 nM, or 2 nM. In selected embodiments, the anti-IL-6 antibody has a KD for binding human IL-6 of less than 1 nM, 750 pM, or 500 pM. In specific embodiments, the anti-IL-6 antibody has a KD for binding human IL-6 of no more than 500 pM, 400 pM, 300 pM, 200 pM, or 100 pM.

In typical embodiments, the anti-IL-6 antibody has an elimination half-life following intravenous administration of at least 7 days. In certain embodiments, the anti-IL-6 antibody has an elimination half-life of at least 14 days, at least 21 days, or at least 30 days.

In some embodiments, the anti-IL-6 antibody has a human IgG constant region with at least one amino acid substitution that extends serum half-life as compared to the unsubstituted human IgG constant domain.

In certain embodiments, the IgG constant domain comprises substitutions at residues 252, 254, and 256, wherein the amino acid substitution at amino acid residue 252 is a substitution with tyrosine, the amino acid substitution at amino acid residue 254 is a substitution with threonine, and the amino acid substitution at amino acid residue 256 is a substitution with glutamic acid (“YTE”). See U.S. Pat. No. 7,083,784, incorporated herein by reference in its entirety. In certain extended half-life embodiments, the IgG constant domain comprises substitutions selected from T250Q/M428L (Hinton et al., J. Immunology 176:346-356 (2006)); N434A (Yeung et al., J. Immunology 182:7663-7671 (2009)); or T307A/E380A/N434A (Petkova et al., International Immunology, 18: 1759-1769 (2006)).

In some embodiments, the elimination half-life of the anti-IL-6 antibody or antigen-binding fragment thereof is increased by utilizing the FcRN-binding properties of human serum albumin. In certain embodiments, the antibody is conjugated to albumin (Smith et al., Bioconjug. Chem., 12: 750-756 (2001)). In some embodiments, the anti-IL-6 antibody is fused to bacterial albumin-binding domains (Stork et al., Prot. Eng. Design Science 20: 569-76 (2007)). In some embodiments, the anti-IL-6 antibody is fused to an albumin-binding peptide (Nguygen et al., Prot Eng Design Sel 19: 291-297 (2006)). In some embodiments, the anti-IL-6 antibody is bispecific, with one specificity being to IL-6, and one specificity being to human serum albumin (Ablynx, WO 2006/122825 (bispecific Nanobody)).

In some embodiments, the elimination half-life of the anti-IL-6 antibody is increased by PEGylation (Melmed et al., Nature Reviews Drug Discovery 7: 641-642 (2008)); by HPMA copolymer conjugation (Lu et al., Nature Biotechnology 17: 1101-1104 (1999)); by dextran conjugation (Nuclear Medicine Communications, 16: 362-369 (1995)); by conjugation with homo-amino-acid polymers (HAPs; HAPylation) (Schlapschy et al., Prot Eng Design Sel 20: 273-284 (2007)); or by polysialylation (Constantinou et al., Bioconjug. Chem. 20: 924-931 (2009)).

5.8.3.1.2. Ziltivekimab and Derivatives

In certain preferred embodiments, the anti-IL-6 antibody or antigen-binding portion thereof comprises all six CDRs of Ziltivekimab (referred to interchangeably herein as “COR-001”). The ziltivekimab antibody (also known as “MEDI5117”) is described in WO 2010/088444 and US 2012/0034212, the disclosures of which are incorporated herein by reference in their entireties. In particular embodiments, the antibody or antigen-binding portion thereof comprises the ziltivekimab heavy chain V region and light chain V region. In specific embodiments, the antibody is the full-length ziltivekimab antibody.

In various embodiments, the anti-IL-6 antibody is a derivative of ziltivekimab.

In some embodiments, the ziltivekimab derivative includes one or more amino acid substitutions in the ziltivekimab heavy and/or light chain V regions.

In certain embodiments, the ziltivekimab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of ziltivekimab, while retaining specificity for human IL-6. In some embodiments, the ziltivekimab derivative comprises a Fab, a Fab', a F(ab′)2, an Fv, a single chain Fv (scFv), or an (scFv)2 region of ziltivekimab.

In certain embodiments, the ziltivekimab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of ziltivekimab. The percent sequence identity is determined using BLAST algorithms using default parameters.

In certain embodiments, the ziltivekimab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of ziltivekimab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.3. Siltuximab and Derivatives

In certain embodiments, the anti-IL-6 antibody or antigen-binding portion thereof comprises all six CDRs of siltuximab. In particular embodiments, the antibody or antigen-binding portion thereof comprises the siltuximab heavy chain V region and light chain V region. In specific embodiments, the antibody is the full-length siltuximab antibody.

In various embodiments, the anti-IL-6 antibody is a derivative of siltuximab.

In some embodiments, the siltuximab derivative includes one or more amino acid substitutions in the siltuximab heavy and/or light chain V regions.

In certain embodiments, the siltuximab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of siltuximab, while retaining specificity for human IL-6.

In certain embodiments, the siltuximab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of siltuximab. The percent sequence identity is determined using BLAST algorithms using default parameters.

In certain embodiments, the siltuximab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of siltuximab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.4. Gerilimzumab and Derivatives

In certain embodiments, the anti-IL-6 antibody or antigen-binding portion thereof comprises all six CDRs of gerilimzumab. In particular embodiments, the antibody or antigen-binding portion thereof comprises the gerilimzumab heavy chain V region and light chain V region. In specific embodiments, the antibody is the full-length gerilimzumab antibody.

In various embodiments, the anti-IL-6 antibody is a derivative of gerilimzumab.

In some embodiments, the gerilimzumab derivative includes one or more amino acid substitutions in the gerilimzumab heavy and/or light chain V regions.

In certain embodiments, the gerilimzumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of gerilimzumab, while retaining specificity for human IL-6.

In certain embodiments, the gerilimzumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of gerilimzumab. The percent sequence identity is determined using BLAST algorithms using default parameters.

In certain embodiments, the gerilimzumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of gerilimzumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.5. Sirukumab and Derivatives

In certain embodiments, the anti-IL-6 antibody or antigen-binding portion thereof comprises all six CDRs of sirukumab. In particular embodiments, the antibody or antigen-binding portion thereof comprises the sirukumab heavy chain V region and light chain V region.

In various embodiments, the anti-IL-6 antibody is a derivative of sirukumab.

In some embodiments, the sirukumab derivative includes one or more amino acid substitutions in the sirukumab heavy and/or light chain V regions.

In certain embodiments, the sirukumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of sirukumab, while retaining specificity for human IL-6.

In certain embodiments, the sirukumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of sirukumab. The percent sequence identity is determined using BLAST algorithms using default parameters.

In certain embodiments, the sirukumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sirukumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.6. Clazakizumab and Derivatives

In certain embodiments, the anti-IL-6 antibody or antigen-binding portion thereof comprises all six CDRs of clazakizumab. In particular embodiments, the antibody or antigen-binding portion thereof comprises the clazakizumab heavy chain V region and light chain V region. In specific embodiments, the antibody is the full-length clazakizumab antibody.

In various embodiments, the anti-IL-6 antibody is a derivative of clazakizumab.

In some embodiments, the clazakizumab derivative includes one or more amino acid substitutions in the clazakizumab heavy and/or light chain V regions.

In certain embodiments, the clazakizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of clazakizumab, while retaining specificity for human IL-6.

In certain embodiments, the clazakizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of clazakizumab. The percent sequence identity is determined using BLAST algorithms using default parameters.

In certain embodiments, the clazakizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of clazakizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.7. Olokizumab and Derivatives

In certain embodiments, the anti-IL-6 antibody or antigen-binding portion thereof comprises all six CDRs of olokizumab. In particular embodiments, the antibody or antigen-binding portion thereof comprises the olokizumab heavy chain V region and light chain V region. In specific embodiments, the antibody is the full-length olokizumab antibody.

In various embodiments, the anti-IL-6 antibody is a derivative of olokizumab.

In some embodiments, the olokizumab derivative includes one or more amino acid substitutions in the olokizumab heavy and/or light chain V regions.

In certain embodiments, the olokizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the olokizumab anti-IL-6 antibody, while retaining specificity for human IL-6.

In certain embodiments, the olokizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of olokizumab. The percent sequence identity is determined using BLAST algorithms using default parameters.

In certain embodiments, the olokizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of olokizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL-6).

5.8.3.1.8. Other Anti-IL-6 Antibodies and Derivatives

In certain embodiments, the anti-IL-6 antibody or antigen-binding portion thereof comprises all six CDRs of an antibody selected from the group consisting of: VX30 (VOP-R003; Vaccinex), EB-007 (EBI-029; Eleven Bio), and FM101 (Femta Pharmaceuticals, Lonza). In particular embodiments, the antibody or antigen-binding portion thereof comprises the heavy chain V region and light chain V region of an antibody selected from the group consisting of: VX30 (VOP-R003; Vaccinex), EB-007 (EBI-029; Eleven Bio), and FM101 (Femta Pharmaceuticals, Lonza). In specific embodiments, the antibody is a full-length antibody selected from the group consisting of: VX30 (VOP-R003; Vaccinex), EB-007 (EBI-029; Eleven Bio), and FM101 (Femta Pharmaceuticals, Lonza).

In various embodiments, the anti-IL-6 antibody is a derivative of an antibody selected from the group consisting of: VX30 (VOP-R003; Vaccinex), EB-007 (EBI-029; Eleven Bio), and FM101 (Femta Pharmaceuticals, Lonza).

5.8.3.1.9. Ziltivekimab

In certain preferred embodiments, the IL-6 antibody or antigen-binding fragment thereof is ziltivekimab or antigen binding fragment thereof. In certain embodiments, ziltivekimab is administered intravenously to a patient with CRS or at risk of CRS. In certain embodiments, ziltivekimab is administered intravenously to a patient with CRS or at risk of CRS, wherein the patient has SARS-CoV-2 infection. In certain embodiments, ziltivekimab is administered intravenously to a patient with COVID-19.

In some embodiments, ziltivekimab is administered at a dose of 2-300 mg. In some embodiments, ziltivekimab is administered at an intravenous flat dose of 2-300 mg, such as 2-5 mg, 2-10 mg, 2-20 mg, 2-30 mg, 2-40 mg, 2-50 mg, 2-100 mg, 2-150 mg, 2-200 mg, 2-250 mg, 2-300 mg, 5-10 mg, 5-20 mg, 5-30 mg, 5-40 mg, 5-50 mg, 5-100 mg, 5-150 mg, 5-200 mg, 5-250 mg, 5-300 mg, 10-20 mg, 10-30 mg, 10-40 mg, 10-50 mg, 10-100 mg, 10-150 mg, 10-200 mg, 10-250 mg, 10-300 mg, 20-30 mg, 20-40 mg, 20-50 mg, 20-100 mg, 20-150 mg, 20-200 mg, 20-250 mg, 20-300 mg, 30-40 mg, 30-50 mg, 30-100 mg, 30-150 mg, 30-200 mg, 30-250 mg, 30-300 mg, 40-50 mg, 40-100 mg, 40-150 mg, 40-200 mg, 40-250 mg, 40-300 mg, 50-100 mg, 50-150 mg, 50-200 mg, 50-250 mg, 50-300 mg, 100-150 mg, 100-200 mg, 100-250 mg, 100-300 mg, 150-200 mg, 150-250 mg, 150-300 mg, 200-250 mg, 200-300 mg, or 250-300 mg. In certain embodiments, ziltivekimab is administered at an intravenous flat dose of 2-100 mg.

In some embodiments, ziltivekimab is administered at an intravenous flat dose of about 2 mg, 5 mg, 10 mg, 15 mg, 20 mg, 30 mg, 40 mg, 50 mg, 60 mg, 70 mg, 100 mg, 150 mg, 200 mg, 250 mg, or 300 mg. In certain embodiments, ziltivekimab is administered at an intravenous flat dose of about 10 mg. In certain embodiments, ziltivekimab is administered at an intravenous flat dose of about 15 mg. In certain embodiments, ziltivekimab is administered at an intravenous flat dose of about 20 mg. In certain embodiments, ziltivekimab is administered at an intravenous flat dose of about 30 mg. In certain embodiments, ziltivekimab is administered at an intravenous flat dose of about 40 mg. In certain embodiments, ziltivekimab is administered at an intravenous flat dose of about 50 mg.

In some embodiments, ziltivekimab is administered at an intravenous flat dose of 2 mg, 5 mg, 10 mg, 15 mg, 20 mg, 30 mg, 40 mg, 50 mg, 60 mg, 70 mg, 100 mg, 150 mg, 200 mg, 250 mg, or 300 mg. In certain embodiments, ziltivekimab is administered at an intravenous flat dose of 10 mg. In certain embodiments, ziltivekimab is administered at an intravenous flat dose of 15 mg. In certain embodiments, ziltivekimab is administered at an intravenous flat dose of 20 mg. In certain embodiments, ziltivekimab is administered at an intravenous flat dose of 30 mg. In certain embodiments, ziltivekimab is administered at an intravenous flat dose of 40 mg. In certain embodiments, ziltivekimab is administered at an intravenous flat dose of 50 mg.

In some embodiments, multiple doses of ziltivekimab are administered to the patient with or at risk for CRS. In certain embodiments, ziltivekimab is administered at a first dose of 2-100 mg intravenously, followed by at least a second intravenous dose of 2-100 mg. In certain embodiments, ziltivekimab is administered at a first dose of about 10 mg intravenously, followed by a second intravenous dose of about 30 mg. In certain embodiments, ziltivekimab is administered at a first dose of about 15 mg intravenously, followed by a second intravenous dose of about 30 mg. In certain embodiments, ziltivekimab is administered at a first dose of about 30 mg intravenously, followed by a second intravenous dose of about 30 mg. In various embodiments, the second dose is administered 24 hours to 5 days after the first dose, such as 24 hours to 48 hours, 24 hours to 72 hours, or 24 hours to 4 days after the first dose is administered. In certain embodiments, the second dose is administered 24-48 hours after the administration of the first dose. In certain embodiments, the second dose is administered about 24 hours, 48 hours, 72 hours, 4 days, or 5 days after the administration of the first dose. In certain preferred embodiments, the second dose is administered 48 hours after the administration of the first dose.

5.8.3.1.10. JAK and STAT Inhibitors

In various embodiments, the IL-6 antagonist is an inhibitor of the JAK signaling pathway. In some embodiments, the JAK inhibitor is a JAK1-specific inhibitor. In some embodiments, the JAK inhibitor is a JAK3-specific inhibitor. In some embodiments, the JAK inhibitor is a pan-JAK inhibitor.

In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, INCB-039110, ABT-494, INCB-047986 and AC-410.

In various embodiments, the IL-6 antagonist is a STAT3 inhibitor. In a specific embodiment, the inhibitor is AZD9150 (AstraZeneca, Isis Pharmaceuticals), a STAT3 antisense molecule.

5.8.3.2. Small Molecule Inhibitors

In typical embodiments, small molecule JAK inhibitors and STAT inhibitors are administered orally.

In various embodiments, the inhibitor is administered once or twice a day at an oral dose of 0.1-1 mg, 1-10 mg, 10-20 mg, 20-30 mg, 30-40 mg, or 40-50 mg. In some embodiments, the inhibitor is administered once or twice a day at a dose of 50-60 mg, 60-70 mg, 70-80 mg, 80-90 mg, or 90-100 mg. In some embodiments, the inhibitor is administered at a dose of 0.1, 0.5, 1, 2, 5, 10, 15, 20, 25, 30, 35, 40, 45, or 50 mg PO once or twice a day. In some embodiments, the inhibitor is administered at a dose of 75 mg or 100 mg PO once or twice a day.

5.8.3.3. AAdditional IL-6 Antagonists

In various embodiments, the IL-6 antagonist is an antagonist peptide.

In certain embodiments, the IL-6 antagonist is C326 (an IL-6 inhibitor by Avidia, also known as AMG220), or FE301, a recombinant protein inhibitor of IL-6 (Ferring International Center S.A., Conaris Research Institute AG). In some embodiments, the anti-IL-6 antagonist comprises soluble gp130, FE301 (Conaris/Ferring).

5.8.3.4. Anti-Viral Agents

In some embodiments, the second therapeutic agent is an anti-viral agent, and the method of the present disclosure further comprises administering an effective amount of an anti-viral agent.

In some embodiments, the anti-viral agent is selected from the group consisting of: favipiravir, remdesivir, and a combination of lopinavir and ritonavir.

In some embodiments, the anti-viral agent is favipiravir.

In some embodiments, the anti-viral agent is remdesivir.

In some embodiments, the anti-viral agent is a combination of lopinavir and ritonavir.

5.8.3.5. Antibacterial Agents

In some embodiments, the second therapeutic agent is an antibacterial agent selected from the group consisting of azithromycin, tobramycin, aztreonam, ciprofloxacin, meropenem, cefepime, cetadizine, imipenem, piperacillin-tazobactam, amikacin, gentamicin, and levofloxacin.

5.8.3.6. Granulocyte-Macrophage Colony-Stimulating factor (GM-CSF) antagonist

In some embodiments, the second therapeutic agent is a GM-CSF antagonist. In certain embodiments, the GM-CSF is gemsilumab.

5.8.3.7. Angiotensin Receptor Blocker (ARB)

In some embodiments, the second therapeutic agent is an ARB, and the method of the present disclosure further comprises administering an effective amount of an ARB.

In particular embodiments, the ARB is selected from losartan, valsartan, azilsartan, candesartan, eprosartan, irgesartan, 1651mesartan, and telmisartan.

5.8.3.8. Hydroxychloroquine and Chloroquine

In some embodiments, the second therapeutic agent is an anti-malarial agent, and the method of the present disclosure further comprises administering an effective amount of an anti-malarial agent.

In some embodiments, the anti-malarial agent is hydroxychloroquine. In someembodiments, the anti-malarial agent is chloroquine.

5.8.3.9. COVID-19 Immune Serum or Plasma

In some embodiments, the second therapeutic agent is a COVID-19 immune serum or plasma, and the method of the present disclosure further comprises administering an effective amount of COVID-19 immune serum or plasma.

5.8.3.10. COVID-19 Vaccine

In some embodiments, the second therapeutic agent is a COVID-19 vaccine, and the method of the present disclosure further comprises administering an effective amount of a 44COVID-19 vaccine.

### 5.9. EXAMPLES

The following examples are provided by way of illustration not limitation.

### 5.9.1. Example 1

### Mixtures of Cannabinoids and Terpenes for Treating Hyperinflammation Associated with CRS or MAS

The cannabinoid-containing complex mixtures of the present disclosure were designed to reduce the expression of specific pro-inflammatory cytokines implicated in the pathology of CRS or MAS, including, but not limited to, COVID-19, ARDS, and sHLH, while preserving specialized anti-viral immune functions.

Activated CD14+CD16+monocytes, Helper CD4+ T-Cells, Cytotoxic CD8+ T-cells, and plasmacytoid Dendritic cells were targeted due to their importance in fighting viral infections combined with their sensitivity to cannabinoids.

The goal of this study was to reduce virally-induced hyperinflammation while preserving anti-viral immune functions, where the cannabinoid and or terpene mixtures described in the present disclosure provide the following:

1. Reduce the levels of pro-inflammatory cytokines that contribute to clinical pathology in patients who have, or who are at risk for developing CRS, such as COVID-19 patients, and/or patients with ARD or sHLH. The proinflammatory cytokines include, but are not limited to IL-1β, IL-6, TNFα, and IFNγ (optional);

2. Increase cytotoxic CD8+ T-cell proliferation and stimulation of appropriate T-cell activity; and

3. Preserve the anti-viral response function of the plasmacytoid dendritic cells by protection of exogenous IFNα production and/or supplementation with recombinant interferons such as IFNα. In addition, some formulations contain additional compounds designed to address lymphopenia, which leads to immune compromised state.

Terpenes and cannabinoids were screened for their ability to modulate target immune cells by measuring the release of key cytokines, biomarkers, and pro-inflammatory processes associated with hyperinflammation or CRS, with the expectation that cannabis-based compounds with the desired immunomodulatory properties will be effective in treating CRS or MAS. These CCCM mixtures can be used as an adjunctive therapy to be delivered with clinically-approved IFN products for the most beneficial patient results—to reduced cytokine storm (or CRS) and to preserve and activate anti-viral immunity.

The cannabinoids and/or terpenes provided herein downregulated the production of IL-1β, IL-6, and TNFα, which have been shown to correlate with severe hyperinflammation/cytokine storms, and they addressed lymphopenia by specifically increasing proliferation of cytotoxic CD8+ T-cells. Some proposed mixtures included an IFNγ-decreasing compound because of the tissue damaging potential and role of IFNγ in MAS. In addition, the single compounds were screened to remove potential agents that would reduce normal anti-viral immune functions that are necessary for fighting the disease. In particular, these mixtures were screened for their ability to preserve IFNα production.

### Materials and Methods

### Peripheral Blood Mononuclear Cell Isolation

Leukocyte packs were purchased from the Gulf Coast Regional Blood Center (Houston, Tex.). Blood was diluted 1:1 with Hanks Balanced Salt Solution from Gibco™ (Grand Island, N.Y.). Diluted blood was layered onto 15 mL Ficoll Paque Plus (GE Healthcare Life Sciences, Pittsburgh, Pa.) using SepMate 50 mL conical tubes by StemCell Technologies (Vancouver, BC, Canada) and centrifuged at 1300×g for 25 min at 4° C. The buffy coat was carefully removed from the plasma and re-suspended in HBSS and washed twice. Subsequent PBMC were resuspended in complete Roswell Park Memorial Institute (C-RPMI) Media from Gibco™ containing 5% Human AB Serum (Sigma-Aldrich, St. Louis, Mo.), 1% Penicillin-Streptomycin (Gibco™), and 0.035% β-mercaptoethanol. PBMC were cultured in 48 or 96 well plates at a density of 5×106 eukocyte packs were purchased from the Gulf Coast Regional Blocultured in 48 or 96 well plates at a density of 5×106 cells/mL in 600 or 200 μl of complete RPMI media respectively.

### Compound Preparation

The α-pinene (aP), trans nerolidol (tN), cannabidivarin (CBDV), cannabidiol (CBD), cannabinol (CBN), cannabichromene (CBC), and cannabigerol (CBG) were purchased from Sigma-Aldrich (St. Louis, MO). D-limonene (Lim) was purchased from MP Biomedical (Solon, Ohio). Linalool (Lin) was purchased from Tokyo Chemical Industry (Portland, Oreg.) and phytol was purchased from Agilent (Santa Clara, Calif.). (6aR,10aR)-delta-9-tetrahydrocannabinol (Δ9-tetrahydrocannabinol or THC) was supplied by the National Institute of Drug Abuse (NIDA). The terpenes Lim, Lin, Phytol A, and tN were dissolved in dimethyl sulfoxide (DMSO). The cannabinoids CBC, CBD, CBDV, CBG, and CBN were supplied in methanol. The methanol was evaporated under nitrogen and the precipitate was resuspended in DMSO. THC and aP were dissolved in ethanol. Compounds were diluted in complete RPMI to a final DMSO or ethanol concentration of 0.031%. The concentrations of the compounds were 0.001, 0.01, 0.1, 1.0, and 10 μM. In viability experiments, non-cannabinoid compounds were also diluted to a final concentration of 50 μM, with a final DMSO or ethanol concentration of 0.025% or 0.031%.

### Cell Culture and Stimulation

In viability experiments, PBMC with treatments were cultured with GBS compounds as prepared above on flat bottom 48 or 96 well plates at 37° C. in 5% CO2 for 18 or 6 hours, respectively. In experiments examining pDC, following 6 hours incubation, cells were harvested using ice cold FACS buffer. In experiments examining monocytes and T cells, cells were harvested following 18 hours incubation using CellStripper (Corning, Corning, N.Y.) for 15-30 minutes at 37° C. An aliquot of 200 μl of cell suspension was used for flow cytometry, while the remainder was used to determine the cell concentration using a Coulter Particle-Counter (Beckman-Coulter Inc., Brea, Calif.).

In immunological activity experiments, PBMC suspensions were pre-treated with cannabinoid and/or terpene compounds for 30 minutes before treatment with designated stimuli, cultured in 96 well tissue culture plates, and incubated at 37° C. and 5.0% CO2. In pDC experiments, cell suspensions were stimulated with CpG-ODN Type A 2216 (15 μg/mL) (InvivoGen©, San Diego, Calif.) following treatment with cannabinoids. Golgi transport inhibitors Monensin (2 μM)/Brefeldin A (3.0 μg/mL) (Thermo Fisher Scientific, Waltham, Mass.) were added to culture 3 hours post-stimulation, and after 7 hours of stimulation cells were harvested for evaluation by flow cytometry for IFNα and TNFα. In monocyte experiments, cell suspensions were stimulated with 1000 ng/mL LPS (S. typhosa, Sigma Aldrich, St. Louis, Mo.) for 18 hours before harvesting supernatants for IL-1β, Golgi blocked for 6 hours before harvesting cells for assessment of IL-6, or cultured for 48 hours before harvesting cells for assessment of HLA-DR, CD80, and CD86. In phagocytosis experiments, cell suspensions were treated with 3.0 mg/mL of pHrodo Green BioParticles (Thermo Fisher). In T cell experiments, cells were plated on 96 well tissue culture plates pre-coated with 5.0 μg/mL anti-CD3 (clone UCHT1, Biolegend, San Diego, Calif.) and stimulated with 5.0 μg/mL anti-CD28 (clone 28.2, Biolegend) and supplemented with 5.0 ng/mL IL-2 (Roche Applied Science, Indianapolis, Ind.). CD25 and CD69 were assessed by flow cytometry 24 hours post-stimulation. In proliferation experiments, cell suspensions were incubated with CFSE Cell Division Tracker (Biolegend) according to manufacturer protocols, harvested 4 days post-stimulation and assessed by flow cytometry. Cell suspensions were further stimulated with 50 ng/mL phorbol 12-myristate 13-acetate (PMA) and 1000 ng/mL ionomycin (Io) at 4 days post-stimulation for 18 hours, Golgi blocked for 6 hours, and harvested for IL-4 and IFNγ assessment by flow cytometry.

### Flow Cytometry

In brief, pDC, monocyte and T cell were identified using antibodies directed against unique epitopes for each cell type and analyzed by flow cytometry. Cells were fixed in BD CytoFix and resuspended in FACS buffer for extracellular staining. To determine the changes in cell viability in mixed PBMC, gates were first set on singlet events, then CD45+ cells, then populations identified by CD3+ (T cells) and CD14+ (monocytes), and then 7-AAD- for viability. Likewise, for pDC viability experiments, pDC were identified as CD303+ and viable by exclusion of Near IR L/D stain (Life Technologies, Carlsbad, Calif.). For intracellular staining, fixed cells were permeabilized in BD Permwash and incubated with appropriate antibodies and 7% human AB serum. pDC were identified as CD303+and assessed for intracellular IFNα and TNFα. Monocytes were identified as CD14+ and assessed for extracellular HLA-DR, CD80, CD86, and intracellular TNFα and IL-6. In phagocytosis experiments, PBMC were identified as CD45+ and monocytes were identified based on side scatter and then phagocytosing monocytes. T cells were identified as CD3+ and T cell subsets identified by CD4+ and CD8+ and assessed for extracellular CD25 and CD69 and intracellular IFNγ and IL-4. To analyze T cell proliferation, FlowJo 10.0 proliferation tool was used by setting the undivided peak of CFSE stained cells in each sample.

### IL-1β Quantification

To quantify IL-1β produced by monocytes, supernatants were collected from LPS stimulated cell suspensions after 18 hours and frozen at −80° C. ELISA MAX from Biolegend was used as per manufacturer protocol to quantify IL-1β to a sensitivity of 0.5 μg/mL.

### Data Acquisition and Analysis

Flow Cytometry data were acquired on a BD FACS Canto II and analyzed using FlowJo 10.0 (TreeStar Inc, Ashland, Oreg.). Graphpad Prism was used for statistical analysis and graph generation. Data were analyzed by repeated-measure one-way ANOVA with Dunnett's posttest, and significance determined at P<0.05. In the event a sample parameter could not be measured by flow cytometry analysis software, the sample was excluded and analyzed by a mixed repeated-measure one-way ANOVA with Dunnett's posttest. Where appropriate, data were normalized to VH.

Results

Viability/Toxicity

Treatment with GBS Compounds did not induce cell death in PBMC, monocytes, T cells, or pDC or show other signs of toxicity.

PBMCs were treated for 18 hours with single compounds in a toxicity study parallel to the range of concentrations used in the immune modulation activity assays: 0.001 μM, 0.01 μM, 0.1 μM, 1.0 μM, 10.0 μM, and 50.0 μM, and analyzed by flow cytometry. PBMC were identified as CD45+ cells, and within that population monocytes were identified as CD14+ cells and T cells as CD3+ cells. Separately, PBMC were treated for 6 hours with single compounds and analyzed by flow cytometry, identifying pDC cells as CD303+.

Immune Modulating Activity

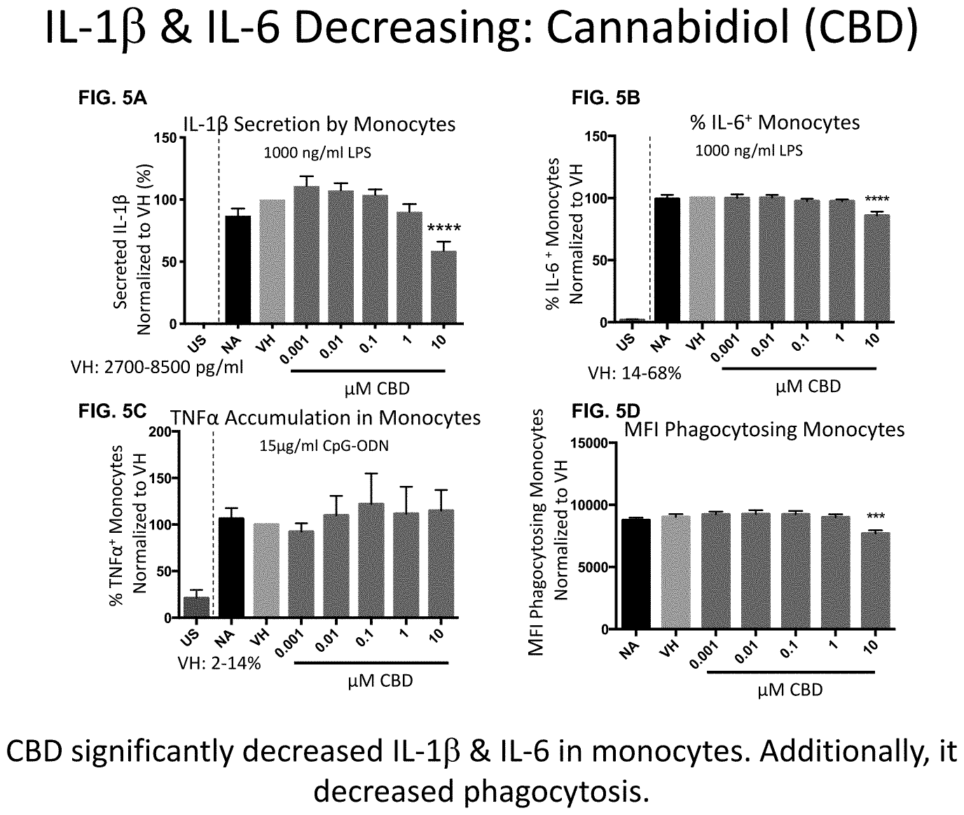

Cannabidiol (CBD) suppressed monocyte pro-inflammatory cytokines and phagocytosis, as shown in FIGS. 5A-5D. Treatment with CBD significantly decreased monocyte production of both pro-inflammatory IL-1β and IL-6 at 10 μM (FIGS. 5A, 5B). CBD also significantly decreased the MFI of phagocytosis by monocytes at 10 μM (FIG. 5D).

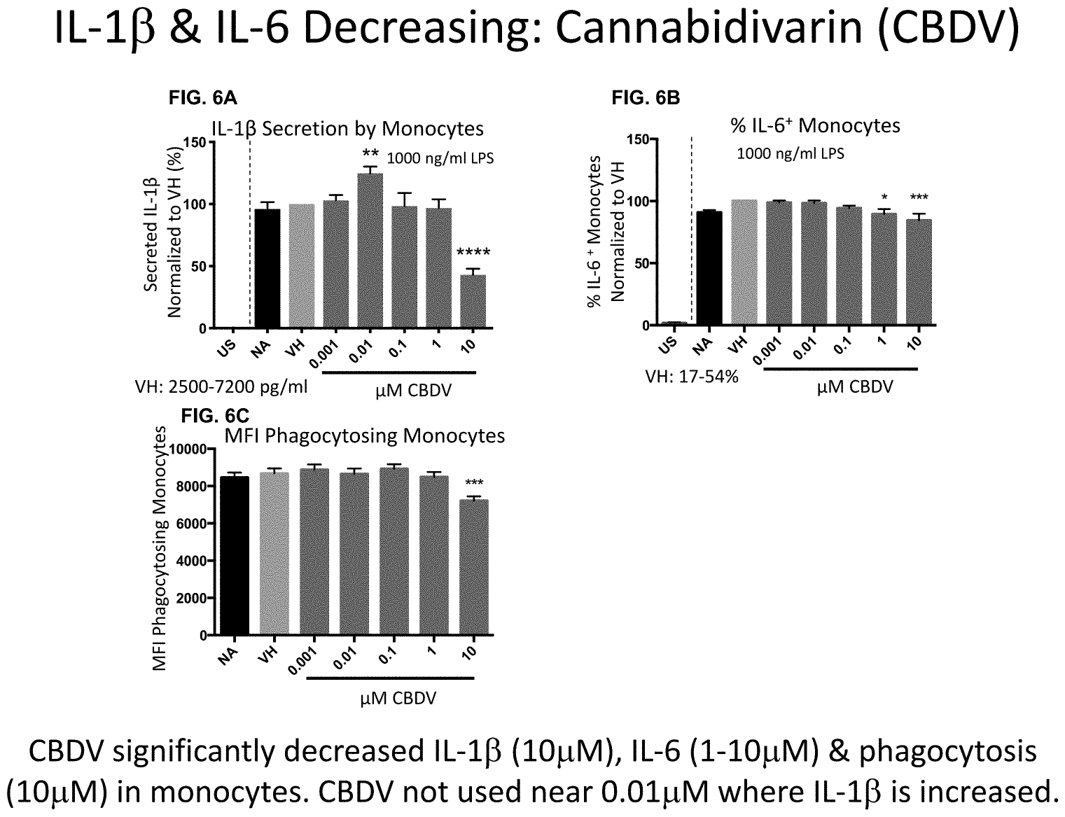

Cannabidivarin (CBDV) decreased both IL-1β and IL-6 levels in monocytes; as well as, decreasing phagocytosis, as shown in FIGS. 6A-6C. CBDV significantly decreased IL-1β at 10 β, IL-6 at 1-10 μM, and phagocytosis at 10 μM in monocytes. Significantly, CBDV will not be formulated within the CCCM at molar concentrations (or equivalent dosages) near 0.01 μM CBDV which is the concentration at which CBDV significantly increases IL-1β in monocytes. The trend reversed to show CBDV-decreasing IL-1β at higher concentrations of CBDV, and there was a statistically significant decrease in IL-1β at 10 μM. IL-6 was significantly decreased in the range of 1-10μ CBDV.

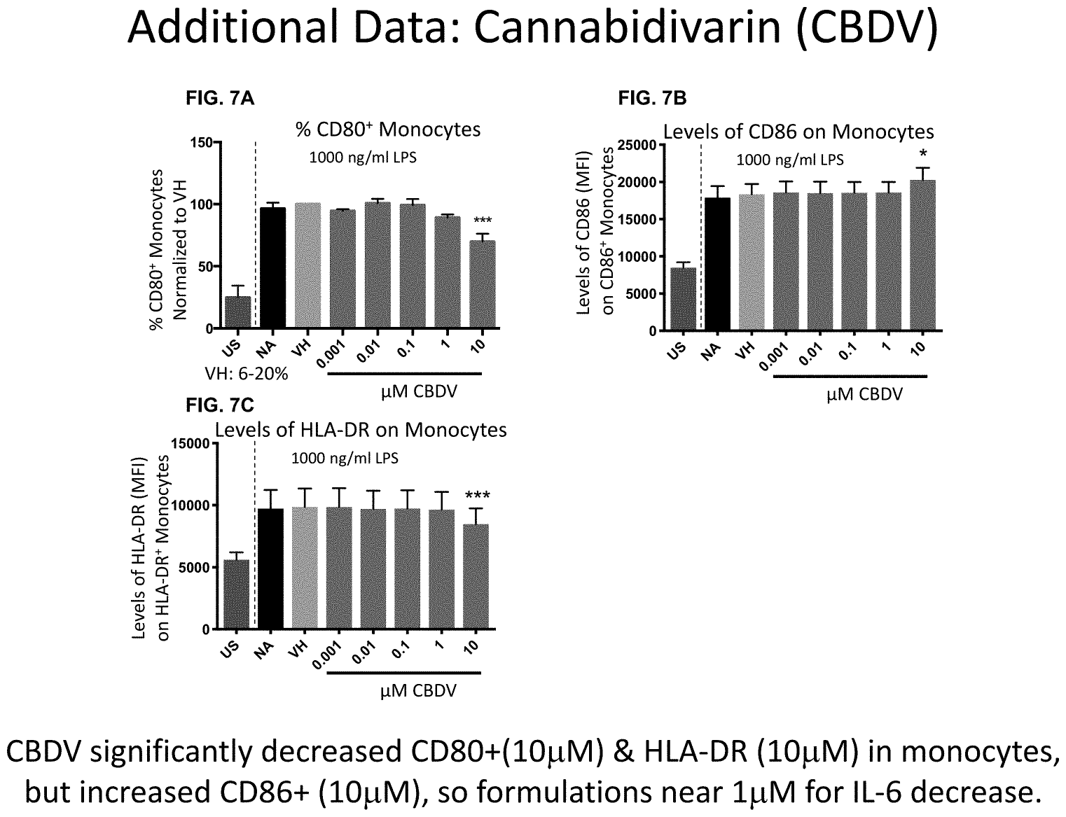

Cannabidivarin (CBDV) significantly affected co-stimulatory markers in monocytes, as shown in FIGS. 7A-7C. CD80+was decreased at 10 μM and HLA-DR was decreased at 10 μM, but CBDV increased the co-stimulatory marker CD86+ (10 μM). This additional data is relevant to the formulation of therapeutic mixtures. The therapeutic range for CBDV was centered around 1 μM to optimize its IL-6 decreasing potential (FIG. 6B), while avoiding the CBDV concentration (10 μM) where CD86+ was increased.

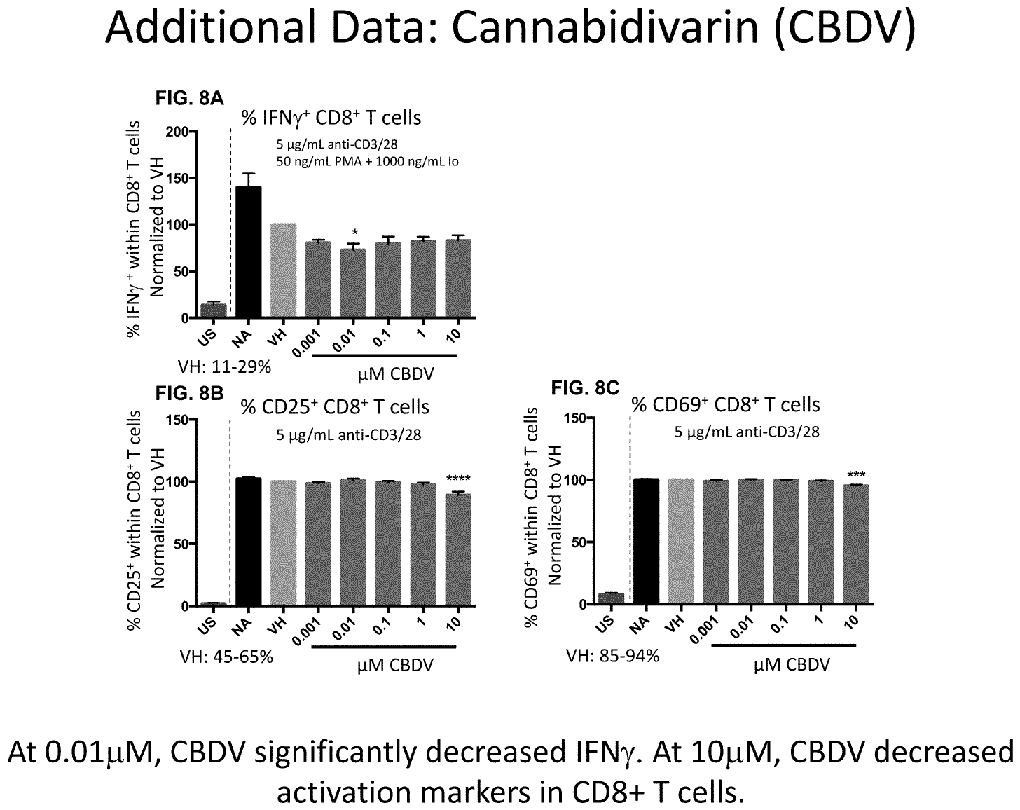

Cannabidivarin (CBDV) significantly decreased IFNγ, as well as decreasing activation markers in T cells, as illustrated in FIGS. 8A-8C. This additional data on the effects of CBDV on CD4+ and CD8+ T cells helps to establish its therapeutic range. At 10 μM, CBDV also decreased CD25+ and CD69+ T cell activation markers in CD8+ T cells. At 0.01 μM, CBDV significantly decreased IFNγ, but CBDV also increased IL-1β in FIG. 6A at the same concentration, which is contra-indicated. Therefore, the therapeutic range for CBDV was centered around 1 μM to optimize its IL-6 (FIG. 6B) decreasing potential.

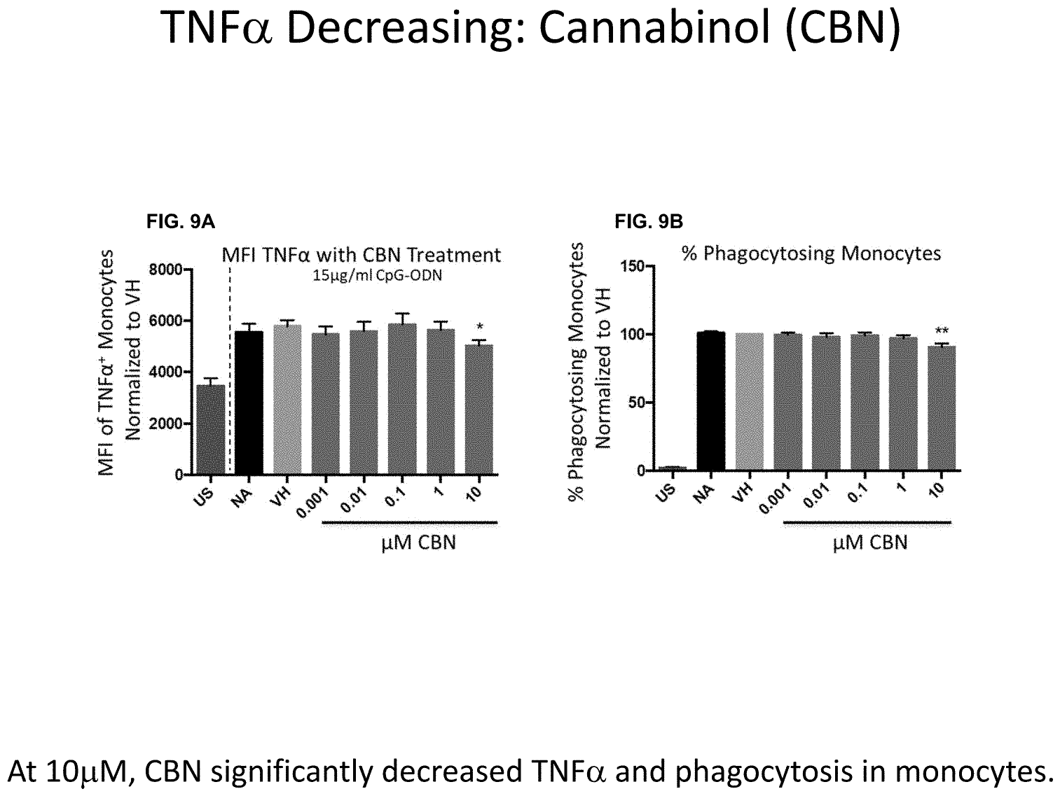

Cannabinol (CBN) significantly decreased TNFα and phagocytosis in monocytes, as seen in FIGS. 9A-9B. At 10 μM, CBN significantly decreased TNFcc, as well as decreasing phagocytosis in monocytes.

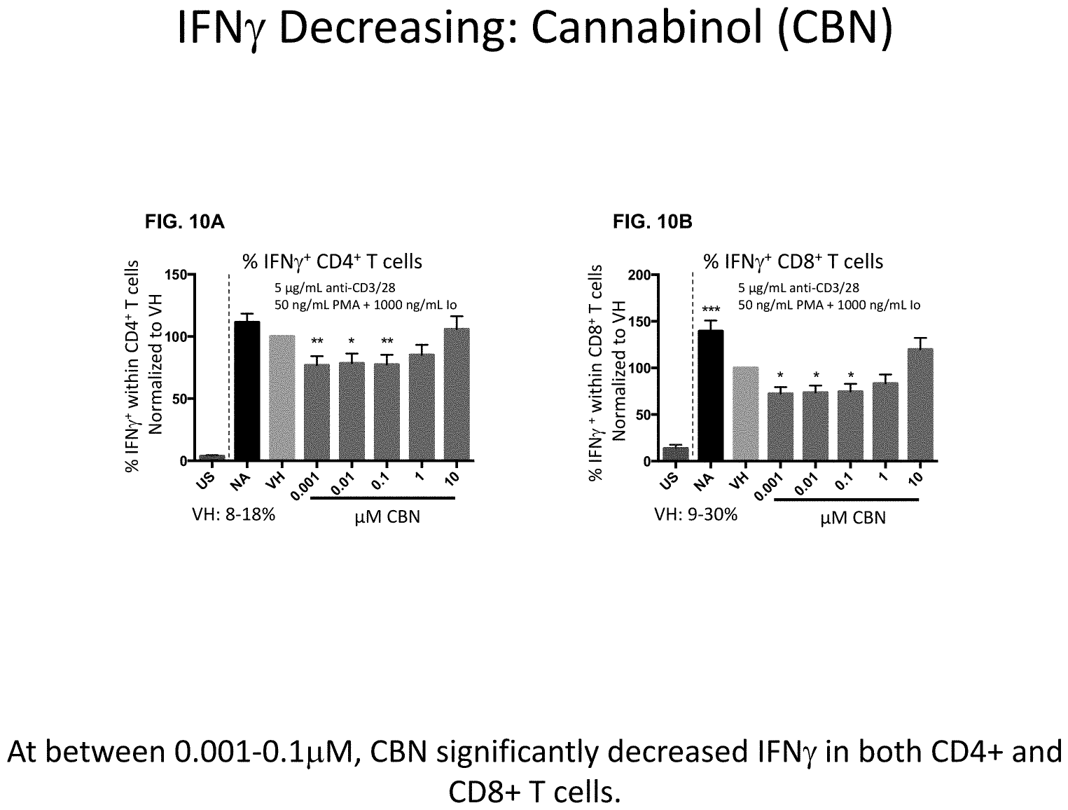

Cannabinol (CBN) significantly decreased IFNγ in both CD4+ T cells and CD8+ T cells, as shown in FIGS. 10A-10B. Treatment with CBN at between 0.001-0.1 μM, CBN significantly decreased IFNγ in both CD4+ and CD8+ T cells.

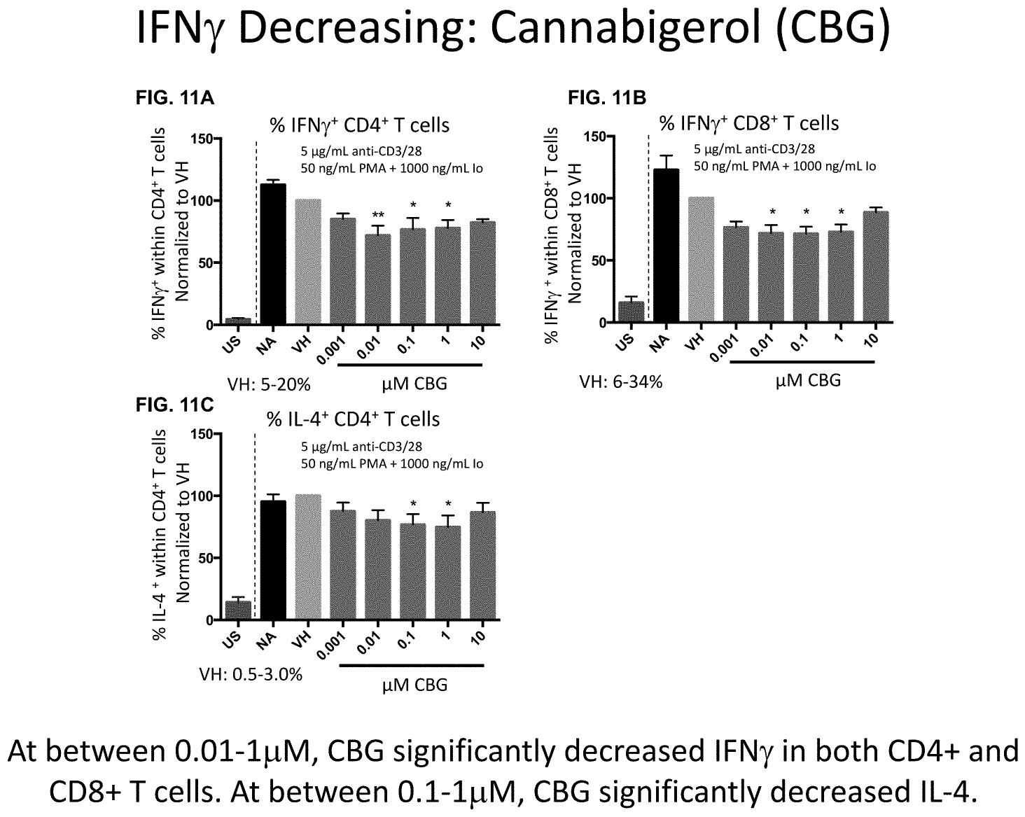

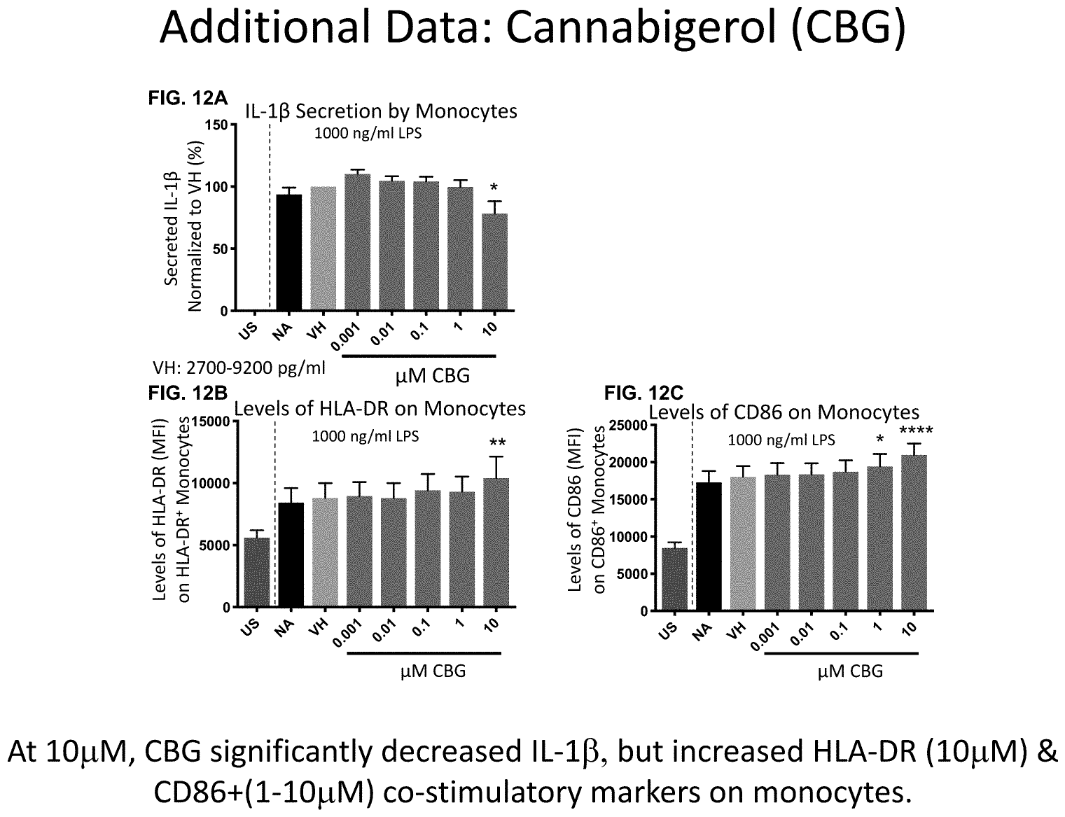

Cannabigerol (CBG) significantly decreased IFNγ in T cells, as illustrated in FIGS. 11A-11C. Treatment with CBG at between 0.01-1 μM significantly decreased IFNγ in both CD4+ and CD8+ T cells. Treatment with CBG at between 0.1-1 μM also significantly decreased IL-4 levels in CD4+ T cells.

Cannabigerol (CBG) significantly decreased IL-1β, but increased co-stimulatory markers in monocytes, as shown in FIGS. 12A-12C. This additional data is relevant to the selection of the therapeutic range for CBG. At 10 μM, CBG significantly decreased in monocytes. Conversely, CBG treatment enhanced two of the three costimulatory factors measured in monocytes. Treatment with CBG at 10 μM enhanced the HLA-DR costimulatory marker, while treatment with CBG at between 1-10 μM enhanced the CD86+(1-10 μM) co-stimulatory marker. The therapeutic range for CBG was selected to optimize the IFNγ decreasing potential (FIGS. 11A-11C) in the CCCM, while avoiding the concentrations that triggered an increase in the co-stimulatory markers, as depicted here.

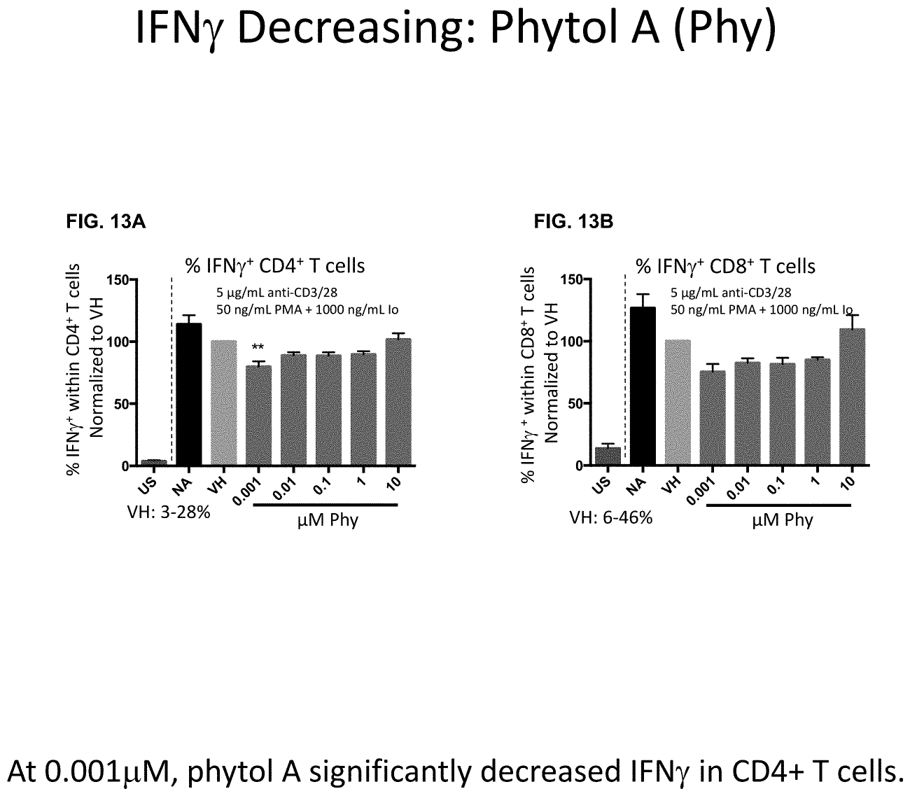

Phytol A decreased IFNγ production in CD4+ T cells, as shown in FIGS. 13A-13B. Phytol A significantly decreased CD4+ T cell production of inflammatory IFNγ at a concentration of 0.001 μM (FIG. 13A). While visually the same trend was observed in CD8+ T cells at the same concentration (FIG. 13B), the decrease was not statistically significant in CD8+ T cells.

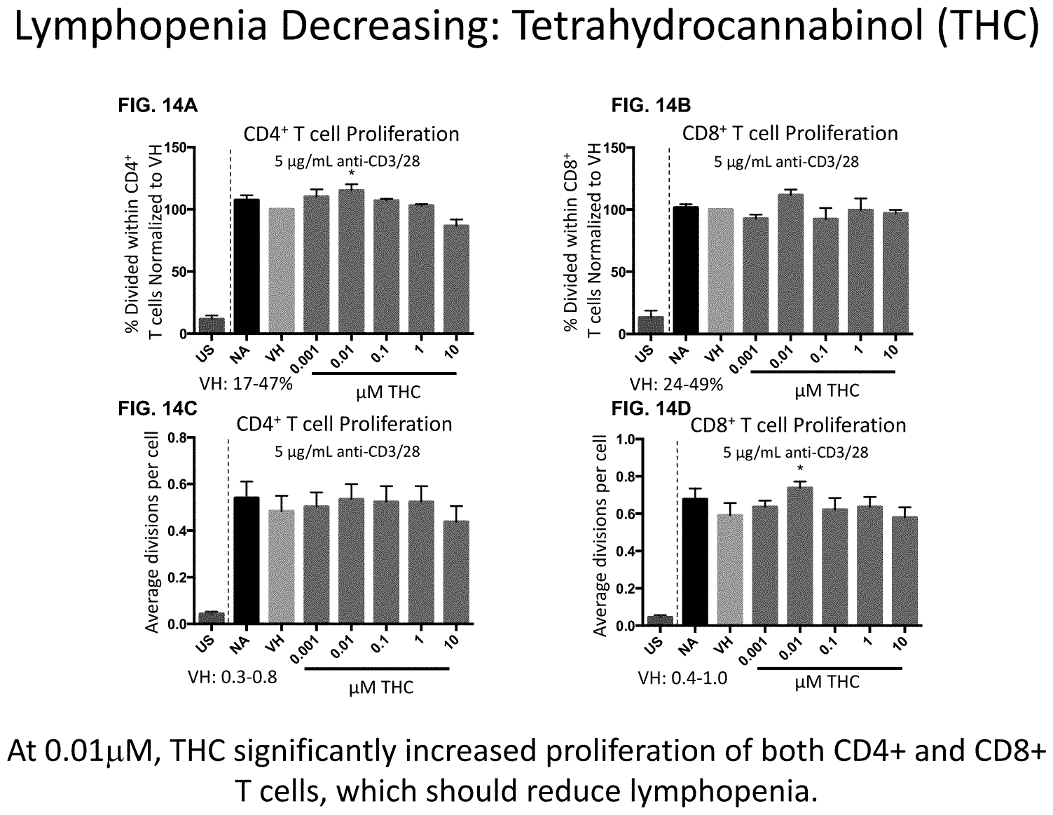

Δ-9-Tetrahydrocannabinol (THC) significantly increased the proliferation of T cells, as shown in FIGS. 14A-14D. At 0.01 μM, THC significantly increased proliferation of both CD4+ and CD8+ T cells, which should reduce lymphopenia in COVID-19 and CRS patients.

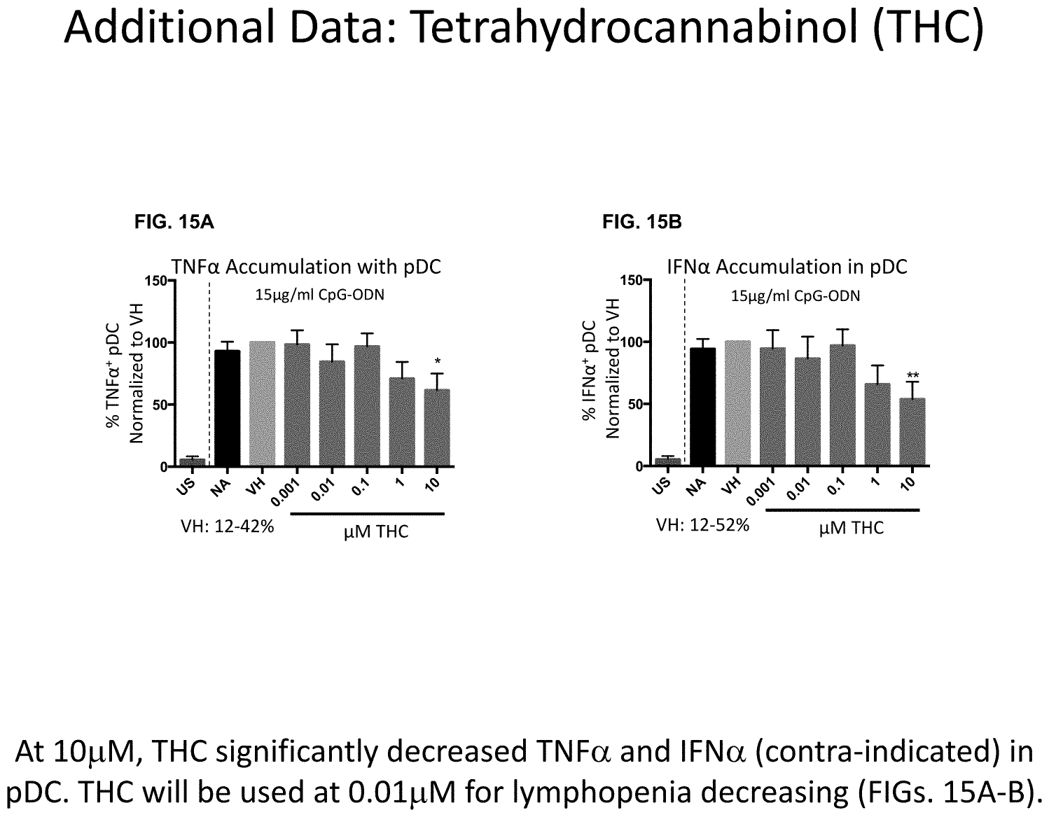

Δ-9-Tetrahydrocannabinol (THC) significantly decreased both TNFα and IFNα in plasmacytoid dendritic cells (pDC), as shown in FIGS. 15A-15B. This additional data is relevant to establishing the therapeutic range of THC within the CCCM. At 10 μM, THC significantly decreased both TNFα and IFNα in plasmacytoid dendritic cells (pDC). Decreasing TNFα levels is therapeutically desirable, however, decreasing IFNα is contra-indicated. Therefore, we will be using THC at a lower concentration to preserve IFNα production in pDC that is an essential trigger for anti-viral immunity. THC will be used at 0.01 μM for its lymphopenia decreasing (FIGS. 14A-B) effects.

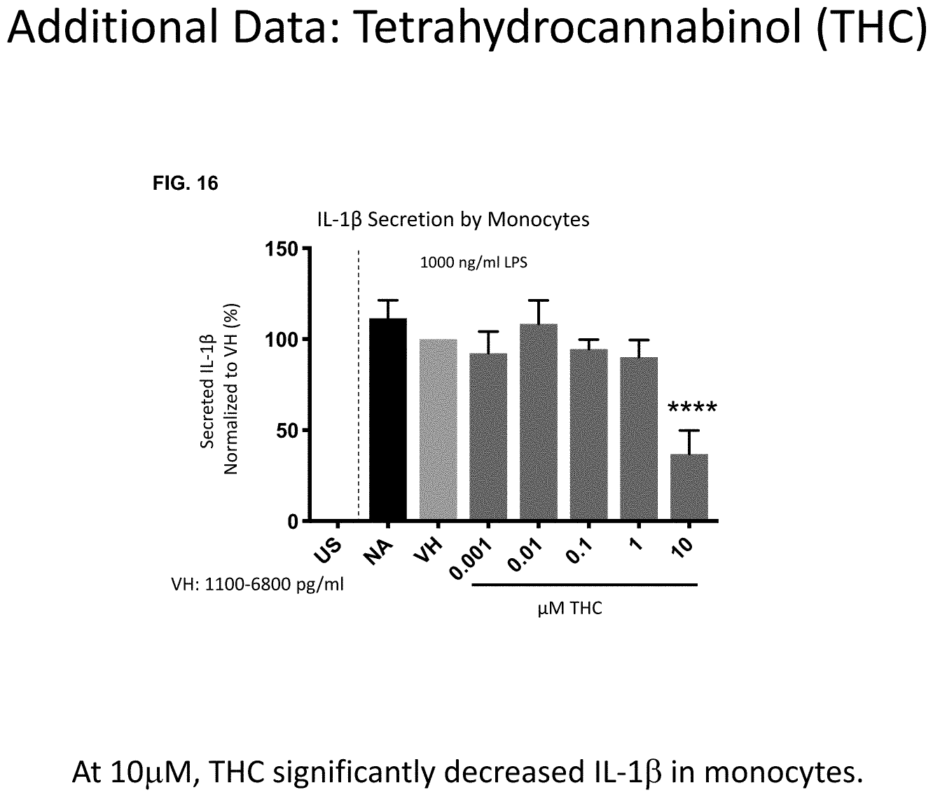

Δ-9-Tetrahydrocannabinol (THC) significantly decreased IL-1β levels in monocytes, as illustrated in FIG. 16. At 10 μM, THC significantly decreased IL-1β levels in monocytes; however, THC will be used at 0.01 μM within the CCCM for its lymphopenia decreasing (FIGS. 14A-B) effects.

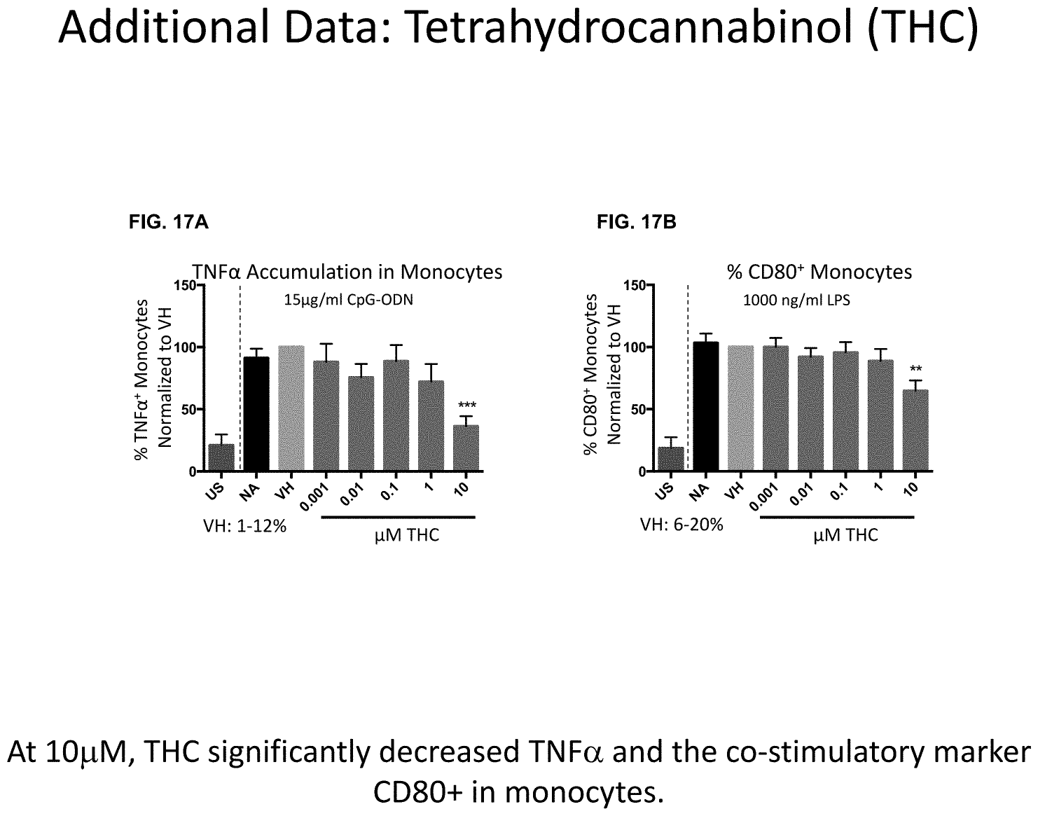

Δ-9-Tetrahydrocannabinol (THC) significantly decreased TNFα and the co-stimulatory marker, CD80+, as shown in FIGS. 17A-17B. At 10 μM, THC significantly decreased TNFα and the co-stimulatory marker, CD80+, in monocytes. Nevertheless, THC will be used at 0.01 μM within the CCCM for its lymphopenia decreasing (FIGS. 14A-B) effects.

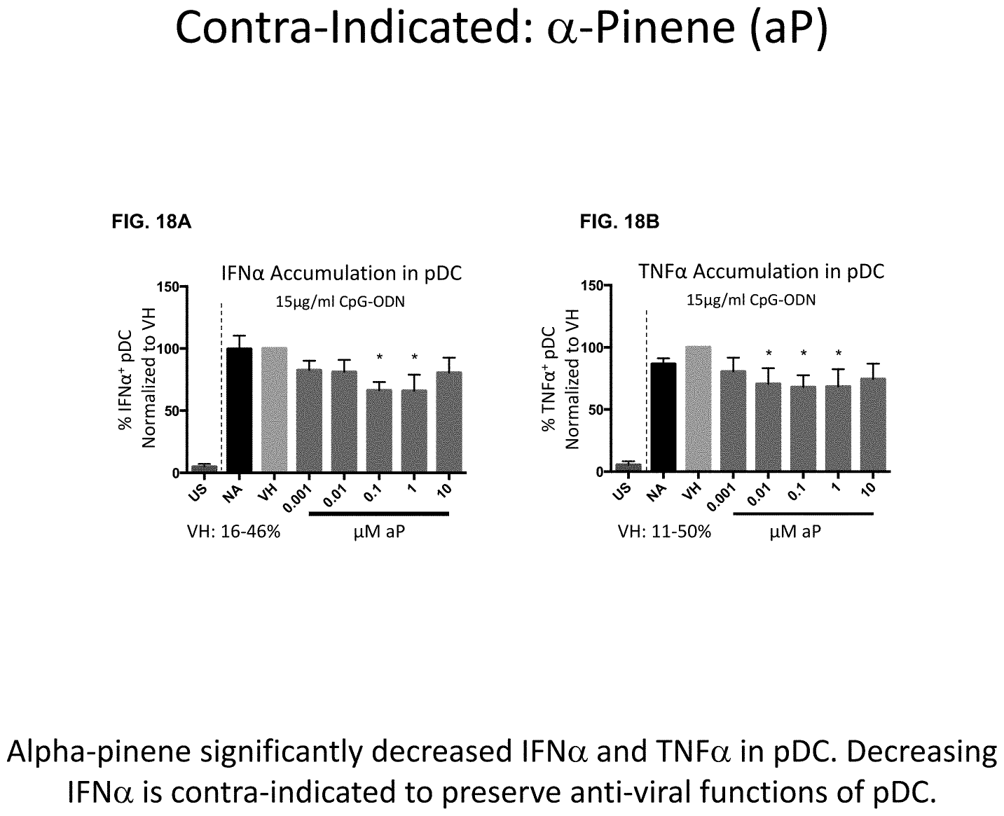

FIGS. 18A-18B illustrate why alpha-Pinene (aP) was contra-indicated. Alpha-Pinene significantly decreased IFNα levels in plasmacytoid dendritic cells (pDC), which potentially decreases the pDC signal necessary to trigger anti-viral immunity. α-Pinene significantly suppressed pDC production of inflammatory cytokines IFNα and TNFα in response to CpG-ODN after 7 hours of stimulation. The activity was at the mid-to-low concentrations of α-pinene, 0.01, 0.1, and 1.0 μM for IFNα (FIG. 18A) and 0.1 and 1.0 μM for TNFα (FIG. 18B) on pDCs. Lowering TNFα is therapeutically desired, but not at the expense of the IFNα. Because both happen at the same concentration range, aP may not be used in the cannabinoid containing complex mixtures.

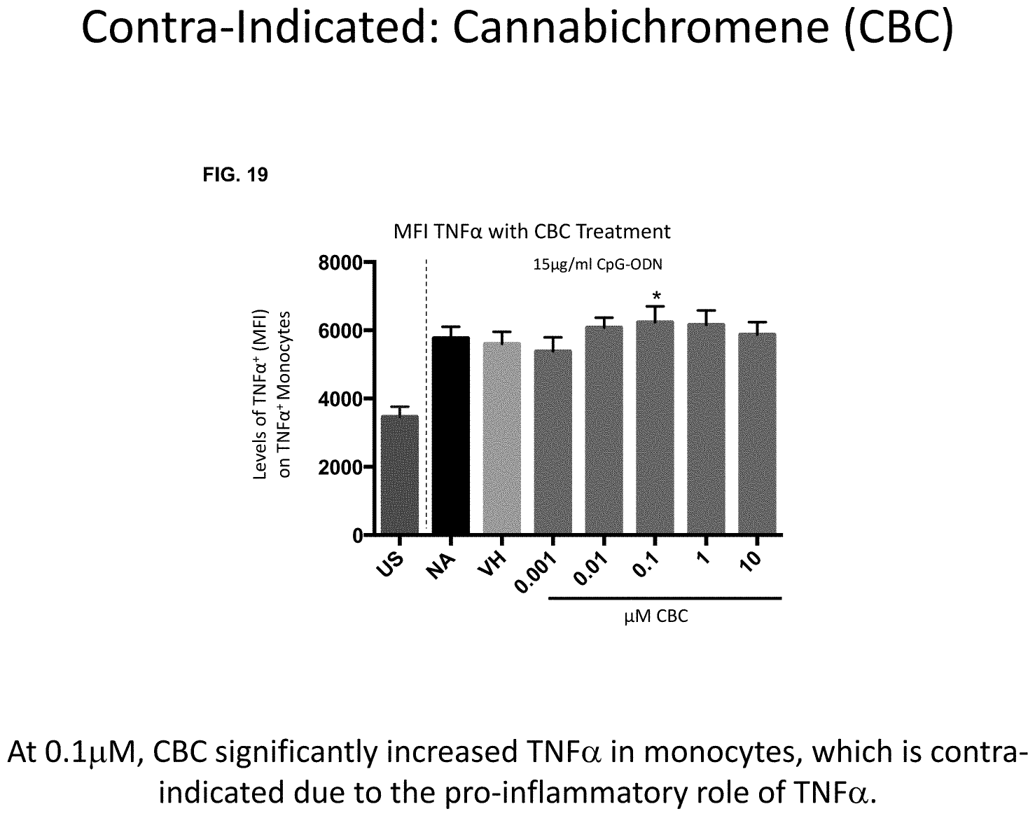

Cannabichromene (CBC) significantly increased TNFα in monocytes, as depicted in FIG. 19. At 0.1 μM, CBC significantly increased TNFα in monocytes, which is not therapeutically desired due to the pro-inflammatory role of this cytokine. Therefore, CBC was contra-indicated in CCCM for therapeutic treatment of CRS/MAS.

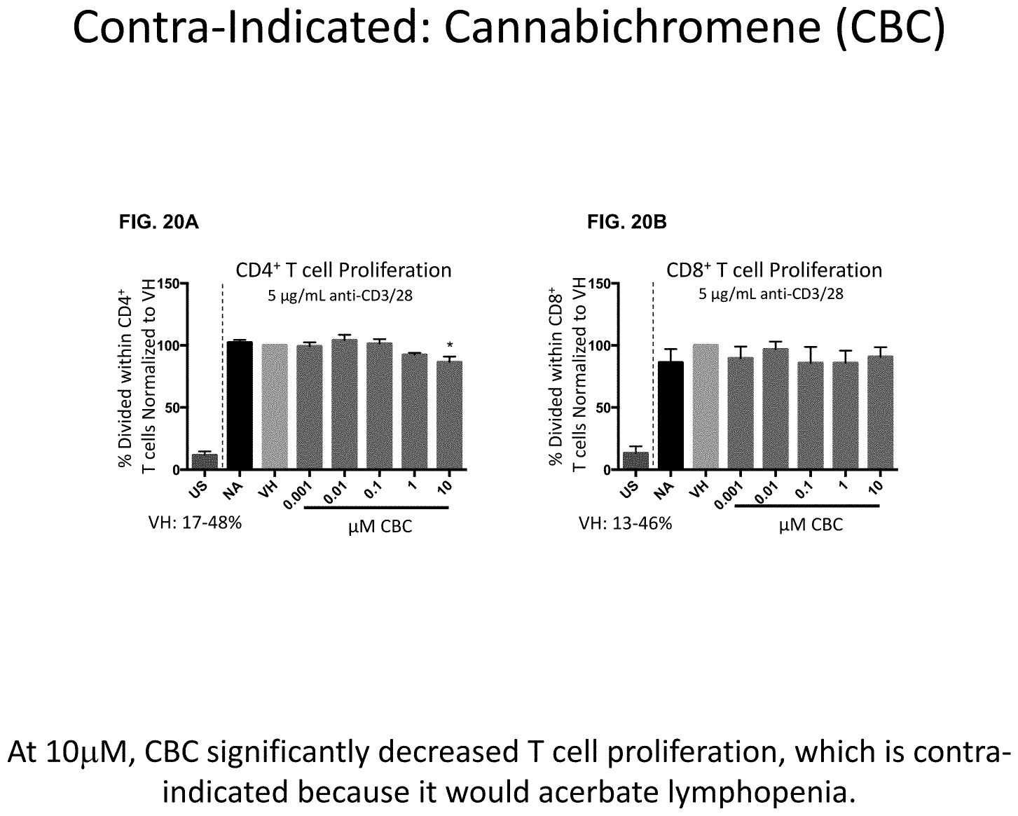

Cannabichromene (CBC) significantly decreased T cell proliferation, as illustrated in FIGS. 20A-20B. At 10 μM, CBC significantly decreased T cell proliferation in CD4+ T cells, which would acerbate Lymphopenia. The effect of CBC on CD8+ T cells was trending downward with increasing concentration of CBC, but the decrease was not statistically significant for CD8+ T cells. This additional data further illustrates why cannabichromene was contra-indicated in CCCM for therapeutic treatment of CRS/MAS.

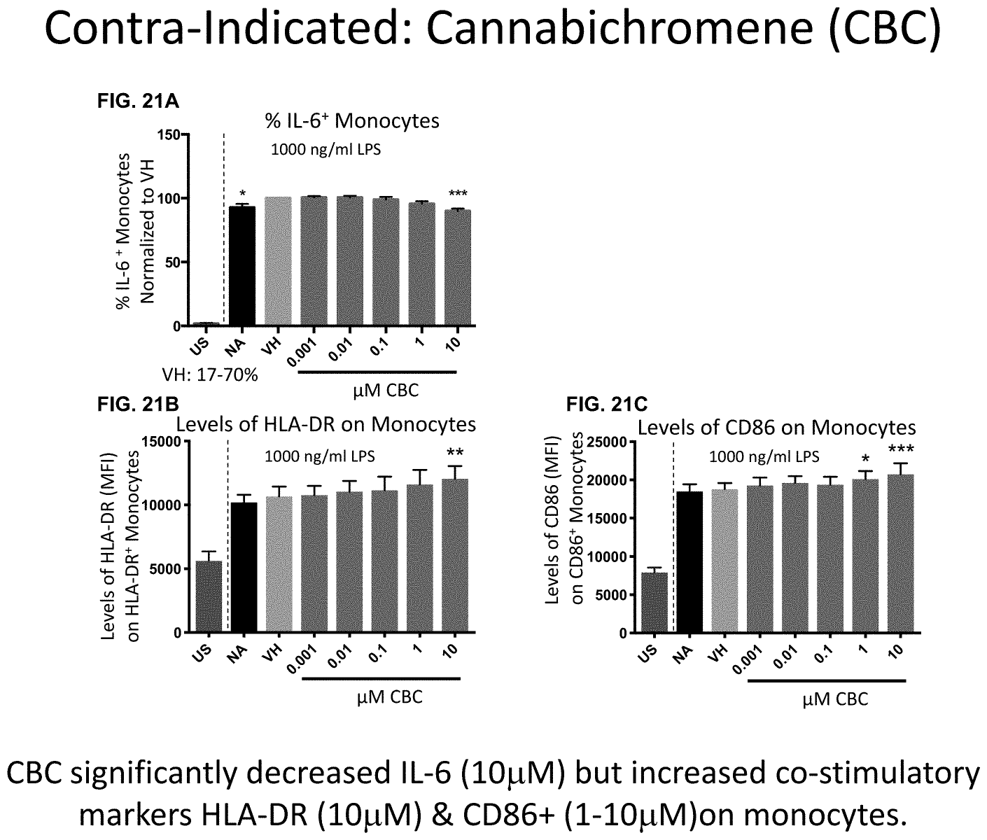

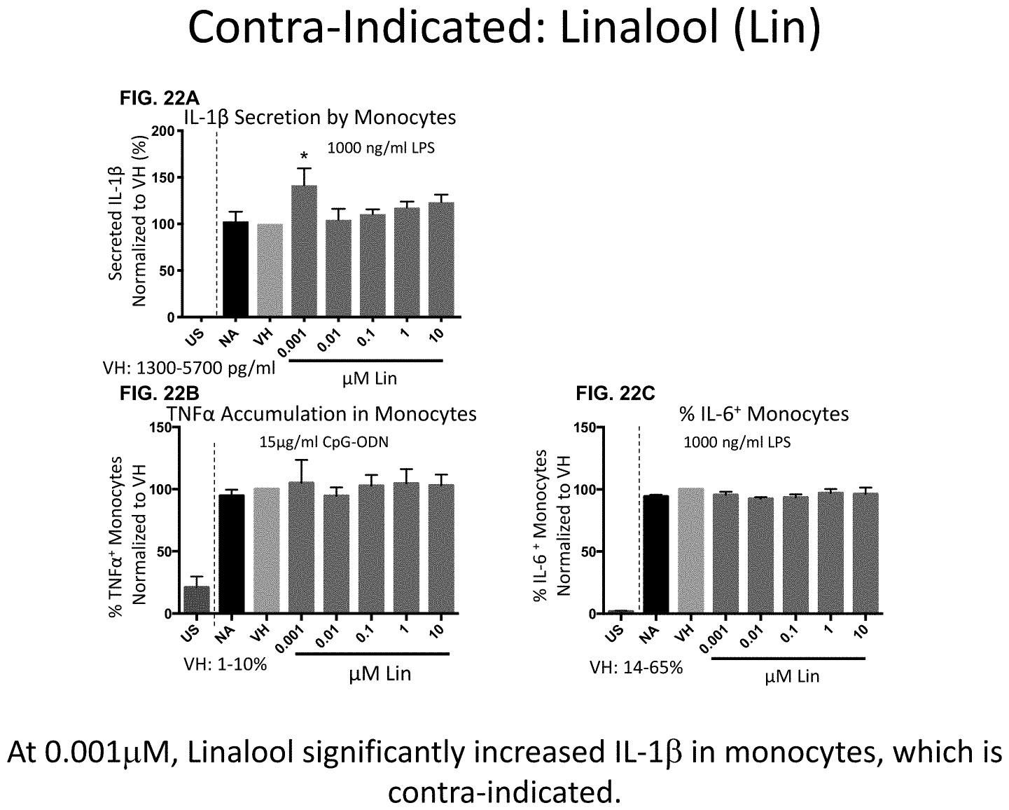

Cannabichromene (CBC) had mixed effects on pro-inflammatory processes in monocytes, as illustrated in FIGS. 21A-21C. Treatment with 1004 CBC significantly decreased IL-6 levels in monocytes, but it also increased the co-stimulatory markers HLA-DR (10 μM) & CD86+(1-10 μM) on monocytes. This additional data provides another reason that CBC is contra-indicated for use within CCCM for the treatment of CRS/MAS.

Linalool (Lin) significantly increased IL-1β in monocytes, as depicted in FIGS. 22A-22C. Because linalool enhanced the pro-inflammatory cytokine IL-1β secretion by monocytes, it is not included in the CCCM for the treatment of CRS/MAS (contra-indicated). Linalool treatment at a concentration of 0.001 μM significantly increased inflammatory cytokine IL-1β secretion in monocytes (FIG. 22A). It did not exhibit any effects on IL-1β production at any other concentration and it did not have any statistically significant effects on any other immunological functional responses measured in the immune modulation survey.

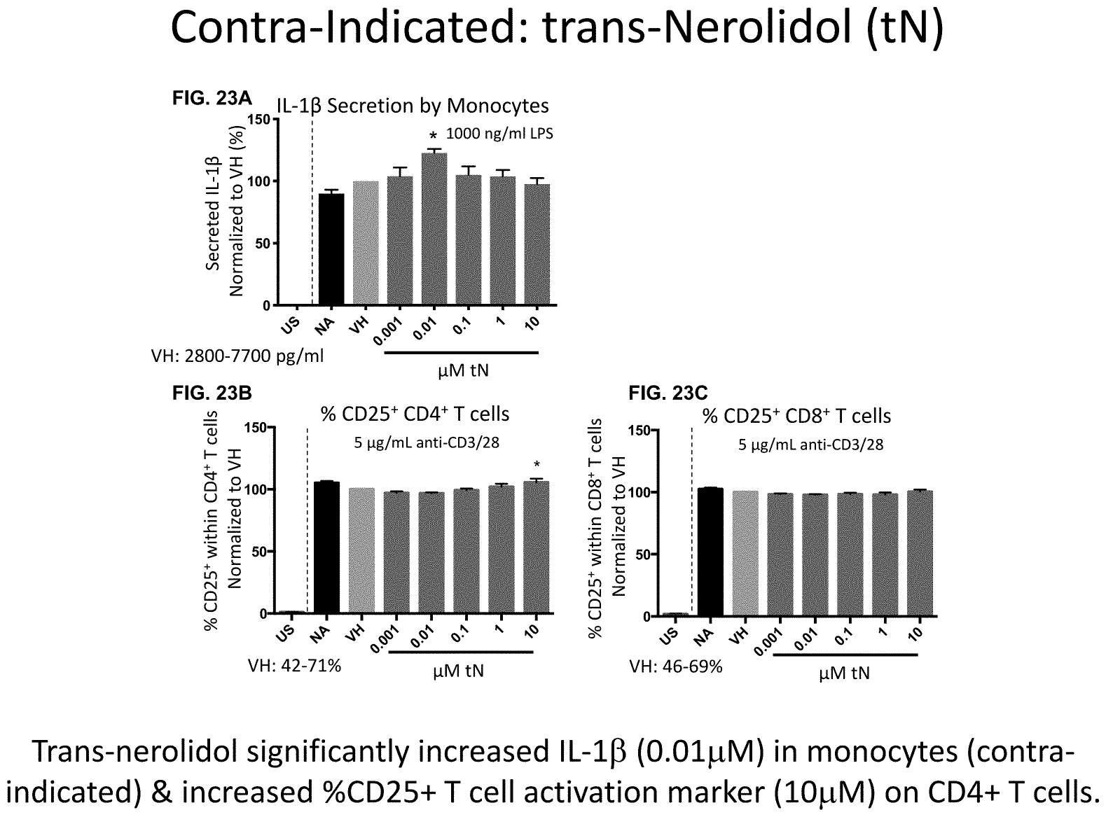

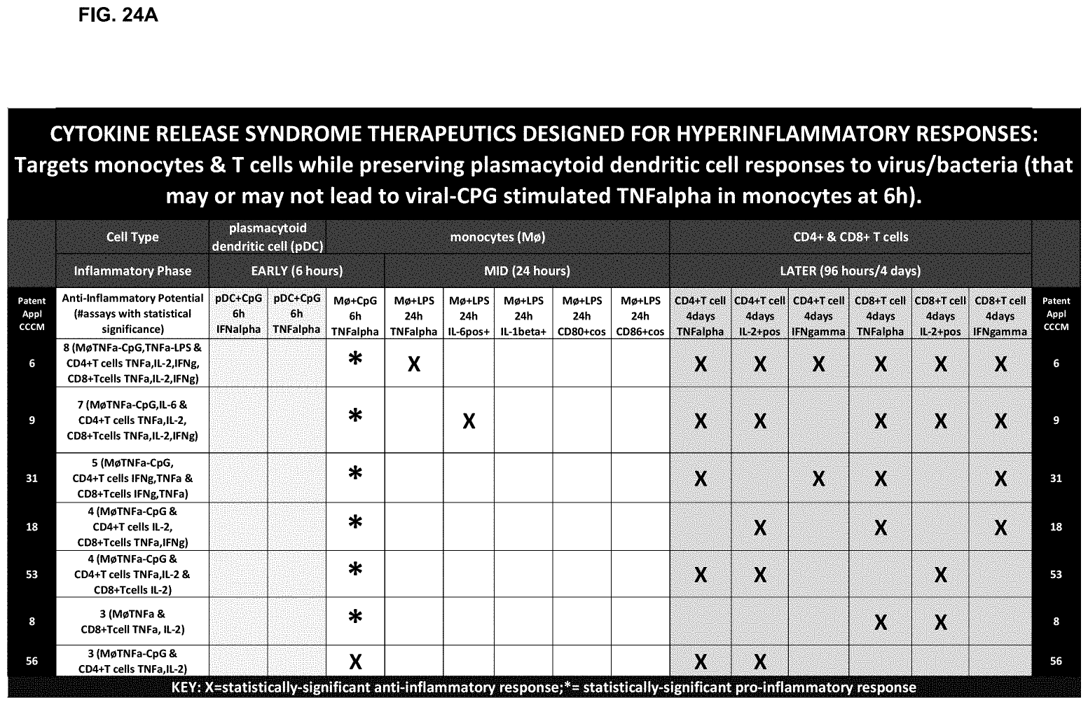

Trans-Nerolidol (tN) enhanced IL-β secretion in monocytes and CD4+ CD25+ T cell activation, as depicted in FIGS. 23A-23C. Treatment with trans-nerolidol enhanced the secretion of inflammatory IL-1β by monocytes at the concentration of 0.01 μM (FIG. 23A). In addition, tN increased the % CD25+ in CD4+ T cells (FIG. 23B), a marker of T cell activation. Because tN significantly increased IL-1β levels in monocytes and levels of the activation marker CD25+ in CD4+ T cells, it is contra-indicated within CCCM for the treatment of CRS/MAS.

### Conclusions

This broad survey of immune modulating activity across multiple human immune cell types within the PBMC revealed that cannabis-derived compounds can be used to achieve one of the present inventor's therapeutic goals, which is to downregulate the production of the pro-inflammatory cytokines IL-1β, IL-6, TNFα, and IFNγ, which have been shown to correlate with severe hyperinflammation/CRS/MAS, and to decrease lymphopenia by specifically increasing proliferation of T-cells. In addition, the proposed CCCM mixtures were screened to remove potential agents that would reduce normal anti-viral immune functions that are necessary for fighting viral-based diseases, such as influenzas and COVID-19 (among others). In particular, the proposed CCCM were screened for their ability to preserve IFNα production in pDC.

### 5.9.2. Example 2

### Predicted Immunomodulatory Effects of Cannabinoid Containing Complex Mixtures (CCCM)

Cannabinoid (and/or terpene) containing compound mixtures are formulated and their immunomodulatory effect was predicted based on the individual immunomodulatory effects on Peripheral Blood Mononuclear Cells as described in Example 1.

### Compound Preparation

The α-pinene (aP), trans nerolidol (tN), CBDV, CBD, CBN, cannabichromene (CBC), and CBG are purchased from Sigma-Aldrich (St. Louis, Mo.). D-limonene (Lim) is purchased from MP Biomedical (Solon, Ohio). Linalool (Lin) is purchased from Tokyo Chemical Industry (Portland, Oreg.) and phytol is purchased from Agilent (Santa Clara, Calif.). (6aR,10aR)-delta-9-tetrahydrocannabinol (Δ9-tetrahydrocannabinol or THC) is supplied by the National Institute of Drug Abuse (NIDA).

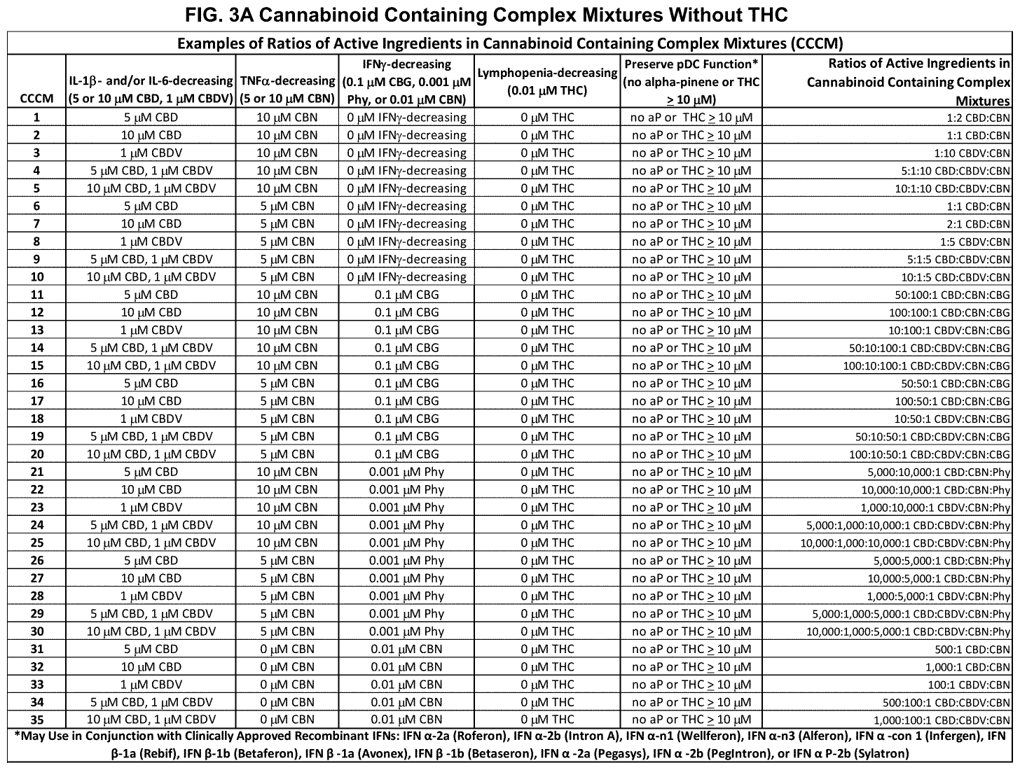

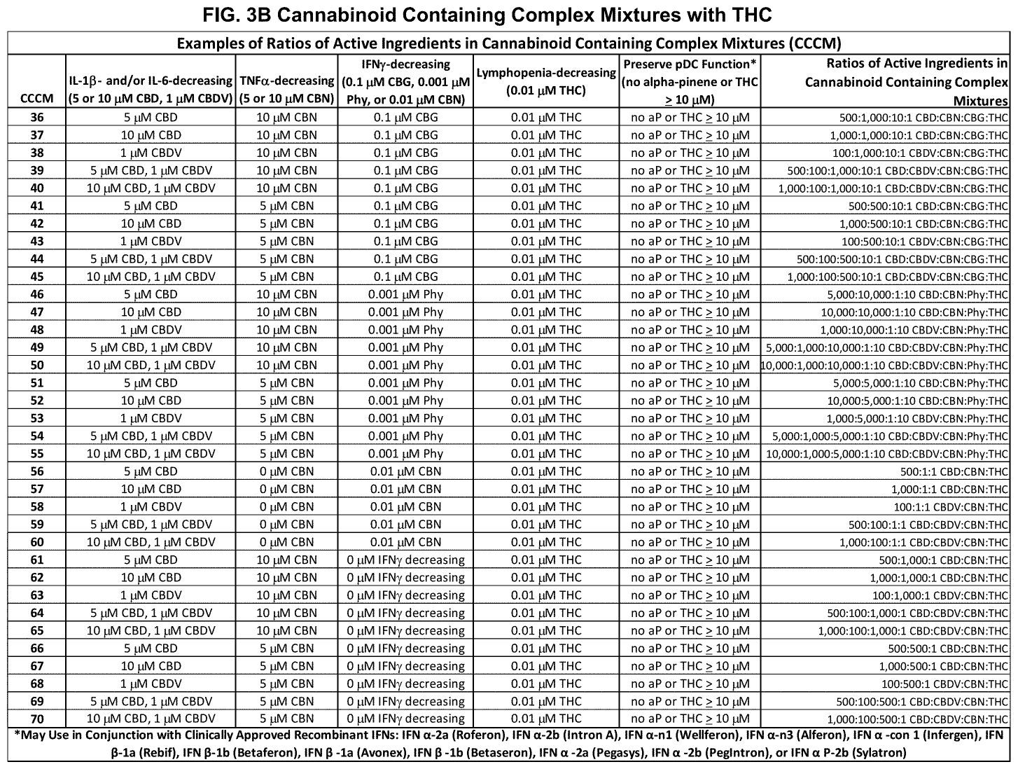

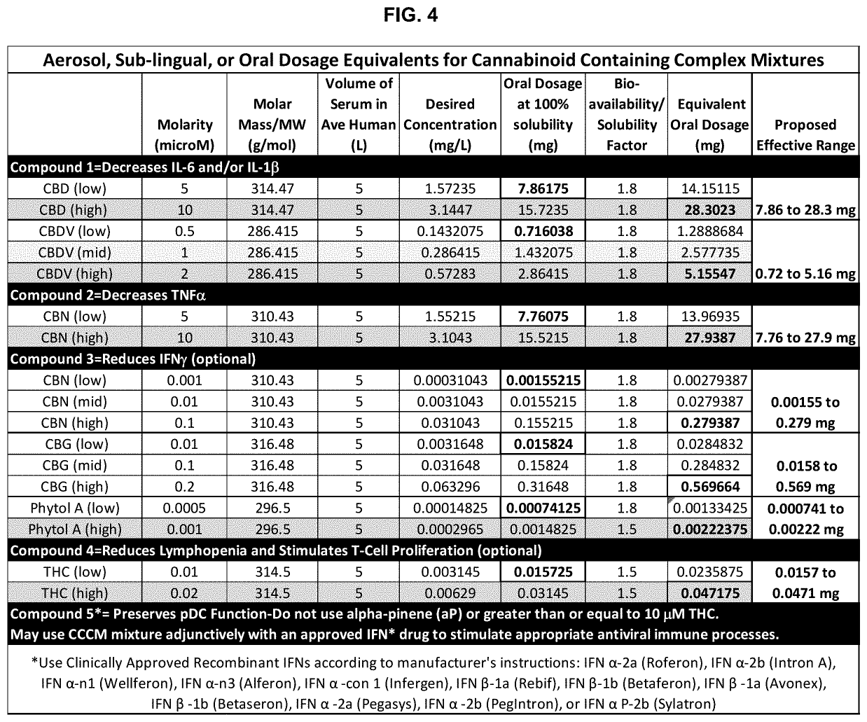

The following cannabinoid and/or terpene compound mixtures are generated by mixing each of the (i) IL-1β and/or IL-6-decreasing cannabinoid or terpene, (ii) TNFα-decreasing cannabinoid, (iii) optionally an IFNγ-decreasing cannabinoid or terpene, and (iv) optionally a helper CD4+ T cell or cytotoxic CD8+ Lymphopenia-reducing cannabinoid at the molar ratios provided in FIG. 3A and 3B and Table 1 below.

TABLE 1
Complex mixtures for treatment of CRS or MAS

| | IL-1β and/or IL-6- | TNFα- | IFNγ-decreasing | Lymphopenia- |
|---|---|---|---|---|
| | decreasing | decreasing | cannabinoid | reducing |
| | cannabinoid | cannabinoid | or terpene | cannabinoid |
| CCCM | (molar concentration) | (molar concentration) | (molar concentration) | (molar concentration) |
| CCCM 1 | CBN (5 μM, | CBN (5 μM, | | |
| | or 10 μM | or 10 μM) | | |
| CCCM 2 | CBD (5 μM, | CBN (5 μM, | | |
| | or 10 μM); , | or 10 μM) | | |
| | CBDV (0.5 μM | | | |
| | 1 μM, or 2 μM) | | | |
| CCCM 3 | CBD (5 μM, | CBN (5 μM, | | |
| | or 10 μM); , | or 10 μM) | | |
| | CBDV (0.5 μM | | | |
| | 1 μM, or 2 μM) | | | |
| CCCM 4 | CBD (5 μM, | CBN (5 μM, | CBG (0.01 μM, | |
| | or 10 μM) | or 10 μM) | 0.1 μM, or 0.2 μM) | |
| CCCM 5 | CBDV (0.5 μM, | CBN (5 μM, | CBG (0.01 μM, | |
| | 1 μM, or 2 μM) | or 10 μM) | 0.1 μM, or 0.2 μM) | |
| CCCM 6 | CBD (5 μM, | CBN (5 μM, | CBG (0.01 μM, | |
| | or 10 μM); , | or 10 μM) | 0.1 μM, or 0.2 μM) | |
| | CBDV (0.5 μM | | | |
| | 1 μM, or 2 μM) | | | |
| CCCM 7 | CBD (5 μM, | CBN (5 μM, | Phytol A (0.0005 μM, | |
| | or 10 μM) | or 10 μM) | or 0.001 μM) | |
| CCCM 8 | CBDV (0.5 μM, | CBN (5 μM, | Phytol A (0.0005 μM, | |
| | 1 μM, or 2 μM) | or 10 μM) | or 0.001 μM) | |
| CCCM 9 | CBD (5 μM, | CBN (5 μM, | Phytol A (0.0005 μM, | |
| | or 10 μM); , | or 10 μM) | or 0.001 μM) | |
| | CBDV (0.5 μM | | | |
| | 1 μM, or 2 μM) | | | |
| CCCM 10 | CBD (5 μM, | | CBN (0.01 μM) | |
| | or 10 μM) | | | |
| CCCM 11 | CBDV (0.5 μM, | | CBN (0.01 μM) | |
| | 1 μM, or 2 μM) | | | |
| CCCM 12 | CBD (5 μM, | | CBN (0.01 μM) | |
| | or 10 μM); , | | | |
| | CBDV (0.5 μM | | | |
| | 1 μM, or 2 μM) | | | |
| CCCM 13 | CBD (5 μM, | CBN (5 μM, | CBG (0.01 μM, | THC (0.01 μM, |
| | or 10 μM) | or 10 μM) | 0.1 μM, or 0.2 μM) | or 0.02 μM) |
| CCCM 14 | CBDV (0.5 μM, | CBN (5 μM, | CBG (0.01 μM, | THC (0.01 μM, |
| | 1 μM, or 2 μM) | or 10 μM) | 0.1 μM, or 0.2 μM) | or 0.02 μM) |
| CCCM 15 | CBD (5 μM, | CBN (5 μM, | CBG (0.01 μM, | THC (0.01 μM, |
| | or 10 μM); , | or 10 μM) | 0.1 μM, or 0.2 μM) | or 0.02 μM) |
| | CBDV (0.5 μM | | | |
| | 1 μM, or 2 μM) | | | |
| CCCM 16 | CBD (5 μM, | CBN (5 μM, | Phytol A (0.0005 μM, | THC (0.01 μM, |
| | or 10 μM) | or 10 μM) | or 0.001 μM) | or 0.02 μM) |
| CCCM 17 | CBDV (0.5 μM, | CBN (5 μM, | Phytol A (0.0005 μM, | THC (0.01 μM, |
| | 1 μM, or 2 μM) | or 10 μM) | or 0.001 μM) | or 0.02 μM) |
| CCCM 18 | CBD (5 μM, | CBN (5 μM, | Phytol A (0.0005 μM, | THC (0.01 μM, |
| | or 10 μM); , | or 10 μM) | or 0.001 μM) | or 0.02 μM) |
| | CBDV (0.5 μM | | | |
| | 1 μM, or 2 μM) | | | |
| CCCM 19 | CBD (5 μM, | | CBN (0.01 μM) | THC (0.01 μM, |
| | or 10 μM) | | | or 0.02 μM) |
| CCCM 20 | CBDV (0.5 μM, | | CBN (0.01 μM) | THC (0.01 μM, |
| | 1 μM, or 2 μM) | | | or 0.02 μM) |
| CCCM 21 | CBD (5 μM, | | CBN (0.01 μM) | THC (0.01 μM, |
| | or 10 μM); , | | | or 0.02 μM) |
| | CBDV (0.5 μM | | | |
| | 1 μM, or 2 μM) | | | |

PBMC are treated with one of the mixtures provided in Table 1. They are tested in three treatment groups for each compound mixture, assessing IL-1β and/or IL-6 activity, TNFa activity, IFNγ-activity, or T-cell activity. To test effects of IL-1β, IL-6 and TNFα activity from PBMC, cells are incubated with one of the mixtures for 6 hours, and harvested using ice cold FACS buffer. To test effects of IL-1β, IL-6 and TNFα activity from PBMC, and activity of T cells, cells are incubated with one of the mixtures for 18 hours, and harvested using CellStripper (Corning, Corning, N.Y.) for 15-30 minutes at 37° C. An aliquot of 200 μl of cell suspension is used for flow cytometry, while the remainder is used to determine the cell concentration using a Coulter Particle-Counter (Beckman-Coulter Inc., Brea, Calif.).

### Results

Some of the cannabinoid containing complex mixtures 1-20, tested at various effective dosages as shown in Table 1, have a decreasing effect on secretion of the pro-inflammatory cytokines, TNFα, IL-1β and/or IL-6 in monocytes, TNFα in pDC, and IFNγ in T cells; as well as pro-activating and proliferating effects on helper CD4+ T cell and cytotoxic CD8+ T cells. The results also show that exogenous IFNα production and/or supplementation with recombinant interferons such as IFNα preserved the anti-viral response function of the pDCs.

Specifically, the results show that the cannabinoid containing complex mixtures have the following effects:

1) IL-1β and/or IL-6-decreasing Effects:

reduce the level of IL-6 secreted by activated CD14⁺ CD16⁺ monocytes,

reduce phagocytosis by CD14⁺CD16⁺ monocytes,

reduce the level of IL-1β secreted by activated CD14⁺ CD16⁺ monocytes,

reduce the level of HLA-DR, CD80, and CD86 co-stimulatory molecules expressed by CD14⁺CD16⁺ monocytes,

suppress the expression of the activation marker CD25 by cytotoxic CD8⁺ T cells, and

suppress the expression of the activation marker CD69 by cytotoxic CD8⁺ T cells.

2) TNFα-decreasing Effects:

reduce the level of TNFα secreted by CD14⁺CD16⁺ monocytes.

reduce the level of TNFα secreted by pDC.

3) IFNγ-decreasing Effects:

reduce the level of IFNγ secreted by T cells.

4) Lymphopenia-reducing Effects:

increase proliferation of helper CD4⁺ T cells increase proliferation of cytotoxic CD8⁺ T cells,

Immunomodulatory effects of some complex mixtures suggest that they would be effective in treating CRS or MAS.

The cannabinoid containing complex mixtures have immunomodulatory effects that include IL-1β and/or IL-6-reducing effects, TNFα-reducing effects, IFNγ-reducing effects, and lymphopenia-reducing effects—and are effective in treating inflammatory disorders, such as CRS and MAS.

### 5.9.3. Example 3

### Immunomodulatory Effects of Cannabinoid Containing Complex Mixtures (CCCM) Tested on Employing Human Primary Leukocytes to Evaluate the Immunomodulatory Activity of Complex Mixtures

The immunomodulatory effectiveness of 24 cannabinoid containing complex mixtures (CCCM) on suppressing hyperinflammation are described in FIGS. 24A-24E. The immunodulatory effectiveness of these 24 CCCM were evaluated in an appropriate human model utilizing the co-culture of native, human peripheral blood mononuclear cell (PBMC) immune cells. The list of the amounts and ratios of these components within these representative (24) cannabinoid mixtures are presented within the tables of FIG. 3A-3B. Cytokine and inflammatory markers were measured in pDCs, monocytes, and CD4+ and CD8+ T cells, and the statistically significant immunomodulatory activities are shown in FIGS. 24A-24E.

PBMCs were isolated from 6 healthy human donors. A blood sample was withdrawn from each donor in the morning. The blood sample was then processed to isolate a buffy coat within the blood sample containing PBMCs. Following isolation, PBMCs within the buffy coat were cultured in a standard culturing system. All experiments were performed on the cultured PBMCs.

### Materials and Methods

### Peripheral Blood Mononuclear Cell Isolation

Blood from 6 healthy human donors was diluted 1:1 with Hanks Balanced Salt Solution from Gibco™ (Grand Island, N.Y.). Diluted blood was layered onto 15 mL Ficoll Paque Plus (GE Healthcare Life Sciences, Pittsburgh, Pa.) using SepMate 50 mL conical tubes by StemCell Technologies (Vancouver, BC, Canada) and centrifuged at 1300×g for 25 min at 4° C. The buffy coat was carefully removed from the plasma and re-suspended in HBSS and washed twice. Subsequent PBMC were resuspended in complete Roswell Park Memorial Institute (C-RPMI) Media from Gibco™ containing 5% Human AB Serum (Sigma-Aldrich, St. Louis, Mo.), 1% Penicillin-Streptomycin (Gibco™), and 0.035% β-mercaptoethanol. PBMC were cultured in 48 or 96 well plates at a density of 5×106 cells/mL in 600 or 200 μl of complete RPMI media respectively.

Cultured PBMCs were separated into 4 different groups:
Untreated (Unt): cultured PBMCs (Untreated);
Inflammatory Stimulus (Stim): cultured PBMCs treated with an inflammatory stimulus containing viral-CpG; bacterial-LPS; or anti-CD3, anti-CD28 antibodies followed by PMA/ionomycin. Treatment with an inflammatory stimulus was performed to mimic hyperinflammation in the sample;
Positive Control (Pos. Cont.): cultured PBMCs treated with the inflammatory stimulus containing viral-CpG; bacterial-LPS; or anti-CD3, anti-CD28 antibodies followed by PMA/ionomycin, and a vehicle containing DMSO or methanol;
CCCM Treatment Group: each CCCM was assigned a “number” indicating the CCCM tested, e.g., “1” denotes CCCM 1 of Table 3A): Cultured PBMCs treated with a CCCM for 30 minutes, followed by an inflammatory stimulus containing viral-CpG; bacterial-LPS; or anti-CD3, anti-CD28 antibodies and restimulated with PMA/ionomycin. A total of 24 CCCMs were tested.

### Treatment Groups:

Cannabidivarin (CBDV), cannabidiol (CBD) cannabinol (CBN) and phytol were purchased from Sigma-Aldrich (St. Louis, Mo.). The cannabinoids CBDV, CBD, and CBN were supplied in methanol. The methanol was evaporated under nitrogen and was resuspended in dimethyl sulfoxide (DMSO). Δ9-tetrahydrocannabinol (THC) was supplied by the National Institute of Drug Abuse (NIDA) in ethanol. The ethanol was evaporated under nitrogen and was resuspended in DMSO. Phytol was directly dissolved in DMSO. Compounds/mixtures were diluted in complete RPMI to a final DMSO concentration in culture of 0.04%.

The 4 different groups (Unt., Stim., Pos. Control, and CCCM Treatment-number) were further categorized into 4 cell-specific groups:
pDCs;
Monocytes,
CD4⁺ T cells; and
CD8⁺ T cells.

For monocytes and pDCs, the goal of the study was to assess immunomodulation of blood-derived monocyte and plasmacytoid dendric cell function and cytokine production by cannabis mixtures. For CD4⁺ T cells, and CD8⁺ T cells, the goal of the study was to determine the immunomodulatory activity of cannabis mixtures on T cell cytokine production.

Cultured PBMC suspensions were pre-treated with CCCMs for 30 minutes before treatment with designated inflammatory stimuli. They were also measured over different time points in the development of an inflammatory response; namely, 6 hours, 24 hours, and 96 hours (4 days) after stimulation of a hyperinflammatory response. Details of the types of inflammatory markers tested for immunological effects for the 4-cell specific groups are shown below:
pDC and Monocytes: markers IFNα⁺ and TNFα⁺ in pDCs, and TNFα⁺ in monocytes, were measured 6 hours post-treatment with inflammatory stimulus CpG in groups where an inflammatory stimulus was used.
Monocytes: markers CD80⁺, CD86⁺, IL-10⁺, IL-6⁺, and TNFα⁺ in monocytes were measured 24 hours post-treatment with inflammatory stimulus bacterial-LPS in groups where an inflammatory stimulus was used.
CD4+ T cells: markers IFNγ, IL-2, TNFα in CD4⁺ T cells were measured 4 days after CD3/CD28 activation & restimulation with PMA/IO in groups where an inflammatory stimulus was used.
CD8+ T cells: markers IFNγ, IL-2, TNFα in CD8⁺ T cells were measured 4 days after CD3/CD28 activation & restimulation with PMA/I0 in groups where an inflammatory stimulus was used.

All cytokines were evaluated by intercellular staining as provided above. Extracellular staining was done for the specific markers on each of the cell types along with the costimulatory molecules CD80, CD86 and MHC II.

### Cell Culture and Stimulation

Cell viability Experiments

In viability experiments, PBMC with treatments were cultured with CCCMs as prepared above on flat bottom 48 or 96 well plates at 37° C. in 5% CO2 for 6, 24 hours, or 4 days.

In experiments examining pDC and in 1 experiment examining monocytes, following 6 hours post treatment with CCCM and inflammatory stimulus viral-CpG, cells were harvested using ice cold FACS buffer.

In experiments examining Monocytes, cells were harvested following 24 hours post treatment with a CCCM and an inflammatory stimlulus bacterial-LPS using Cell Stripper (Corning, Corning, N.Y.) for 15-30 minutes at 37° C.

In experiments examining T cells, cells were harvested following 4 days post incubation with a CCCM and an inflammatory stimlulus with anti-CD3 and anti-CD28 antibodies and restimulation with PMA/ionomycin.

An aliquot of 200 μl of cell suspension was used for flow cytometry, while the remainder was used to determine the cell concentration using a Coulter Particle-Counter (Beckman-Coulter Inc., Brea, Calif.).

### Immunological Activity Experiments

In immunological activity experiments, PBMC suspensions were pre-treated with CCCMs for 30 minutes before treatment with designated inflammatory stimuli, cultured in 96 well tissue culture plates, and incubated at 37° C. and 5.0% CO₂.

pDCs

In pDC experiments, PBMC suspensions were pre-treated with a cannabinoid-based therapeutic mixture (CCCM) for 30 minutes before treatment with 15 μ/mL CpG-ODN Type A 2216 (InvivoGen©, San Diego, Calif.), the designated viral stimuli, then, cultured in 96 well tissue culture plates and incubated at 37° C. and 5.0% CO2. These cell suspensions were also treated with 3.0 μg/mL Golgi transport inhibitors Monensin (2 μM)/Brefeldin A (Thermo Fisher Scientific, Waltham, Mass.) 3 hours post-stimulation, and after 6 hours of stimulation, cells were harvested for evaluation by flow cytometry for IFNα or TNFα. Cells were fixed in BD CytoFix. Then these cells were permeabilized in BD Permwash and incubated with appropriate antibodies for intercellular staining of IFNα and TNFα. Using flow cytometry, these pDC were identified as CD123+ and CD303+ and assessed for intracellular IFNα and TNFα. Untreated (Unt.) samples were physically manipulated the same as the other experimental cell groups, but they were untreated by any of the compounds. Stimulated cells (Stim.) were not pre-incubated with any of the cannabinoid-based therapeutic mixtures, but they were stimulated and processed for IFNα and TNFα measurements the same as for the cannabinoid-based therapeutic mixture groups. The Positive Control (Pos. Cont.) group was pre-incubated with the vehicle control from the cannabinoid-based therapeutic mixtures, then it was stimulated and processed as for the cannabinoid-based therapeutic mixtures.

### Monocytes:

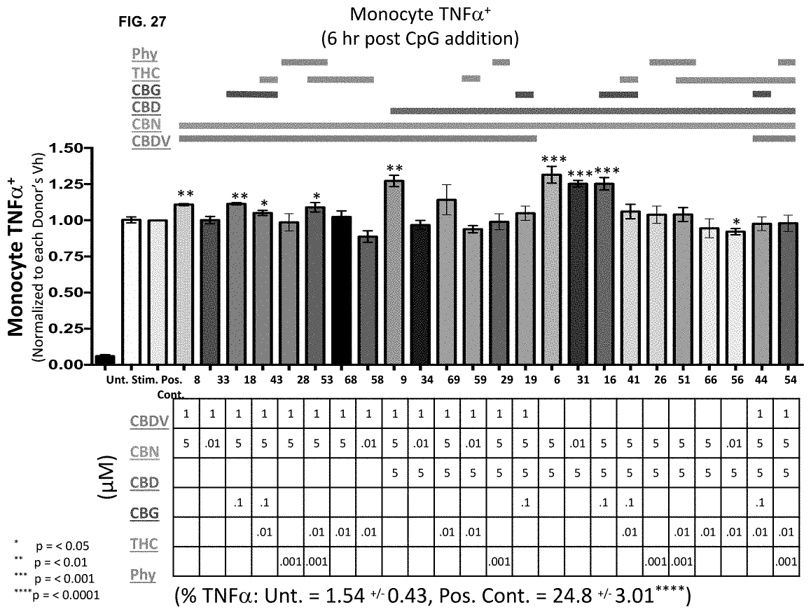

In 1 monocyte experiment (monocytes assessed for expression levels of TNFα 6 hours after CpG stimulation in FIG. 27), PBMC suspensions were pre-treated with our cannabinoid-based therapeutic mixtures for 30 minutes before treatment with 15 μg/mL CpG-ODN Type A 2216 (InvivoGen©, San Diego, Calif.), the designated viral stimuli, then, cultured in 96 well tissue culture plates and incubated at 37° C. and 5.0% CO2. These cell suspensions were also treated with 3.0 μg/mL Golgi transport inhibitors Monensin (2 μM)/Brefeldin A (Thermo Fisher Scientific, Waltham, Mass.) 3 hours post-stimulation, and after 6 hours of stimulation cells were harvested for evaluation by flow cytometry for TNFα. Harvested cells were fixed in BD CytoFix. Then these cells were permeabilized in BD Permwash and incubated with appropriate antibodies for intercellular staining of TNFα. Using flow cytometry, these monocytes were identified as CD14+ and assessed for intracellular TNFα. Untreated (Unt.) samples were physically manipulated the same as the other experimental cell groups, but they were untreated by any of the compounds. Stimulated cells (Stim.) were not pre-incubated with any of the cannabinoid-based therapeutic mixtures, but they were stimulated and processed for TNFα measurements the same as for the cannabinoid-based therapeutic mixture groups. The Positive Control (Pos. Cont.) group was pre-incubated with the vehicle control from the cannabinoid-based therapeutic mixtures, then it was stimulated and processed as for the cannabinoid-based therapeutic mixtures.

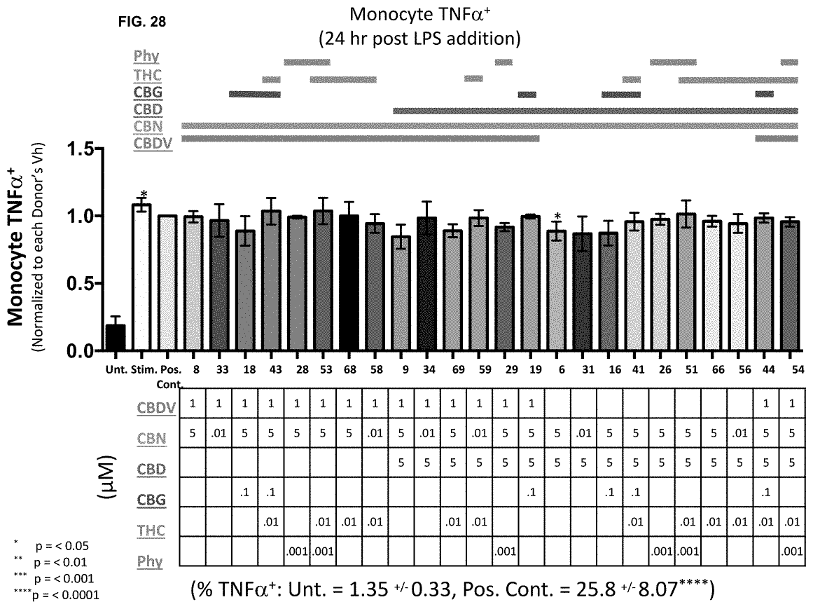

In all other monocyte experiments (FIGS. 28-32), PBMC suspensions were cultured in 96 well tissue culture plates and incubated at 37° C. and 5.0% CO2. The designated cells were pre-treated with our cannabinoid-based therapeutic mixtures for 30 minutes before stimulation with 1000 ng/mL LPS (S. typhosa, Sigma Aldrich, St. Louis, Mo.), the designated inflammatory stimuli. These cell suspensions were also treated with 3.0 μg/mL Golgi transport inhibitors Monensin (2 μM)/Brefeldin A (Thermo Fisher Scientific, Waltham, Mass.) 18 hours post-stimulation, and after 24 hours of stimulation, cells were harvested for evaluation by flow cytometry for TNFα, IL-6, IL-1β, CD80, and CD86. At 18 hours post stimulation, the Golgi transport inhibitor was added so the cells will accumulate most proteins that they are secreting including cytokines. This allowed for a more sensitive measurement of cytokine production during that particular time point. Then the cells are allowed to incubate for 6 hours prior to harvesting. The cells where in culture for a total of 24 hours. The LPS was in the culture for the whole 24 hours. Cells were fixed in BD CytoFix. Then these cells were permeabilized in BD Permwash and incubated with appropriate antibodies for intercellular staining of TNFα, IL-6, IL-1β, and extracellular CD80+ and CD86+. Using flow cytometry, these monocytes were identified as CD14+, and assessed for intracellular levels of TNFα, IL-6, IL-1β, and extracellular CD80+ and CD86+ expression. Untreated (Unt.) samples were physically manipulated the same as the other experimental cell groups, but they were untreated by any of the compounds. Stimulated cells (Stim.) were not pre-incubated with any of the cannabinoid-based therapeutic mixtures, but they were stimulated and processed for TNFα, IL-6, IL-1β, and extracellular CD80+ and CD86+ measurements the same as for the cannabinoid-based therapeutic mixture groups. The Positive Control (Pos. Cont.) group was pre-incubated with the vehicle control from the cannabinoid-based therapeutic mixtures, then it was stimulated and processed as for the cannabinoid-based therapeutic mixtures.

CD4+ and CD8+ T Cells:

In CD4+ and CD8+ T cell experiments, PBMC suspensions were pre-treated with our cannabinoid-based therapeutic mixtures for 30 minutes prior to stimulation. The designated stimulation of the T cell populations in the PBMC involved adding the PBMC suspension to 96 well tissue culture plates pre-coated with 5.0 μg/mL anti-CD3 (clone UCHT1, Biolegend, San Diego, Calif.), stimulated with 5.0 μg/mL anti-CD28 (clone 28.2, Biolegend), supplemented with 5.0 ng/mL IL-2 (Roche Applied Science, Indianapolis, Ind.), and incubated at 37° C. and 5.0% CO2. After 4 days of incubation with the stimulus (and cannabinoid-based therapeutic mixtures in the treatment groups), the PBMC were further stimulated with 50 ng/mL phorbol 12-myristate 13-acetate (PMA) and 1000 ng/mL ionomycin (Io), and then Golgi transport inhibitors were also added for 4 hours before the cells were harvested. Cells were fixed in BD CytoFix. Then these cells were permeabilized in BD Permwash and incubated with appropriate antibodies for intercellular staining of TNFα, IL-2, and IFNγ. Using flow cytometry, these T cells were identified as CD3+ and the T cell subset was identified by CD4+ or CD8+ expression, and then assessed for intracellular TNFα, IL-2, and IFNγ. Untreated (Unt.) samples were physically manipulated the same as the other experimental cell groups, but they were untreated by any of the compounds. Stimulated cells (Stim.) were not pre-incubated with any of the cannabinoid-based therapeutic mixtures, but they were stimulated and processed for TNFα, IL-2, and IFNγ measurements the same as for the cannabinoid-based therapeutic mixture groups. The Positive Control (Pos. Cont.) group was pre-incubated with the vehicle control from the cannabinoid-based therapeutic mixtures, then it was stimulated and processed as for the cannabinoid-based therapeutic mixtures.

### Flow Cytometry

pDC, monocyte and T cell were identified using antibodies directed against unique epitopes for each cell type and analyzed by flow cytometry. Cells were fixed in BD CytoFix and resuspended in FACS buffer for extracellular staining. To determine cell viability in mixed PBMC, exclusion of Near IR L/D stain (Life Technologies, Carlsbad, Calif.) was used. Fixed cells were permeabilized in BD Permwash and incubated with appropriate antibodies for intercellular staining. pDC were identified as CD123+ and CD303+ and assessed for intracellular IFNα and TNFα. Monocytes were identified as CD14+ and assessed for extracellular HLA-DR, CD80, CD86, and intracellular IL-1β, IL-6 and TNFα. T cells were identified as CD3+and T cell subsets identified by CD4+ and CD8+expression, and assessed for intracellular IFNγ, TNFα and IL-2.

For intracellular staining, fixed cells were permeabilized in BD Permwash and incubated with appropriate antibodies and 7% human AB serum. pDC were identified as CD303+ and assessed for intracellular IFNα and TNFα. Monocytes were identified as CD14+ and assessed for extracellular CD80, CD86, and intracellular TNFα, IL-6, and IL-1β.

### Results

### Cell Viability

Cell viability after treatment with each CCCM was assessed. No significant decrease in cell viability was observed with any of the mixtures at the concentrations that were evaluated.

### Immunological Activity

pDCs:

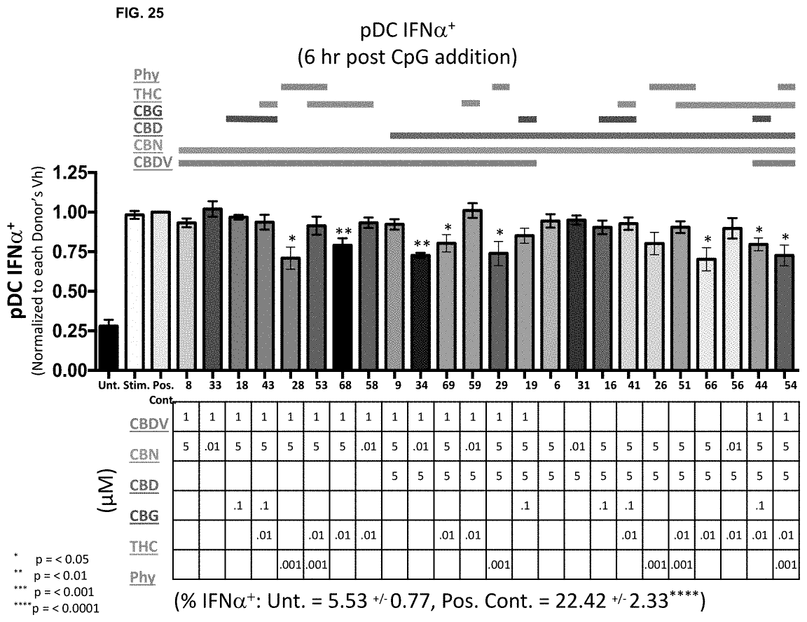

As shown in FIG. 25, in pDC cells 6 hours after treatment with inflammatory stimulus CpG, a statistically significant reduction in IFNα expression compared to the positive control (vehicle+inflammatory stimulus) was shown in:
CCCM 66 containing 5 μMCBN, 5 μM CBD, 0.01 THC;
CCCM 28 containing 1 μM CBDV, 5 μM CBN, 0.001 μM phytol;
CCCM 34 containing 1 μM CBDV, 0.01 μM CBN, and 5 μM CBD,
CCCM 54 containing 1 μM CBDV, 5 μM CBN 5 μM CBD, 0.01 μM THC, 0.001 μM Phytol.
CCCM 29 containing 1 μM CBDV, 0.01 μM CBN, 5 μM CBD, and 0.001 μM phytol;
CCCM 44 containing 1 μM CBDV, 5 μM CBN, 5 μM CBD, 0.1 μM CBD, and 0.01 μM THC; and
CCCM 69 containing 1 μM CBDV, 5 μM CBN, 5 μM CBD and 0.01 μM THC;

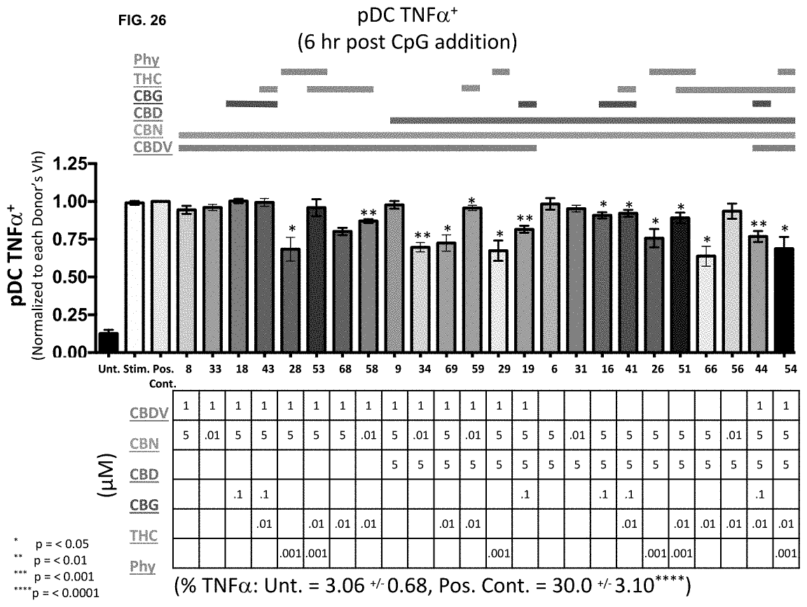

As shown in FIG. 26, in pDC cells 6 hours after treatment with inflammatory stimulus CpG, a stastistical significant reduction in TFNα expression, compared to the positive control (vehicle+inflammatory stimulus), was shown in:
CCCM 66 containing 5 μM CBN, 5 μM CBD, 0.01 THC;
CCCM 28 containing 1 μM CBDV, 5 μM CBN, 0.001 μM phytol;
CCCM 19 containing 1 μM CBDV, 5 μM CBN, 5 μM CBD, and 0.1 μM CBG;
CCCM 16 containing 5 mM CBD 5 μM CBN, 0.1 mM CBG;
CCCM 59 containing 1 mM CBDV, 5 μM CBD, 0.01 μM CBN, 0.01 μM THC;
CCCM 34 containing 1 μM CBDV, 0.01 μM CBN, and 5 μM CBD,
CCCM 54 containing 1 μM CBDV, 5 μM CBN 5 μM CBD, 0.01 μM THC, 0.001 μM Phytol;
CCCM 51 containing 5 μM CBD 5 μM CBN, 0.001 μM Phytol A, 0.01 μM THC
CCCM 29 containing 1 μM CBDV, 0.01 μM CBN, 5 μM CBD, and 0.001 μM phytol;
CCCM 44 containing 1 μM CBDV, 5 μM CBN, 5 μM CBD, 0.1 μM CBD, and 0.01 μM THC; and
CCCM 58 containing 1 μM CBDV, 0.01 μM CBN, 0.01 μM THC
CCCM 69 containing 1 μM CBDV, 5 μM CBN, 5 μM CBD and 0.01 μM THC;
CCCM 26 containing 5 μM CBD 5 μM CBN, 0.001 μM Phytol;
CCCM 41 containing 5 μM CBD 5 μM CBN, 0.1 μM CBG, 0.01 μM THC;
CCCM 68 containing 1 μM CBDV 5 μM CBN, 0.01 μM THC

Monocytes:

As shown in FIG. 27, in Monocyte cells 6 hours after treatment with inflammatory stimulus CpG, statistical significance was shown in the modulation of TFNα expression, compared to the positive control (vehicle+inflammatory stimulus), in:
CCCM 8 containing 1 μM CBDV, 5 μM CBN;
CCCM 18 containing 1 μM CBDV, 5 μM CBN, and 0.1 μM CBG;
CCCM 43 containing 1 μM CBDV, 5 μM CBN, 0.1 μM CBG, and 0.01 μM THC;
CCCM 53 containing 1 μM CBDV, 5 μM CBN, and 0.01 μM THC;
CCCM 9 containing 1 μM CBDV, 5 μM CBN, and 5 μM CBD;
CCCM 6 containing 5 μM CBN, 5 μM CBD;
CCCM 31 containing 0.01 μM CBN, 5 μM CBD;
CCCM 16 containing 5 μM CBN, 5 μM CBD, 0.1 μM CBG;
CCCM 56 containing 0.01 μM CBN, 5 μM CBD, 0.01 μM THC.

However, only a statistically significant reduction in TFNα expression was shown in CCCM 56 containing 5 μM CBD, 0.01 μM CBN, and 0.01 mM THC.

As shown in FIG. 28, in Monocyte cells 24 hours after treatment with inflammatory stimulus LPS, a statistically significant reduction in TFNα expression compared to the positive control (vehicle+inflammatory stimulus), is shown in CCCM 6 containing 5 μM CBD and 5 μM CBN.

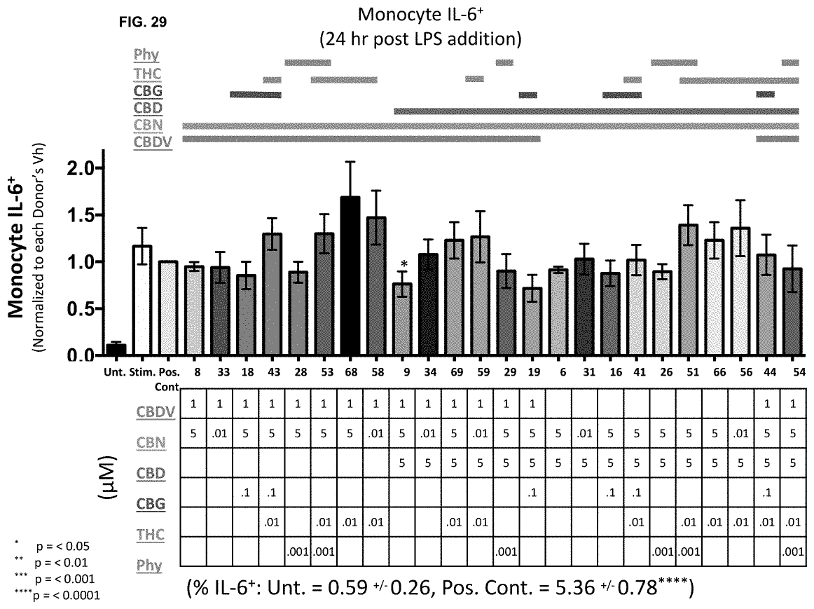

As shown in FIG. 29, in Monocyte cells 24 hours after treatment with inflammatory stimulus LPS, a statistically significant reduction in IL-6 expression compared to the positive control (vehicle+inflammatory stimulus), is shown in CCCM 9 containing 1 μM CBDV, 5 μM CBD, 5 μM CBN.

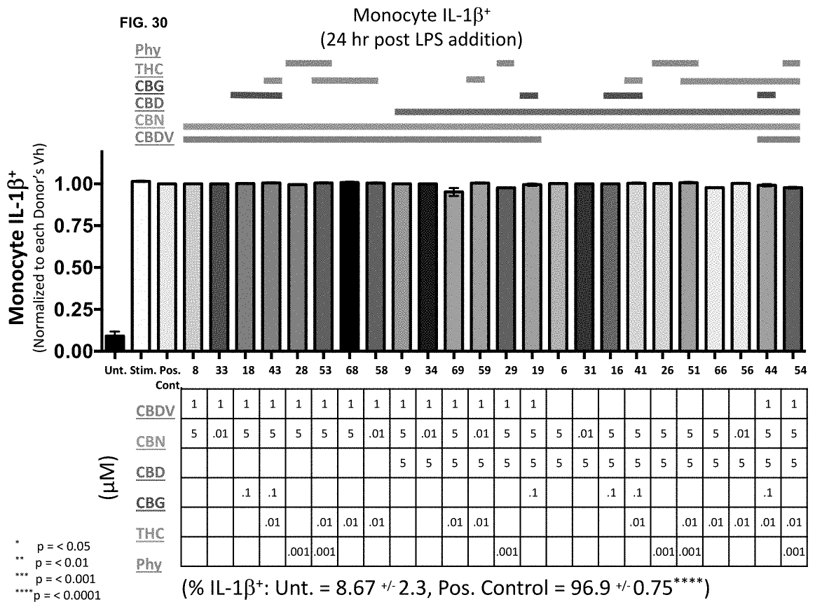

As shown in FIG. 30, in Monocyte cells 24 hours after treatment with inflammatory stimulus LPS, there was no statistical significance in the modulation in IL-1β+ expression compared to the positive control (vehicle+inflammatory stimulus).

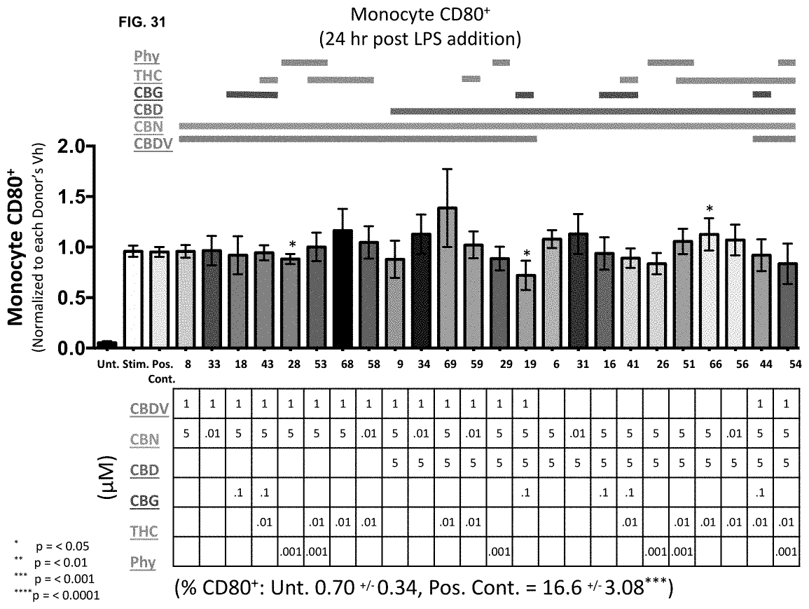

As shown in FIG. 31, in Monocyte cells 24 hours after treatment with inflammatory stimulus LPS, a statistically significance was shown in the modulation of costimulatory molecule CD80+secretion compared to the positive control (vehicle+inflammatory stimulus), for the following mixtures:
CCCM 19 containing 1 μM CBDV, 5 μM CBD, 5 μM CBN, and 0.1 μM CBG;
CCCM 28 containing 1 μM CBDV, 5 μM CBN, 0.001 μM Phytol A;
CCCM 66 containing 5 μM CBD, 5 μM CBN, and 0.01 μM THC.

However, only a CCCM 19 and CCCM 38 showed a statistical significant reduction in CD80+secretion compared to the positive control (vehicle+inflammatory stimulus).

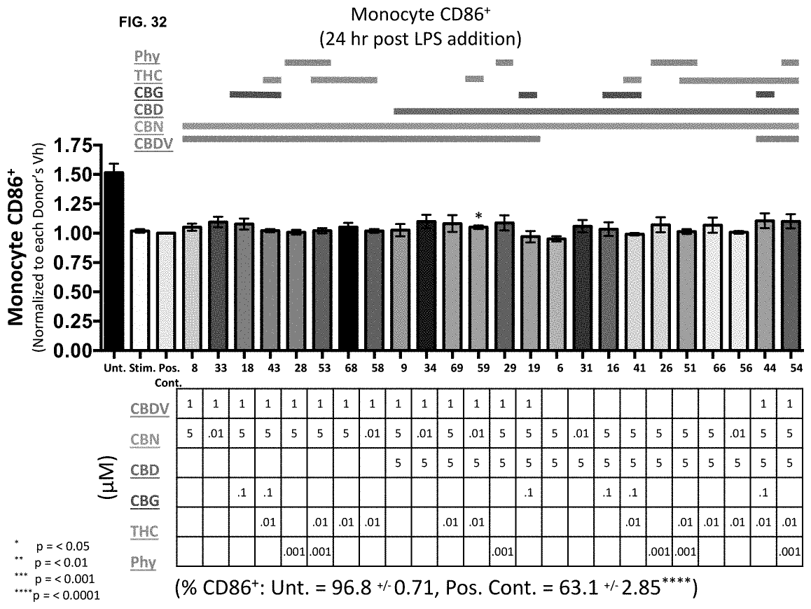

As shown in FIG. 32, in Monocyte cells 24 hours after treatment with inflammatory stimulus LPS, a statistically significance was shown in the modulation of costimulatory molecule CD86+secretion compared to the positive control (vehicle+inflammatory stimulus), in CCCM 59 containing 1 μM CBDV, 5 μM CBD, 0.01 μM CBN, and 0.01 μM THC.

CD4+ T Cells

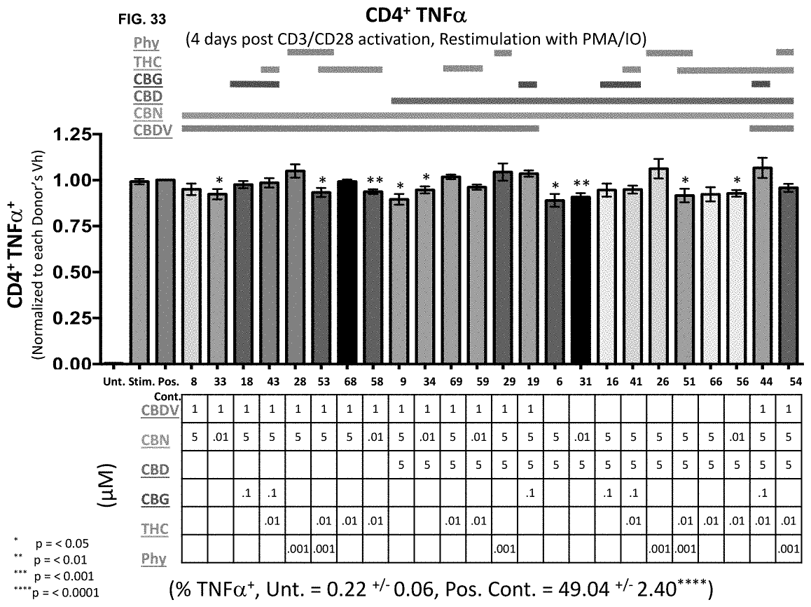

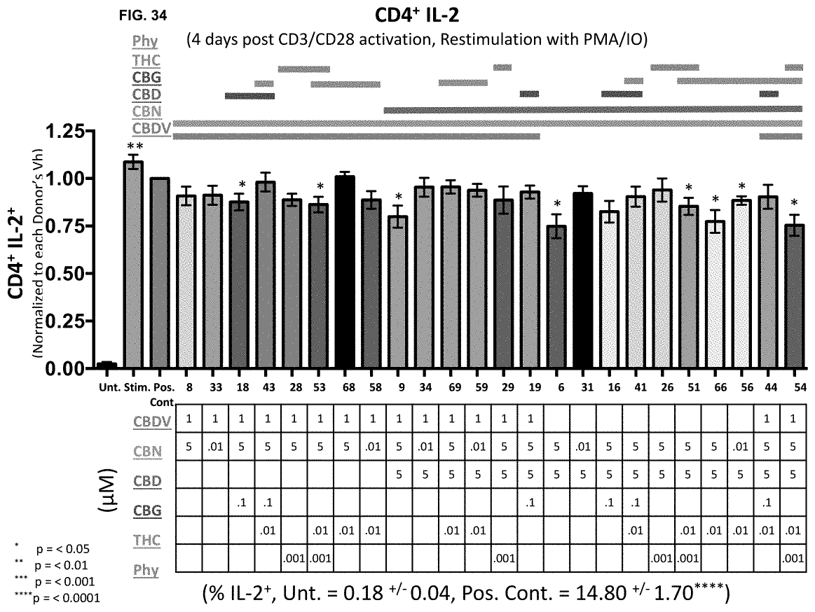

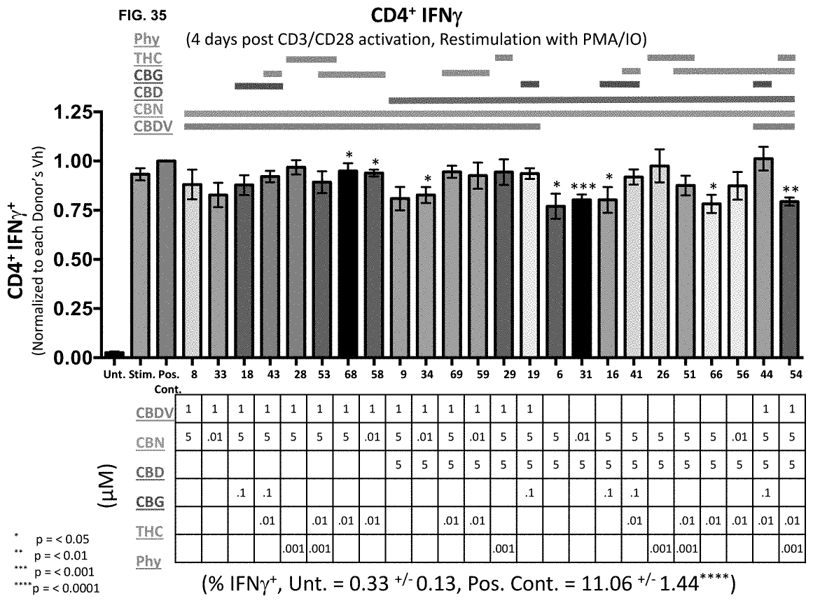

As shown in FIG. 33, in CD4+ T cells 4 days after treatment with inflammatory stimulus, a statistically significant reduction in TFNα expression compared to the positive control (vehicle+inflammatory stimulus), is shown in:
CCCM 6 containing 5 μM CBD, 5 μM CBN;
CCCM 9 containing 1 μM CBDV, 5 μM CBD, 5 μM CBN;
CCCM 33 containing 1 μM CBDV, 0.01 μM CBN;
CCCM 31 containing 5 μM CBD, 0.01 μM CBN;
CCCM 34 containing 1 μM CBDV, 5 μM CBD, 0.01 μM CBN;
CCCM 53 containing 1 μM CBDV, 5 μM CBN, 0.001 μM Phytol A, 0.01 μM THC;
CCCM 51 containing 5 μM CBD, 5, μM CBN, 0.001 μM Phytol A, 0.01 μM THC;
CCCM 58 containing 1 μM CBDV, 0.01 μM CBN, 0.01 μM THC;
CCCM 56 containing 5 μM CBD, 0.01 μM CBN, 0.01 μM THC;

As shown in FIG. 33, in CD4+ T cells cells 4 days after treatment with inflammatory stimulus, a statistically significant reduction was shown in IL-2 expression compared to the positive control (vehicle+inflammatory stimulus), is shown in:
CCCM 6 containing 5 μM CBD, 5 μM CBN;
CCCM 9 containing 1 μM CBDV, 5 μM CBD, 5 μM CBN;
CCCM 18 containing 1 μM CBDV, 5 μM CBN, 0.1 μM CBG;
CCCM 53 containing 1 μM CBDV, 5 μM CBN, 0.001 μM Phytol A, 0.01 μM THC
CCCM 51 containing 5 μM CBD, 5 μM CBN, 0.001 μM Phytol A, 0.01 μM THC
CCCM 54 containing 1 μM CBDV, 5 μM CBD, 5 μM CBN, 0.001 μM Phy 0.01 μM THC
CCCM 56 containing 5 μM CBD, 0.01 μM CBN, 0.01 μM THC
CCCM 66 containing 5 μM CBD, 5 μM CBN, 0.01 μM THC

As shown in FIG. 34, in CD4+ T cells 4 days after treatment with inflammatory stimulus, a statistically significant reduction in IFNγ expression expression compared to the positive control (vehicle+inflammatory stimulus), is shown in:
CCCM 6 containing 5 μM CBD, 5 μM CBN;
CCCM 16 containing 5 μM CBD, 5 μM CBN, 0.1 μM CBG;
CCCM 31 containing 5 μM CBD, 0.01 μM CBN;
CCCM 34 containing 1 μM CBDV, 5 μM CBD, 0.01 μM CBN;
CCCM 54 containing 1 μM CBDV, 5 μM CBD, 5 μM CBN, 0.001 μM Phytol A, 0.01 mM THC;
CCCM 58 containing 1 μM CBDV, 0.01 μM M CBN, 0.01 μM THC;
CCCM 66 containing 5 μM CBD, 5 μM CBN, 0.01 μM THC

CD8+ T Cells

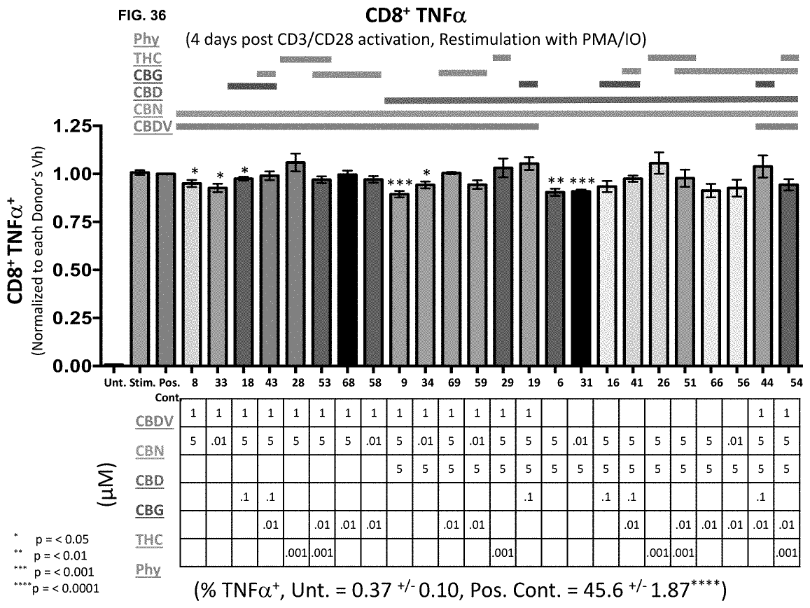

As shown in FIG. 36, in CD8+ T cells 4 days after treatment with inflammatory stimulus, a statistically significant reduction in TFNα expression expression compared to the positive control (vehicle+inflammatory stimulus), is shown in:
CCCM 8 containing 1 μM CBDV, 5 μM CBN;
CCCM 6 containing 5 μM CBD, 5 μM CBN;
CCCM 9 containing 1 μM CBDV, 5 μM CBD, 5 μM CBN
CCCM 18 containing 1 μM CBDV, 5 μM CBN, 0.1 μM CBG
CCCM 33 containing 1 μM CBDV 0 μM CBN, 0.01 μM CBN
CCCM 31 containing 5 μM CBD, 0.01 μM CBN
CCCM 34 containing 1 μM CBDV, 5 μM CBD, 0.01 μM CBN;

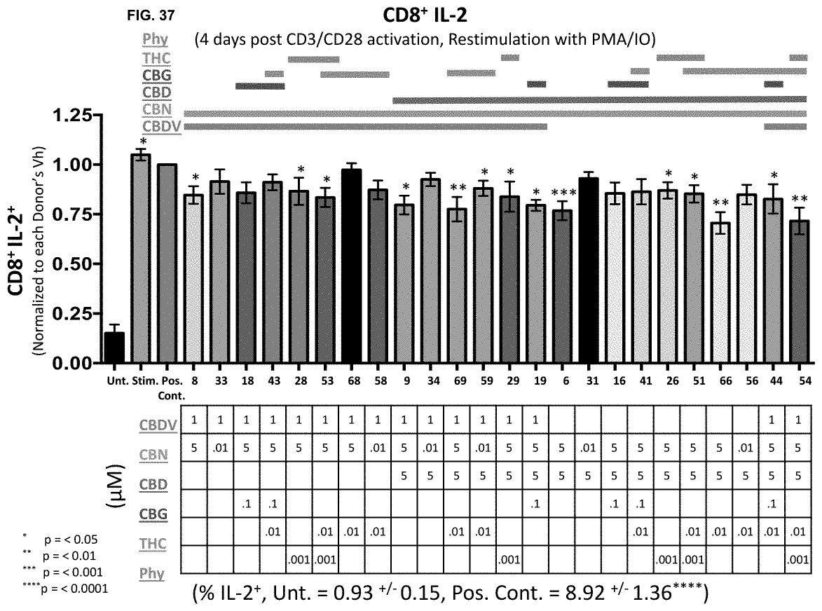

As shown in FIG. 37, in CD8+ T cells 4 days after treatment with inflammatory stimulus, a statistically significant reduction in IL-2 expression compared to the positive control (vehicle+inflammatory stimulus), is shown in:
CCCM 8 containing 1 μM CBDV, 5 μM CBN
CCCM 6 containing 5 μM CBD, 5 μM CBN,
CCCM 9 containing 1 μM CBDV, 5 μM CBD, 5 μM CBN
CCCM 19 containing 1 μM CBDV, 5 μM CBD, 5 μM CBN, 0.1 μM CBG
CCCM 28 containing 1 μM CBDV, 5 μM CBN, 0.001 μM Phy
CCCM 26 containing 5 μM CBD, 5 μM CBN, 0.001 μM Phy
CCCM 29 containing 1 μM CBDV, 5 mM CBD, 5 μM CBN, 0.001 μM Phy
CCCM 44 containing 1 μM CBDV, 5 μM CBD, 5 μM CBN, 0.1 μM CBG, 0.01 μM THC
CCCM 53 containing 1 μM CBDV, 5 μM CBN, 0.001 μM Phytol A, 0.01 μM THC
CCCM 51 containing 5 μM CBD, 5 μM CBN, 0.001 μM Phytol A, 0.01 μM THC
CCCM 54 containing 1 μM CBDV, 5 μM CBD, 5 μM CBN, 0.001 μM Phytol A, 0.01 μM THC
CCCM 59 containing 1 μM CBDV, 5 μM CBD, 0.01 μM CBN 0.01 μM THC
CCCM 66 containing 5 μM CBD, 5 μM CBN, 0.01 μM THC
CCCM 69 containing 1 μM CBDV, 5 μM CBD, 5 μM CBN, 0.01 μM THC

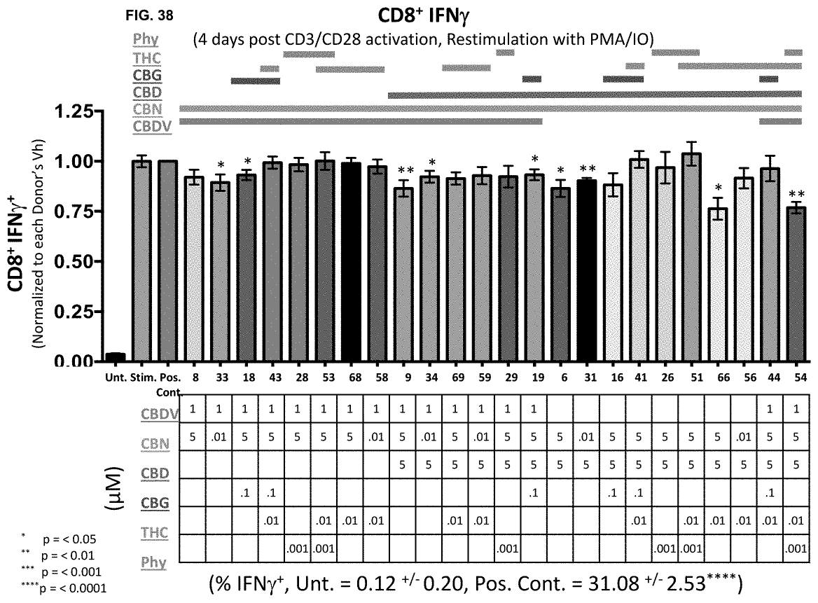

As shown in FIG. 38, in CD8+ T cells 4 days after treatment with inflammatory stimulus, a statistically significant reduction in IFNγ expression compared to the positive control (vehicle+inflammatory stimulus), is shown in:
CCCM 6 containing 5 μM CBD, 5 μM CBN;
CCCM 9 containing 1 μM CBDV, 5 mM CBD, 5 μM CBN
CCCM 18 containing 1 μM CBDV, 5 μM CBN, 0.1 μM CBG
CCCM 19 containing 1 μM CBDV, 5 μM CBD, 5 μM CBN, 0.1 μM CBG
CCCM 33 containing 1 μM CBDV, 0.01 μM CBN
CCCM 31 containing 5 μM CBD, 0.01 μM CBN
CCCM 34 containing 1 μM CBDV, 5 μM CBD, 0.01 μM CBN
CCCM 54 containing 1 μM CBDV, 5 μM CBD, 5 μM CBN, 0.001 μM Phytol A, 0.01 mM THC
CCCM 66 containing 5 μM CBD, 5 μM CBN, 0.01 μM THC

Further, the complexity of the cannabinoid and terpene mixtures was evaluated. The present inventors surprisingly found that, for certain mixtures, there was sensitivity to changes in components and the relative amounts of components in the Cannabinoid Containing Complex Mixtures relative to their effectiveness as immunomodulating agents.

FIG. 39 shows boxes around particular mixtures pointing to the sensitivity of changes to the components or relative amounts of particular components. Using the graph of the effects of the cannabinoid containing complex mixtures on levels of interferon alpha (IFNα) in the plasmacytoid dendritic cells (pDC) as an example, note that CCCM mixture 8 containing 1 μM of CBDV and 5 μM of CBN is trending toward a reduction in IFNα, but is not significantly changed relative to the positive control. As seen in the results for CCCM mixture 28, adding just 0.001 μM of the terpene Phytol to the 1 μM of CBDV and 5 μM of CBN (like in CCCM mixture 8), causes a statistically significant reduction in IFNα. As seen in CCCM mixture 68, adding 0.01 μM of THC to the 1 μM of CBDV and 5 μM of CBN (CCCM mixture 8) results in a IFNα level that is trending down, but it is not a statistically significant reduction in IFNα. As seen in CCCM mixture 58, changing the level of CBN from 5 μM to 0.01 μM CBN dramatically reduces the anti-inflammatory potential of the mixture.

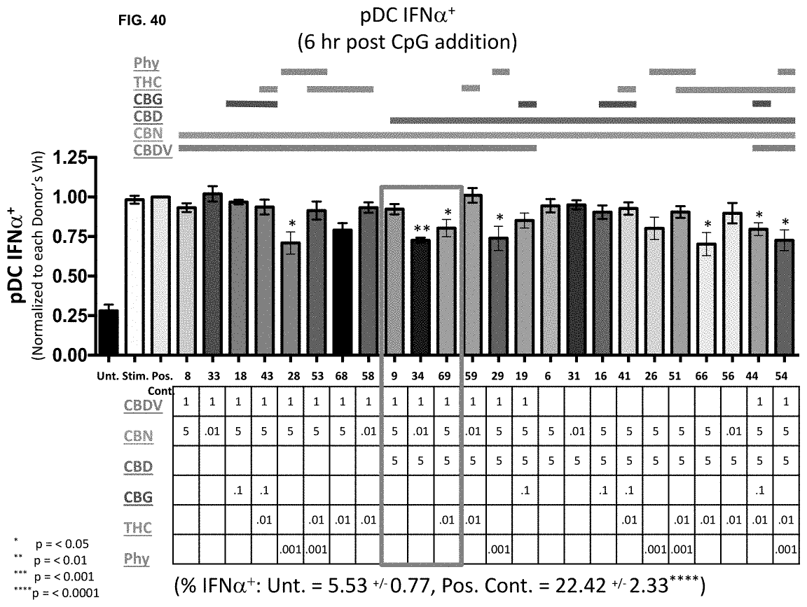

FIG. 40 shows boxes around particular mixtures pointing to the sensitivity of changes to the components or relative amounts of particular components. Again, using the graph of the effects of the cannabinoid containing complex mixtures on levels of interferon alpha (IFNa) in the plasmacytoid dendritic cells (pDC) as an example, note that CCCM mixture 9 containing 1 mM of CBDV and 5 mM of CBN and 5 mM CBD has an IFNa level that is not significantly changed relative to the positive control. However, if the 5 mM CBN in CCCM mixture is changed to 0.01 mM CBN as seen in CCCM mixture 34 that contains 1 mM of CBDV and 0.01 mM of CBN and 5 mM CBD, there is a statistically significant reduction in IFNa relative to the positive control for CCCM mixture 34. This demonstrated how important the ratios of the active pharmaceutical ingredients are in maintaining the efficacy of the active ingredients. As seen in CCCM mixture 69, adding 0.01 mM of THC to the 1 mM of CBDV and 5 mM of CBN and 5 mM CBD (CCCM mixture 9) results in an IFNa level that is less anti-inflammatory, but CCCM mixture 69 still causes a statistically significant reduction in IFNa. The specific ingredients and the number of ingredients are also relevant to the effectiveness of the CCCM mixture.

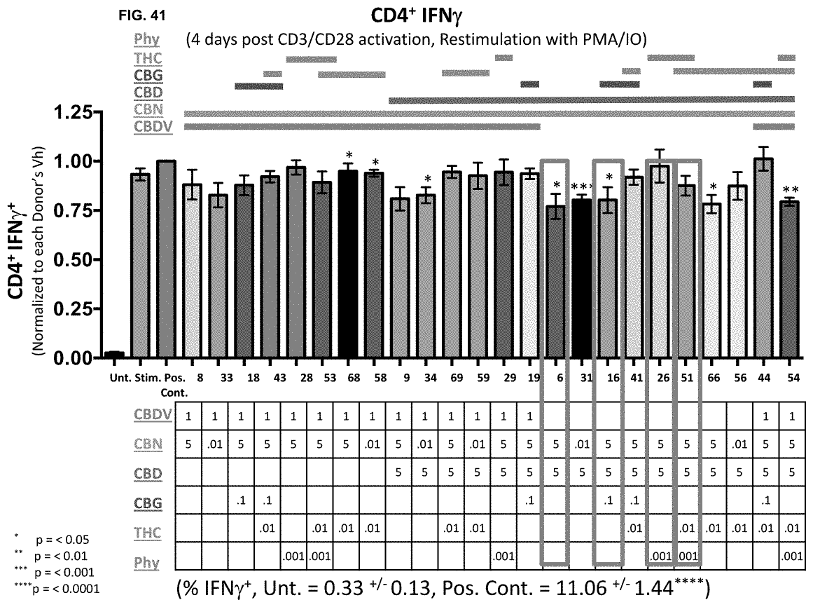

FIG. 41 shows boxes around particular mixtures pointing to the sensitivity of changes to the components or relative amounts of particular components. Using the graph of the effects of the cannabinoid containing complex mixtures on levels of interferon gamma (IFNg) in CD4+ T cells as an example, note that CCCM mixture 6 containing 5 mM of CBN and 5 mM CBD produced a statistically significant reduction in IFNg in the CD4+ T cells. If 0.1 mM of CBG is added to the mixture of 5 mM of CBN and 5 mM CBD, as in CCCM mixture 16, CCCM mixture 16 still produced a statistically significant reduction in IFNg in the CD4+ T cells. However in CCCM mixture 26, 0.001 mM Phytol is added to the 5 mM of CBN and 5 mM CBD (as in CCCM mixture 6), CCCM mixture 26 does not produced a statistically significant reduction in IFNg in the CD4+ T cells. When 0.01 mM THC is added to the same components that were in CCCM mixture 26 (that did not cause a statistically significant reduction), the resultant CCCM mixture 51, which contains 5 mM of CBN, 5 mM CBD, 0.01 mM THC, 0.001 mM Phytol A, produced a statistically significant reduction in IFNg in the CD4+ T cells.

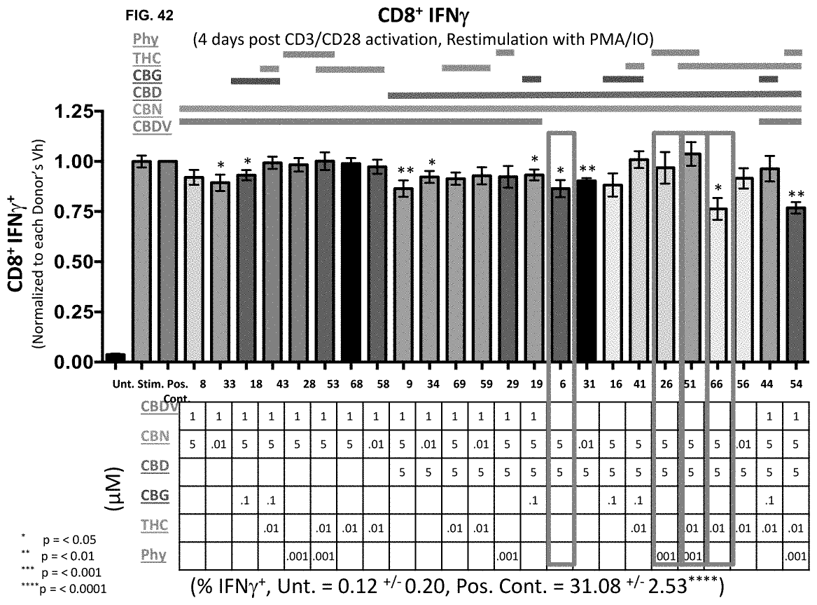

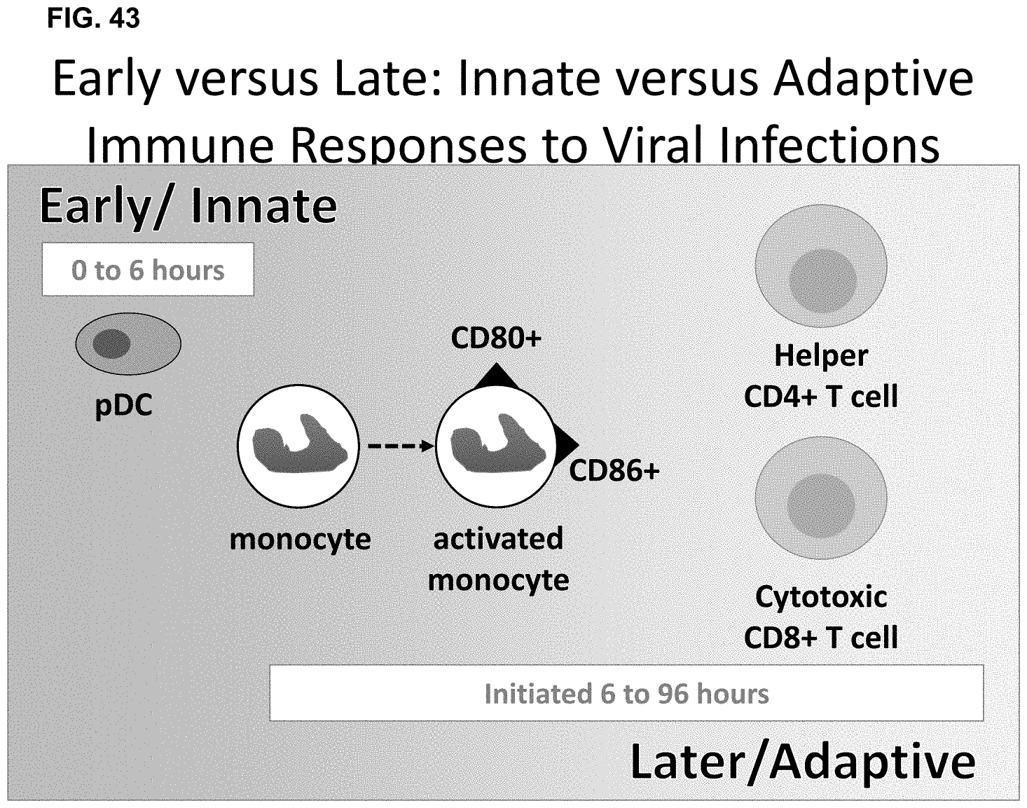

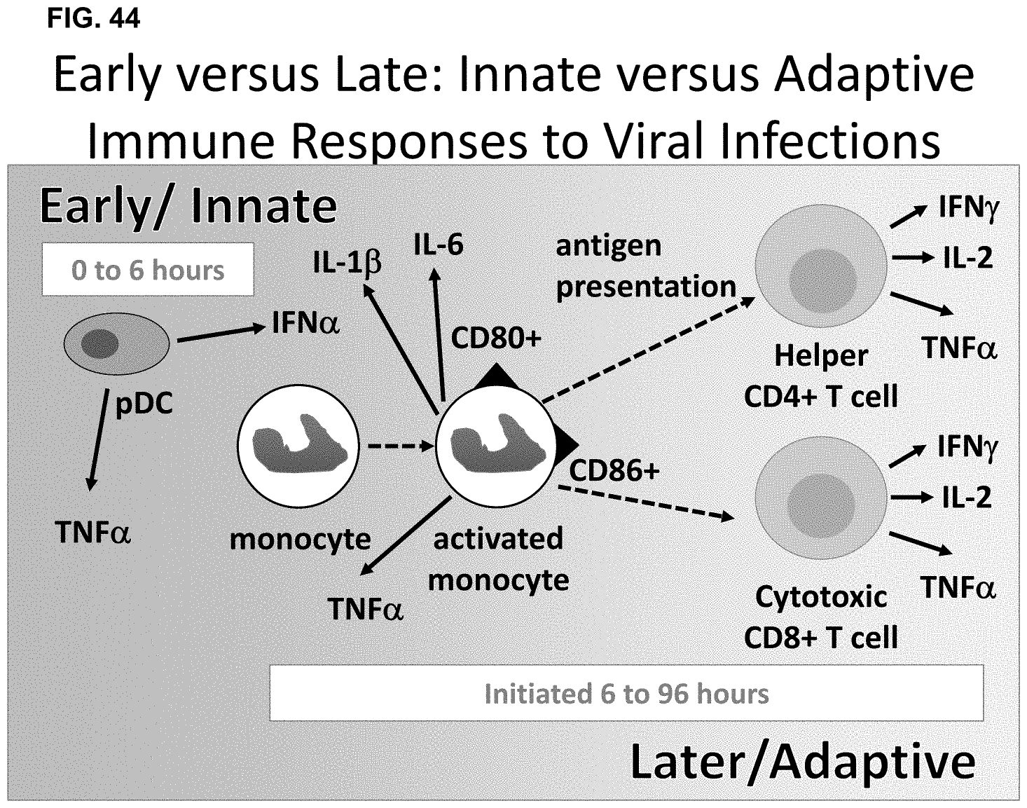

FIG. 42 shows boxes around particular mixtures pointing to the sensitivity of changes to the components or relative amounts of particular components. Using the graph of the effects of the cannabinoid containing complex mixtures on levels of interferon gamma (IFNg) in CD8+ T cells as an example, note that CCCM mixture 6 containing 5 mM of CBN and 5 mM CBD produced a statistically significant reduction in IFNg in the CD4+ T cells. However, in CCCM mixture 26, 0.001 mM Phytol is added to the 5 mM of CBN and 5 mM CBD (as in CCCM mixture 6), CCCM mixture 26 does not produced a statistically significant reduction in IFNg in the CD8+ T cells. When 0.01 mM THC is added to the same components that were in CCCM mixture 26 (that did not cause a statistically significant reduction), the resultant CCCM mixture 51, which contains 5 mM of CBN, 5 mM CBD, 0.01 mM THC, 0.001 mM Phytol A, it still did not produce a statistically significant reduction in IFNg in the CD8+ T cells. If the 0.001 mM Phytol from CCCM mixture 51 were removed, as in CCCM mixture 66, which contains 5 mM of CBN, 5 mM CBD, and 0.01 mM THC, CCCM mixture 66 does produce a statistically significant reduction in IFNg in the CD8+ T cells.

Taken together, these examples illustrate that the compositions and relative amounts of cannabinoids and terpenes in the mixtures had to be carefully deduced from rigorous experimentation on both individual ingredients and the mixtures as included in this application.

### 6. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 7. EQUIVALENTS

The present disclosure provides, inter alia, compositions of cannabinoid and/or terpene-containing complex mixtures. The present disclosure also provides method of treating neurodegenerative diseases by administering the cannabinoid and/or terpene-containing complex mixtures. While various specific embodiments have been illustrated and described, the above specification is not restrictive. It will be appreciated that various changes can be made without departing from the spirit and scope of the invention(s). Many variations will become apparent to those skilled in the art upon review of this specification.

1. An active pharmaceutical ingredient, comprising:
(a) a T-cell-modulating cannabinoid or terpene; and
(b) a monocyte-modulating cannabinoid or terpene
wherein the T-cell modulating cannabinoid or terpene is a CD4⁺ T-cell modulating cannabinoid or terpene, a CD8⁺ T-cell modulating cannabinoid or terpene, or a CD4⁺ and CD8⁺ T-cell modulating cannabinoid or terpene.

2. (canceled)

3. The active pharmaceutical ingredient of claim 1, wherein the T-cell modulating cannabinoid or terpene comprises a single cannabinoid or terpene.

4. (canceled)

5. The active pharmaceutical ingredient of claim 1, wherein the monocyte-modulating cannabinoid or terpene comprises a single cannabinoid or terpene.

6. (canceled)

7. (canceled)

8. The active pharmaceutical ingredient of claim 1, wherein the T-cell modulating cannabinoid or terpene is selected from one or more of: cannabidiol (CBD), Cannabidivarin (CBDV), Cannabinol (CBN), phytol A, and tetrahydrocannabinol (THC), the monocyte modulating cannabinoid or terpene is selected from one or more of cannabidiol (CBD), Cannabidivarin (CBDV), Cannabinol (CBN), Phytol A, and tetrahydrocannabinol (THC), or both.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A unit dosage form comprising the active pharmaceutical ingredient of claim 1, wherein the T-cell modulating cannabinoid or terpene is present in an amount sufficient to achieve a mean peak concentration (Cₘₐₓ) in plasma or target tissue of at least 0.001 μM, when administered, wherein the monocyte-modulating cannabinoid or terpene is present in an amount sufficient to achieve a mean peak concentration (Cmax) in plasma and/or target tissue of at least 0.001 μM, when administered, or both.

24. (canceled)

25. (canceled)

26. (canceled)

27. A method of making an active pharmaceutical ingredient of claim 1, comprising steps, in any order, of mixing:
at least one T-cell modulating cannabinoid or terpene; and
at least one monocyte-cell modulating cannabinoid or terpene,
wherein each of the T-cell modulating cannabinoid or terpene and monocyte-modulating cannabinoid or terpene is synthetic or biosynthetic.

28. (canceled)

29. A pharmaceutical composition comprising the active ingredient of claim 1 and a pharmaceutically acceptable carrier or diluent, wherein the composition comprises an oil, an emulsion, a gel, or an aerosol, wherein the composition comprises a nanoparticle or nanoemulsion encapsulating the active ingredient, and wherein the active ingredient is present in the pharmaceutical composition at a concentration of at least 0.01 mg/ml.

30. (canceled)

31. (canceled)

32. (canceled)

33. A method of treating a patient who has, or who is at risk for developing, cytokine release syndrome (CRS) and/or Macrophage Activation Syndrome (MAS), the method comprising:
administering an effective amount of the active pharmaceutical ingredient of claim 1 to the patient who has, or who is at risk for developing, CRS or MAS.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. An active pharmaceutical ingredient, comprising:
two or more T-cell-modulating cannabinoids, wherein the two or more T-cell-decreasing cannabinoids is a CD4⁺ T-cell decreasing cannabinoid or terpene, a CD8⁺ T-cell reducing cannabinoid or terpene, or a CD4⁺ and CD8⁺ T-cell modulating cannabinoid or terpene.

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A unit dosage form comprising the active pharmaceutical ingredient claim 74, wherein at least one of the two or more T-cell modulating cannabinoids are present in an amount sufficient to achieve a mean peak concentration (Cₘₐₓ) in plasma or target tissue of at least 0.01 μM when administered or at least 1 μM when administered.

87. (canceled)

88. (canceled)

89. (canceled)

90. (canceled)

91. (canceled)

92. (canceled)

93. (canceled)

94. A method of making an active pharmaceutical ingredient of claim 74, comprising steps, in any order, of mixing:
two or more T-cell modulating cannabinoids
wherein each of the two or more T-cell modulating cannabinoids is synthetic or biosynthetic.

95. (canceled)

96. (canceled)

97. (canceled)

98. A pharmaceutical composition comprising the active ingredient of claim 74 and a pharmaceutically acceptable carrier or diluent, wherein the composition is an oil, an emulsion, a gel, or an aerosol, and wherein the composition comprises a nanoparticle or nanoemulsion encapsulating the active ingredient, and wherein the active ingredient is present in the pharmaceutical composition at a concentration at least 0.01 mg/ml.

99. (canceled)

100. (canceled)

101. (canceled)

102. (canceled)

103. (canceled)

104. (canceled)

105. (canceled)

106. (canceled)

107. A method of treating a patient who has, or who is at risk for developing, cytokine release syndrome (CRS) and/or Macrophage Activation Syndrome (MAS), the method comprising:
administering an effective amount of the active ingredient of claim 74 to the patient who has, or who is at risk for developing, CRS or MAS.

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. An active pharmaceutical ingredient comprising:
(a) a plasmacytoid dendritic cell-(pDC) modulating cannabinoid or terpene;
(b) a monocyte-modulating cannabinoid or terpene; and
(c) a T-cell-modulating cannabinoid or terpene,
wherein the T-cell modulating cannabinoid or terpene is a CD4+ T-cell modulating cannabinoid or terpene, a CD8+ T-cell modulating cannabinoid or terpene, or a CD4+ and CD8+ T-cell modulating cannabinoid or terpene.

137. The active pharmaceutical ingredient of claim 136, wherein the pDC modulating cannabinoid or terpene comprises a single cannabinoid or terpene.

138. (canceled)

139. The active pharmaceutical ingredient of claim 136, wherein the monocyte-modulating cannabinoid or terpene comprises a single cannabinoid or terpene.

140. (canceled)

141. (canceled)

142. The active pharmaceutical ingredient of claim 136, wherein the T-cell modulating cannabinoid or terpene comprises a single cannabinoid or terpene.

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. A unit dosage form comprising the active pharmaceutical ingredient of claim 136, wherein the pDC modulating cannabinoid or terpene is present in an amount sufficient to achieve a mean peak concentration (Cₘₐₓ) in plasma or target tissue of at least 0.001 μM, when administered; the monocyte-modulating cannabinoid or terpene is present in an amount sufficient to achieve a mean peak concentration (Cₘₐₓ) in plasma or target tissue of at least 0.001 μM, when administered; the T-cell modulating cannabinoid or terpene is present in an amount sufficient to achieve a mean peak concentration (Cₘₐₓ) in plasma or target tissue of at least 0.001 μM, when administered, or any combination thereof.

165. (canceled)

166. (canceled)

167. (canceled)

168. (canceled)

169. (canceled)

170. A method of making an active pharmaceutical ingredient, comprising steps, in any order, of mixing:
at least one pDC modulating cannabinoid or terpene;
at least one monocyte-cell modulating cannabinoid or terpene; and
at least one T-cell modulating cannabinoid or terpene
wherein each of the pDC modulating cannabinoid or terpene, T-cell modulating cannabinoid or terpene, and monocyte-modulating cannabinoid or terpene is synthetic or biosynthetic.

171. (canceled)

172. (canceled)

173. (canceled)

174. (canceled)

175. A pharmaceutical composition comprising the active ingredient of claim 136 and a pharmaceutically acceptable carrier or diluent, wherein the composition is an oil, an emulsion, a gel, or an aerosol, wherein the composition comprises a nanoparticle or nanoemulsion encapsulating the active ingredient, and wherein the active ingredient is present in the pharmaceutical composition at a concentration of at least 0.01 mg/ml.

176. (canceled)

177. (canceled)

178. (canceled)

179. (canceled)

180. (canceled)

181. (canceled)

182. (canceled)

183. (canceled)

184. A method of modulating an immune response, the method comprising:
administering an effective amount of the active pharmaceutical ingredient of claim 136 to the patient who requires modulation of an immune response.

185. (canceled)

186. (canceled)

187. (canceled)

188. (canceled)

189. (canceled)

190. (canceled)

191. (canceled)

192. (canceled)

193. (canceled)

194. (canceled)

195. (canceled)

196. (canceled)

197. (canceled)

198. (canceled)

199. (canceled)

200. (canceled)

201. (canceled)

202. (canceled)

203. (canceled)

204. (canceled)

205. (canceled)

206. (canceled)

207. (canceled)

208. (canceled)

209. (canceled)

210. (canceled)

211. (canceled)

212. (canceled)

213. (canceled)

214. (canceled)

215. (canceled)

216. An active pharmaceutical ingredient comprising:
(a) a plasmacytoid dendritic cell-(pDC) modulating cannabinoid or terpene; and
(b) optionally a T-cell-modulating cannabinoid or terpene.

217. The active pharmaceutical ingredient of claim 216, wherein the pDC modulating cannabinoid or terpene comprises a single cannabinoid or terpene.

218. (canceled)

219. The active pharmaceutical ingredient of claim 216, wherein the T-cell modulating cannabinoid or terpene is a CD4⁺ T-cell modulating cannabinoid or terpene, a CD8⁺ T-cell modulating cannabinoid or terpene, or a CD4⁺ and CD8⁺ T-cell modulating cannabinoid or terpene.

220. The active pharmaceutical ingredient of claim 216, wherein the T-cell modulating cannabinoid or terpene comprises a single cannabinoid or terpene.

221. (canceled)

222. (canceled)

223. (canceled)

224. (canceled)

225. (canceled)

226. (canceled)

227. (canceled)

228. (canceled)

229. (canceled)

230. (canceled)

231. (canceled)

232. (canceled)

233. (canceled)

234. (canceled)

235. (canceled)

236. (canceled)

237. (canceled)

238. (canceled)

239. (canceled)

240. (canceled)

241. A unit dosage form comprising the active pharmaceutical ingredient of claim 216, wherein the pDC modulating cannabinoid or terpene is present in an amount sufficient to achieve a mean peak concentration (Cₘₐₓ) in plasma or target tissue of at least 0.001 μM, when administered, wherein the optional T-cell modulating cannabinoid or terpene is present in an amount sufficient to achieve a mean peak concentration0 (Cₘₐₓ) in plasma or target tissue of at least 0.001 μM, when administered.

242. (canceled)

243. (canceled)

244. (canceled)

245. (canceled)

246. A method of making an active pharmaceutical ingredient of claim 216, comprising steps, in any order, of mixing:
at least one pDC modulating cannabinoid or terpene; and
optionally at least one T-cell modulating cannabinoid or terpene,
wherein each of the pDC modulating cannabinoid or terpene, and optional T-cell modulating cannabinoid or terpene is synthetic or biosynthetic.

247. (canceled)

248. (canceled)

249. (canceled)

250. A pharmaceutical composition comprising the active ingredient of claim 216 and a pharmaceutically acceptable carrier or diluent, wherein the composition is an oil, an emulsion, a gel, or an aerosol, wherein the composition comprises a nanoparticle or nanoemulsion encapsulating the active ingredient, and wherein the active ingredient is present in the pharmaceutical composition at a concentration of at least 0.01 mg/ml.

251. (canceled)

252. (canceled)

253. (canceled)

254. (canceled)

255. (canceled)

256. (canceled)

257. (canceled)

258. (canceled)

259. A method of suppressing an immune response in a patient, the method comprising:
administering an effective amount of the active pharmaceutical ingredient of claim 216 to the patient who has inflammation.

260. (canceled)

261. (canceled)

262. (canceled)

263. (canceled)

264. (canceled)

265. (canceled)

266. (canceled)

267. (canceled)

268. (canceled)

269. (canceled)

270. (canceled)

271. (canceled)

272. (canceled)

273. (canceled)

274. (canceled)

275. (canceled)

276. (canceled)

277. (canceled)

278. (canceled)

279. (canceled)

280. (canceled)

281. (canceled)

282. (canceled)

283. (canceled)

284. (canceled)

285. An active pharmaceutical ingredient comprising two or more monocyte-modulating cannabinoids, wherein the active pharmaceutical ingredient comprises two or more of cannabidiol (CBD), Cannabidivarin (CBDV), Cannabinol (CBN), Cannabigerol (CBG), phytol A, and tetrahydrocannabinol (THC).

286. (canceled)

287. (canceled)

288. (canceled)

289. (canceled)

290. (canceled)

291. A unit dosage form comprising the active pharmaceutical ingredient of claim 285, wherein at least one of the two or more monocyte modulating cannabinoids are present in an amount sufficient to achieve a mean peak concentration (Cₘₐₓ) in plasma and/or target tissue of at least 0.001 μM, when administered.

292. (canceled)

293. A method of making an active pharmaceutical ingredient of claim 285, comprising steps, in any order, of mixing:
two or more monocyte-modulating cannabinoids,
wherein each of the two or more monocyte-modulating cannabinoids is synthetic or biosynthetic.

294. A pharmaceutical composition comprising the active ingredient of claim 285 and a pharmaceutically acceptable carrier or diluent, wherein the composition is an oil, an emulsion, a gel, or an aerosol, wherein the composition comprises a nanoparticle or nanoemulsion encapsulating the active ingredient, and wherein the active ingredient is present in the pharmaceutical composition at a concentration of at least 0.01 mg/ml.

295. (canceled)

296. (canceled)

297. (canceled)

298. (canceled)

299. (canceled)

300. (canceled)

301. (canceled)

302. (canceled)

303. A method of inducing a proinflammatory response in a patient the method comprising:
administering an effective amount of the active pharmaceutical ingredient of claim 285 to the patient who has a localized infection, wherein the effective amount of the active pharmaceutical ingredient reduces the level of TNFα secreted by CD14⁺CD16⁺ monocytes.

304. (canceled)
